SEQUENCE LISTING (32 sequences)

SEQ ID NO: 1 - protein, 168 aa, artificial
  W83 Kpg K1
GTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFIDLDEVEI

SEQ ID NO: 2 - protein, 169 aa, artificial
  381 Kpg K1
TTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPEAIRGRIQGTWRQKTVDLPAGTKYVAFRHFQSTDMFYIDLDEVEI

SEQ ID NO: 3 - protein, 169 aa, artificial; identical to SEQ ID NO: 2
  HG66 Kpg K1

SEQ ID NO: 4 - protein, 169 aa, artificial; identical to SEQ ID NO: 2
  HG66 Rpg R1

SEQ ID NO: 5 - protein, 173 aa, artificial
  381 Hag A2
TTTLSESFENGIPASWKTIDADGDGNNWTTTPPPGGTSFAGHNSAICASSASYINFEGPQNPDNYLVTPELSLPNGGTLTFWVCAQDANYASEHYAVYASSTGNDASNFANALLEEVLTAKTVVTAPEAIRGTRVQGTWYQKTVQLPAGTKYVAFRHFGCTDFFWINLDDVEI

SEQ ID NO: 6 - protein, 173 aa, artificial; identical to SEQ ID NO: 5
  381 Hag A4

SEQ ID NO: 7 - protein, 173 aa, artificial; identical to SEQ ID NO: 5
  381 Hag A6

SEQ ID NO: 8 - protein, 173 aa, artificial
  W83 Hag A1-A4
TTTLSESFENGIPASWKTIDADGDGNNWTTTPPPGGTSFAGHNSAICVSSASYINFEGPQNPDNYLVTPELSLPNGGTLTFWVCAQDANYASEHYAVYASSTGNDASNFANALLEEVLTAKTVVTAPEAIRGTRVQGTWYQKTVQLPAGTKYVAFRHFGCTDFFWINLDDVEI

SEQ ID NO: 9 - protein, 173 aa, artificial; identical to SEQ ID NO: 8
  W83 Hag A1-A6

SEQ ID NO: 10 - protein, 173 aa, artificial
  W83v Kpg K1
TTTLSESFENGIPASWKTIDADGDGNNWTTTPPPGGSSFAGHNSAICVSSASYINFEGPQNPDNYLVTPELSLPNGGTLTFWVCAQDANYASEHYAVYASSTGNDASNFADALLEEVLTAKTVVTAPEAIRGTRVQGTWYQKTVQLPAGTKYVAFRHFGCTDFFWINLDDVEI

SEQ ID NO: 11 - protein, 173 aa, artificial
  W83 Hag A1-A2
TTTLSESFENGIPASWKTIDADGDGNNWTTTPPPGGSSFAGHNSAICVSSASYINFEGPQNPDNYLVTPELSLPNGGTLTFWVCAQDANYASEHYAVYASSTGNDASNFANALLEEVLTAKTVVTAPEAIRGTRVQGTWYQKTVQLPAGTKYVAFRHFGCTDFFWINLDDVEI

SEQ ID NO: 12 - protein, 173 aa, artificial
  381 Hag A2-A8
TTTLSESFENGIPASWKTIDADGDGNNWTTTPPPGGTSFAGHNSAICVSSASYINFEGPQNPDNYLVTPELSLPGGGTLTFWVCAQDANYASEHYAVYASSTGNDASNFANALLEEVLTAKTVVTAPEAIRGTRVQGTWYQKTVQLPAGTKYVAFRHFGCTDFFWINLDEVEI

SEQ ID NO: 13 - protein, 173 aa, artificial
  W83 Kpg K3
AEVLNEDFENGIPASWKTIDAHGDGNNWTTTPPPGGSSFAGHNSAICVSSASHINFEGPQNPDNYLVTPELSLPGGGTLTFWVCAQDANYASEHYAVYASSTGNDASNFANALLEEVLTAKTVVTAPEAIRGTRAQGTWYQKTVQLPAGTKYVAFRHFGCTDFFWINLDDVVI

SEQ ID NO: 14 - protein, 173 aa, artificial
  W83v Kpg K3
AEVLNEDFENGIPASWKTIDADGDGNNWTTTPPPGGSSFAGHNSAICVSSASYINFEGPQNPDNYLVTPELSLPGGGTLTFWVCAQDANYASEHYAVYASSTGNDASNFANALLEEVLTAKTVVTAPEAIRGTRAQGTWYQKTVQLPAGTKYVAFRHFGCTDFFWINLDDVVI

SEQ ID NO: 15 - protein, 173 aa, artificial
  W83 Hag A1-A8
AEVLNEDFENGIPASWKTIDADGDGNNWTTTPPPGGSSFAGHNSAICVSSASYINFEGPQNPDNYLVTPELSLPGGGTLTFWVCAQDANYASEHYAVYASSTGNDASNFANALLEEVLTAKTVVTAPEAIRGTRVQGTWYQKTVQLPAGTKYVAFRHFGCTDFFWINLDDVVI

SEQ ID NO: 16 - protein, 173 aa, artificial
  381 Hag A2-A10
AEVLNEDFENGIPSSWKTIDADGDGNNWTTTPPPGGSSFAGHNSAICVSSASYINFEGPQNPDNYLVTPELSLPGGGTLTFWVCAQDANYASEHYAVYASSTGNDASNFANALLEEVLTAKTVVTAPEAIRGTRVQGTWYQKTVQLPAGTKYVAFRHFGCTDFFWINLDDVVI

SEQ ID NO: 17 - protein, 177 aa, artificial
  W83 Hag A1-A1
TYVLNESFDTQTLPNGWTMIDADGDGHNWLSTINVYNTATHTGDGAMFSKSWTASSGAKIDLSPDNYLVTPKFTVPENGKLSYWVSSQEPWTNEHYGVFLSTTGNEAANFTIKLLEETGSGKPAPMNLVKSEGVKAPAPYQERTIDLSAYAGQQVYLAFRHFGCTGIFRLYLDDVAV

SEQ ID NO: 18 - protein, 177 aa, artificial
  381 Hag A2-A1
TYVLNESFDTQTLPNGWTMIDADGDGHNWLSTINVYNTATHTGDGAMFSKSWTASGGAKIDLSPDNYLVTPKVTVPENGKLSYWVSSQVPWTNEHYGVFLSTTGNEAANFTIKLLEETLGSDKPAPMNLVKSEGVKLPAPYQERTIDLSAYAGQQVYLAFRHFNSTGIFRLYLDDVA

SEQ ID NO: 19 - protein, 172 aa, artificial
  W83v Kpg K2
FTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQF

SEQ ID NO: 20 - protein, 172 aa, artificial; identical to SEQ ID NO: 19
  HG66 Rpg R2

SEQ ID NO: 21 - protein, 172 aa, artificial; identical to SEQ ID NO: 19
  W83 Hag A1-A5

SEQ ID NO: 22 - protein, 172 aa, artificial
  381 Kpg K2
FTETFESSTHGEAPAEWTTIDADGDGQDWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQF

SEQ ID NO: 23 - protein, 172 aa, artificial
  HG66 Kpg K2
FTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEADGAKPQSVWIERTVDLPAGTKYVAFRHYNCSDLNYILLDDIQF

SEQ ID NO: 24 - protein, 172 aa, artificial
  W83 Hag A1-A3
FTETFESSTHGEAPAEWTTIDADGDGQGWLCLSSGQLGWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQF

SEQ ID NO: 25 - protein, 172 aa, artificial
  W83 Kpg K2
FTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQF

SEQ ID NO: 26 - protein, 172 aa, artificial; identical to SEQ ID NO: 23
  381 Hag A2-A3

SEQ ID NO: 27 - protein, 172 aa, artificial; identical to SEQ ID NO: 23
  381 Hag A2-A9

SEQ ID NO: 28 - protein, 172 aa, artificial
  381 Hag A2-A5
FTETFESSTHGEAPAEWTTIDADGDGQGWLCLSSGQLGWLTAHGGTNVVASFSWNGMALNPDNYLISKDVTGATKVKYYYAVNDGFPGDHYAVMISKTGTNAGDFTVVFEETPNGINKGGARFGLSTEADGAKPQSVWIERTVDLPAGTKYVAFRHYNCSDLNYILLDDIQF

SEQ ID NO: 29 - protein, 172 aa, artificial; identical to SEQ ID NO: 28
  381 Hag A2-A7

SEQ ID NO: 30 - protein, 172 aa, artificial; identical to SEQ ID NO: 24
  W83 Hag A1-A7

SEQ ID NO: 31 - protein, 166 aa, artificial
  381 Kpg K3
GELLNEDFEGDAIPTGWTALDADGDGNNWDITLNEFTRGERHVLSPLRASNVAISYSSLLQGQEYLPLTPNNFLITPKVEGAKKITYKVGSPGLPQWSHDHYALCISKSGTAAADFEVIFEETMTYTQGGANLTREKDLPAGTKYVAFRHYNCTDVLGIMIDDVVI

SEQ ID NO: 32 - protein, 15 aa, artificial
  Amino acid synthetic K2
ETFESSTHGEAPAEC

(19) United States
(12) Patent Application Publication
(10) Pub. No.: US 2013/0189280 A1
(43) Pub. Date: Jul. 25, 2013

(54) PROTEIN DOMAINS AND USES THEREFOR

(71) Applicants: Charles Collyer, Glebe (AU); Neil Hunter, Pennant Hills (AU)

(72) Inventors: Charles Collyer, Glebe (AU); Neil Hunter, Pennant Hills (AU)

(73) Assignees: SYDNEY WEST AREA HEALTH SERVICE (SWAHS), Kingswood, New South Wales (AU); THE UNIVERSITY OF SYDNEY, Sydney, New South Wales (AU)

(21) Appl. No.: 13/578,770

(22) PCT Filed: Feb. 11, 2011
(86) PCT No.: PCT/AU2011/000153

§ 371 Date: Nov. 29, 2012

(30) Foreign Application Priority Data
Feb. 12, 2010 (AU) 2010900571
Feb. 23, 2010 (AU) 2010900887

(51) Int. Cl. C07K 14/195

(52) U.S. Cl. CPC C07K 14/195
USPC 424/164.1; 530/389.5; 703/12

(57) ABSTRACT

The present disclosure relates generally to a structure-modeling approach to identify therapeutic and diagnostic targets on proteins. Means are provided to generate agents which bind and optionally antagonize a particular domain within a protein referred to as a Cleaved_Adhesin Family Domain. In an embodiment, the disclosure is directed to the control of Porphyromonas gingivalis infection or infection by related microorganisms by targeting selected domains on protease-like molecules having a hemagglutinin region. In another embodiment, the present invention enables the modulation or detection of a protein having a Cleaved_Adhesin domain homologous to those in the protease-like molecules.

### FILING DATA

This application and any patent granted thereon is associated with and claims priority from Australian Provisional Patent Application No. 2010900571, filed on 12 Feb. 2010, entitled “Protein domains and uses therefor” and Australian Provisional Patent Application No. 2010900887, filed on 23 Feb. 2010, entitled “Protein domains and uses therefor-II”, the entire contents of which, are incorporated herein by reference.

### FIELD

The present disclosure relates generally to a structure-modeling approach to identify therapeutic and diagnostic targets on proteins. Means are provided to generate agents which bind and optionally antagonize a particular domain within a protein referred to as a Cleaved_Adhesin Family Domain. In an embodiment, the disclosure is directed to the control of Porphyromonas gingivalis infection or infection by related microorganisms by targeting selected domains on protease-like molecules having a hemagglutinin region. In another embodiment, the present disclosure enables the modulation or detection of a protein having a Cleaved_Adhesin domain homologous to those in the protease-like molecules.

### BACKGROUND

Bibliographic details of the publications referred to by author in this specification are collected alphabetically at the end of the description.

Reference to any prior art in this specification is not, and should not be taken as, an acknowledgment or any form of suggestion that this prior art forms part of the common general knowledge in any country.

Porphyromonas gingivalis is a Gram-negative anaerobic bacterium implicated as a key pathogen in chronic periodontitis, a destructive inflammatory disease of the tissues supporting the dentition (Holt et al., Science 239:55-57, 1988, Socransky et al., J Clin Periodontol 25:134-144, 1998). Porphyromonas gingivalis is deficient in critical gene products necessary for the synthesis of the porphyrin macrocycle of heme (Roper et al., J Biol Chem 275:40316-40323 2000). As a porphyrin auxotroph, the organism must acquire this nutrient from host sources, most apparently as heme, with erythrocytes providing the major potential source. Accordingly, P. gingivalis has mechanisms for attachment and agglutination of erythrocytes, lysis of erythrocytes, capture and degradation of released hemoglobin and subsequent sequestration of heme (Lamont and Jenkinson, Microbiol Mol Biol Rev 62:1244-1263, 1998).

The virulence mechanisms of this pathogen are not fully understood but a group of cysteine proteases, the gingipains, are known to play important roles in hemagglutination, hemolysis and subsequent capture of essential heme (Pike et al., J Bacteriol 178:2876-2882, 1996, Lewis et al., J Bacteriol 181:4905-4913, 1999, Paramaesvaran et al., J Bacteriol 185:2528-2537, 2003). The lysine-specific cysteine protease (Kgp) and arginine-specific proteases A and B (RgpA and RgpB) are gingipains located on the surface of P. gingivalis or in some strains are released (Potempa et al., Infect Immun 63:1176-1182, 1995). Kgp and RgpA are encoded by single loci, kgp and rgpA, respectively, with the encoded proteins consisting of both a catalytic domain and hemagglutinin/adhesin (HA) domains (Pavloff et al., J Biol Chem 272:1595-1600, 1997, Pavloff et al., J Biol Chem 270:1007-1010, 1995, Curtis et al., J Periodontal Res 34:464-472, 1999). However, when extracted from P. gingivalis they are observed to be proteolytically processed while remaining bound in tight molecular complexes. These observations have been widely interpreted to indicate a precise physiological processing of surface gingipains. However, if prior to the extraction from the cell surface, the proteolytic activities of the gingipains are specifically inhibited, then the processing of the extracted products is incomplete. This is observed when using a monoclonal antibody to an adhesin domain epitope common to both RgpA and Kgp which detects a range of higher molecular weight fragments in extracts from pre-inhibited cells (Shi et al., J Biol Chem 274:17955-17960, 1999). This is interpreted to indicate that processing of expressed gingipain is either a continuous process during growth of the organism or that at least part of the autolytic/proteolytic processing results from the extraction process.

Of the four putative HA domains isolated from processed high molecular weight gingipains by SDS-PAGE, Rgp44/Kgp39 has been found to possess hemagglutination activity (Pike et al., J Biol Chem 269:406-411, 1994) while Kgp15/Rgp15 has been proposed to be a hemoglobin (Nakayama et al., Mol Microbiol 27:51-61, 1998) and heme binding receptor (DeCarlo et al., J Bacteriol 181:3784-3791, 1999). Rgp and Kgp have been reported to have hemoglobinase activity and the catalytic activity of Kgp is critical in this function (Shi et al., 1999 supra; Lewis et al., 1999 supra). Mutants defective in Kgp have markedly reduced capacity to sequester heme and are relatively avirulent in animal models (Shi et al., 1999 supra; Lewis et al., 1999 supra; Lewis and Macrina, Infect and Immun 66:4905-4913, 1998). Hemolytic activity of P. gingivalis has been attributed to protease action based on inhibitor profiles (Chu et al., Infect Immun 59:1932-1940, 1991). Kgp deletion mutants have approximately 50% of the hemolytic activity of mutants complemented for Kgp indicating a major contribution by this proteinase (Lewis et al., 1999 supra). The exact role of the Kgp-HA domains remains to be determined.

For the gingipains, only the crystal structure of RgpB, which contains a heavily truncated HA domain, has been determined (Eichinger et al., Embo J 18:5453-5462, 1999) and to date, the proposed structures of the HA domains have been mainly speculative. The widely accepted domain structural model of RgpA and Kgp (FIG. 1a) is based on the sequence analysis of the extracted gingipains from the outer membrane of P. gingivalis by SDS-PAGE, peptide mass fingerprinting and N-terminal sequencing (Pavloff et al., 1995 supra, Potempa et al., J Biol Chem 273:21648-21657, 1998, Veith et al., Biochem J 363:105-115, 2002). However, this domain structure model does not explain the function of these cysteine proteases/adhesive molecules.

There is a need to more accurately define functional domains within particular proteins in order to design highly specific interacting molecules such as for use as antagonists, agonists or diagnostic agents.

### SUMMARY

Porphyromonas gingivalis is an obligately anaerobic bacterium recognized as an etiologic agent of adult periodontis in mammals, such as humans. This microorganism produces a range of protease-like molecules including gingipains (gp) and hemagglutinin (HA) proteins (Hag proteins) such as hemagglutininA (HagA) which are involved in hemolysis of erythrocytes and heme acquisition. Porphyromonas gingivalis is a porphyrin auxotroph, requiring this molecule to grow and persist in a host. The HA region of these protease-like molecules provides a potential therapeutic target to inhibit P. gingivalis from capturing heme and, therefore, to inhibit its growth. However, the previously suggested domains have not adequately explained function and, hence, have likely not been correctly determined.

In accordance with the present disclosure, a structure-modeling approach is used to identify particular domains on protease-like molecules produced by P. gingivalis. In an embodiment, the HA region of the protease-like molecules have been subject to domain modeling based on homology to Cleaved_Adhesin Domain Family proteins (see the Cleaved_Adhesin Family PF07675 in the PFam protein families database [Finn et al., Nucleic Acids, Res 36:D281-288, 2008]). The identified domains within the HA region are referred to herein as “Cleaved_Adhesin domains”. On the lysine gingipain (Kgp) from P. gingivalis, the domains are specifically designated K1, K2 and K3. The crystal structures of K2 and K3 domains from the W83 strain of P. gingivalis have further been determined together with a model for K1. The present disclosure extends, however, to homologs or functionally or structurally equivalent domains on the arginine gingipain (Rgp), and particularly R1 and R2, and on HagA (A1 through A10) [see FIG. 1 and sequences]. Cleaved_Adhesin domains are used as therapeutic targets to identify or generate antagonists or vaccines to specifically inhibit the functions of these molecules. These domains also provide diagnostic targets. This also enables identification of homologous Cleaved_Adhesin domains in a range of other proteins independent of whether the protein comprises an HA region or whether the protein has protease activity. Such domains also be used as therapeutic and diagnostic targets to generate Cleaved_Adhesin domain-interacting molecules. Such molecules include antagonists, agonists and diagnostic agents.

Accordingly, the instant disclosure contemplates a method for the prophylaxis or treatment of infection by a microorganism in a biological environment from where the microorganism acquires iron, heme or porphyrin, the method comprising administering to the environment an effective amount of an agent for a time and under conditions sufficient to antagonize a Cleaved_Adhesin domain within the adhesin and/or carbohydrate binding region of a protease-like molecule produced by the microorganism, the domain associated with hemolysis or hemolytic activity of erythrocytes.

A method is also provided for the prophylaxis or treatment of infection by a microorganism in a mammal from where the microorganism acquires iron, heme or porphyrin, the method comprising administering to the environment an effective amount of an agent for a time and under conditions sufficient to antagonize a Cleaved_Adhesin domain within an HA region of a molecule produced by the microorganism wherein the molecule is a protease-like molecule associated with hemolysis or hemolytic activity of erythrocytes.

Also contemplated is a method for the prophylaxis or treatment of infection by Porphyromonas gingivalis or a related organism in a mammal, the method comprising administering to the mammal an effective amount of an agent for a time and under conditions sufficient to antagonize a Cleaved_Adhesin domain within the HA region of a gingipain or HagA, wherein the antagonism prevents or reduces hemolysis or hemolytic activity of erythrocytes.

The present disclosure further provides a method for prophylaxis or treatment of periodontal, pulmonary, vaginal, urethral or hoof disease resulting from infection by P. gingivalis or related microorganism in a mammal, the method comprising administering to the mammal an effective amount of an agent for a time and under conditions sufficient to antagonize a Cleaved_Adhesin domain within the HA region of a gingipain or HagA, wherein the antagonism prevents or reduces hemolysis or hemolytic activity of erythrocytes.

In an embodiment, a method is provided for the prophylaxis or treatment of infection by a microorganism in a biological environment from where the microorganism acquires iron, heme or porphyrin, the method comprising administering to the environment an effective amount of an agent for a time and under conditions sufficient to antagonize a Cleaved_Adhesin domain within an adhesin and/or carbohydrate binding region of a molecule produced by the microorganism, the domain associated with hemolysis or hemolytic activity of erythrocytes, wherein the domain is defined by Cleaved_Adhesin domain modeling.

Another aspect of the present disclosure provides a method for the treatment or prophylaxis of infection by Porphyromonas gingivalis or a related microorganism in a mammal, the method comprising administering to the mammal an antagonizing effective amount of an agent which antagonizes function of one or more of Cleaved_Adhesin domains K1, K2 and/or K3 on Kgp and/or R1 and/or R2 on Rgp and/or equivalents on HagA including one or more of A1 through A10.

As indicated above, the identification of the Cleaved_Adhesin domains enables identification of similar domains in a range of proteins from organisms not necessarily related to P. gingivalis or from un-related proteins.

Accordingly, a method is provided for identifying a protein or part thereof which comprises a Cleaved_Adhesin domain, the method comprising subjecting amino acid sequences of proteins to Cleaved_Adhesin domain modeling based on the amino acid sequences of one or more of K1, K2, K3, R1, R2 and/or A1 through A10 and selecting amino acid sequences having homology thereto wherein such identified amino acid sequences are regarded as defining a Cleaved_Adhesin domain.

The present disclosure enables the identification of potential modulators of proteins having a Cleaved_Adhesin domain homologous to or comprising a domain selected from K1, K2, K3, R1, R2 and one or more of A1 through A10. In relation to a modulator of Kgp, Rgp or HagA, the modulator includes an antagonist or is a binding protein useful as a diagnostic agent. For other proteins, the modulators in the form of antagonists, agonists and diagnostic agents may be useful. By using the atomic coordinates of K2 or K3 and the model for K1, to identify potential modulators from a larger group, it is possible to reduce the total number of molecules which need to be tested.

The modulators may be identified by a range of means including docking a three dimensional representation of a potential modulator with the three dimensional structure of K2 and/or K3. The computer representation of K2 and K3 is defined by atomic structural coordinates. In an embodiment, one or more modulators are docked into the Cleaved_Adhesin domain structure of K2 and/or K3. The method includes: (a) providing a three dimensional representation of the atomic coordinates of a Cleaved_Adhesin domain comprising or homologous to one or more of. K2 and K3 of Kgp and docking a three dimensional representation of a compound from a computer database with the three dimensional representation of K2 and/or K3; (b) determining a conformation of the resulting complex having a favorable geometric fit and favorable complementary interactions; and (c) identifying compounds that best fit K2 and/or K3 as potential modulators of K2 and/or K3 function and/or as potential diagnostic agents of K2 and/or K3 and/or potential antagonists, agonists or diagnostic agents for protein comprising a homologous Cleaved_Adhesin domain being K1, K2, K3, R1, R2 and one or more of A1 through A10.

The present disclosure further provides an isolated protein or fragment thereof comprising a Cleaved_Adhesin domain identified by the method of subjecting amino acid sequences of proteins to Cleaved_Adhesin domain modeling based on the amino acid sequences of one or more of K1, K2, K3, R1, R2 and/or A1 through A10 and selecting amino acid sequences having homology thereto wherein such identified amino acid sequences are regarded as defining a Cleaved_Adhesin domain.

Hence, provided herein are:

(i) a target on HA-comprising molecules from Porphyromonas gingivalis wherein the target comprises a Cleaved_Adhesin domain for antagonists and diagnostic agents;

(ii) recombinant polypeptide vaccines comprising Cleaved_Adhesin domains from the Porphyromonas gingivalis HA-comprising molecules as well as homologous domains from other proteins;

(iii) antagonists, agonists and diagnostic agents designed using the atomic coordinates surrounding or defining the Cleaved_Adhesin domains of HA-comprising molecules of Porphyromonas gingivalis, K1, K2, K3, R1, R2 and/or A1 through 10.

The atomic coordinates of K2 and K3 have been deposited in the protein Data Bank under 3KM5 and 3M1H, respectively which is incorporated herein by reference. They are also shown in FIGS. 24A and B, respectively. The atomic coordinates are also incorporated herein by reference from the priority applications, Australian Provisional Patent Application No. 2010900571, filed on 12 Feb. 2010, entitled “Protein domains and uses therefor” and Australian Provisional Patent Application No. 2010900887, filed on 23 Feb. 2010, entitled “Protein domains and uses therefor-II”. A model for K1 is also presented herein and in the priority applications. The K1 homology model is based on K3 crystal structure referred to at 1.6 Angstrom. The present disclosure uses these in the design of conformer mimetics, antagonists, agonists, recombinant peptide mimics and diagnostic agents. Part of this disclosure is provided in Li et al., Mol. Microbiol. 76:861-873, 2010 published after the priority dates and the entire contents of which are incorporated by reference.

Nucleotide and amino acid sequences are referred to by a sequence identifier number (SEQ IN NO:). The SEQ ID NOs: correspond numerically to the sequence identifiers <400>1 (SEQ ID NO:1), <400>2 (SEQ ID NO:2), etc.

Table 1 provides a summary of the sequences identified for the Cleaved_Adhesin domains in Kpg K1 from strains W83, 381 and HG66, the Rpg R1 domain from strain HG66 and the Hag A domains A1-A10 from strains 281 and W83 of P. gingivalis. W83v refers to a variant strain of P. gingivalis.

TABLE 1
Summary of sequence identifiers for Cleaved_Adhesin domains
from Kpg, Rpg and HagA from Porphyromonas gingivalis

| | |
|---|---|
| 1 | W83 Kpg K1 |
| 2 | 381 Kpg K1 |
| 3 | HG66 Kpg K1 |
| 4 | HG66 Rpg R1 |
| 5 | 381 Hag A2 |
| 6 | 381 Hag A4 |
| 7 | 381 Hag A6 |
| 8 | W83 Hag A1-A4 |
| 9 | W83 Hag A1-A6 |
| 10 | W83v Kpg K1 |
| 11 | W83 Hag A1-A2 |
| 12 | 381 Hag A2-A8 |
| 13 | W83 Kpg K3 |
| 14 | W83v Kpg K3 |
| 15 | W83 Hag A1-A8 |
| 16 | 381 Hag A2-A10 |
| 17 | W83 Hag A1-A1 |
| 18 | 381 Hag A2-A1 |
| 19 | W83v Kpg K2 |
| 20 | HG66 Rpg R2 |
| 21 | W83 Hag A1-A5 |
| 22 | 381 Kpg K2 |
| 23 | HG66 Kpg K2 |
| 24 | W83 Hag A1-A3 |
| 25 | W83 Kpg K2 |
| 26 | 381 Hag A2-A3 |
| 27 | 381 Hag A2-A9 |
| 28 | 381 Hag A2-A5 |
| 29 | 381 Hag A2-A7 |
| 30 | W83 Hag A1-A7 |
| 31 | 381 Kpg K3 |
| 32 | Amino acid sequence |
| | of antigenic sequence |
| | of loop1 of K2 |

Single and three letter abbreviations are used to define amino acid residues. these are summarized in Table 2.

TABLE 2
Amino Acid Abbreviations

| | Three-letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any residue | Xaa | X |

### BRIEF DESCRIPTION OF THE FIGURES

Some figures contain color representations or entities. Color photographs are available from the Patentee upon request or from an appropriate Patent Office. A fee may be imposed if obtained from a Patent Office.

FIG. 1 is a graphical representation showing the comparison of the domain structural models for Kgp and RgpA from P. gingivalis strain W83. A. The domain model derived from observed fragmentation of extracted proteins (Veith et al., 2002 supra). B. The alternative domain model predicted by multiple sequence analysis and adapted from the Cleaved_Adhesin Domain Family PF07675: The Pfam protein families database) [Finn et al., 2008 supra]. The sequence in the region labeled K3 with a dashed box varies in the P. gingivalis strain HG66 and a homologous domain is not detected.

FIG. 2A is a graphical representation showing the conservation of amino sequences in RgpA, Kgp and the Hag proteins is shown as a number on the line linking each domain which is the percentage of sequence identity between each pair. The multiple amino acid sequence alignment including strain specific variations can be viewed at http://pfam.sanger.ac.uk/family?acc=PF07675. The observed conservation of sequence and the reported structure of K2 defines a model for the overall domain structures of these HA regions which contain multiple Cleaved_Adhesin domains. HagA1 is from P. gingivalis strain W83 and HagA2 is from strains ATCC33277 and 381.

FIG. 2B is a representation of amino acid sequence alignments of the Cleaved_Adhesin domains from Kpg, Rpg and HagA from P. gingivalis strains W83, 381 and HG66.

FIG. 3 is a photographic representation showing the analysis of K2 cleaved with Kgp. A. The boundary between regions HA2/HGP15 and RgpA17/Kgp14 in K2 is Lys 1291. A cartoon representation (green and magenta) shows how this residue lies on the surface of K2 and is readily accessible for proteolysis. If the cleaved protein remained folded three C-terminal β-strands of the smaller fragment (magenta) would link the 44 residues to HA2/HGP15. B. SDS-PAGE indicating that the purified cleaved protein contains at least two fragments. Lane 1 is the Mw standards; lane 2 is the K2 protein which migrates as a 23 kDa species on SDS-PAGE though its calculated molecular weight is 19.3 kDa; lane 3 is the fraction from size exclusion chromatography of the cleaved K2 (as described in Results and Discussion) that runs as peak similar to that of undigested K2; lane 4 is rHA2 which migrates as a 19 kDa species on SDS-PAGE though its calculated molecular weight is 15.5 kDa.

FIG. 4 a photographical representation of the crystal structure refined at 1.4 Å of the K2 domain from Kgp of P. gingivalis strain W83. A. Cartoon representation of the 13-sandwich fold showing the β-strands, the short helix in preceding 138 and the Ca²⁺ ions in red and the loops and turns. B. The surface contributed by the partial model in chain A of loop L8 in red at the ‘head end’ of the β-barrel and shown in the same orientation as drawn in A. C. The ‘jelly-roll’ topology of the Cleaved_Adhesin domain family. D. Secondary structure of K2 and aligned sequences of K1 (residues 982-1150), K2 (residues 1157-1334) and K3 (residues 1427-1599) from W83 Kgp.

FIG. 5 is a photographical representation of the Comparison of K2 and structural homologs. Superpositions of the K2 structure with four structural homologues. K2 is in green and the homologues are in grey. The Ca²⁺ ions in the K2 structure are represented as red spheres. The Ca²⁺ ions from homologues are in magenta and the Na⁺ ion in cyan. The grey spheres indicate the positions of bound polysaccharides at the carbohydrate binding sites in CBMs. Loop regions in the structural homologues known to involved in protein-protein interactions are indicated by circled dotted lines. A. MAM domain of receptor-type tyrosin-protein phosphatase (Protein Data Base [PDB] entry 2v5y, Aricescu et al., 2007 supra). B. Ephrin-binding domain of murine EphB2 receptor tyrosine kinase (PDB entry lkgy, chainA, Himanen et al., Nature 414:933-938, 2001). C. CBM36 in complex with xylotriose (PDB entry lux7, chainA, Jamal-Talabani et al., Structure 12:1177-1187, 2004). D. TpolCBM16-1 in complex with mannopentaose (PDB entry 2zey, chainB, Bae et al., J Biol. Chem. 283:12415-12425, 2008).

FIG. 6 is a photographic representation showing the binding of recombinant K2 domain to glycans. blot showing binding to D-galactose (lane 1), chondroitin sulfate (lane 2), hyaluronan (lane 3) and control (lane 4). As the K2 domain has potential galactose-binding activity based on structural homology to other galactose-binding proteins (FIG. 3), the binding specificity between galactose and K2 was determined using a dot blot assay. A 0.2 μm porosity nitrocellulose membrane was coated with 100 mM of and Tris buffer control (lane 4) for overnight incubation at 4° C. The blot was then blocked with 5% skimmed milk/Tris buffer for 2 hours, washed, and probed with 10 μM K2 polypeptide overnight. Binding was detected with IIB2 mAb (DeCarlo et al., 1999 supra) at 5 μg/ml for 2 hours, followed by rabbit anti-mouse alkaline phosphatase for 2 hours and detected with alkaline-phosphatase substrate kit (Bio-Rad). The optical density (O.D.) for each dot on the blot was measured. A weak positive binding was observed between K2 and D-galactose and a slightly higher binding specificity was observed with chondroitin sulfate (a polymer of N-acetylgalactosamine and D-glucuronic acid). No binding to K2 was observed for hyaluronan (a polymer of D-glucuronic acid and N-acetylglucosamine)), maltose, D-mannose, sucrose or D-glucuronic acid.

FIG. 7 is a photographic representation showing the proteolysis sites of K2 following Kgp treatment. The sites of proteolysis of the purified Kgp cleaved K2 were identified by amino acid sequence analysis of cleaved fragments. Firstly 10 μl and 5 μl each of 0.7 mg/ml Kgp treated K2 sample were boiled in reducing sample buffer followed by separation on 16% w/v Tricine-SDS-PAGE and then stained with Coomassie G-250. The bands less than 7 kDa, K2/1 and K2/2, were excised for N-terminus sequencing using Edman degradation. Unambiguous sequence data was obtained for both peptides. K2/1 was resolved as GGARF indicating cleavage at. Lys-1276 while K2/2 was resolved as PQSV indicating cleavage at Lys-1291.

FIG. 8 is a graphical representation showing the hemolysis induced by the K2 domain. Data represent the mean±SEM from three independent experiments. A. Concentration-dependence of hemolysis induced by K2 polypeptide. Hemoglobin released after addition of K2 to erythrocytes, detectable after 24 hours of incubation. Error bars indicate the SEM. *, P<0.05; **, P<0.01 compared with K2 polypeptide. There is significant loss of ability to induce hemolysis when K2 is cleaved by Kgp as compared to untreated K2. B. Effects of RgpB on hemolysis induced by K2 polypeptide. The hemolytic effect of K2 was assessed by pre-incubating activated RgpB at 4 nM or 20 nM with erythrocytes for 30 minutes at 37° C., followed by the addition of K2 polypeptide or controls (PBS buffer or PBS containing cysteine) to the erythrocytes followed by further incubation. Showing hemoglobin released after 6 h incubation. Error bars indicate the SEM. ***, P<0.001 compared with RgpB alone.

FIG. 9 is a graphical representation showing the binding of gingipain components to human hemoglobin. Hemoglobin (Sigma) was coated onto ELISA plates at 20 μM in carbonate buffer pH 9.0. Using standard ELISA format binding of gingipain domains was detected by monoclonal antibody. Data show means±SEM for triplicates. Kgp bound with an apparent dissociation constant at equilibrium of nM. No binding was detected for K2, cleaved K2 or KPAD, a recombinant unprocessed construct encompassing the entire hemagglutinin/adhesin polypeptide of Kgp (FIG. 1A).

FIGS. 10A and B are representations showing the protein sequences identified among the Cleaved_Adhesin domains in Kgp, Rgp and HagA.

FIGS. 11A through C are cartoon representations of the K1, K2 and K3 structures. (A) Cartoon representation of K2 structure determined by X-ray crystallography at 1.4 angstrom. (B) Cartoon representation K3 structure determined by X-ray crystallography at 1.6 angstrom solved by standard molecular replacement methods using the K2 structure as a search model. (C) Cartoon representation of the superposition of K2 and K3 X-ray structures (K2 in blue, K3 in magenta, green balls for Ca atoms in K2, cyan balls for Ca atoms in K3).

FIG. 11D is a diagrammatic representation of an alignment of K2 and K3 from strain W83 with β-strands and α-helix represented.

FIGS. 12A and B are representations showing the binding of blood group H-trisaccharide by Kgp domains.

FIGS. 13A through C are photographic representations showing the superimposition of K3 on luy0 (carbohydrate binding module (CBM6 cm-2) from cellvibrio mixtus lichenase 5a in complex with glc-1, 3-glc-1, 4-glc-1, 3-glc).

FIGS. 14A through F are representations of the docking of the trisaccharide sugar of the A-blood group antigen into the proposed binding site located on the surface of the K3 structure. The trisaccharide was docked into the site by energy minimisation techniques using the program Haddock (Haddock, J. Am. Chem. Soc. 125:1731-1737, 2003; Dominguez et al., (2003), http://pubs.acs.org/servlet/reprints/DownloadReprint/ja026939x/L3tc). (A) Graphical picture of the surface of the proposed binding site found on K3 with relevant residues shown in stick representation. (B) Relevant residues shown in stick representation with labels and superimposed onto the cartoon representation of the fold of the backbone of K3. (C) The cartoon representation of the fold of the backbone of K3 with relevant loops labeled. (D) Surface of the K3 structure with the trisaccharide sugar of the A-blood group antigen docking into the proposed binding site. (E) Higher resolution picture of the binding shown in D. (F) Rotated view of E.

FIG. 14G is an alignment of amino acid sequences for K1 and K3.

FIGS. 15A through C are representations of a structural comparison of K3 and K2. A. Stereo representation of the observed fold of K3 with secondary structural features defined by a cartoon representation. Ca²⁺ ion positions are indicated by pink balls. B. Superimposition of K2 and K3 shows the structural similarities between the two crystal structures. The K2 structure and its Ca²⁺ ions are in pale green color and the K3 structure and its Ca²⁺ ions are in pink color. 158 residues were aligned together out of 175 residues in matched structures with a Cα rmsd of 1.7 Å. Parts of the extensive loop region at one end of the β-barrel which are the most different are highlighted by a circle. The co-location of Arg1280 (blue) in K2 and Arg1557 (yellow) in K3 is shown in stick representation. The designation of the Ca2+-I and Ca2+-II is indicated. C. Structural alignment of K2 and K3 with corresponding sequence alignments of K3*, A1 and K1. Secondary structural elements are shown above and below accordingly. Secondary structural elements are shown above and below accordingly. K2 has 178 residues (Ala1157-Gly1334) and K3 has 176 residues (Ala1427-Gly1602). Loop region alignments predicted only by sequence similarity are given in green boxes. The conserved residues in sequences are highlighted in red. The residues which coordinate to Ca2+ ions in K3 and K2 are identified by open triangles (Site-I) and solid triangles (site-II) and ligation via main chain carbonyls are further indicated by a star. Kgp-lys proteolysis sites in K3 (Li et al., 2010 supra) are identified by downward arrows. 173×178 mm (438×438 DPI).

FIG. 16 is a diagrammatic representation of detailed view of the arginine anchoring sites for arginines of loops L10 and L8 (R1280 and R1557) as observed in the crystal structures of K3 (green) and K2 (purple) respectively. The salt bridge H-bonding interactions with a conserved aspartic acid residue (D1319 and D1588 respectively) are shown as dashed lines while the additional H-bonds between the guanidinium group and the mainchain carbonyl of the Asp are not shown. The juxtaposition of these two sites is as observed in the overall superposition of the protein modules shown in FIG. 15B and the positions of the arginine atoms overlay with a rmsd of 1.28 R. 70×59 mm (600×600 DP1).

FIGS. 17A and B are representations of the A. Structural differences observed in the extensive loop regions of K2 and K3 with perspective rotated 90 degrees relative to FIG. 15B. The unaligned parts from the superimposition of K2 and K3 are highlighted in red color and labeled according to FIG. 15B, showing the different conformations of these loops. An arginine residue (green) is shown to be located in the same position in K3 and K2 despite being found in non-aligned and different loop conformers. B. Molecular surfaces of K2 and K3 in the same orientation as in the ribbon diagrams of A. L2, 4-L4-5 and L10 in K3 form a hollow (pocket-I) in the surface of K3 while L3, L4 and L8 in K2 form a flat surface in this area. L1 and L2 in K3 form a cleft on surface of K3 (pocket-II) which is not observed in K2 due to a different conformation of L1. 118×167 mm (600×600 DPI).

FIG. 18 is a graphical representation of the thermal stability dependency of K1, K2 and K3-domains on Ca²⁺. Proteins K1 and K2 were incubated in the presence, of SyproOrange, and in the varying concentrations of CaCl2. Folded forms of K1 (top) and K2 (middle) were significantly stabilized by Ca²⁺ titration (Tm enhanced by 15° C. and 8° C., respectively), whilst this effect is more subtle in the case of K3 (bottom, ˜3° C. stabilization). 658×571 mm (96×96 DP1).

FIG. 19 is a graphical representation of the haemolytic activities of the structurally homologous K3 and K1 modules. Various levels of K3 or K1 for up to 3 μM were added to 0.2% erythrocytes for a total volume of 200 μL. Incubation of erythrocytes with K3, heat-treated (H.T.) K3 (20 min at 80° C.), or K1 was done in PBS at 25 C for 48 hours. After the incubation, the microtitre plate was centrifuged then at 1000×g for 10 min and the supernatants (100 μL) transferred into a new microtitre plate. Hemoglobin release was measured by the absorbance at 405 nm in a microtitre plate reader. Results are representative of three separate experiments. Error bars indicate the means and SE. *, P<0.05; **, P<0.01; ***, P<0.001 compared with heat-treated K3 polypeptide. 80×82 mm (300×300 DPI).

FIG. 20 is a graphical representation of interaction of hemoglobin with the K3, K2 and K1 proteins. 96 well ELISA plates were coated with the K3, K2 or K1 proteins (0.4 g/well in PBS) and incubated overnight at 4° C. The wells were blocked with 100 μl of 1% w/v skim milk in PBS for 1 hour. Hemoglobin was added to the plates in various concentrations. Hereafter, anti-human Hb rabbit polyclonal antibody was added, followed by alkaline phosphatase-conjugated goat anti-rabbit IgG. Color development was detected with phosphatase substrate. Data were fitted by non-linear regression using GraphPad Prism 4.0 software (GraphPad Inc., La Jolla, Calif., USA). Apparent Kd values were calculated from the fitted curves. 79×82 mm (300×300 DPI).

FIG. 21 is a graphical representation of the interaction of fibrinogen with K3, K2, and K1 polypeptides. 96 well ELISA plates were coated with K3, K2 or K1 polypeptide (0.4 g/well in PBS) and incubated overnight at 4° C. The wells were blocked with 100 μl of 1% w/v skim milk in PBS for 1 hour. Fibrinogen from human plasma (Sigma) was added to the plates in various concentrations. Thereafter, anti-fibrinogen mAb [FG-21, Sigma] was added, followed by alkaline phosphatase-conjugated rabbit anti-mouse IgG. Color development was detected with phosphatase substrate. 74×77 mm (300×300 DPI).

FIGS. 22A and B are representations of the interaction of rHSA and rHSA-heme with K1, K2 and K3 polypeptides 96 well ELISA plates were coated with K1, K2 or K3 proteins (0.4 μg/well in PBS) and incubated overnight at 4° C. The wells were blocked with 100 μl of 1% w/v skim milk in PBS for 1 hour. Purified rHSA (A) or rHSA-heme (B) was added to the plates in various concentrations. Thereafter, anti-HSA mAb (15C7, ABCAM) was added, followed by alkaline phosphatase-conjugated rabbit anti-mouse IgG. Color development was detected with phosphatase substrate. Data were fitted by non-linear regression using GraphPad Prism 4.0 software (GraphPad Inc., La Jolla, Calif., USA). Apparent Kd values determined for rHSA-heme were 6.8 M for K3 polypeptide and 6.3 M for K2. For rHSA the apparent Kd values were estimate to be >50 M. 173×81 mm (450×450 DPI).

FIGS. 23A through C are representations of the rigid-body modeling of K1K2 and K1K2K3. A. BUNCH rigid-body modeling ensembles of K1K2 (green transparent surface/ribbons) and K1K2K3 (blue transparent surface/ribbons). The refined position of the mass ascribed to the linkers between K1-K2 and K2-K3 are represented as spheres. The bracket indicates that the positions of the K1 and K2 modules can be swapped in the K1K2K3 ensemble to produce shapes with essentially equivalent fits to the data. B. K1K2 and K1K2K3 SAXS data showing the corresponding fits and range of the rigid-body refined models shown in A(K1K2, green line; K1K2K3, blue line). The data have been scaled for clarity. C. A spatial superposition of representative K1K2 (green) and K1K2K3, (blue) models derived from the SAXS data. 173×188 mm (300×300 DPI).

FIGS. 24A and B provide the atomic coordinate of (A) K2 crystal refined at 1.4 Angstrom; and (B) K3 crystal referred ct 1.6 Angstrom. The K3 structure is complexed with A-antigen trisaccharide docked in (by energy minizatin using program Haddock) into putative carbohydrate binding site.

### DETAILED DESCRIPTION

Throughout this specification, unless the context requires otherwise, the word “comprise”, or variations such as “comprises” or “comprising”, will be understood to imply the inclusion of a stated element or integer or step or group of elements or integers or steps but not the exclusion of any other element or integer or step or group of elements or integers or steps.

As used in the subject specification, the singular forms “a”, “an” and “the” include plural aspects unless the context clearly dictates otherwise. Thus, for example, reference to “a domain” includes a single domain, as well as two or more domains; reference to “an antagonist” includes a single antagonist, as well as two or more antagonists; reference to “the disclosure” includes a single aspect or multiple aspects of the disclosure; and so forth.

Adhesin and/or carbohydrate binding regions of proteins expressed by Porphyromonas gingivalis are subject to Cleaved_Adhesin Domain Family modeling (Finn et al., 2008 supra). Particular domains identified are referred to as K1, K2 and K3 on lysine gingipain (Kgp) and their equivalents on arginine gingipain (Rgp) [R1 and R2] and hemagglutinin A (HagA) [A1 through A10, inclusive].

Crystal structure determination of the K2 and K3 domains on Kgp confirms the topology of these domains and indicates that K2 functions as a hemolysin. The atomic coordinates for K2 and K3 are provided in Protein Data Bank under identifiers 3KM5 and 3M1H, respectively the contents of which are incorporated by reference. The present disclosure contemplates, therefore, a domain structure for the hemagglutinin (HA) region of protease-like molecules expressed by microorganisms which acquire iron, heme or porphyrin from biological environments generally, but not exclusively, for growth. The identified Cleaved_Adhesin domains also have homologs in a range of proteins from organisms not necessarily related to P. gingivalis. The identification of these domains also provides scope to generate therapeutic and diagnostic agents for a range of microorganisms and/or which target particular proteins.

The domains identified herein by Cleaved_Adhesin domain modeling (Finn et al., 2008 supra) are referred to as “Cleaved_Adhesin domains”. The Cleaved_Adhesin domains are identified within the hemagglutinin (HA) region of a protease-like molecule expressed on the surface or secreted by a Porphyromonas gingivalis or related microorganism. Homologous domains are identified in a range of proteins as summarized in http://pfam.sanger.ac.uk//family?acc=PF07675#tbaview=tab6.

The protease-like molecules contemplated herein include particular gingipains (gp's) such as lysine gingipain (Kgp) and arginine gingipain (Rgp) and hemagglutininA (HagA). The particular Cleaved_Adhesin domains on Kgp are referred to as K1, K2 and K3. Each of K1, K2 and K3 is defined by the amino acid sequences set forth in SEQ ID NOs:1 to 3, 10, 13, 14, 19, 22, 23, 25 and 31 (see Table 1), for particular strains of P. gingivalis. Reference to “K1”, “K2” and “K3” on Kgp from P. gingivalis includes functional equivalents or homologs on other protease-like molecules from P. gingivalis or from related microorganisms. Such functional equivalents or homologs include domains R1 and R2 on Rgp (defined by SEQ ID NOs:4 and 20). Similar Cleaved_Adhesin domains are also referred to as A1, A2, A3, A4, A5, A6, A7, A8, A9 and A10 in HagA (SEQ ID NOs:5 to 9, 11, 12, 15 to 18, 21, 24 and 26 to 30). A “protease-like molecule” includes a molecule having an HA region and which has amino acid sequence homology or catalytic activity of a cysteine protease. The term extends to HagA and other Hag proteins. The present disclosure extends to Cleaved_Adhesin domain containing proteins having Cleaved_Adhesin domains homologous to those exemplified herein expressed by other strains of Porphrymonas or unrelated microorganisms or present in related or unrelated proteins as summarized at http://pfam.sanger.ac.uk//family?acc=PF07675#tbaview=tab6.

It is proposed to use the K1, K2 and/or K3 domains or their equivalents or homologs such as R1 and R2 and A1 through A10 to identify antagonists of the activity of proteins carrying all or some of these domains. By “antagonizing the activity” includes inhibiting or reducing hemolysin or hemolytic activity of erythrocytes. These domains also provide targets for diagnostic agents to monitor infection and treatment protocols. These domains can also be used to identify other similar Cleaved_Adhesin domains in a range of related and un-related proteins. Such domains are useful targets for antagonists, agonists and diagnostic agents.

Reference to an “equivalent” or “homolog” of K1, K2 and K3 or R1 and R2 or A1 through A10 includes structural or sequence identity as well as domains having conformational, functional or sequence similarity or homology to K1, K2, K3, R1, R2 or A1 through A10. Generally, an “equivalent” or “homolog” includes a domain also deemed to be a Cleaved_Adhesin domain.

Accordingly, Cleaved_Adhesin domains are defined herein within the adhesin/carbohydrate region of a microbial molecule involved in hemolysis or hemolytic activity of erythrocytes and their use in the manufacture of medicaments for the treatment or prophylaxis of infection in the biological environment by the microorganism.

Hence, a method is contemplated for the prophylaxis or treatment of infection by a microorganism in a biological environment from where the microorganism acquires iron, heme or porphyrin, the method comprising administering to the environment an effective amount of an agent for a time and under conditions sufficient to antagonize a Cleaved_Adhesin domain with the adhesin and/or carbohydrate binding region of a protease-like molecule produced by the microorganism, the domain associated with hemolysis or hemolytic activity of erythrocytes.

The term “biological environment” is used in its broadest context to include an environment comprising porphyrin-containing molecules. Particular porphyrin-containing molecules include hemoglobin and its precursors as well as heme such as found in erythrocytes. In an embodiment, the biological environment is a vascular region or cavity or a mucosal membrane in an animal species such as a mammal, reptile, amphibian, fish or bird or is a hoof of a livestock animal comprising erythrocytes or other heme-containing cells. In an embodiment, the animal is a mammal such as a human or livestock animal.

Accordingly, the present disclosure provides a method for the prophylaxis or treatment of infection by a microorganism in a mammal from where the microorganism acquires iron, heme or porphyrin, the method comprising administering to the environment an effective amount of an agent for a time and under conditions sufficient to antagonize a Cleaved_Adhesin domain within an HA region of a molecule produced by the microorganism wherein the molecule is a protease-like molecule associated with hemolysis or hemolytic activity of erythrocytes.

In an embodiment, the disclosure relates to P. gingivalis infection in the oral cavity such as during periodontal disease. The instant disclosure extends to any disease condition resulting from microbial infection and in particular infection by P. gingivalis or a related microorganism involving the acquisition of iron, heme or porphyrin. Such microorganisms are required to acquire iron, heme or porphyrin as they do not possess a biosynthetic pathway for porphyrins. Examples of microorganisms related to P. gingivalis contemplated herein include but are not limited to Salmonella sp., Serratia sp, Yersinia sp, Klebsiella sp, Vibrio sp, Pseudomas sp, E. coli, Haemophilus sp and Bordetella sp. Examples of P. gingivalis or related microorganism infection contemplated by the present disclosure include infection of the oral cavity, nasopharynx, oropharynx, vagina and urethra as well as infection of mucosal membranes and infection of hooves of livestock animals such as sheep, cattle and goats. An “effective amount” means an amount sufficient to prevent or reduce hemolysis or hemolytic activity of erythrocytes. The effective amount may also be determined by an amount sufficient to inhibit growth of a microorganism such as P. gingivalis.

In another aspect, a method is provided for the prophylaxis or treatment of infection by Porphyromonas gingivalis or a related organism in a mammal, the method comprising administering to the mammal an effective amount of an agent for a time and under conditions sufficient to antagonize a Cleaved_Adhesin domain within the HA region of a gingipain or HagA, wherein the antagonism prevents or reduces hemolysis or hemolytic activity of erythrocytes.

The present disclosure also contemplates a method for prophylaxis or treatment of periodontal, pulmonary, vaginal, urethral or hoof disease resulting from infection by P. gingivalis or related microorganism in a mammal, the method comprising administering to the mammal an effective amount of an agent for a time and under conditions sufficient to antagonize, a Cleaved_Adhesin domain within the HA region of a gingipain or HagA, wherein the antagonism prevents or reduces hemolysis or hemolytic activity of erythrocytes.

Reference herein to “Porphyromonas gingivalis” or its abbreviation “P. gingivalis” includes reference to all strains, mutants, derivatives and variants of this organism as well as serological sub-types. The present disclosure further extends to microorganisms related to P. gingivalis at the metabolic, structural, biochemical, immunological and/or disease causing levels. Examples of related microorganisms are those listed above.

The present disclosure provides in an embodiment, a method for the prophylaxis or treatment of infection by a microorganism in a biological environment from where the microorganism acquires iron, heme or porphyrin, the method comprising administering to the environment an effective amount of an agent for a time and under conditions sufficient to antagonize a Cleaved_Adhesion domain within an adhesin and/or carbohydrate binding region of a molecule produced by the microorganism, the domain associated with hemolysis or hemolytic activity of erythrocytes, wherein the domain is defined by Cleaved_Adhesin Domain modeling.

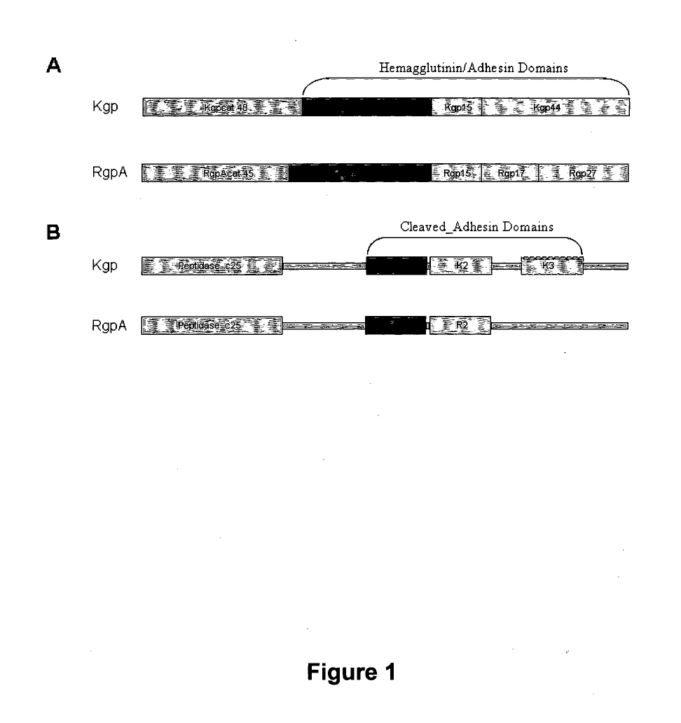

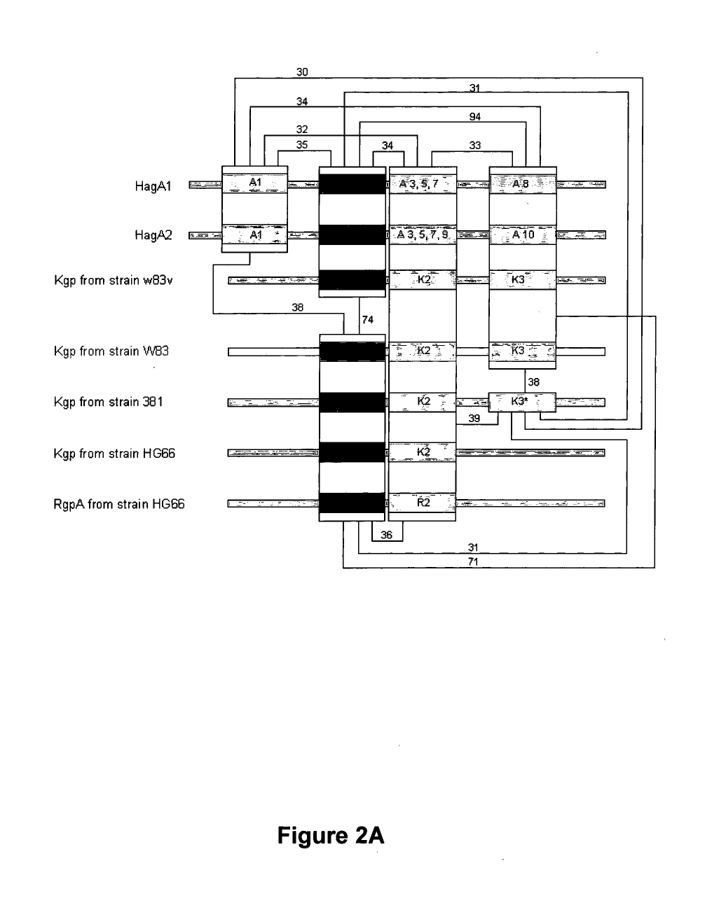

As indicated above, provided are Cleaved_Adhesin domains K1, K2 and K3 on Kgp and their equivalents on Rgp (R1 and R2) and HagA [A 1 through A10] (See FIG. 1). Furthermore, these domains can be used to identify similar Cleaved_Adhesin domains in a variety of proteins not necessarily related to a protease-like molecule or a molecule for P. gingivalis.

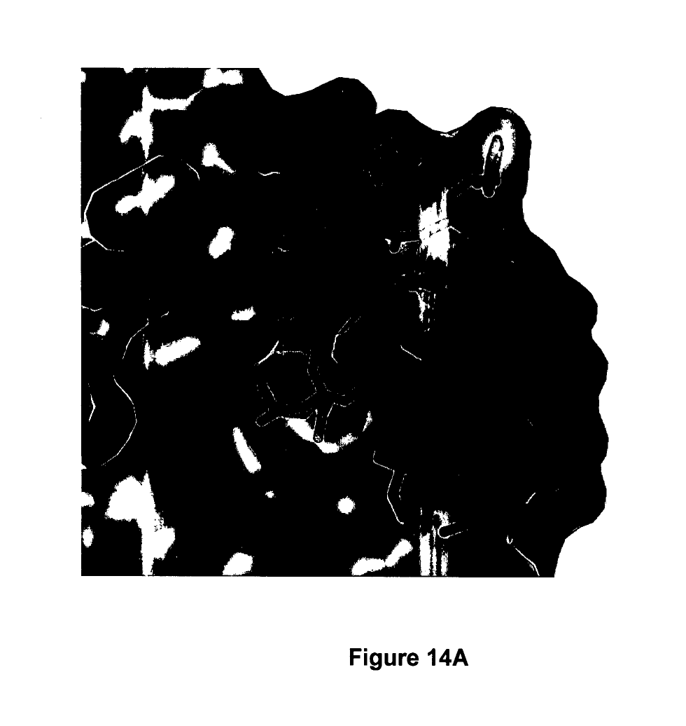

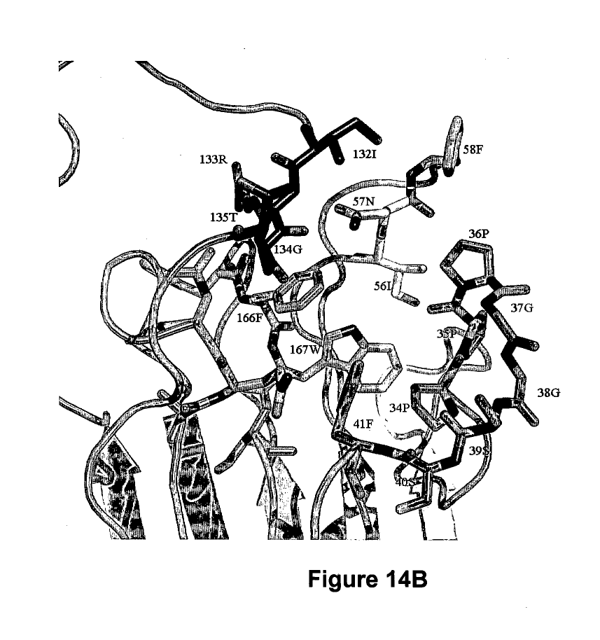

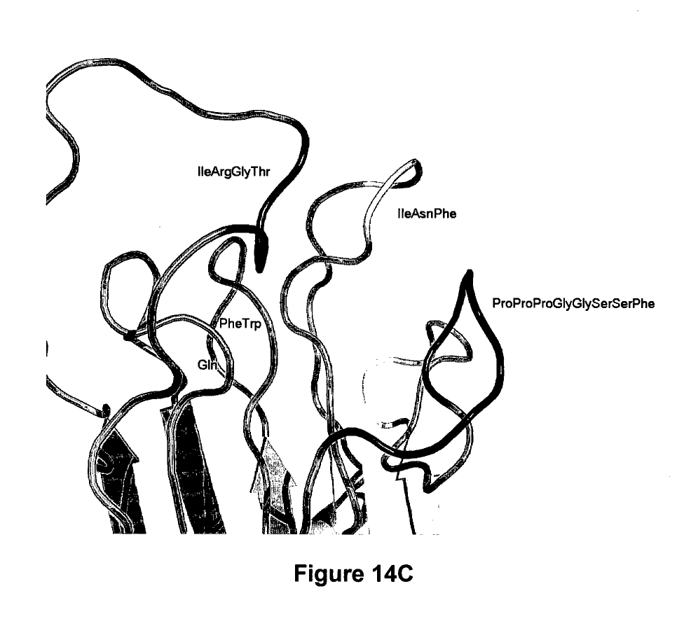

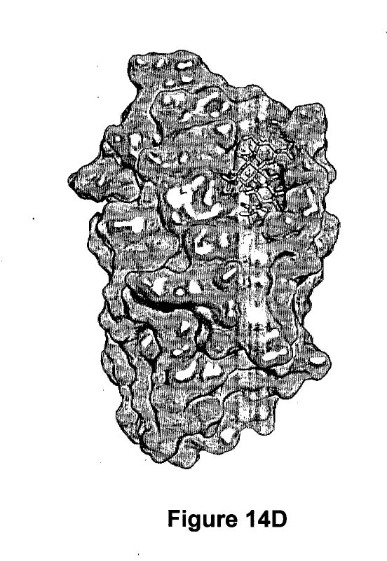

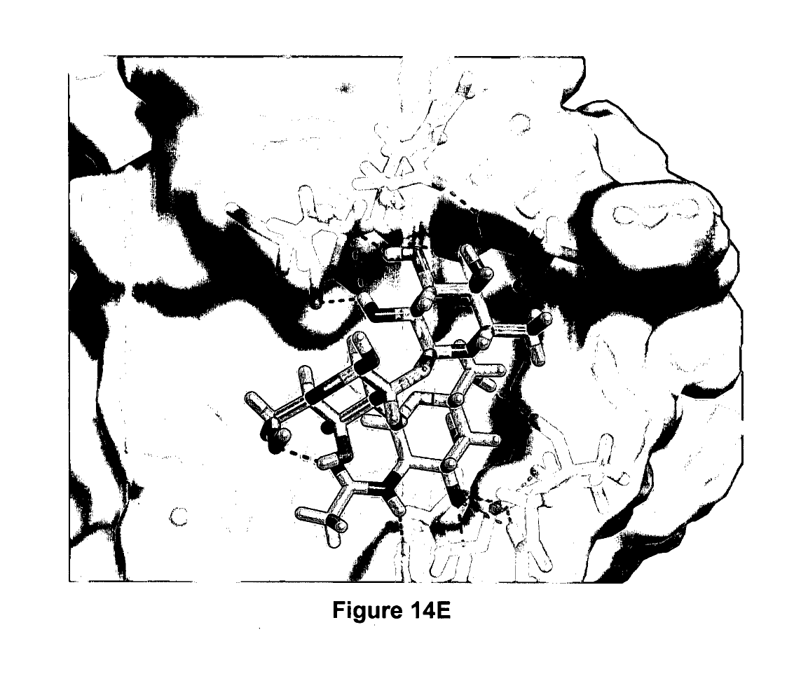

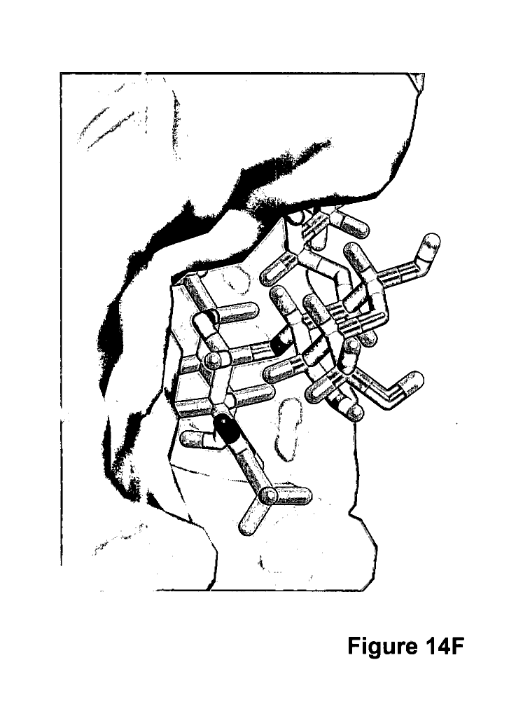

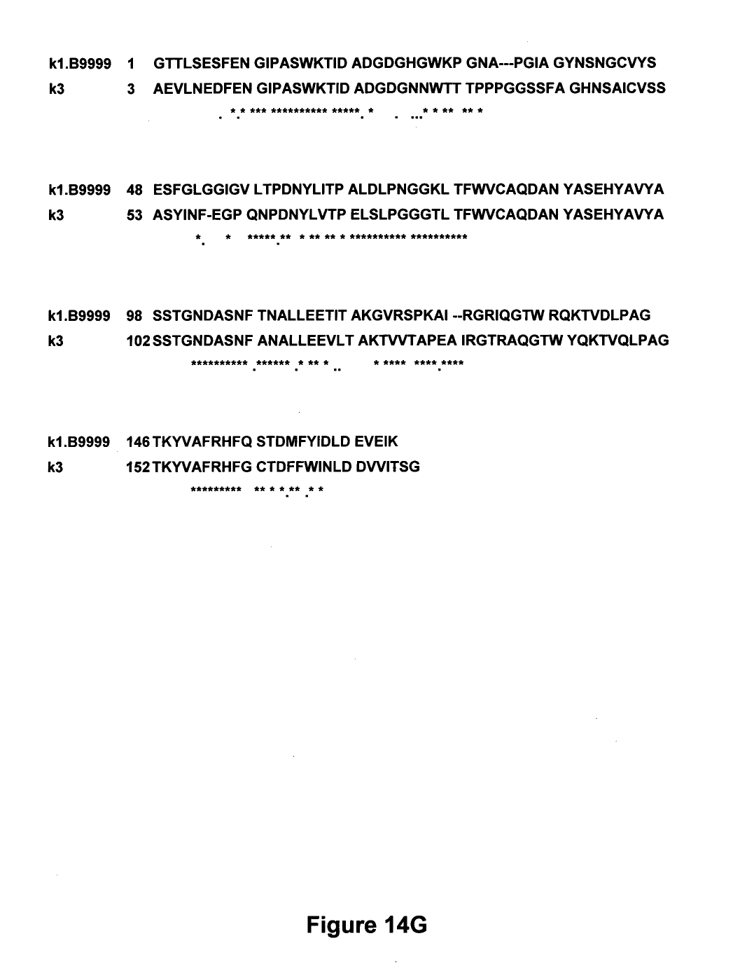

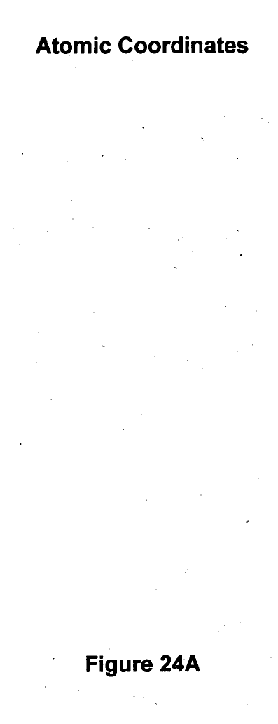

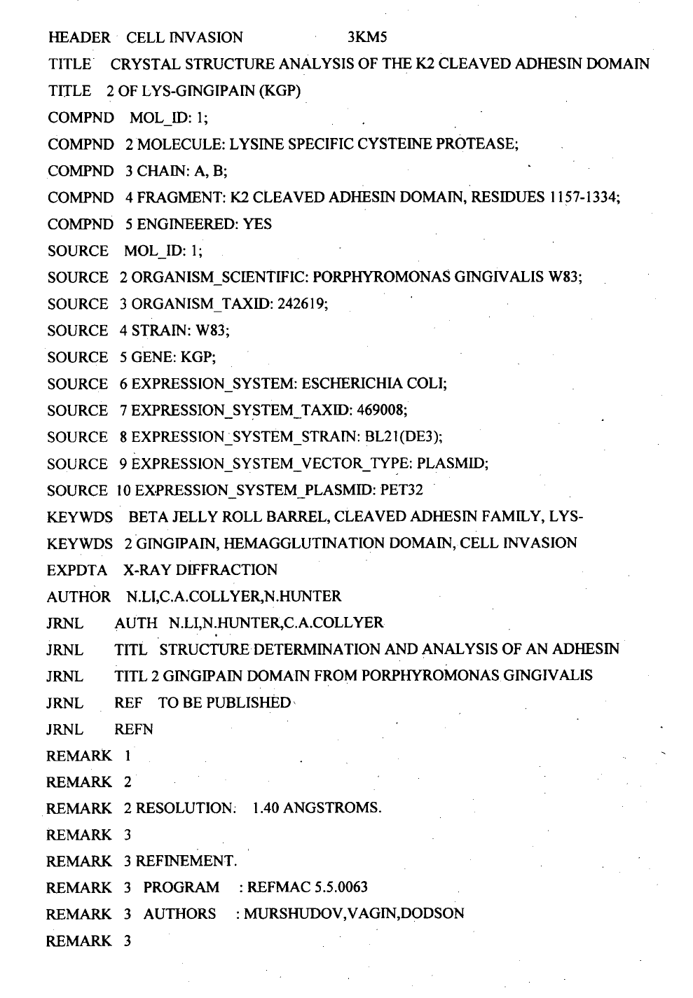

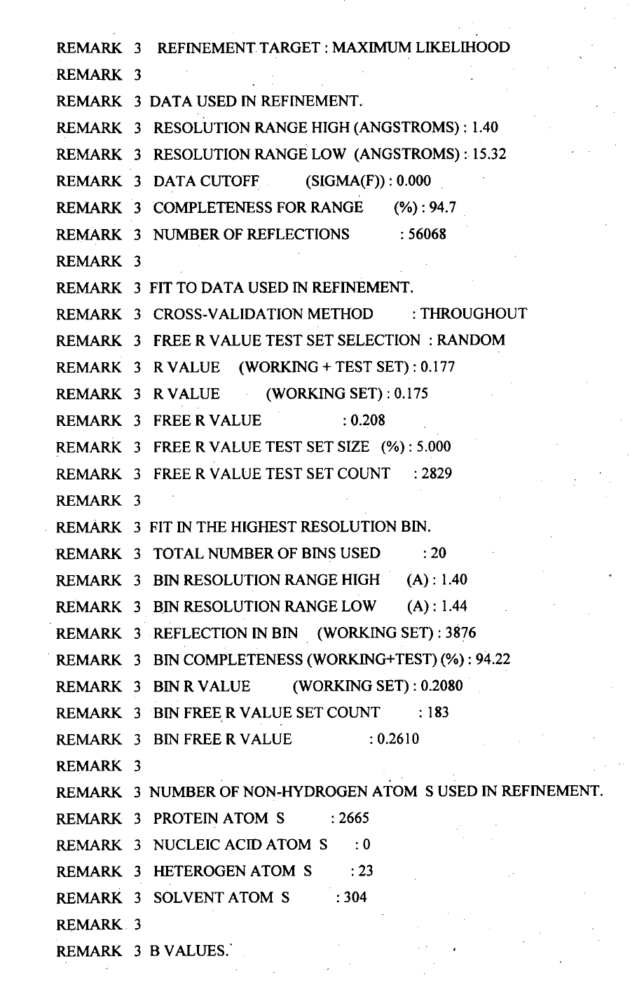

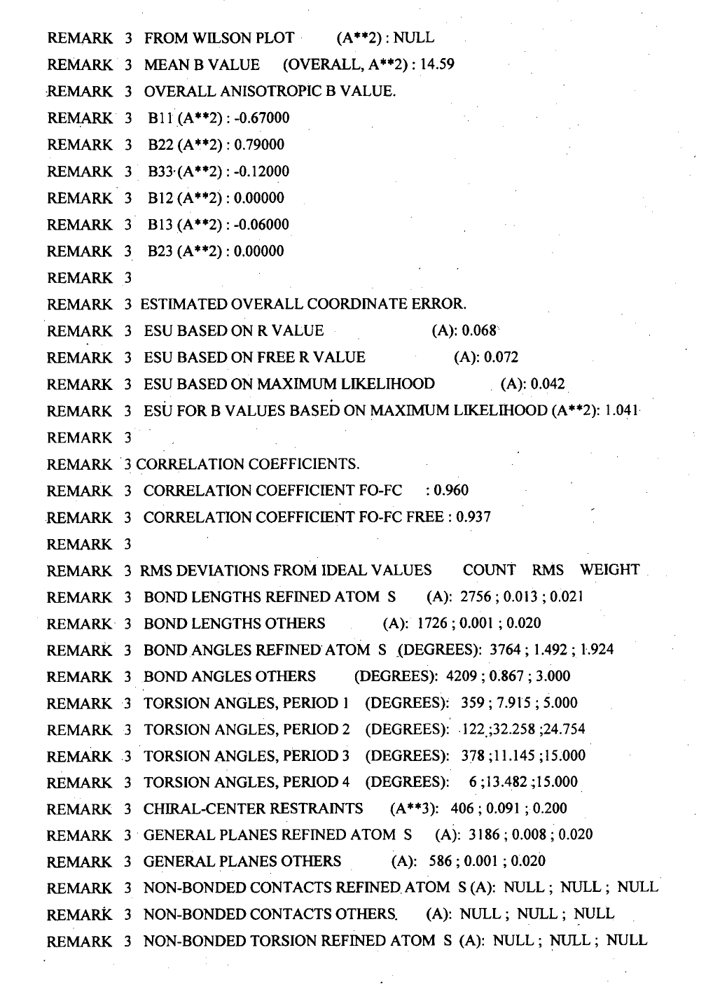

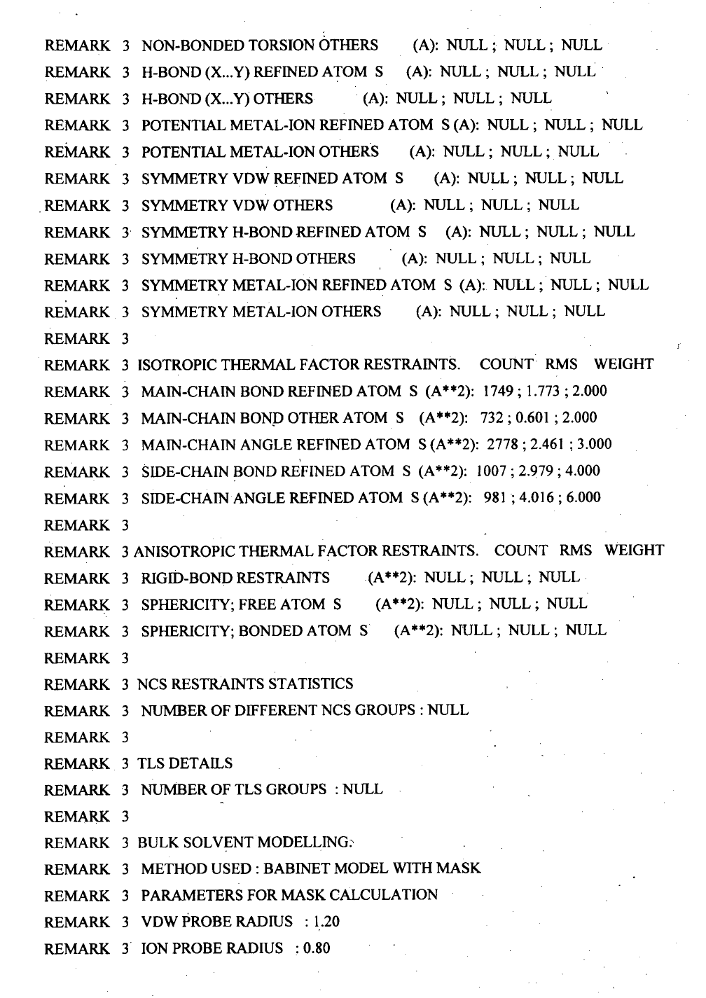

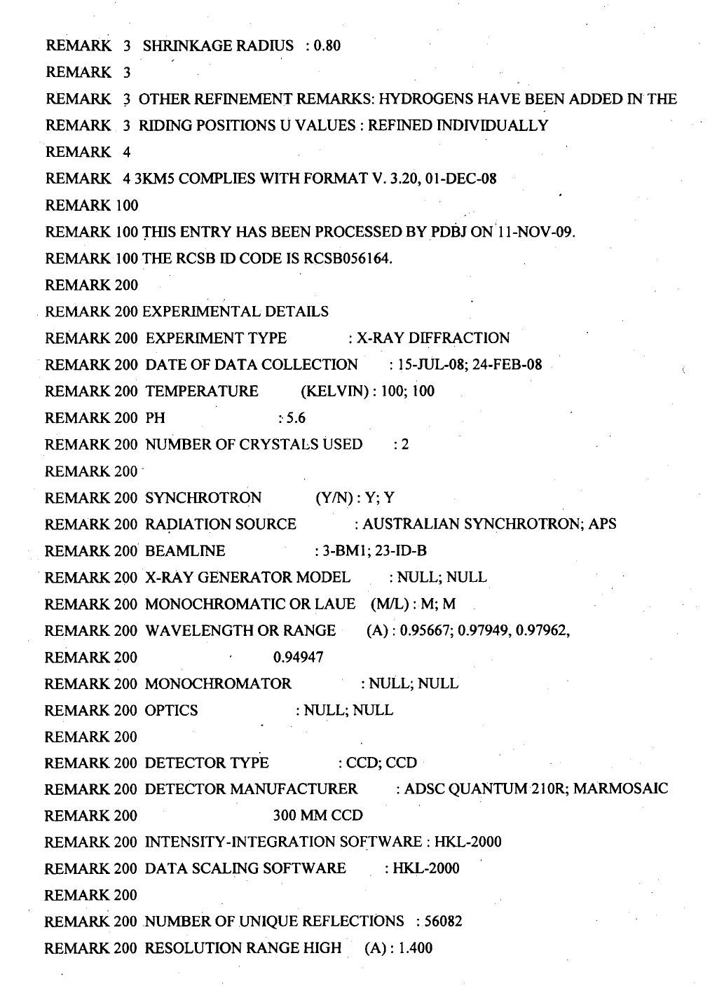

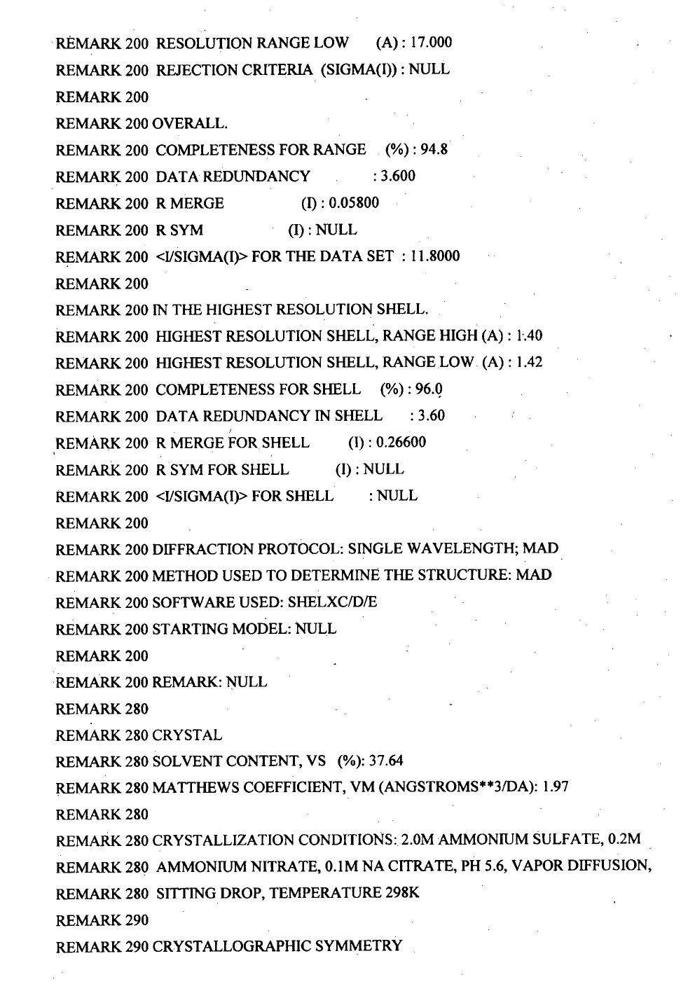

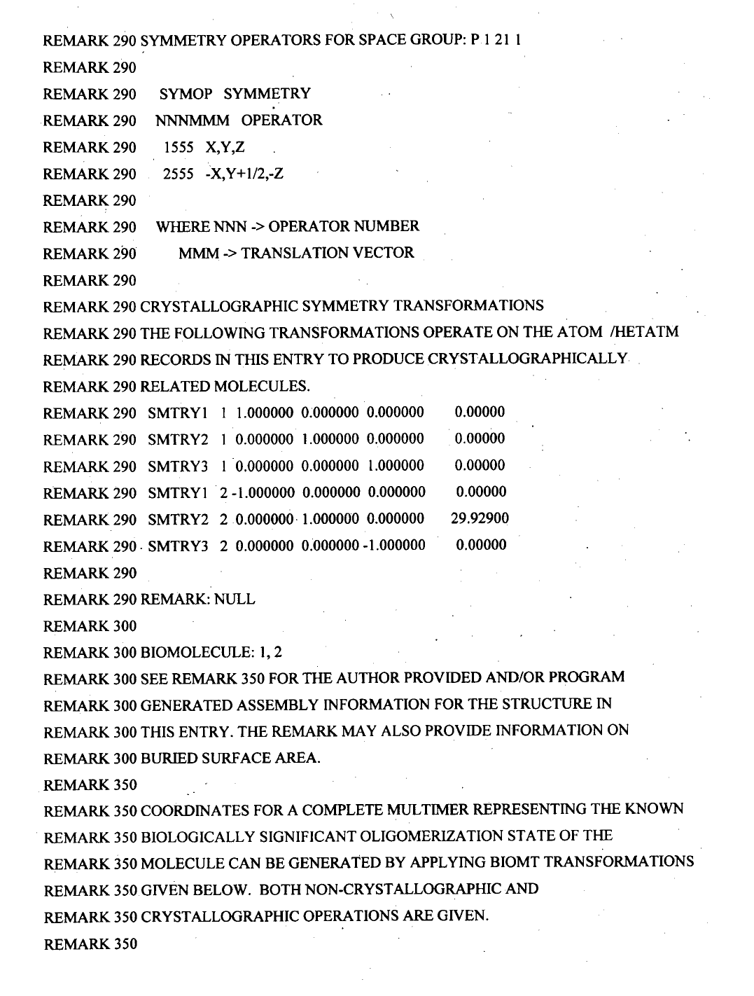

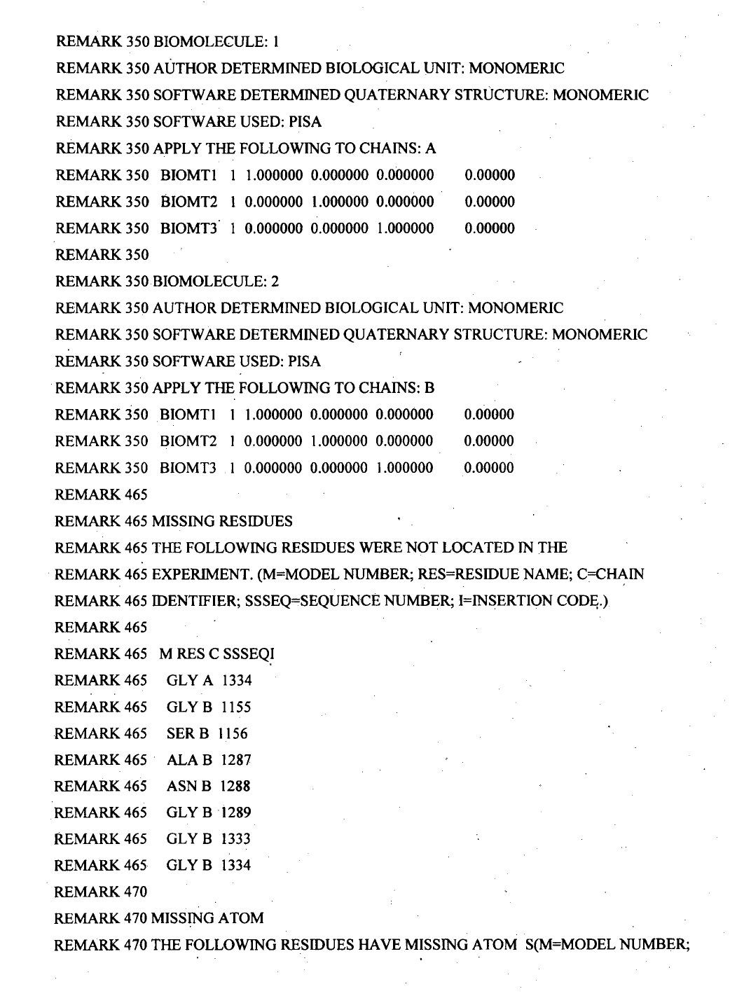

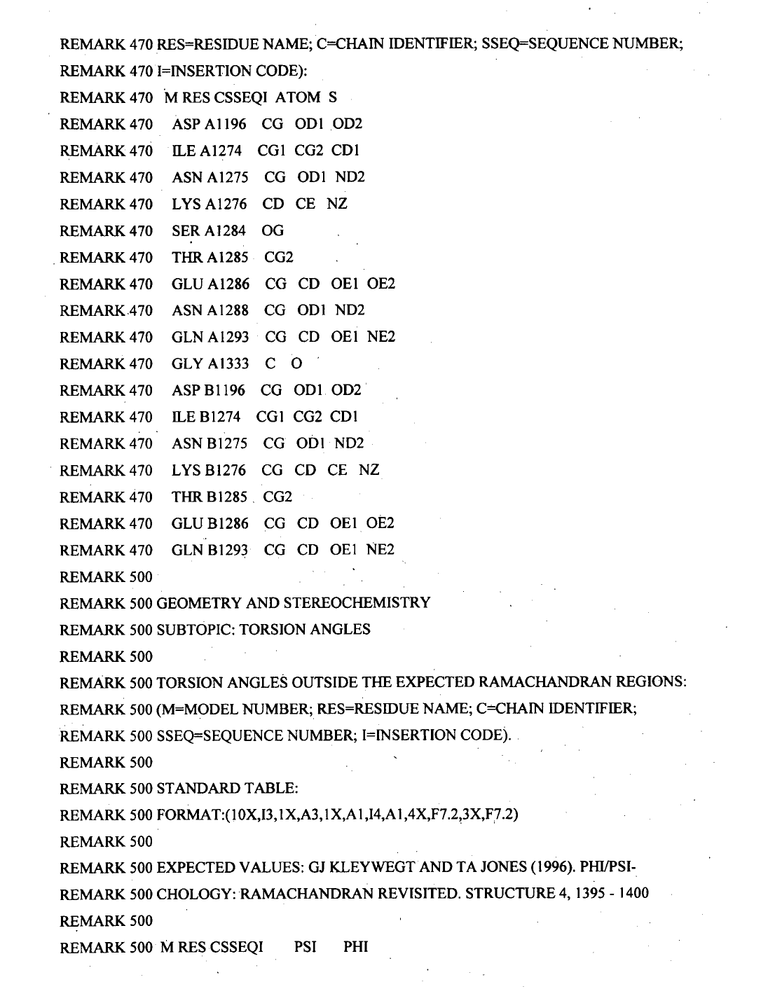

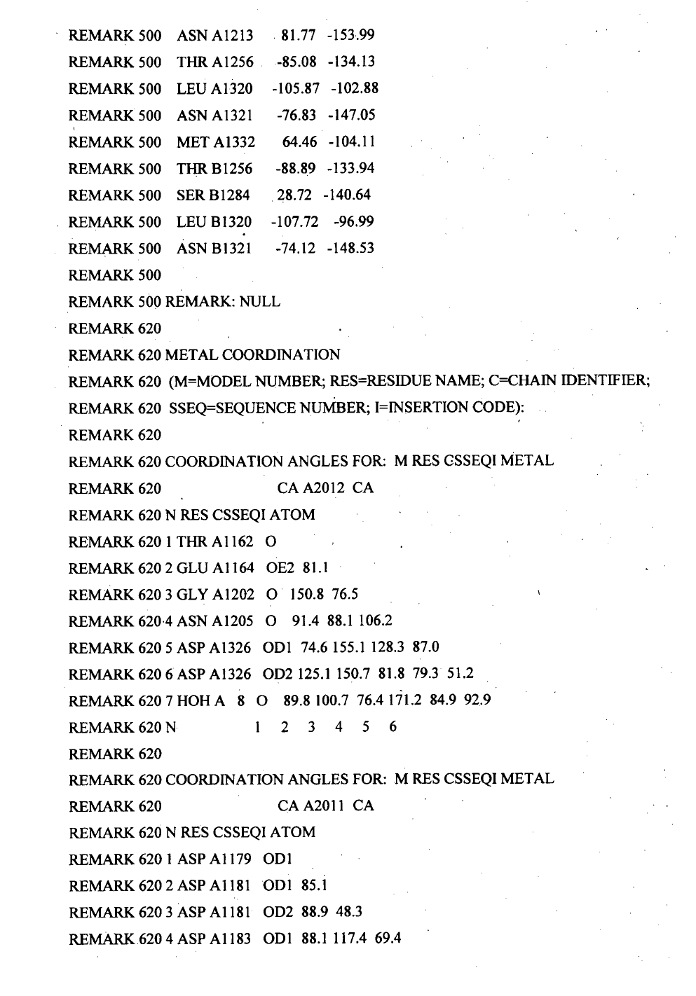

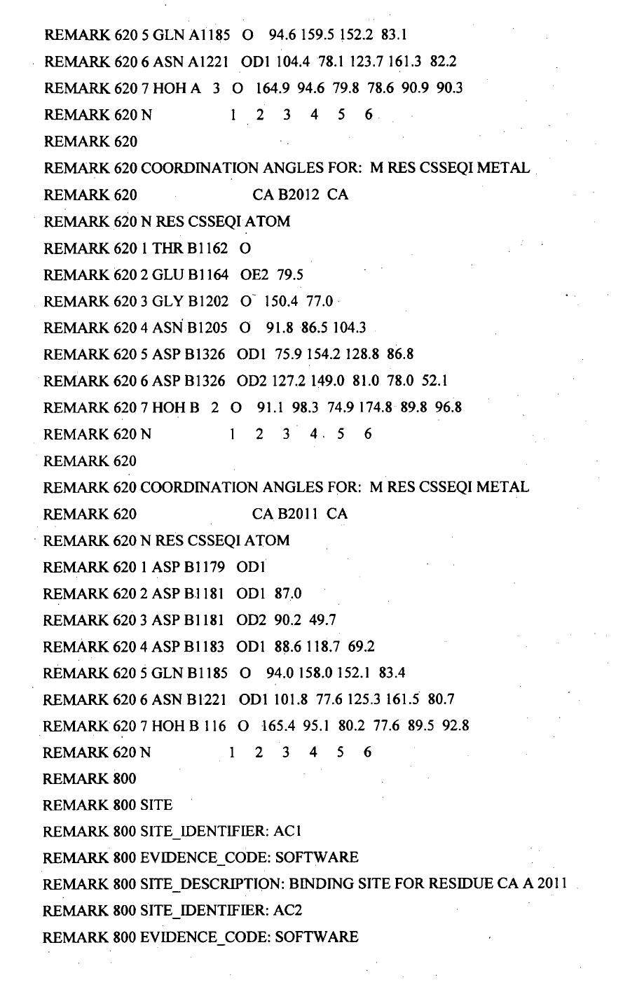

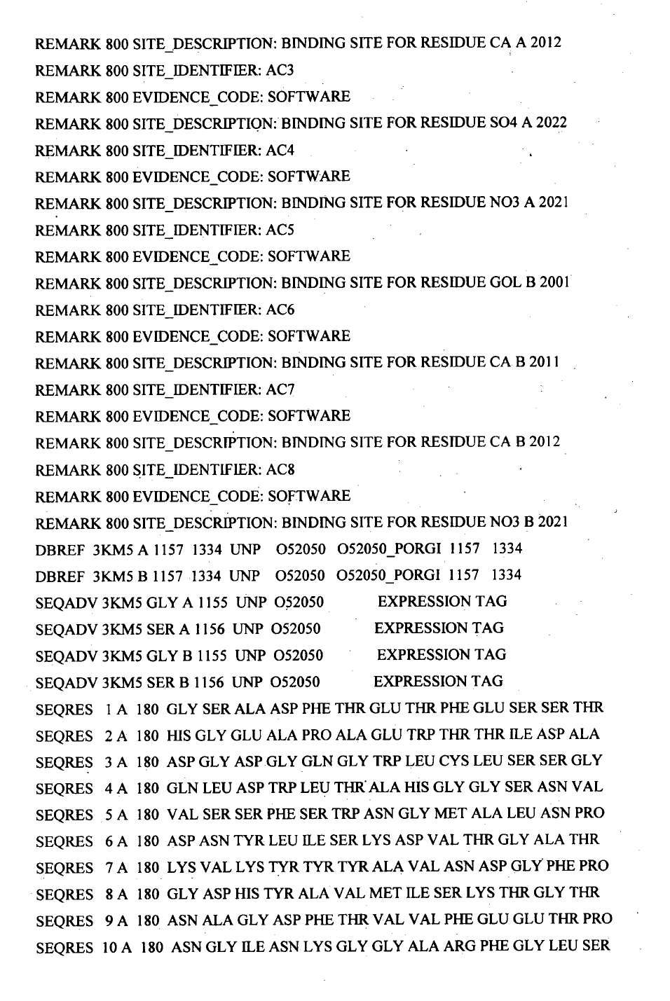

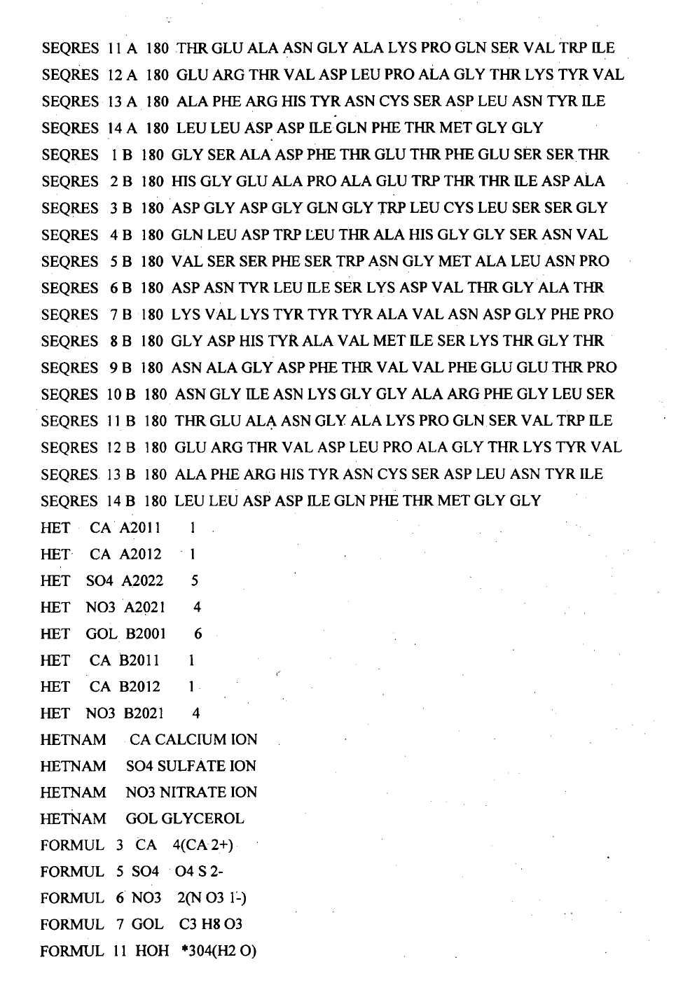

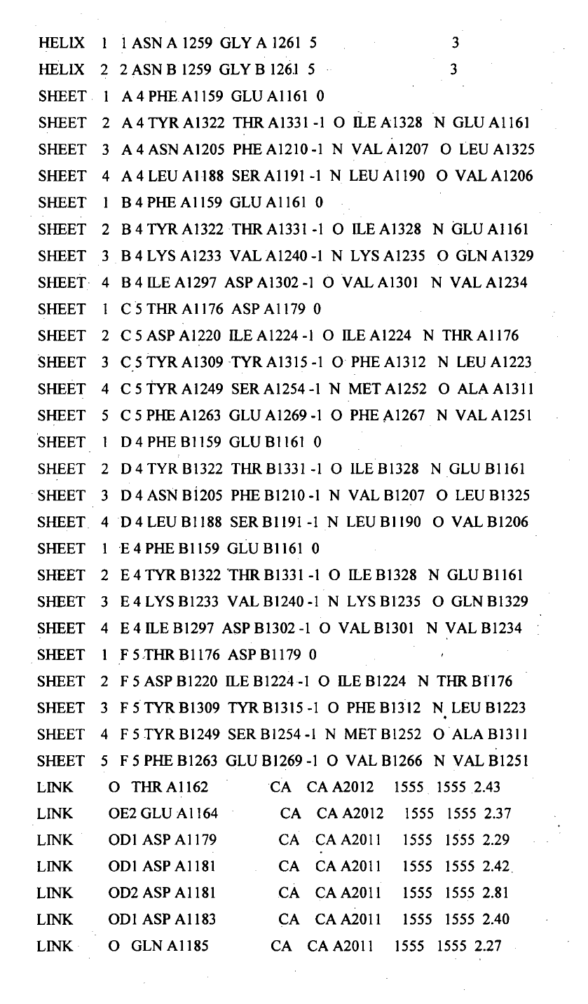

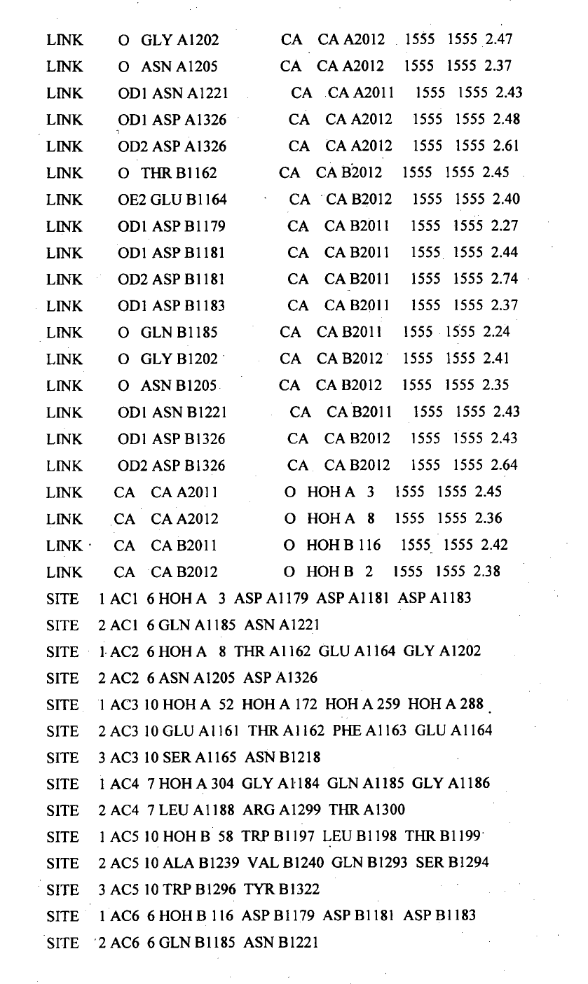

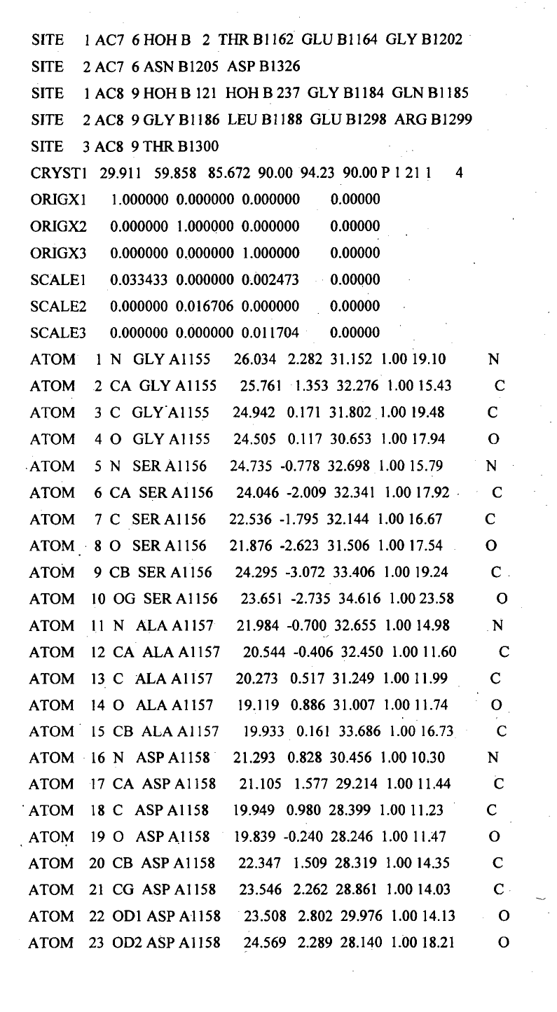

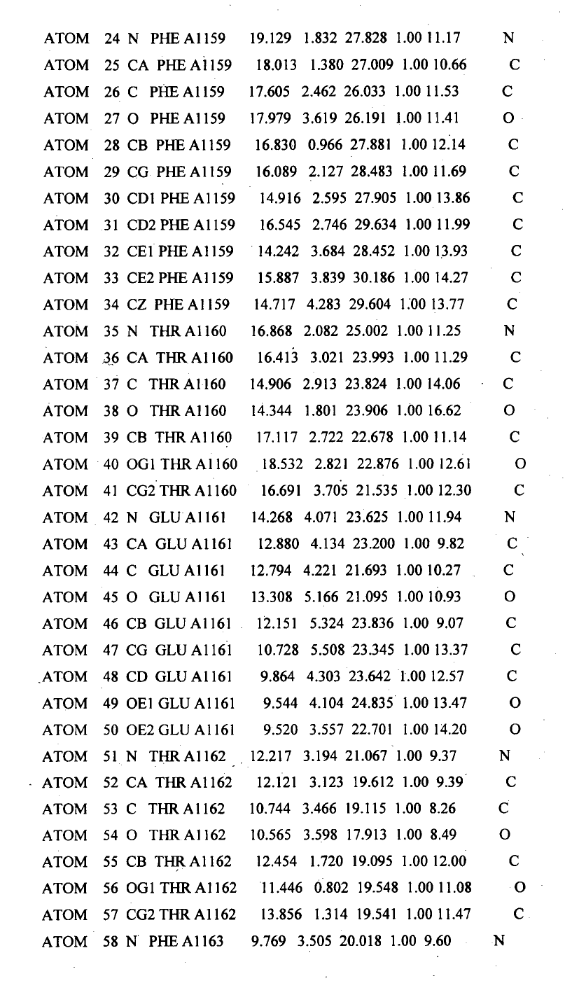

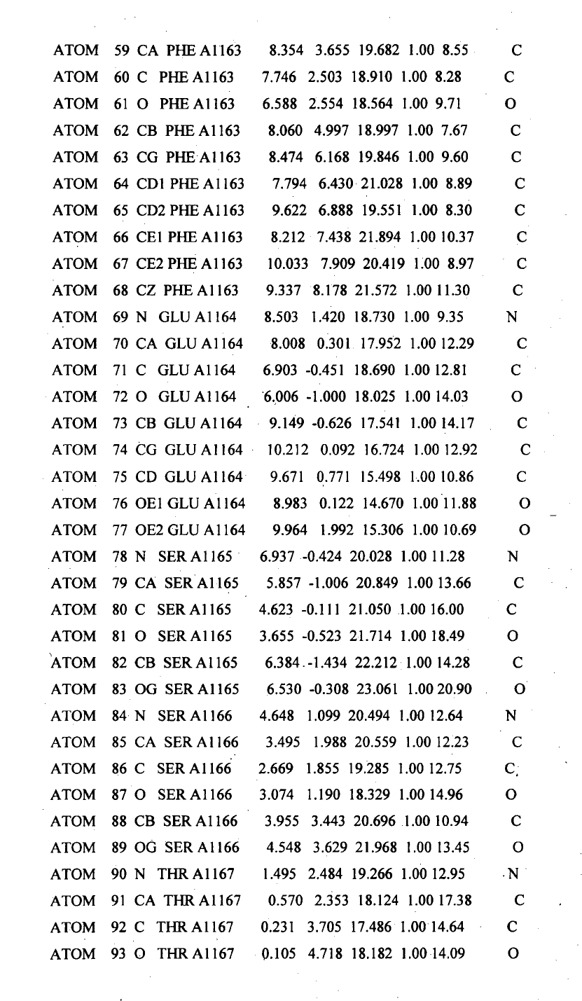

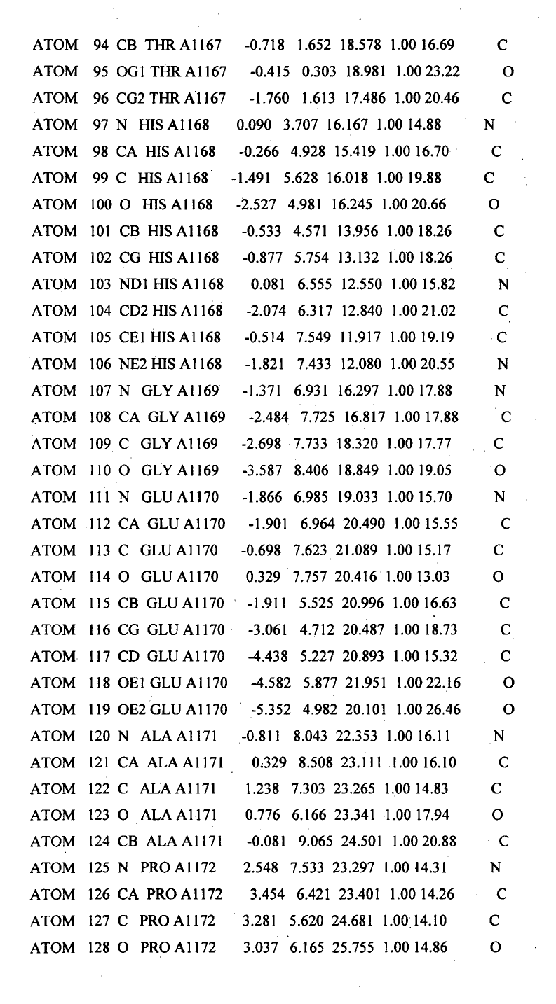

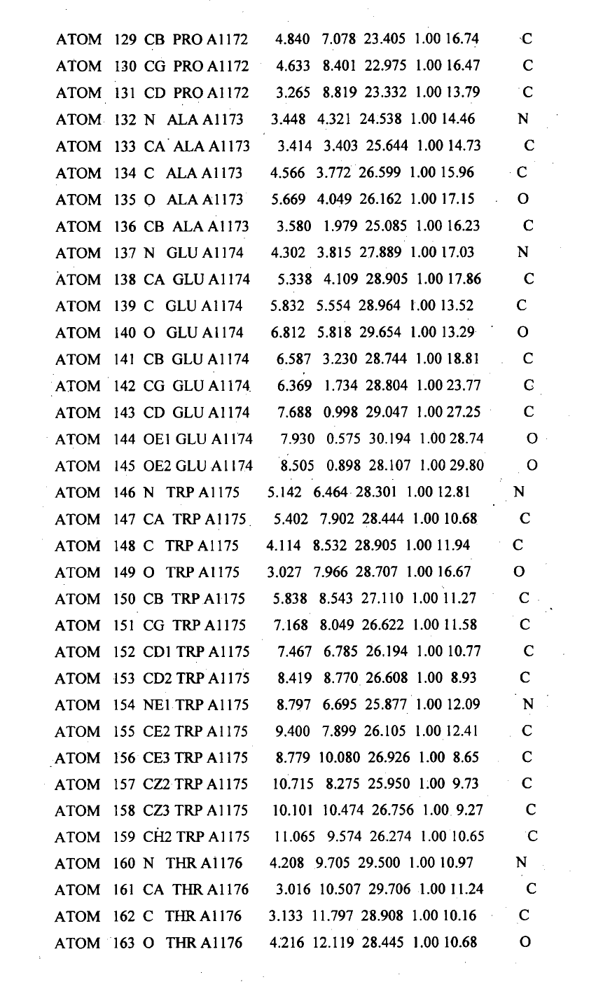

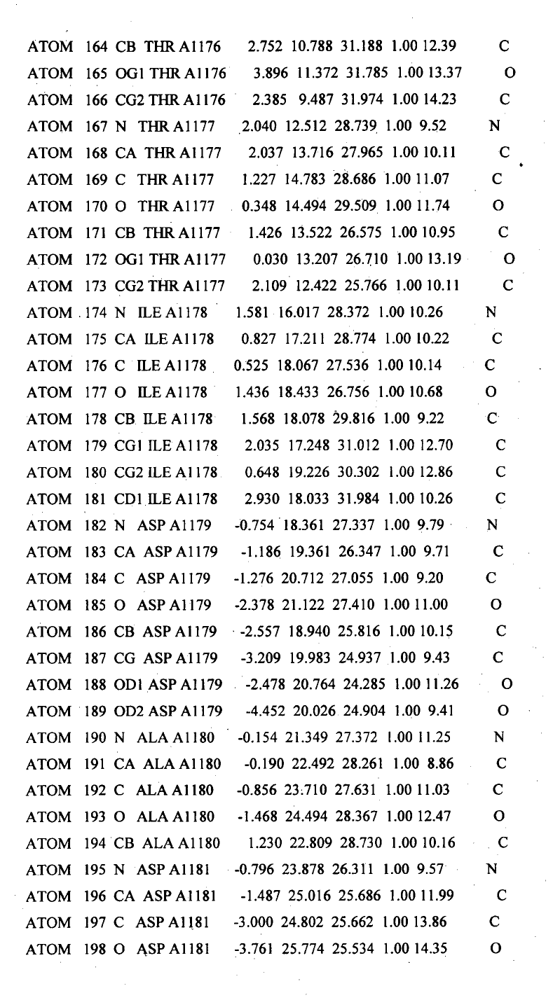

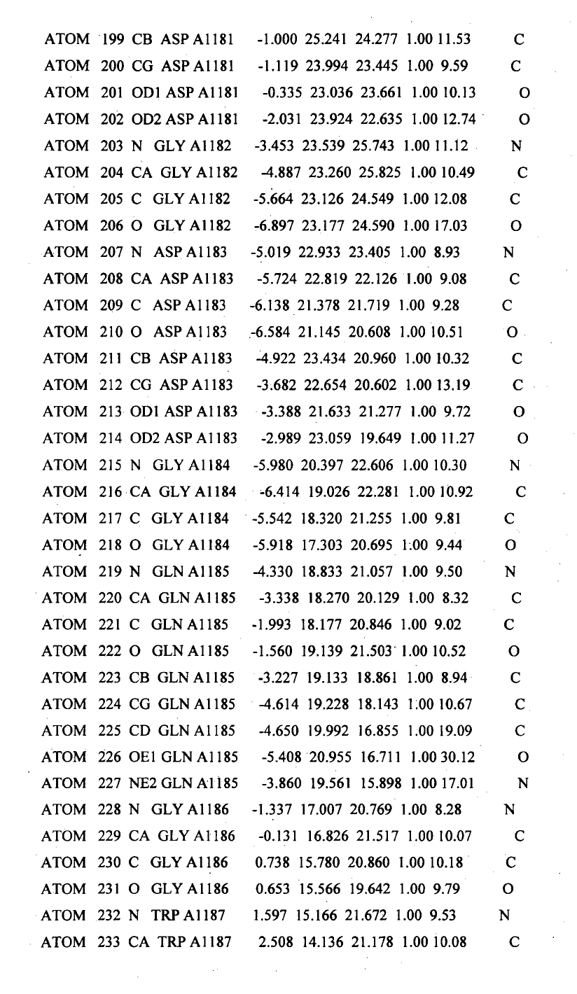

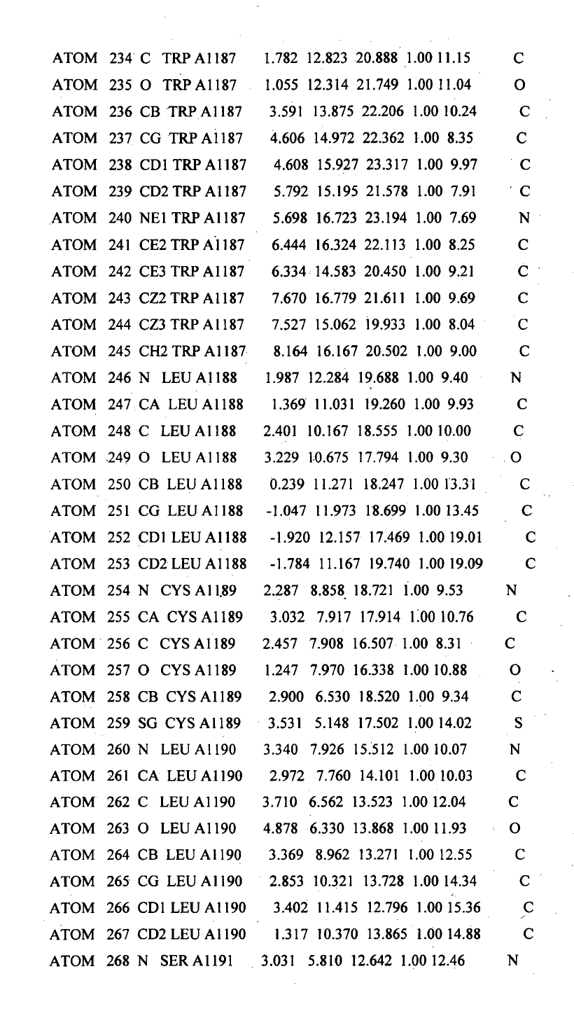

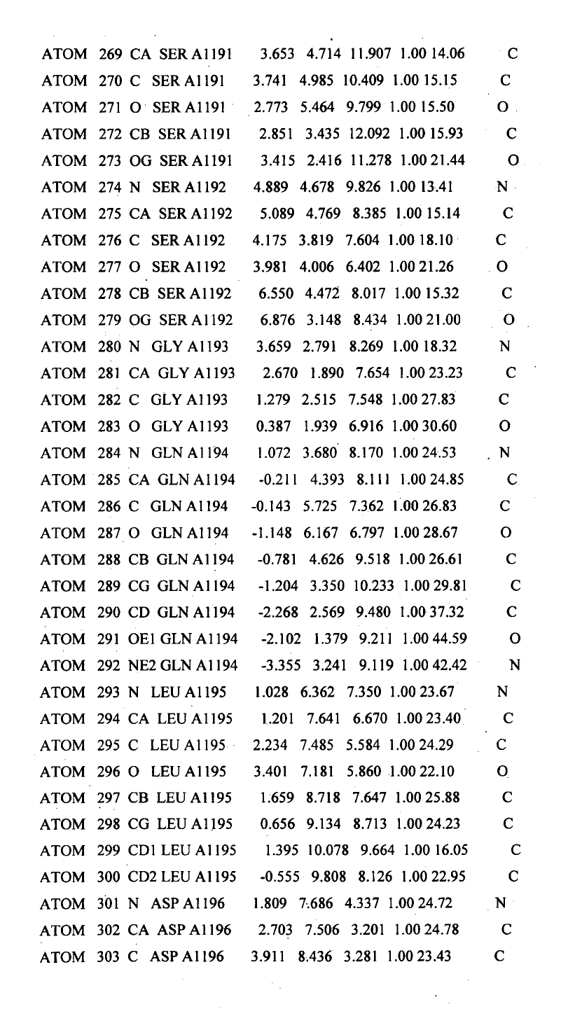

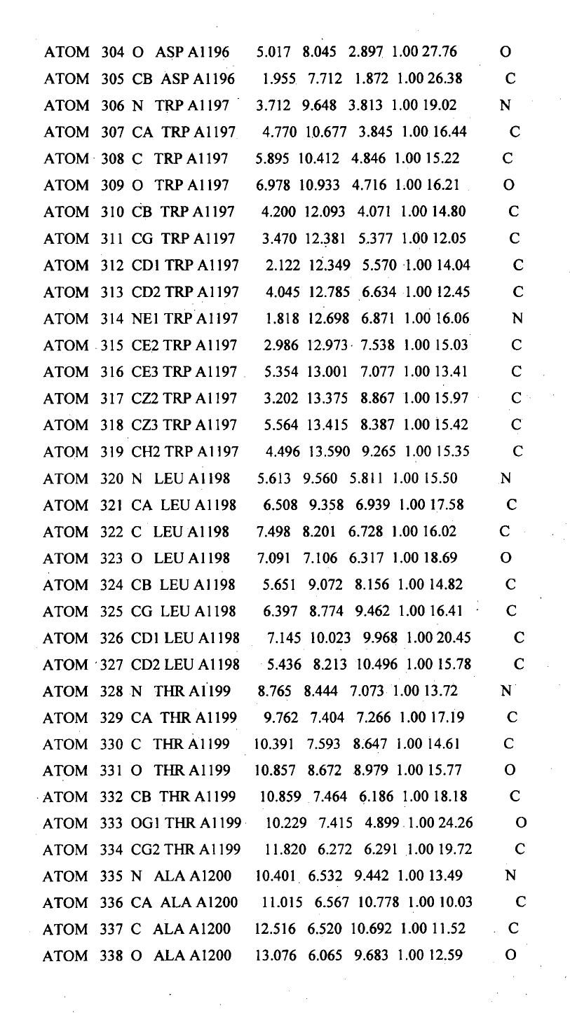

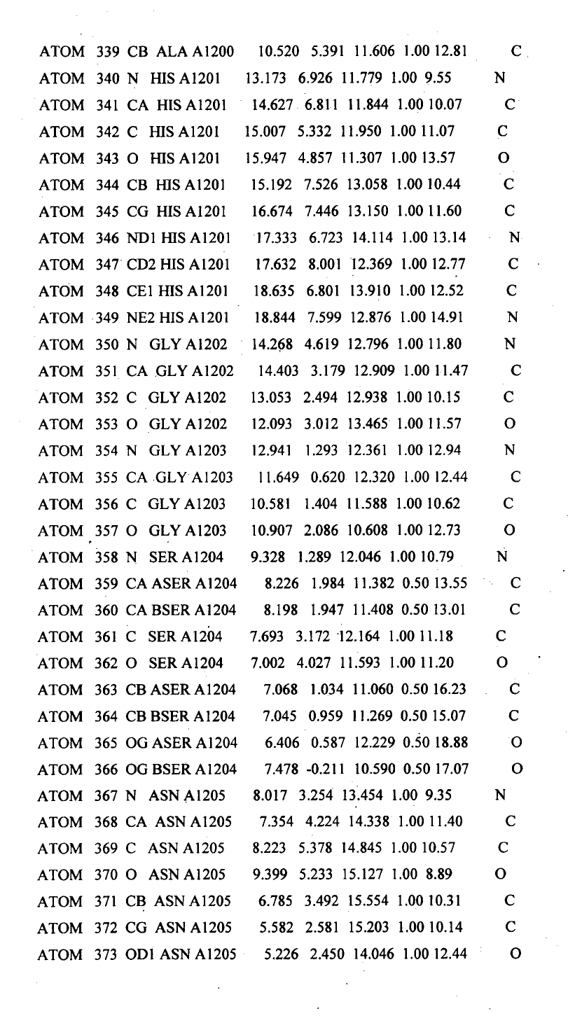

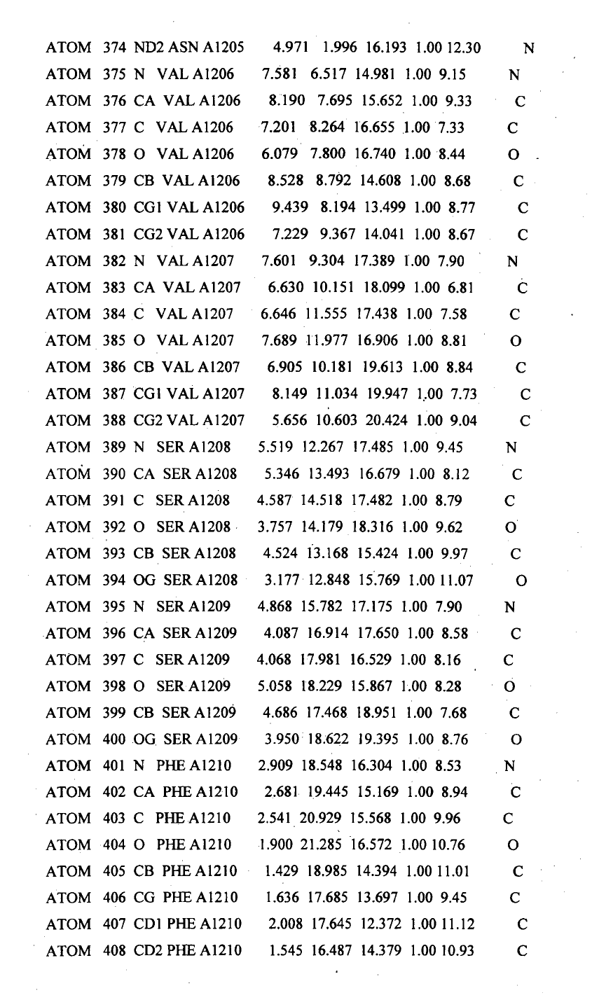

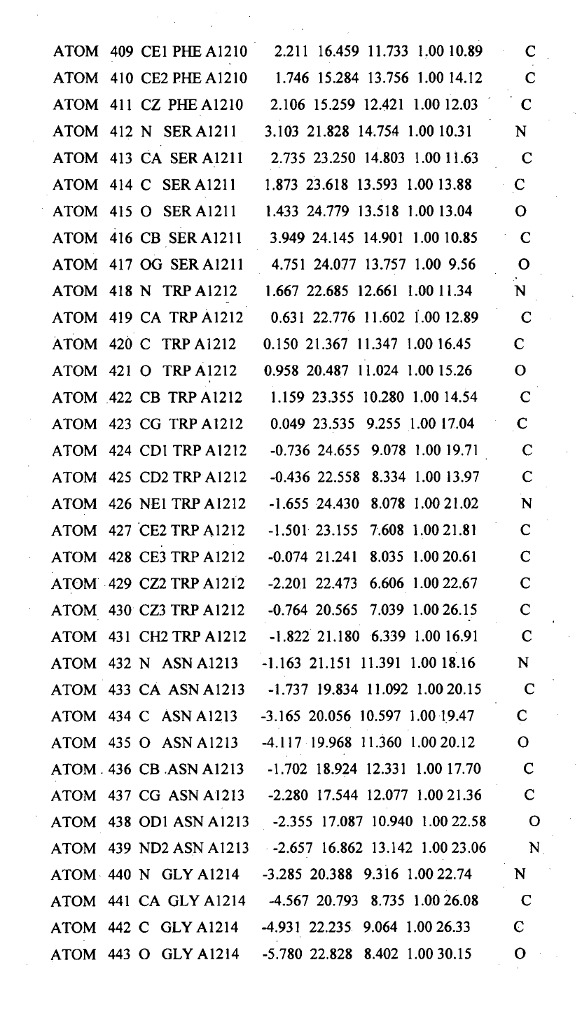

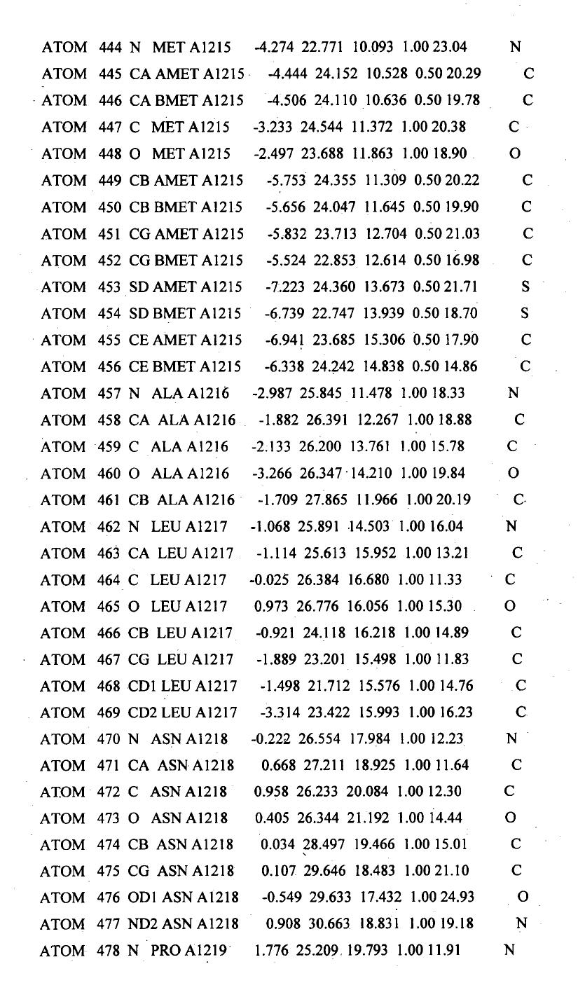

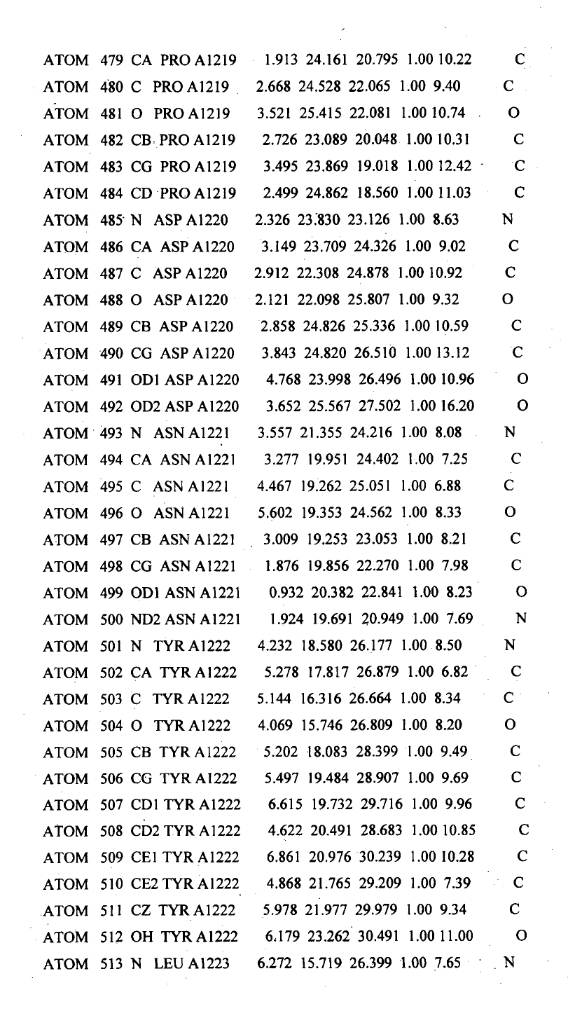

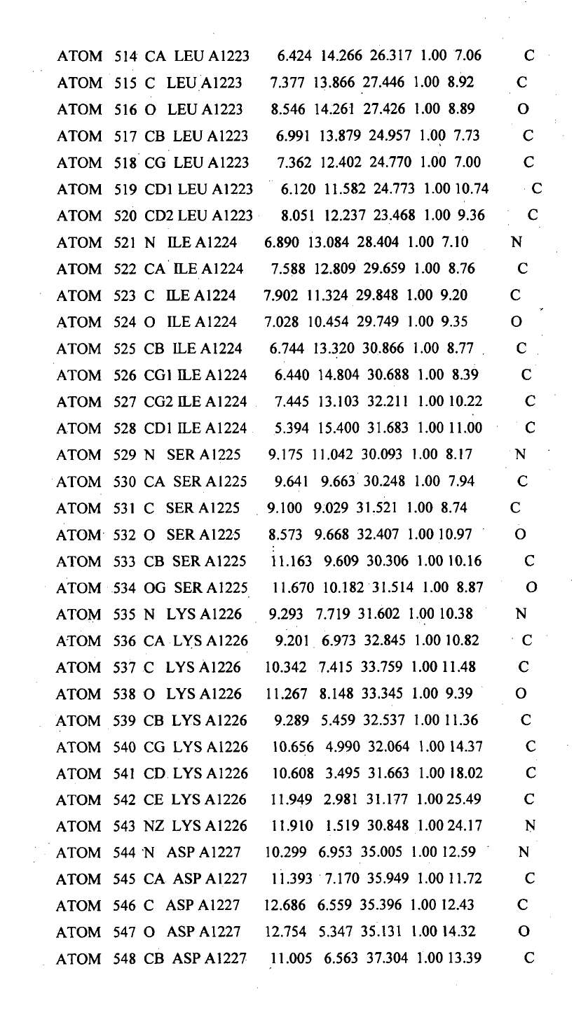

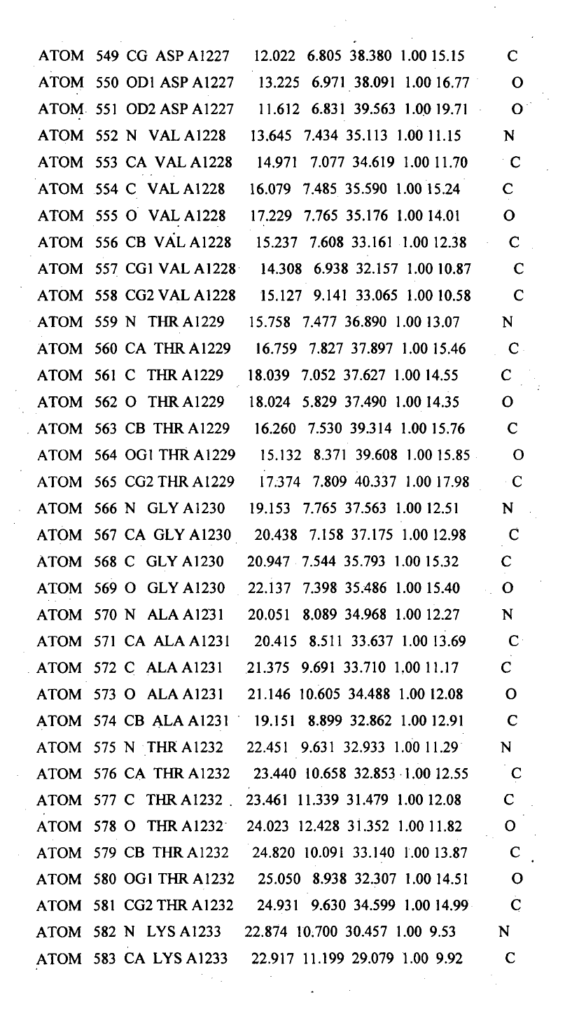

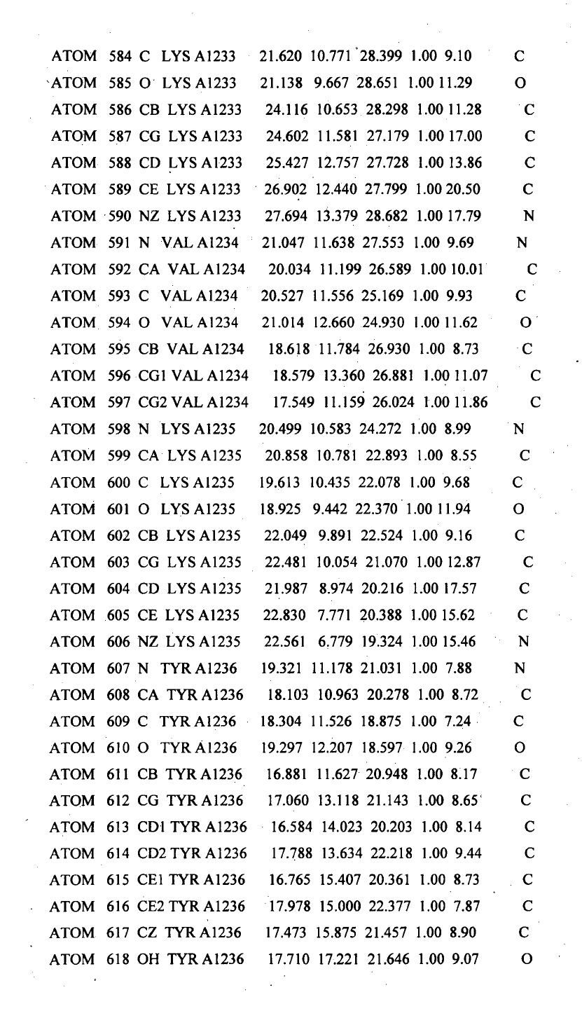

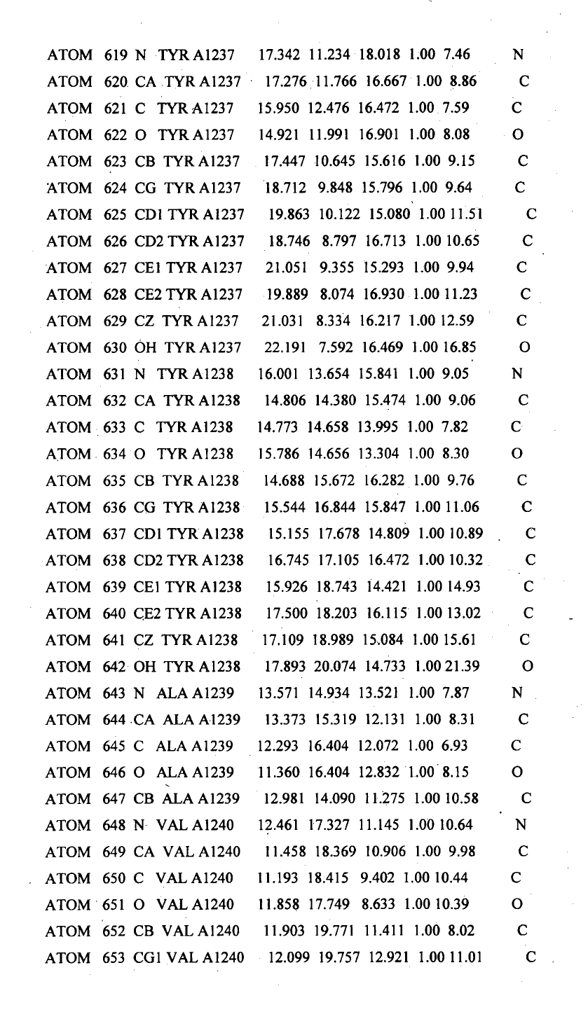

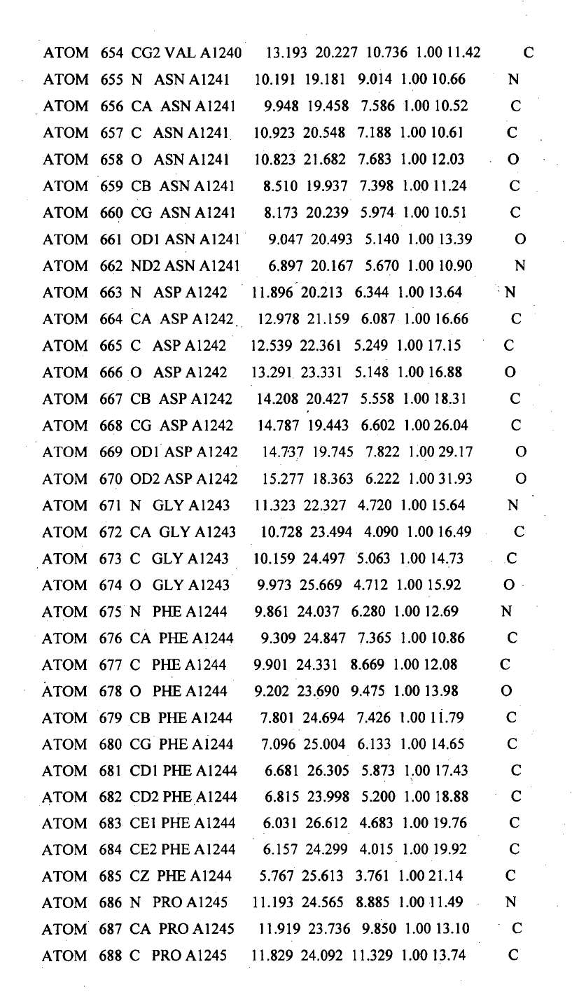

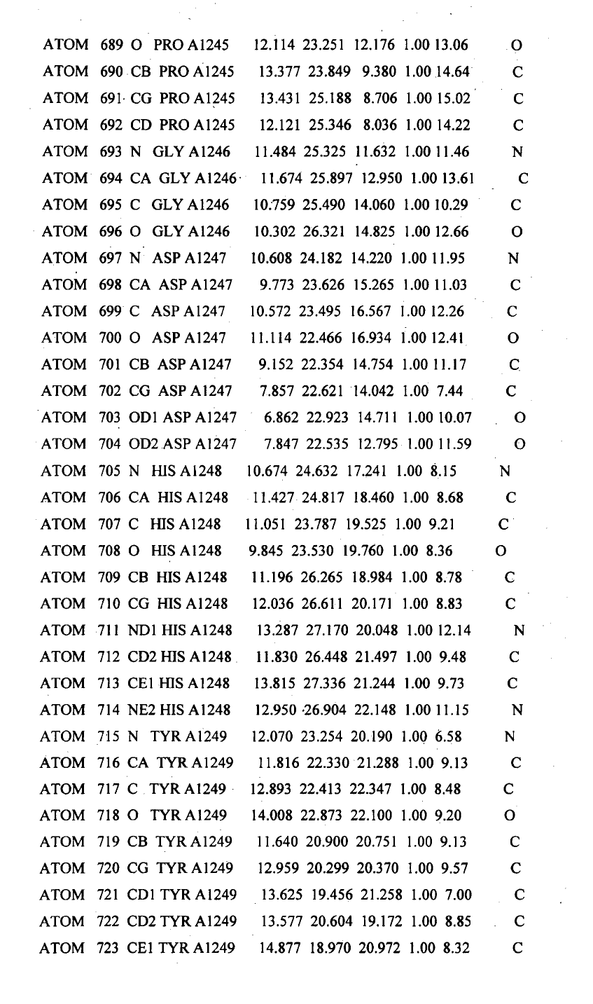

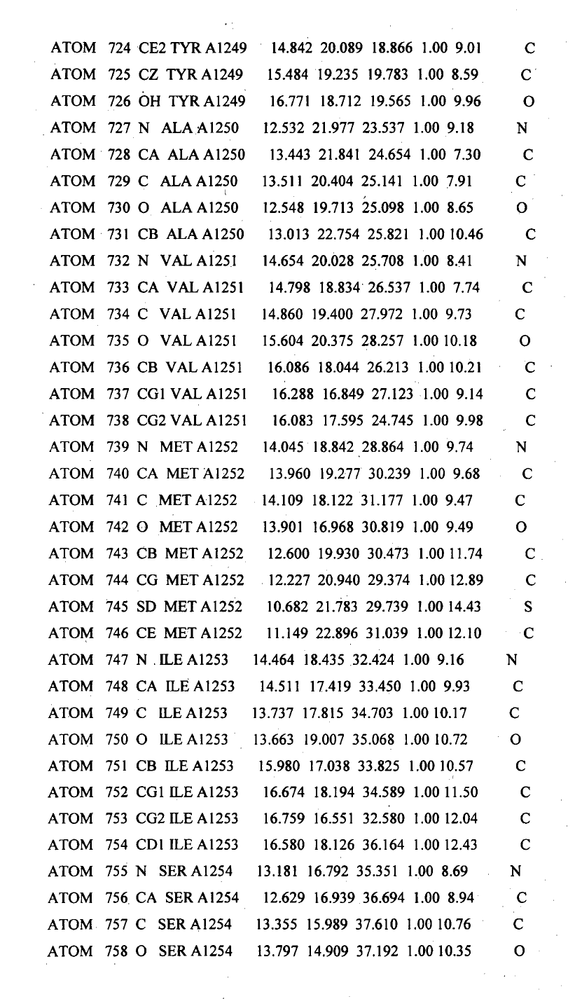

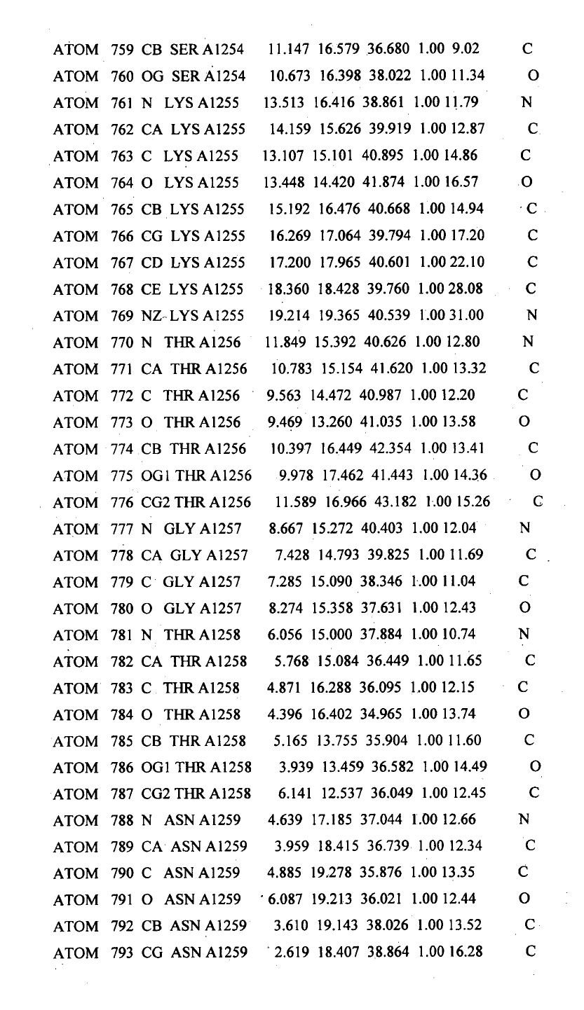

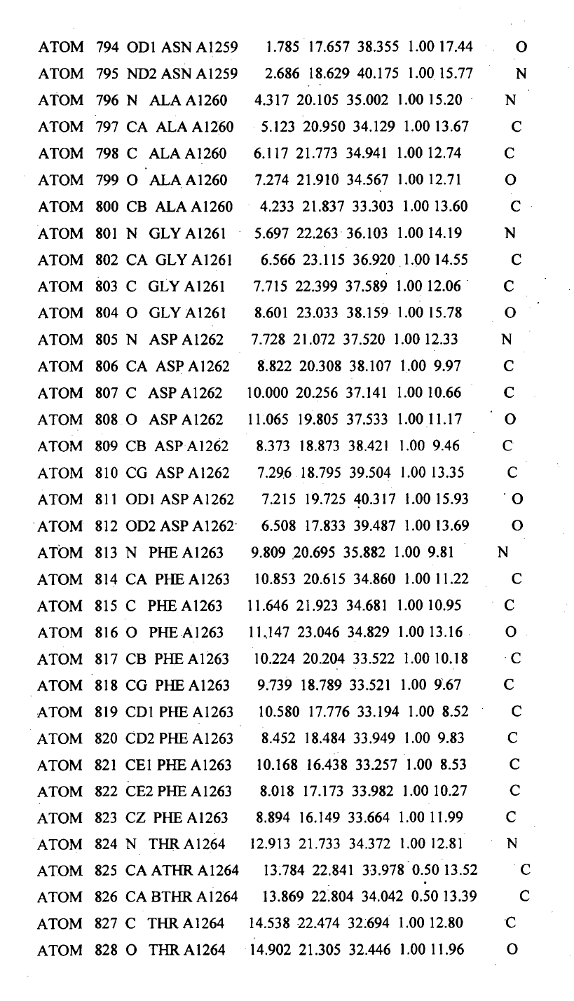

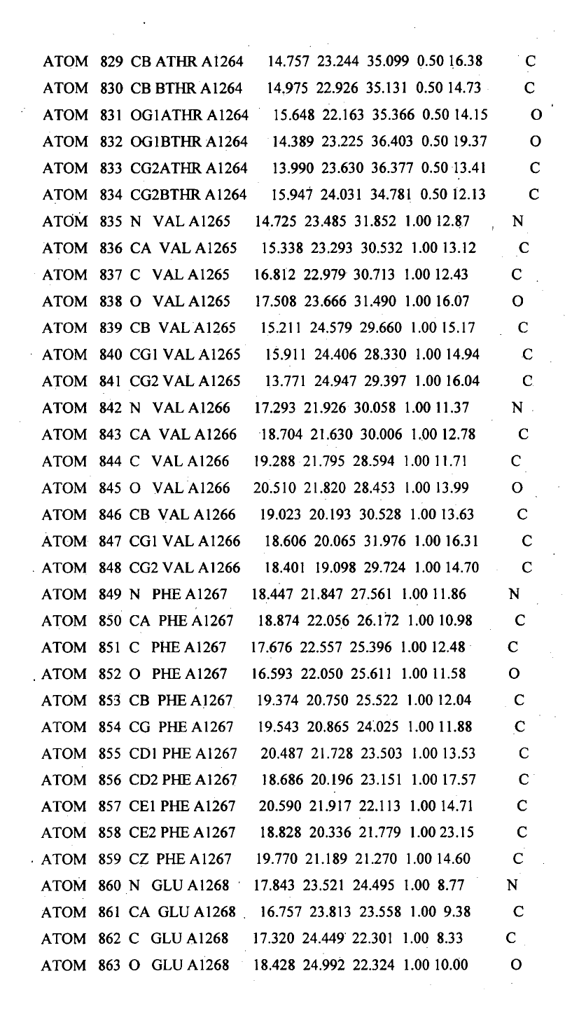

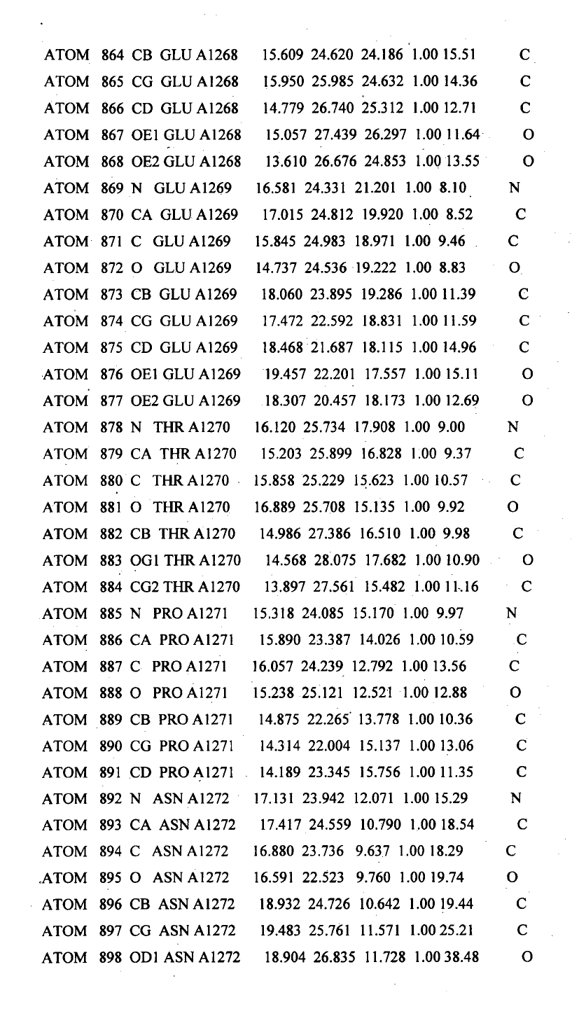

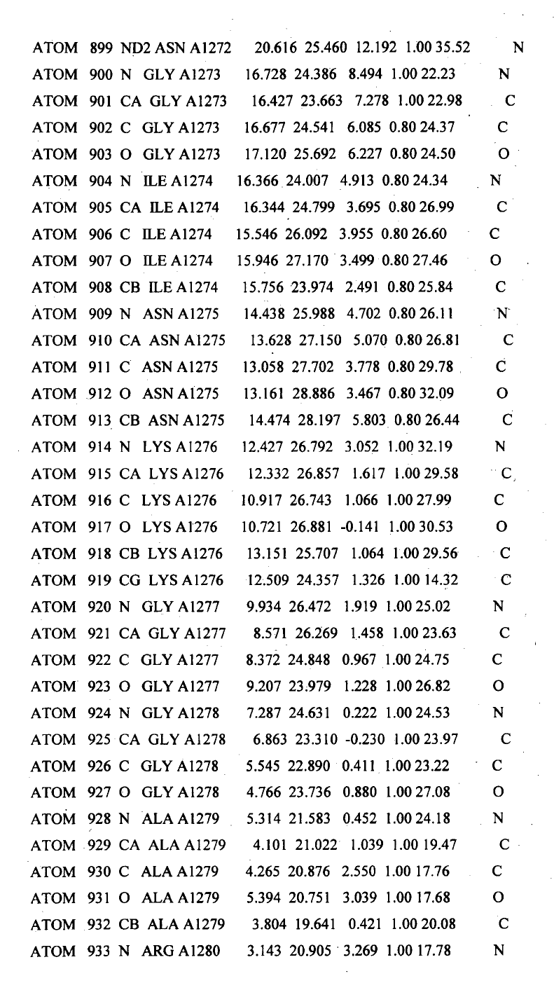

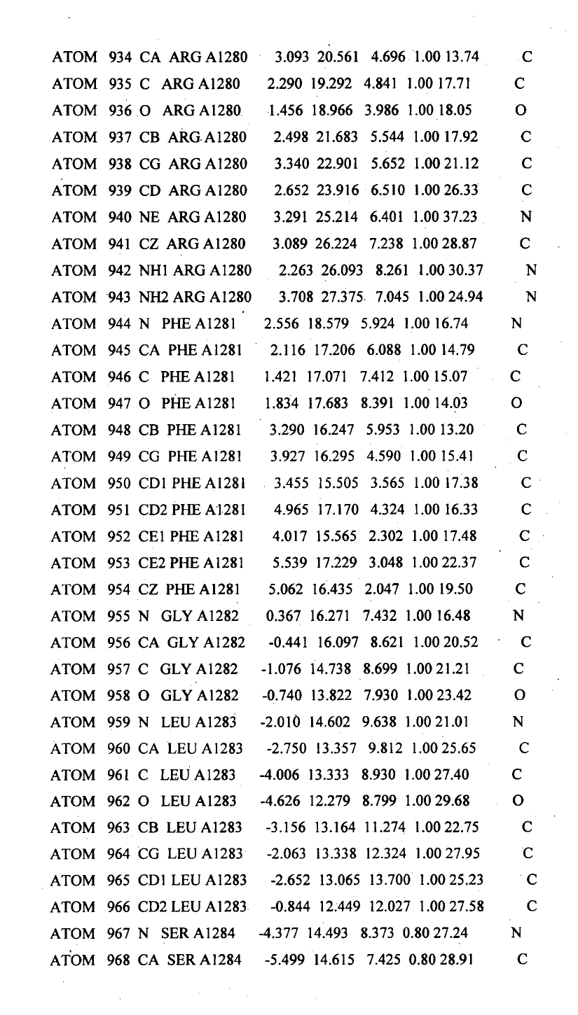

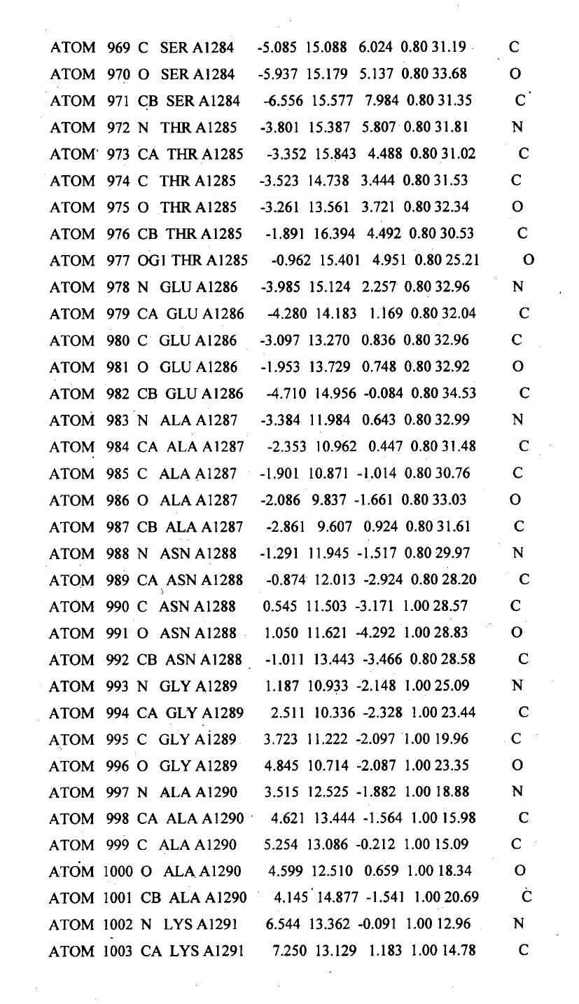

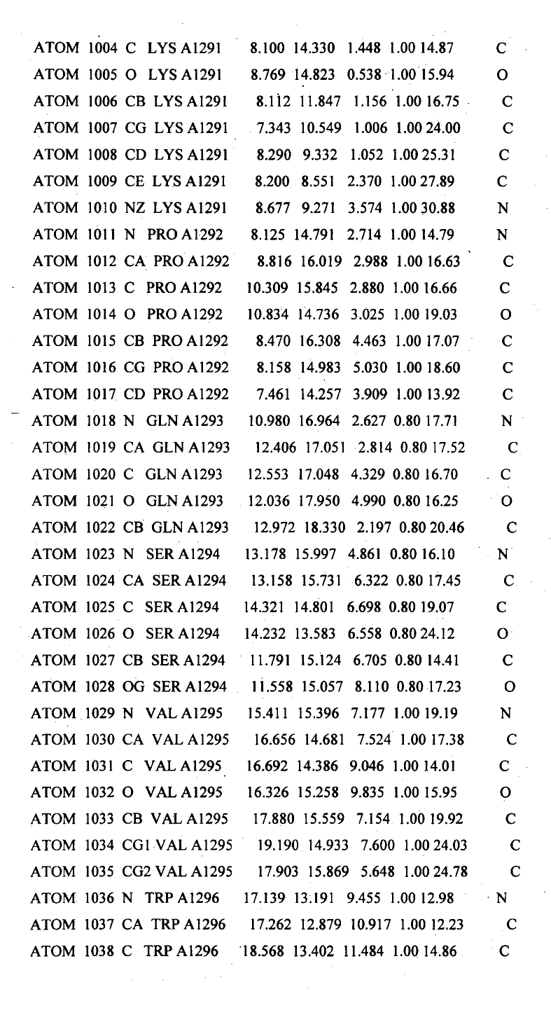

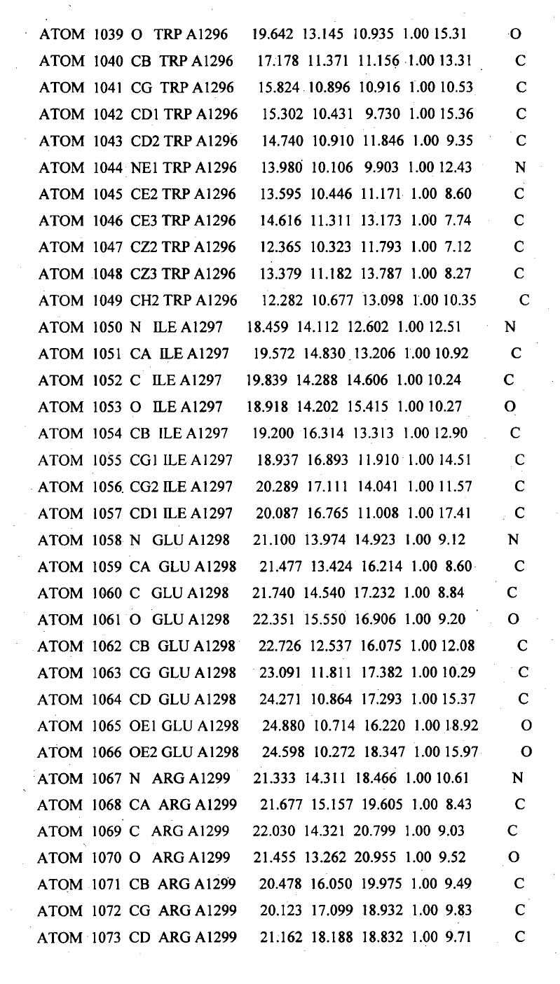

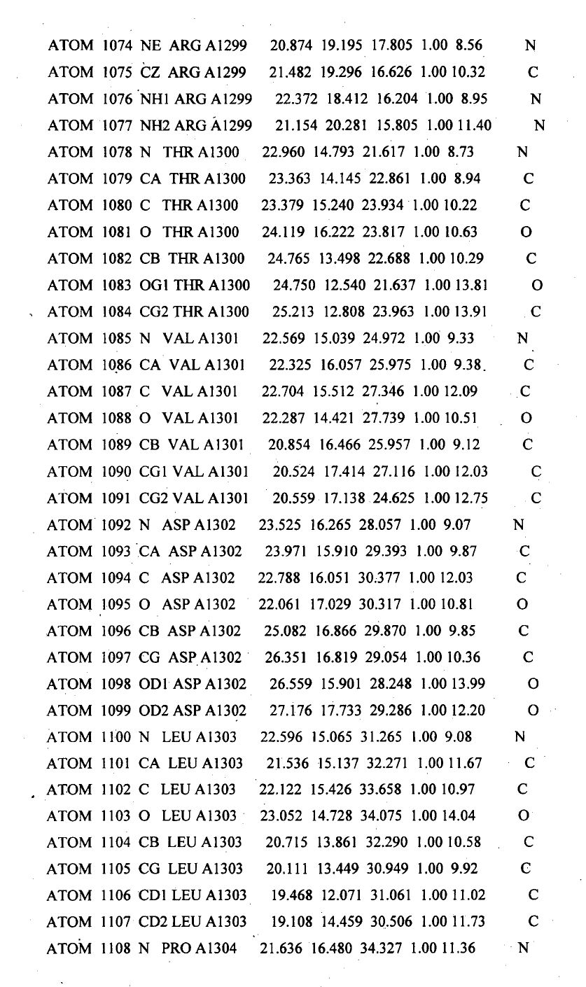

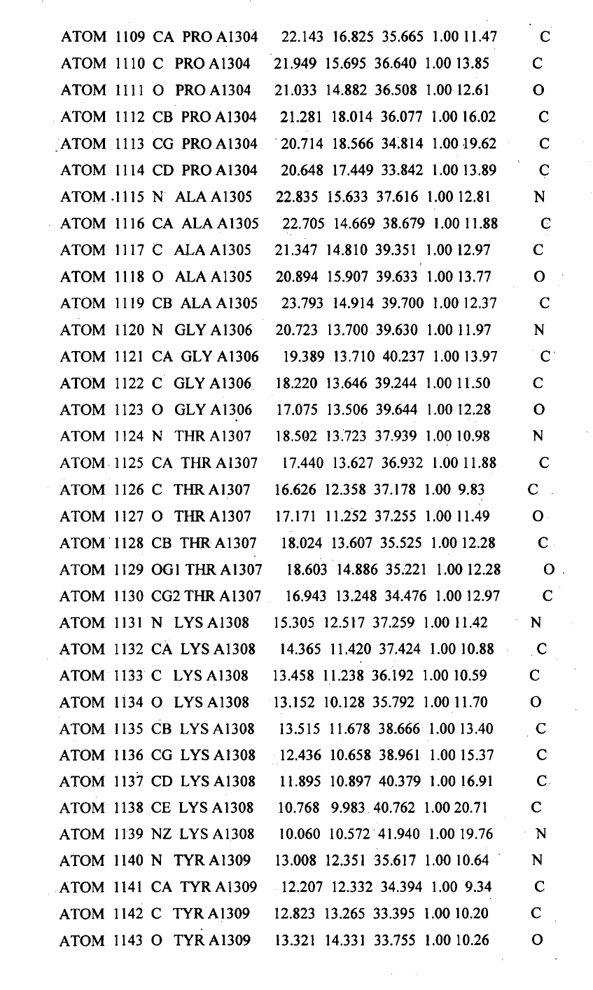

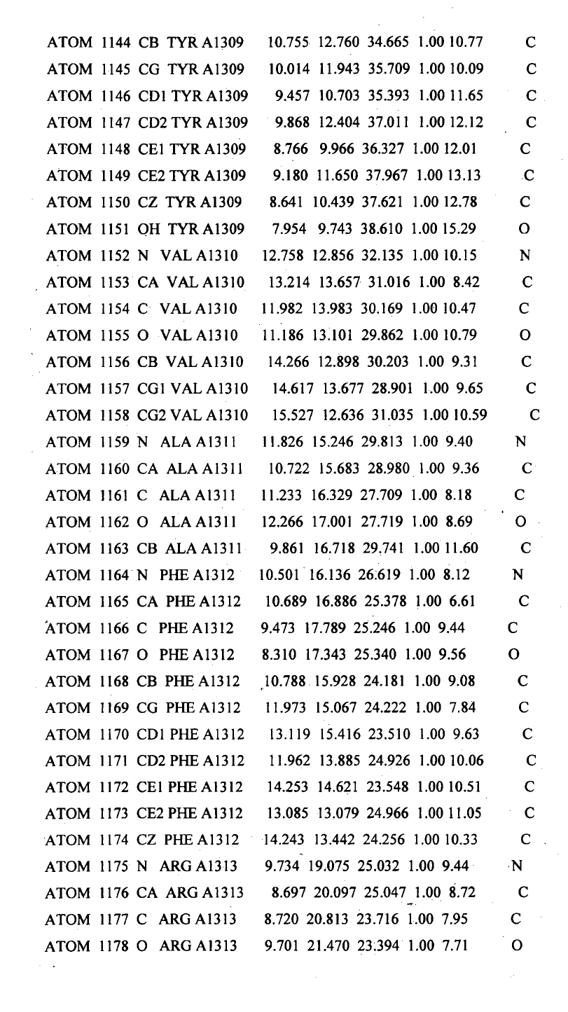

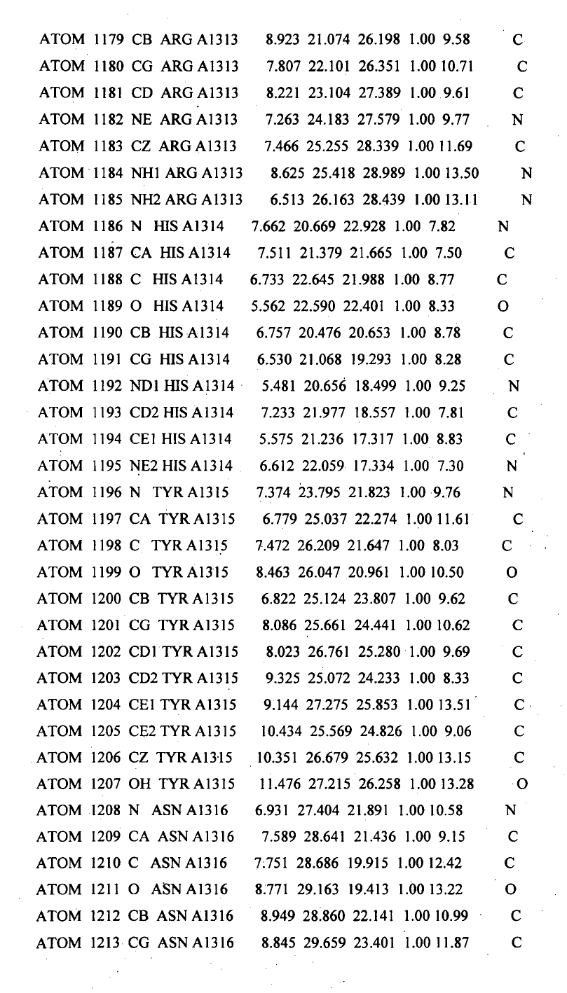

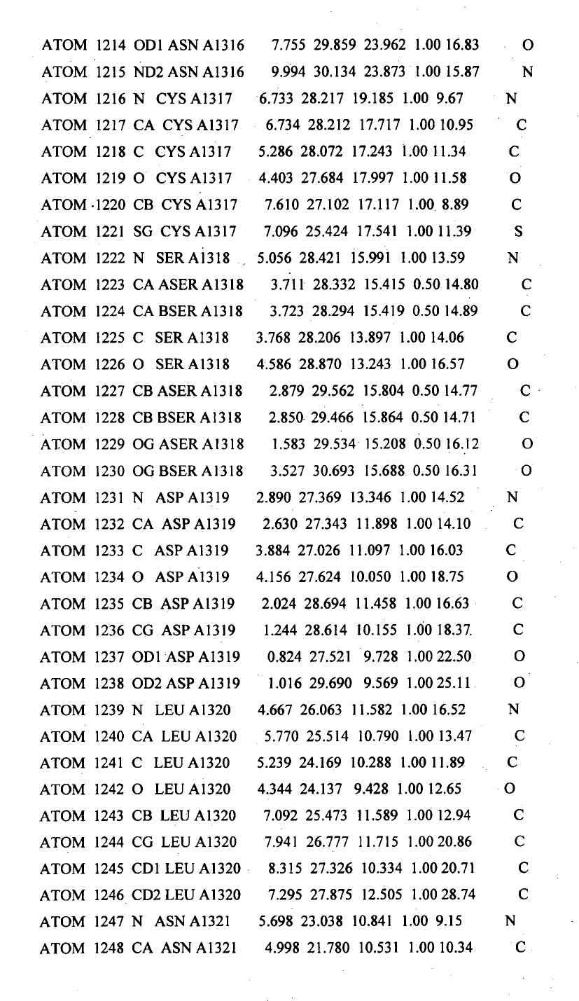

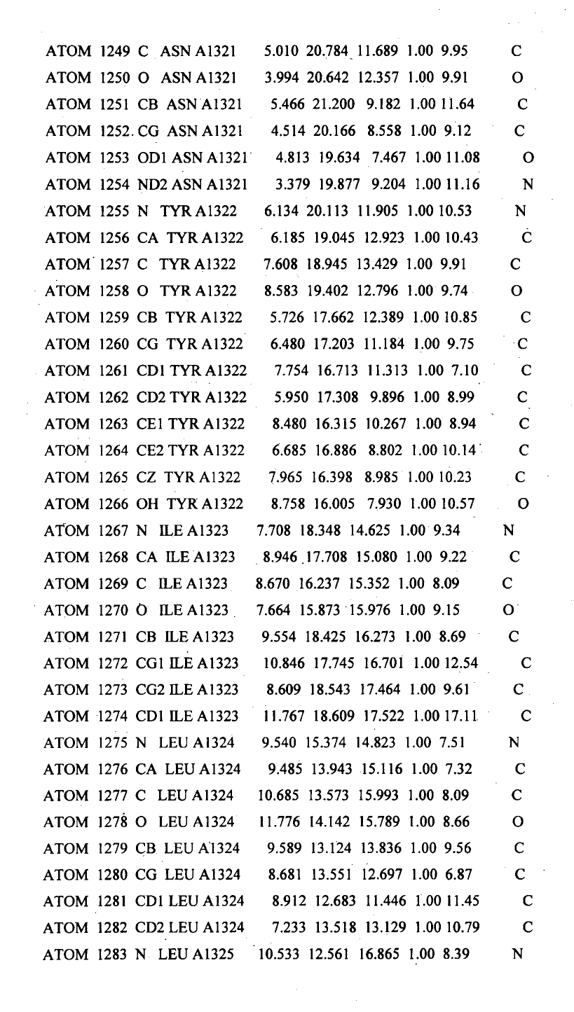

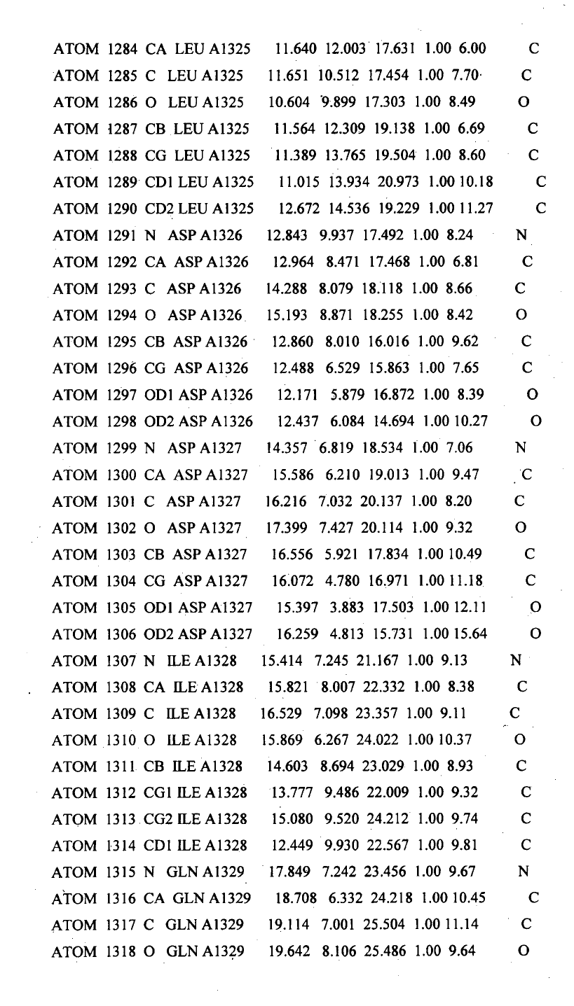

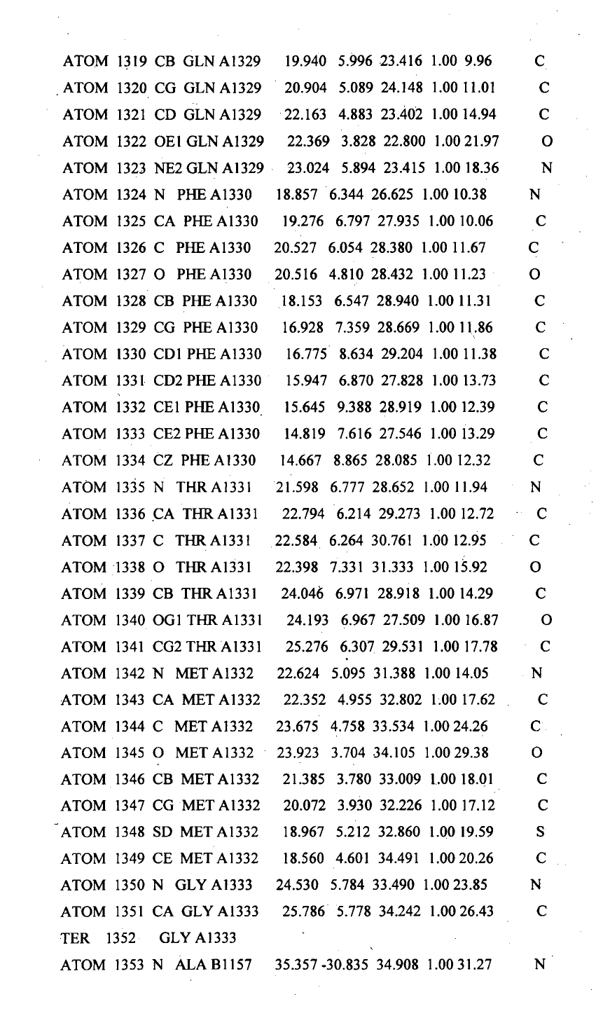

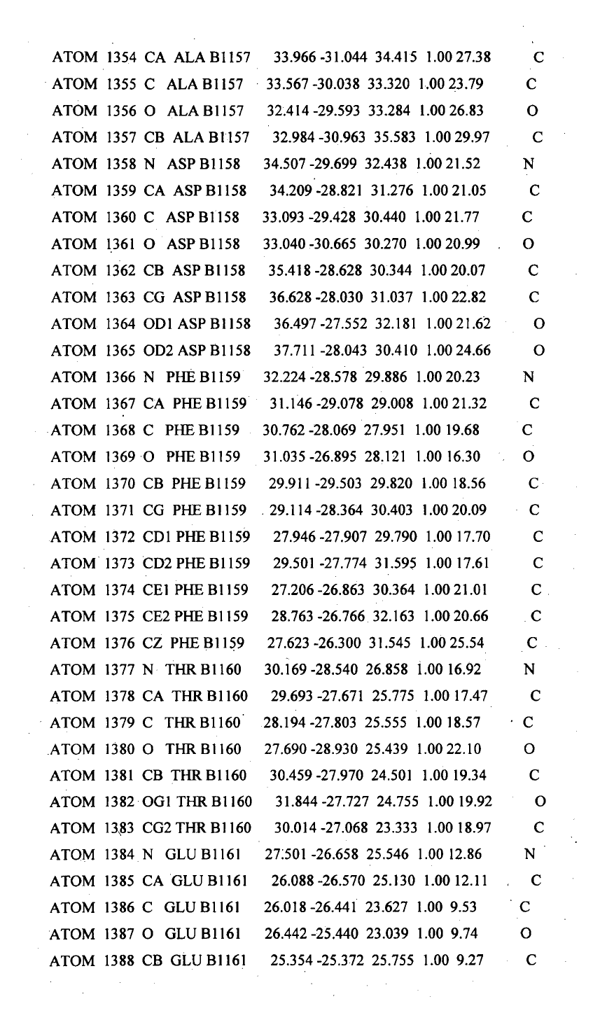

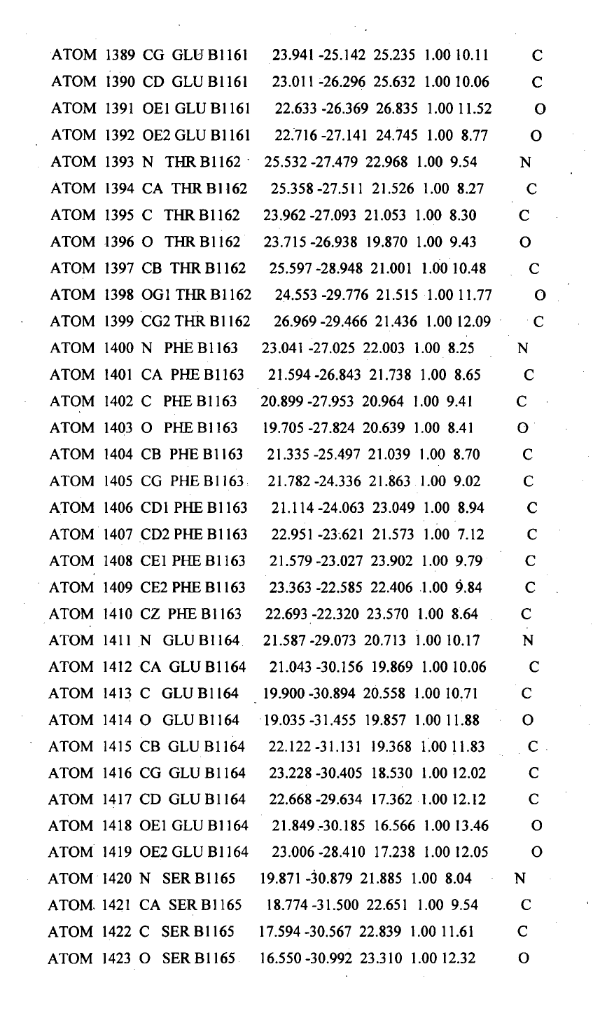

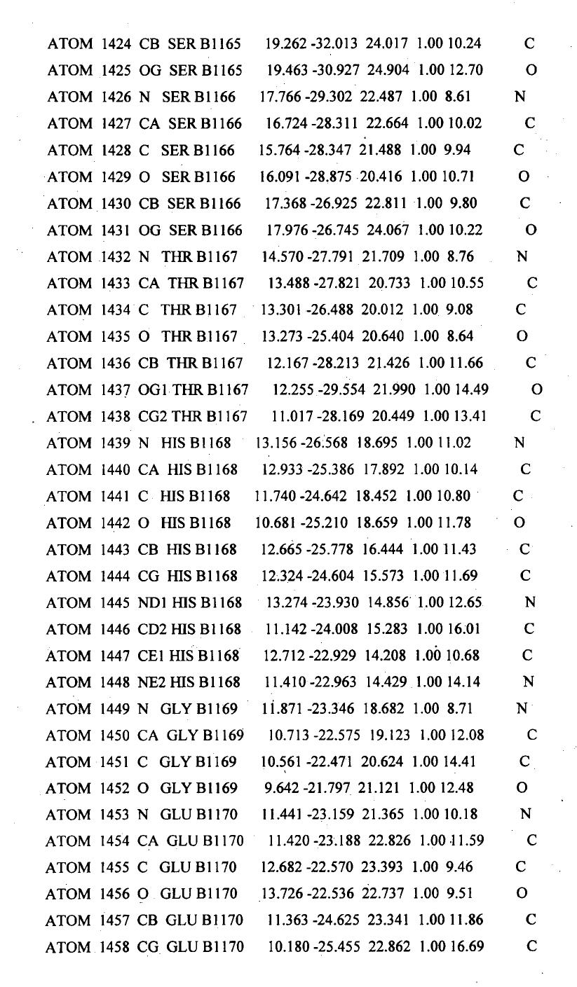

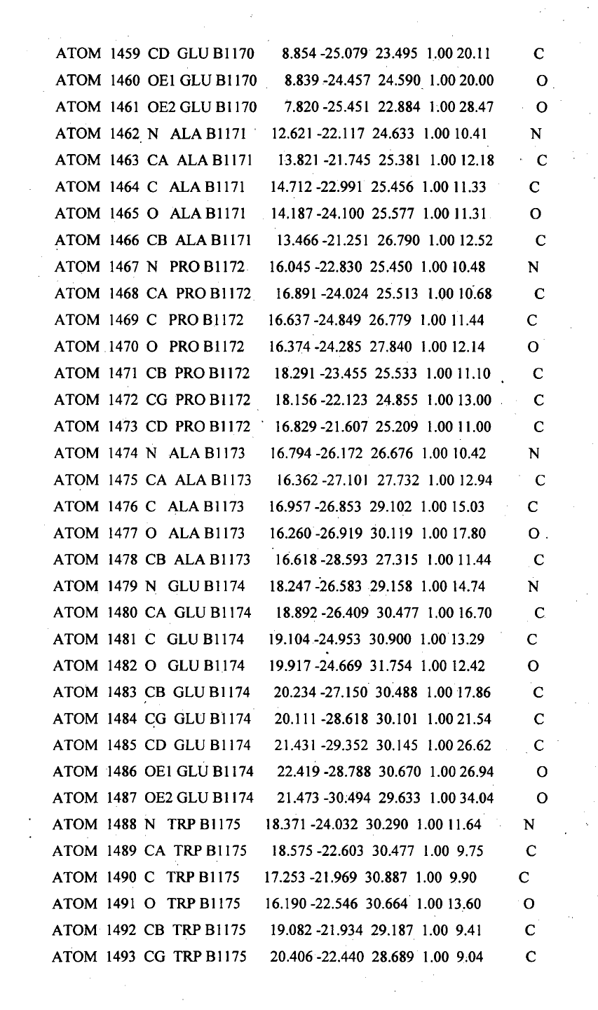

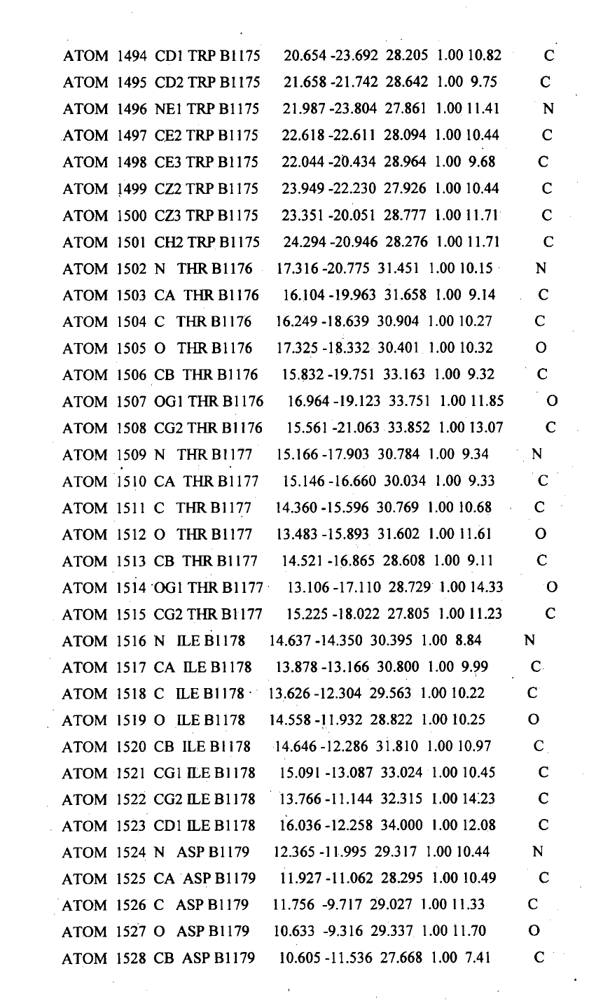

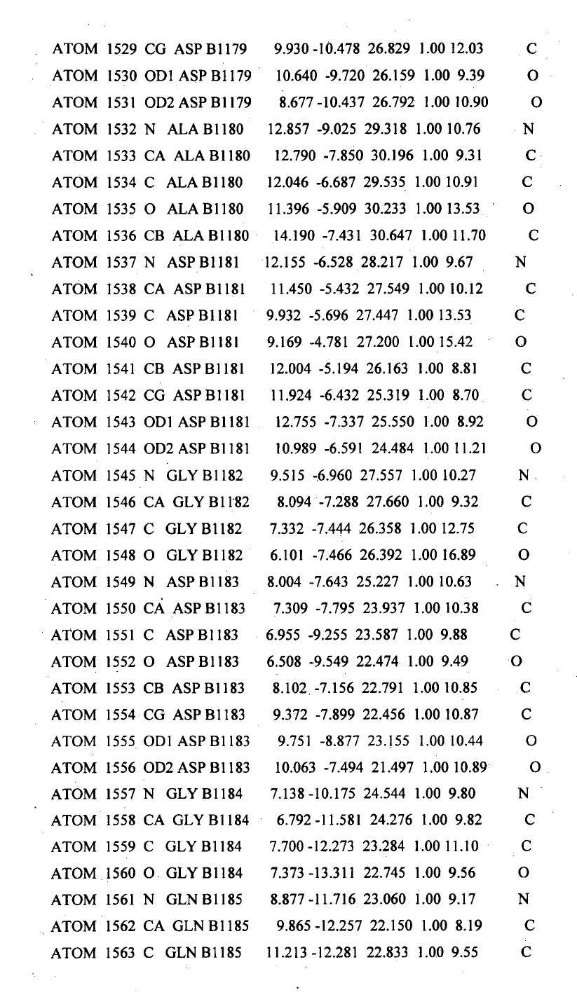

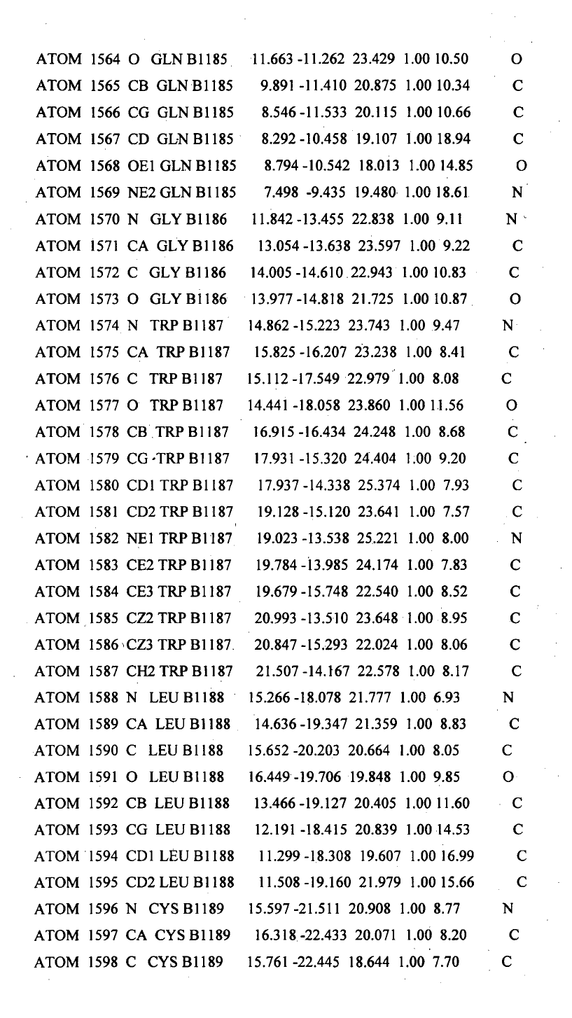

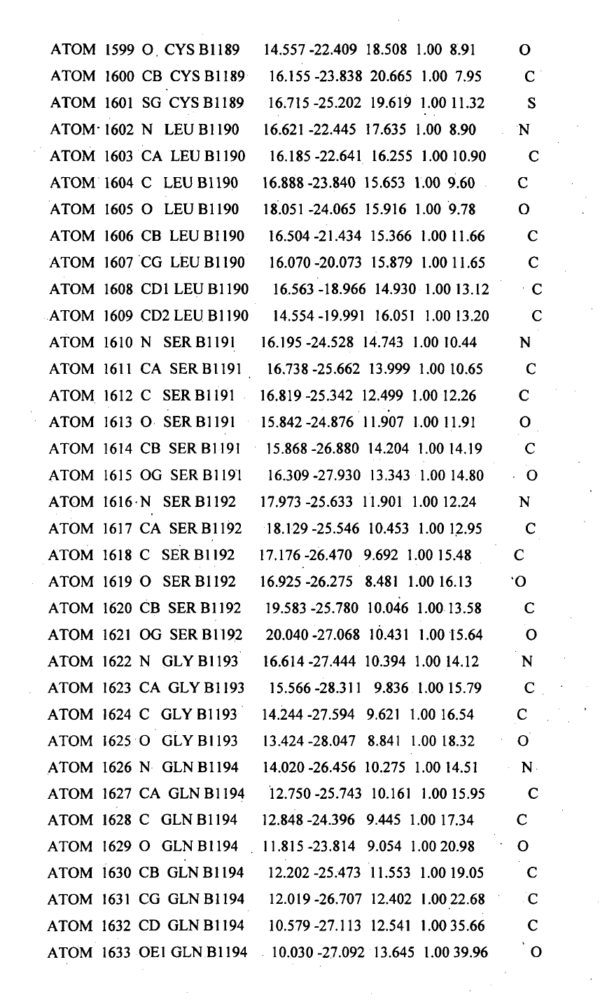

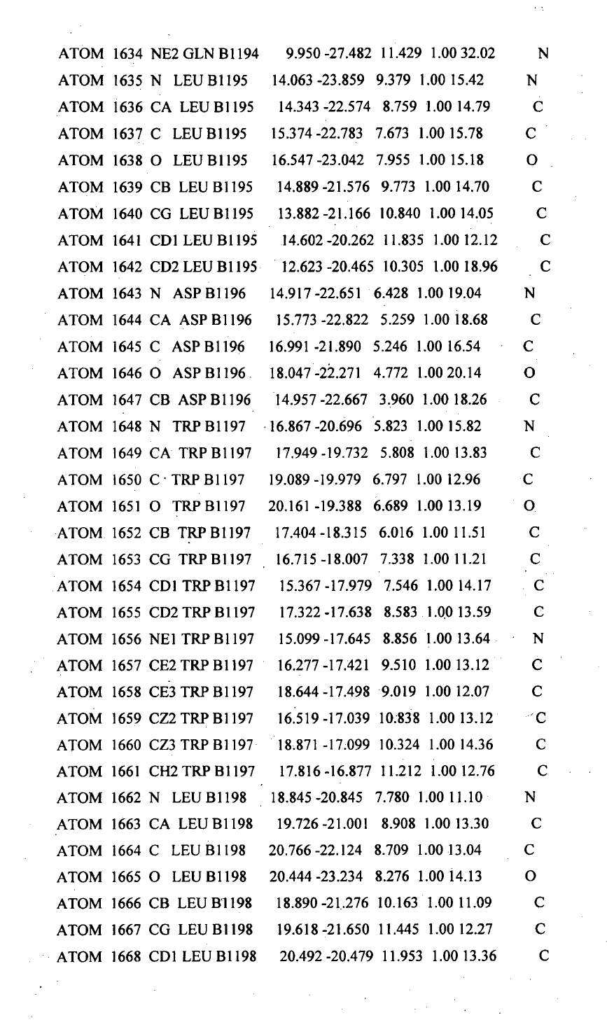

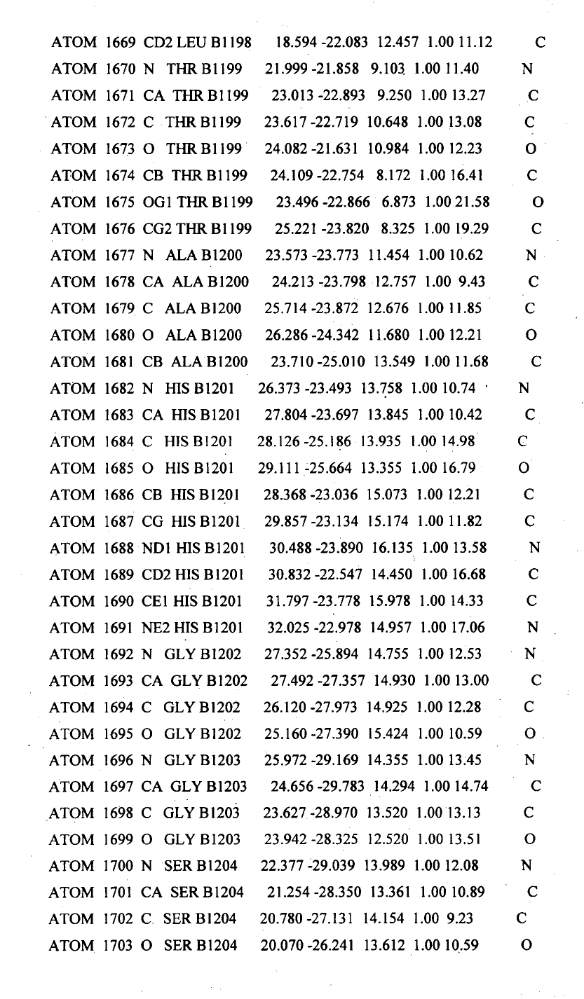

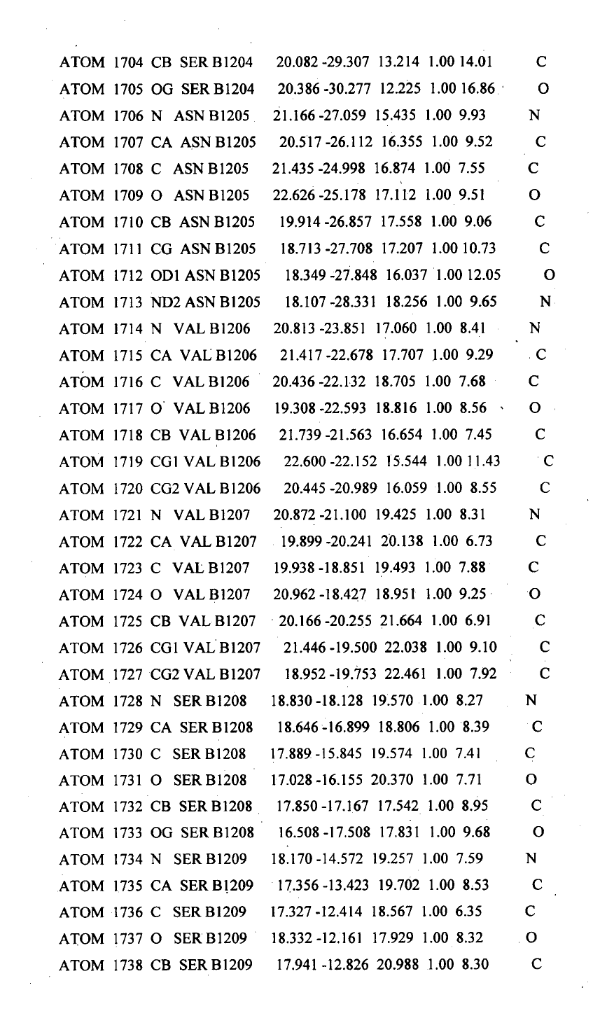

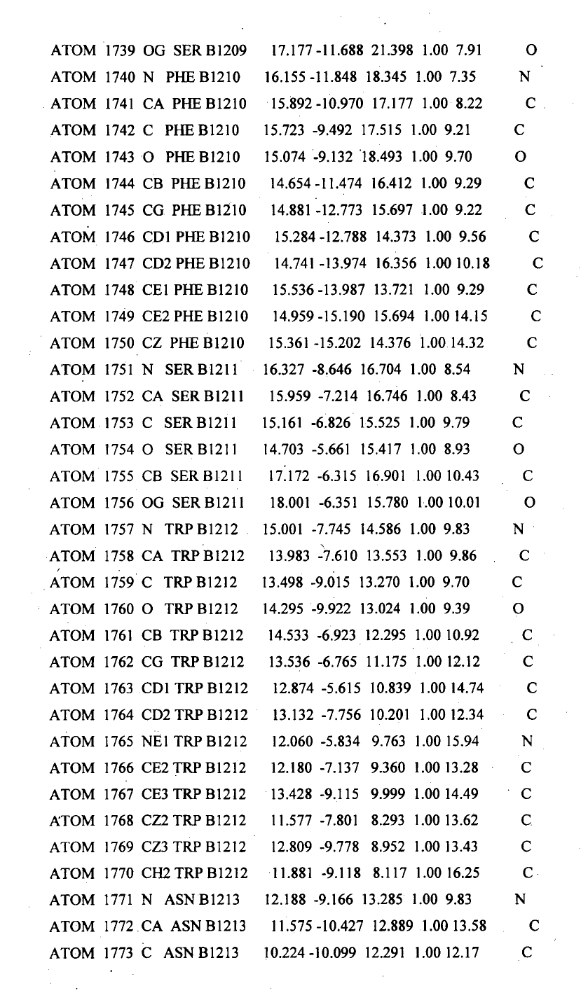

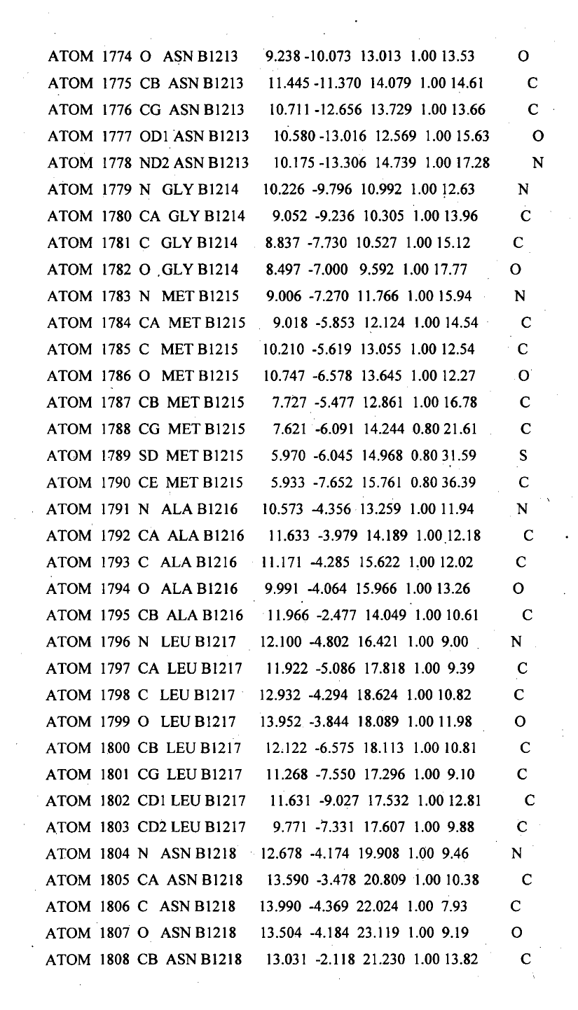

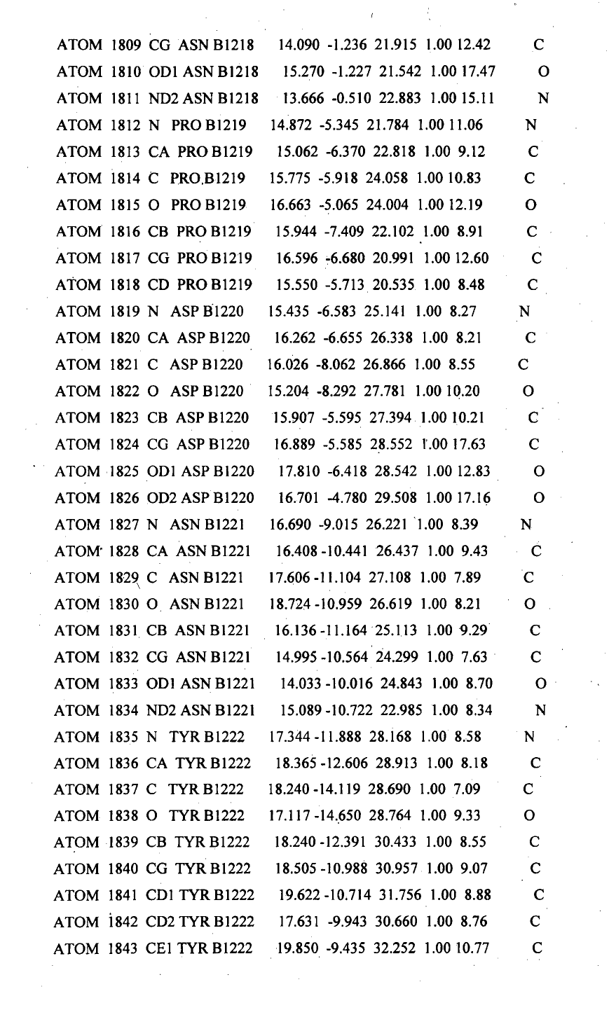

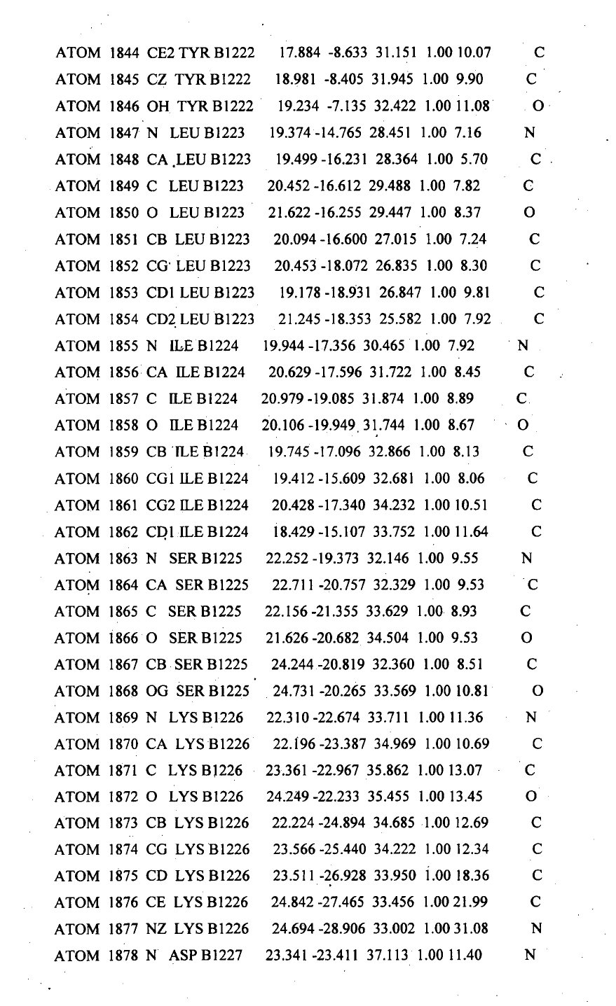

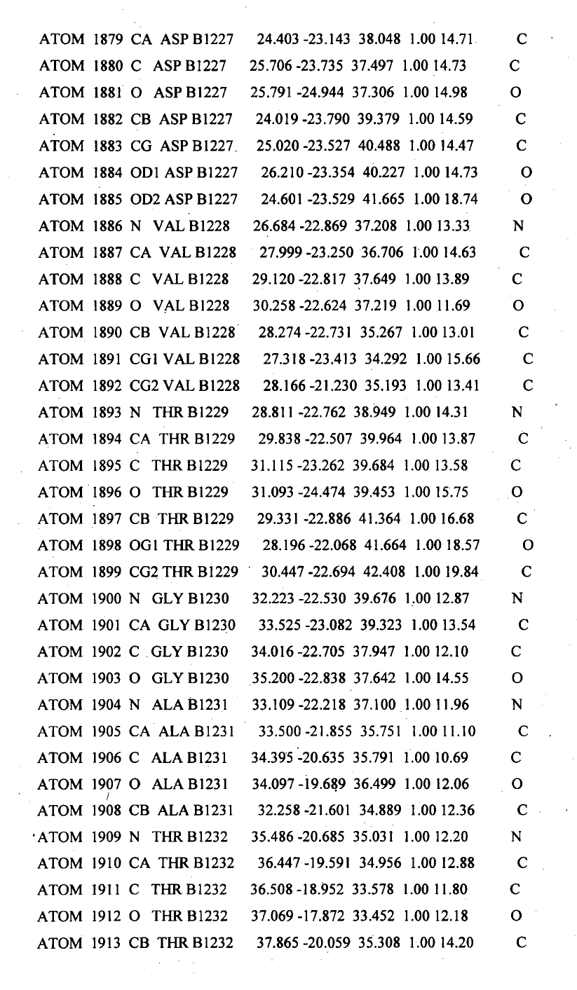

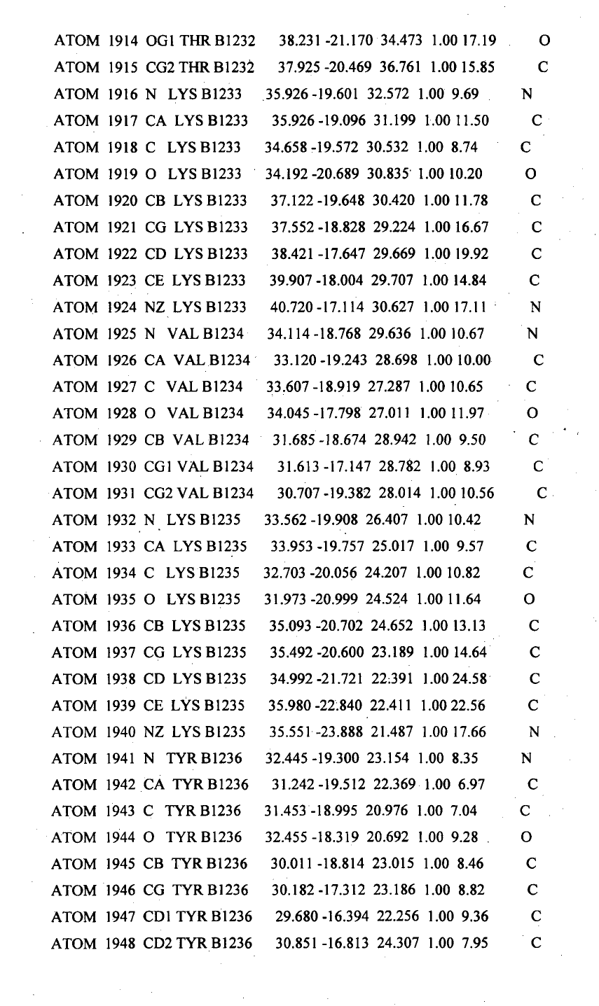

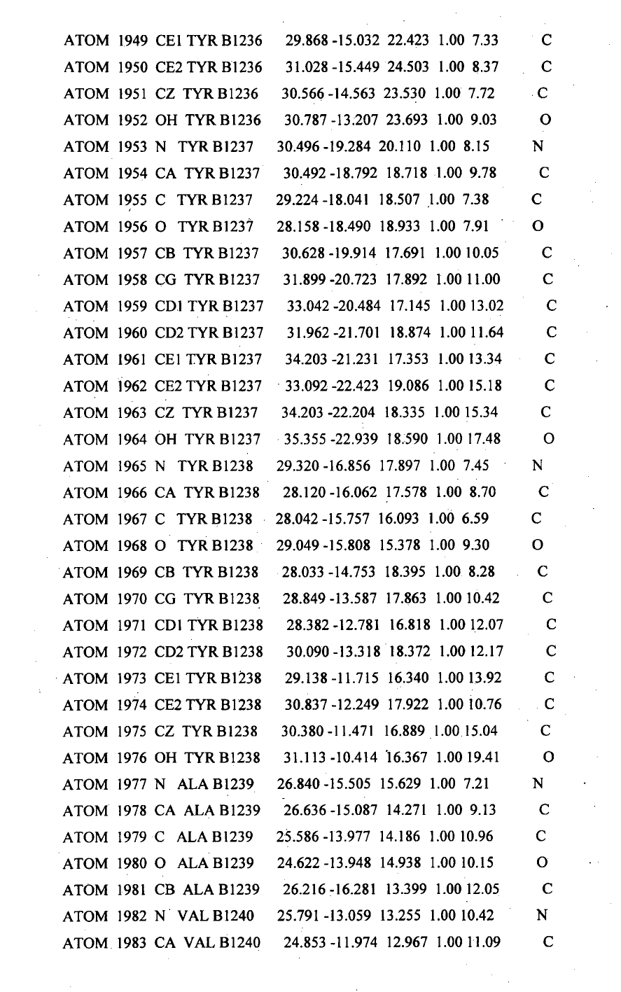

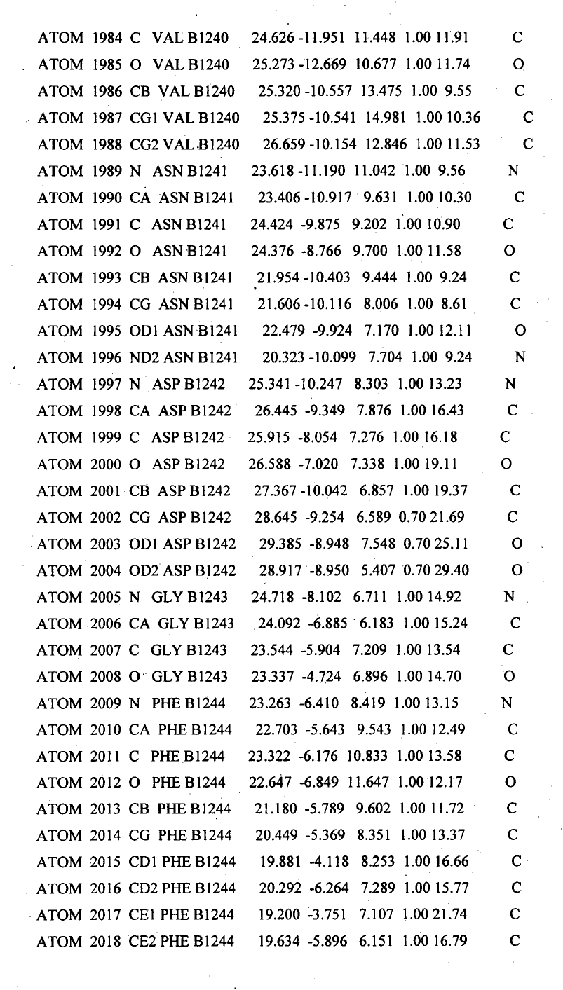

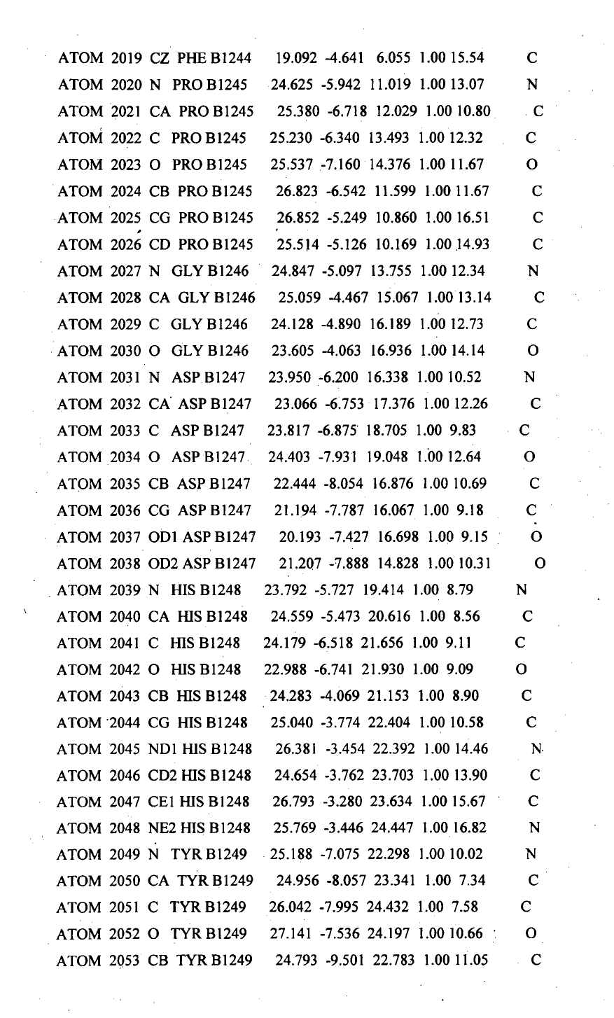

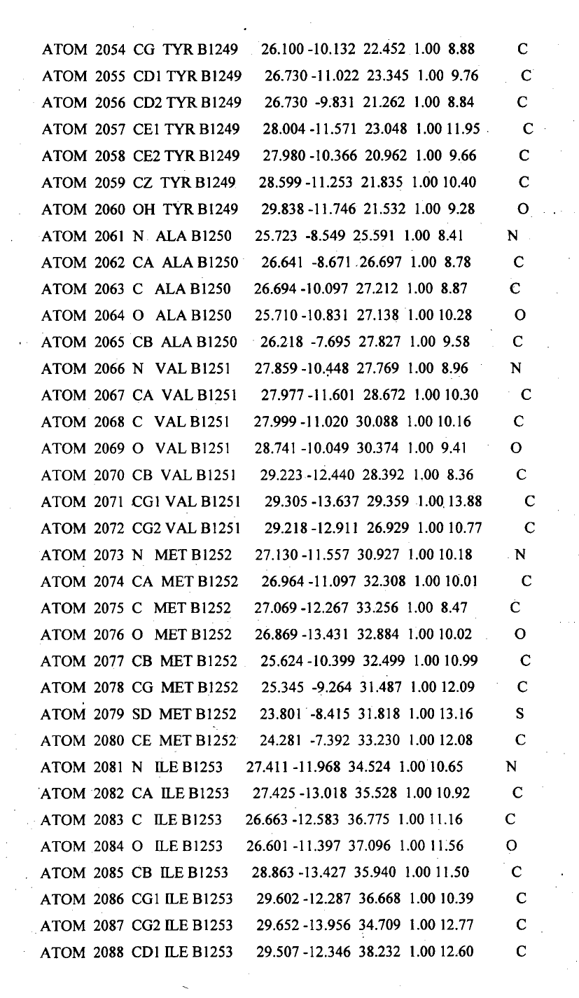

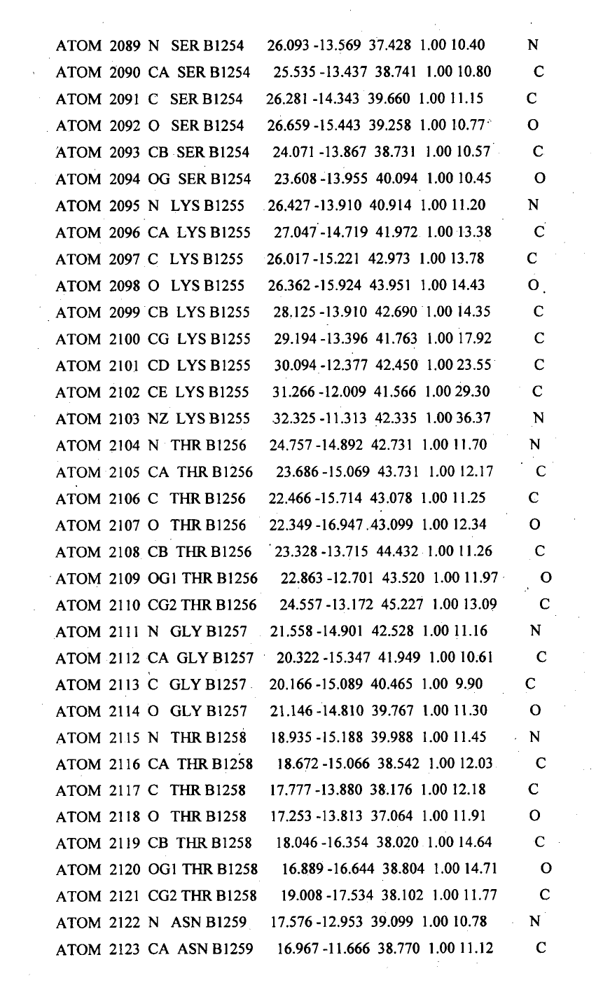

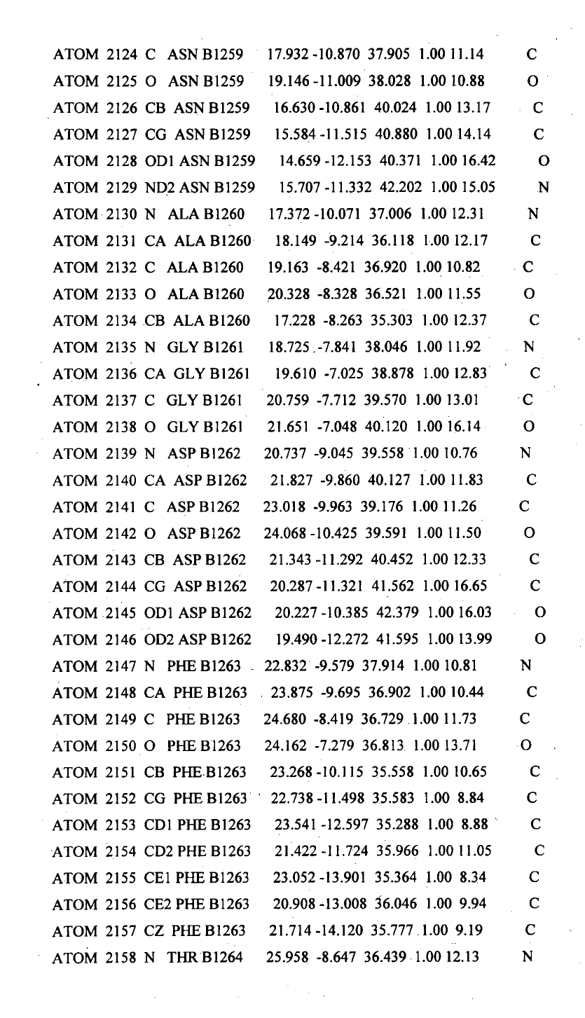

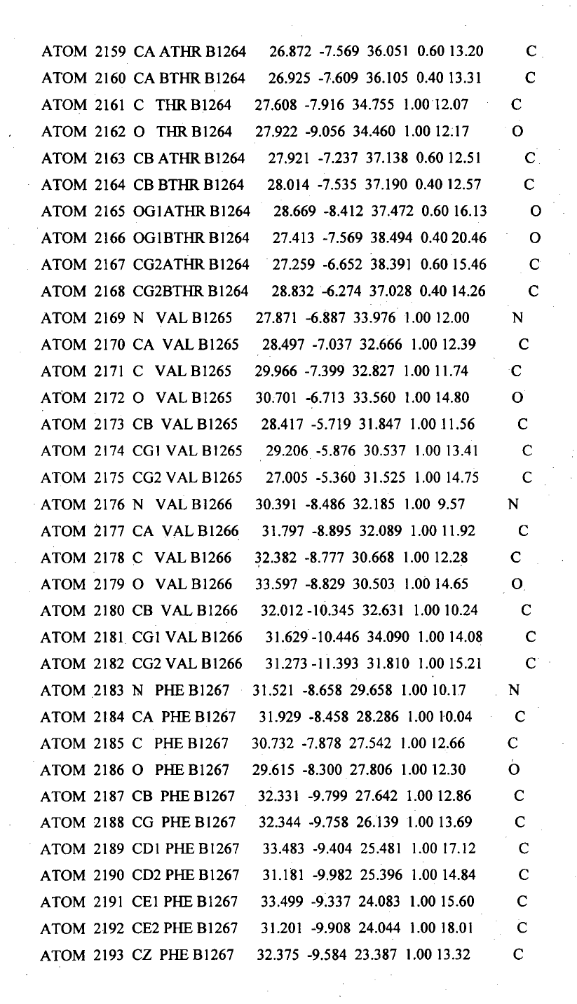

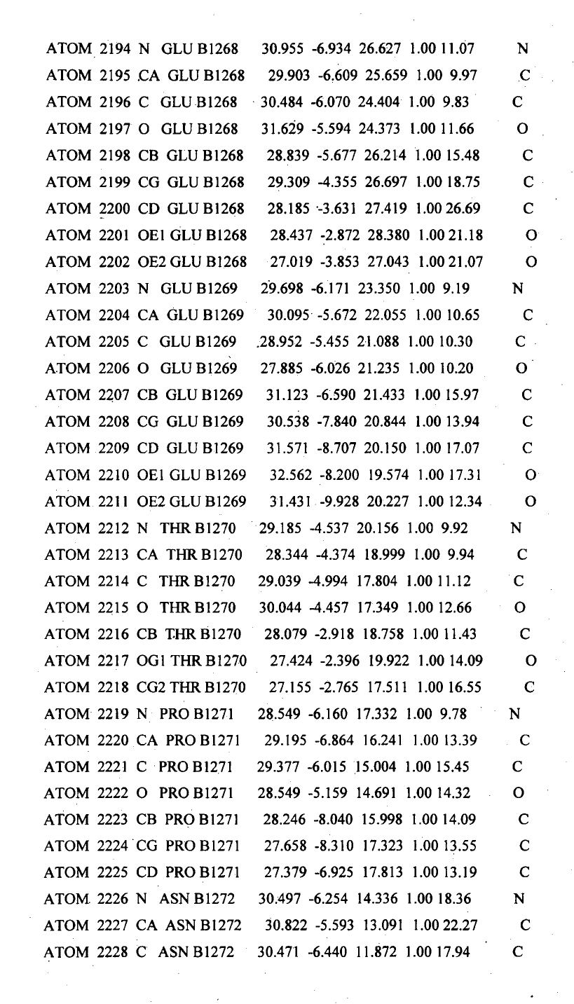

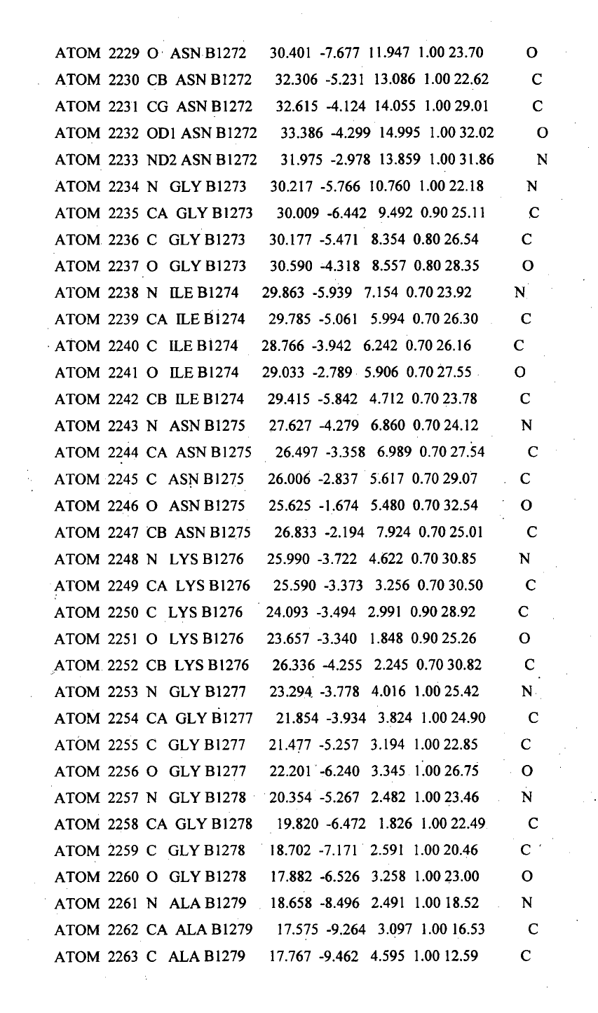

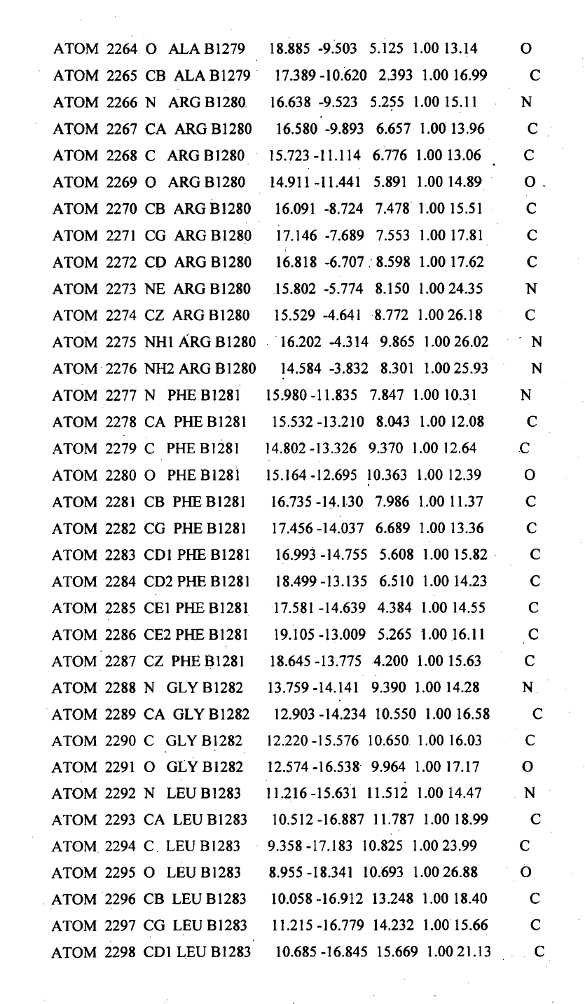

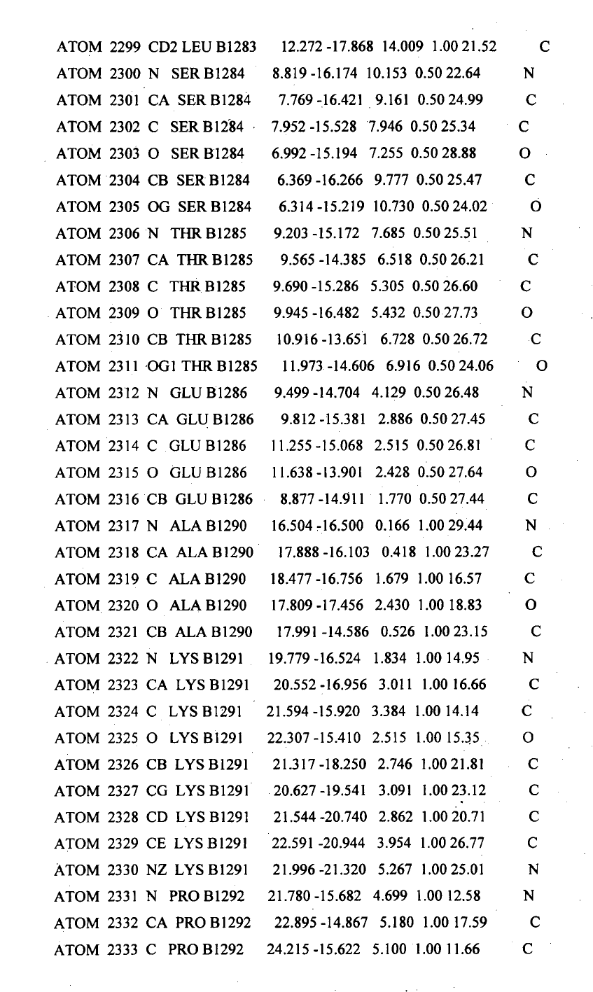

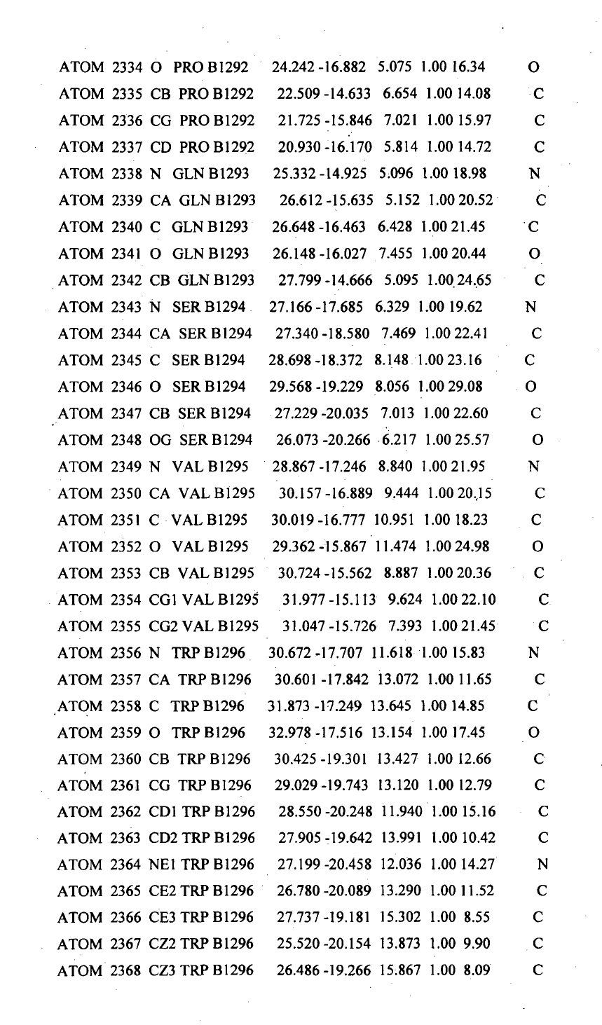

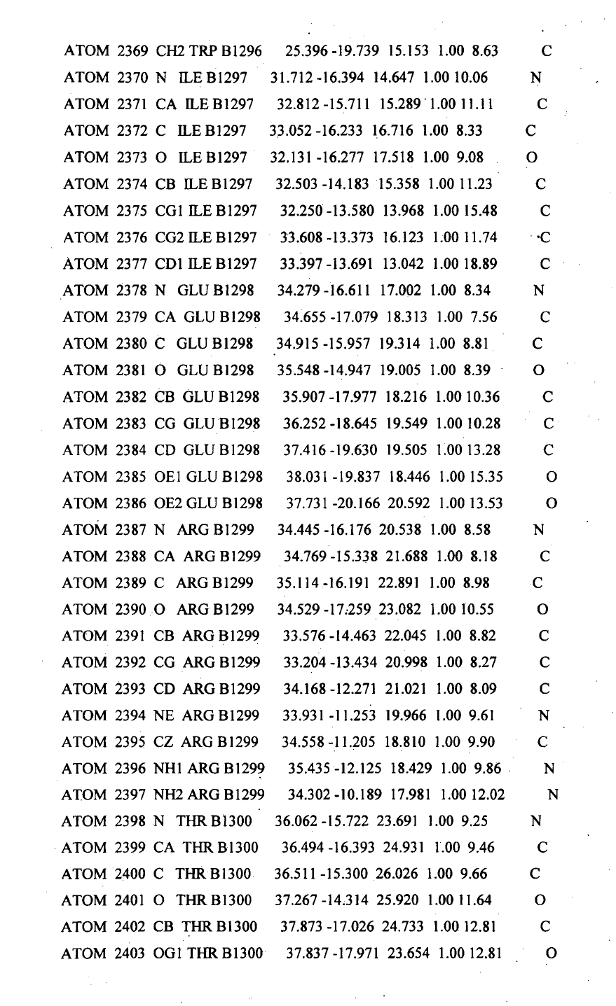

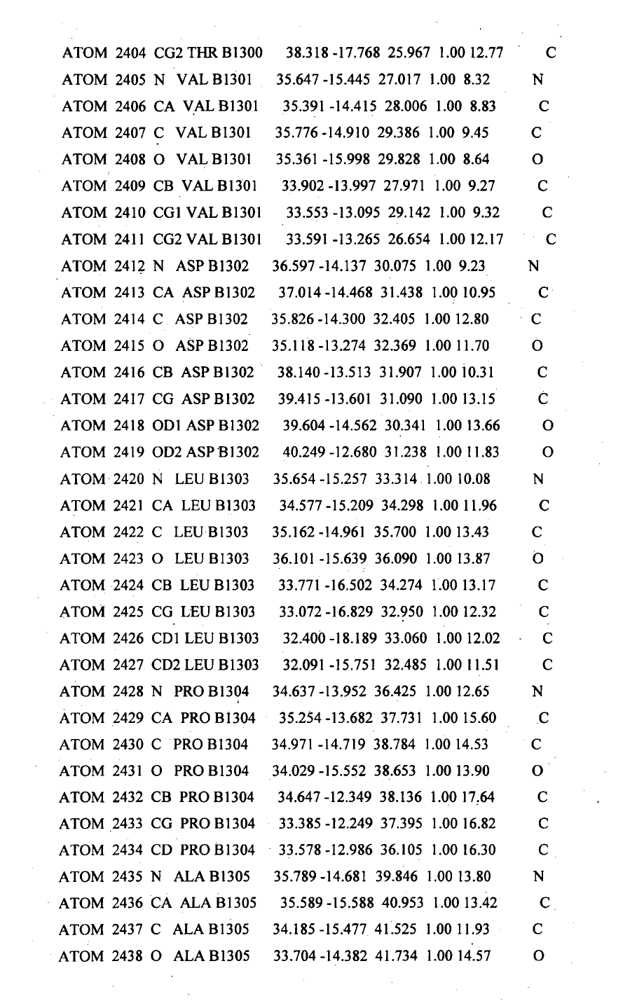

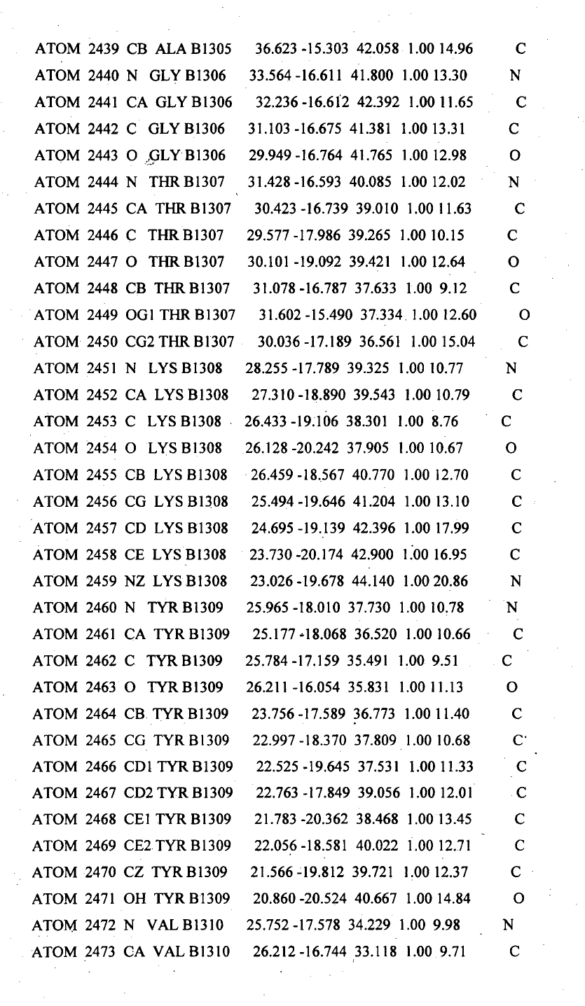

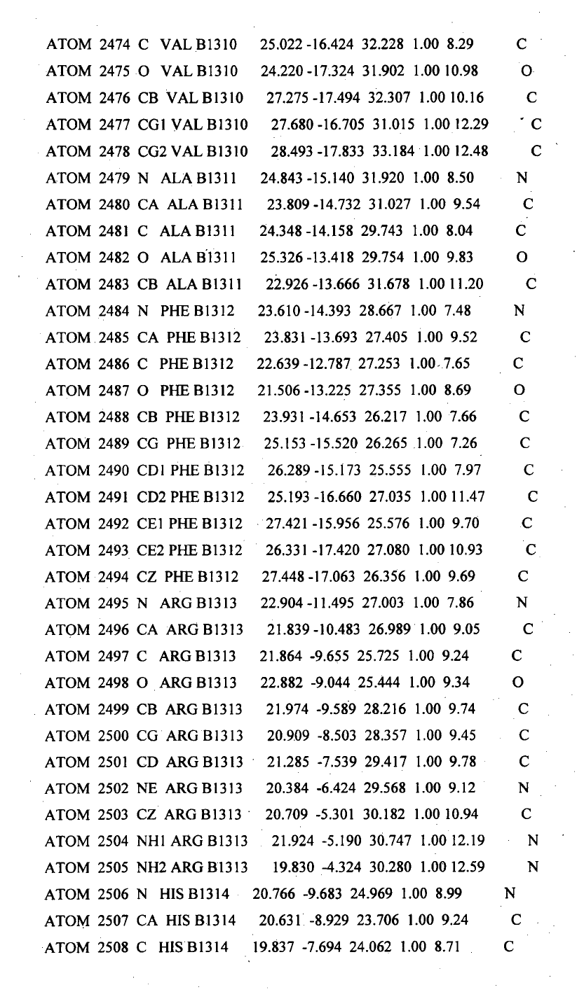

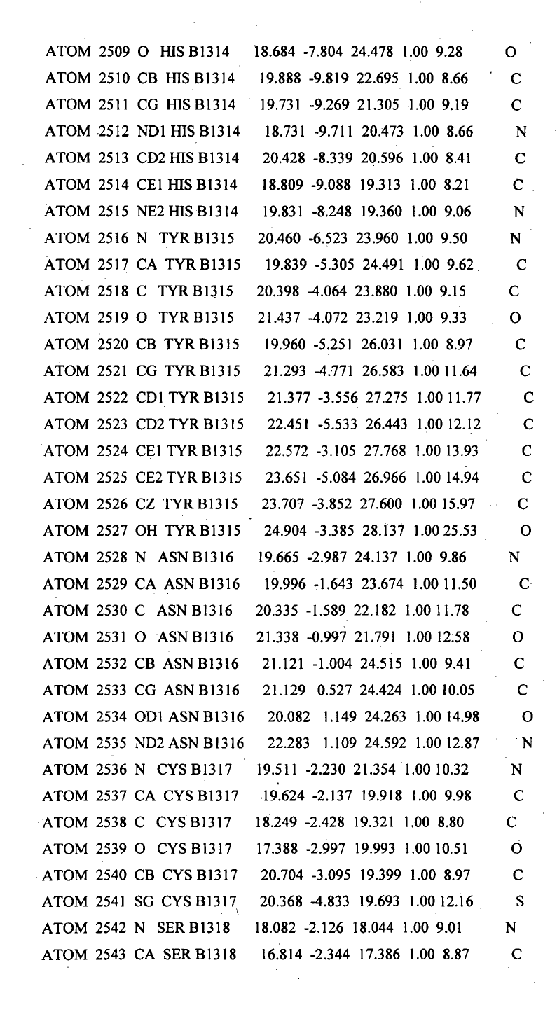

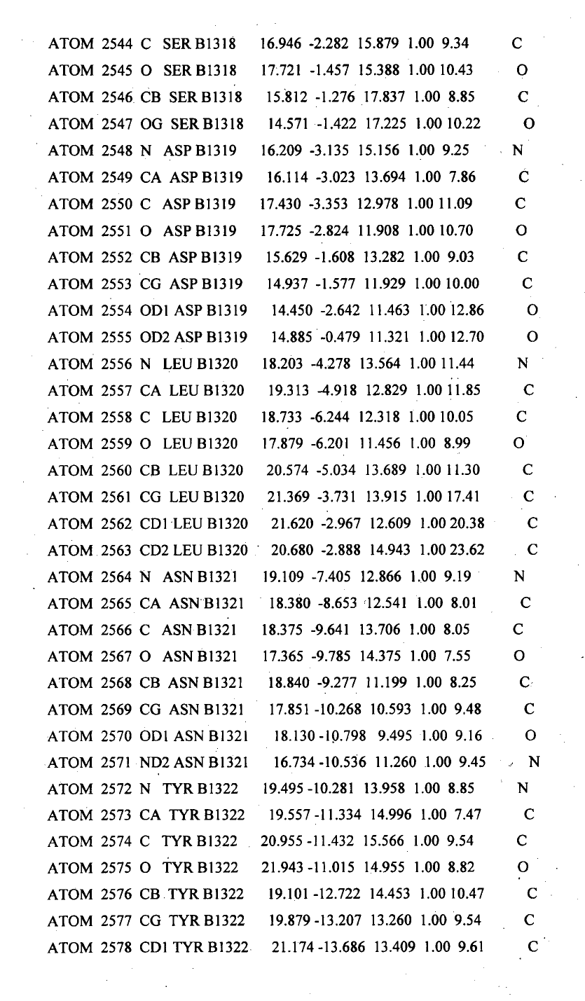

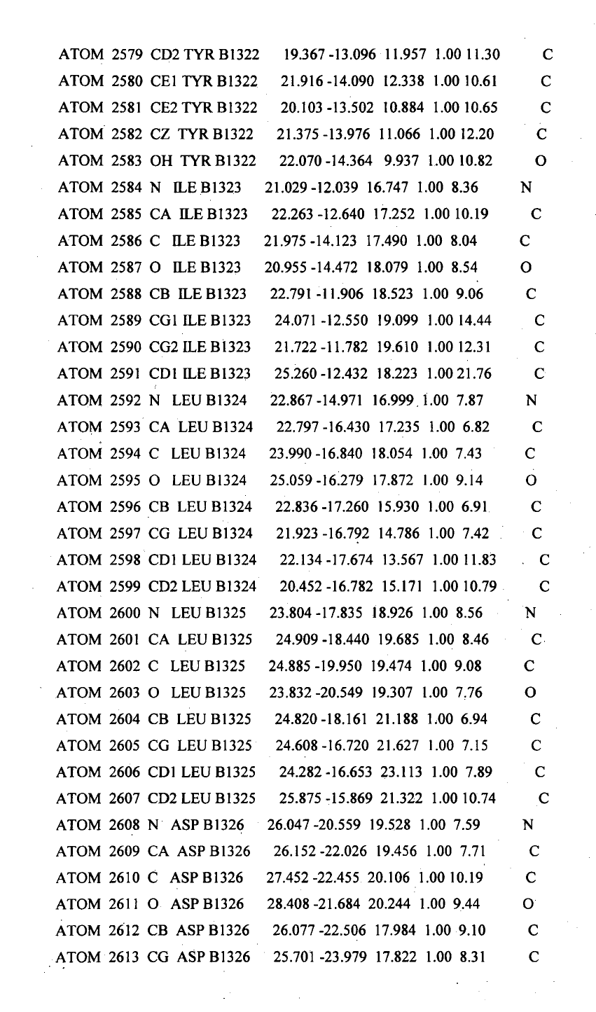

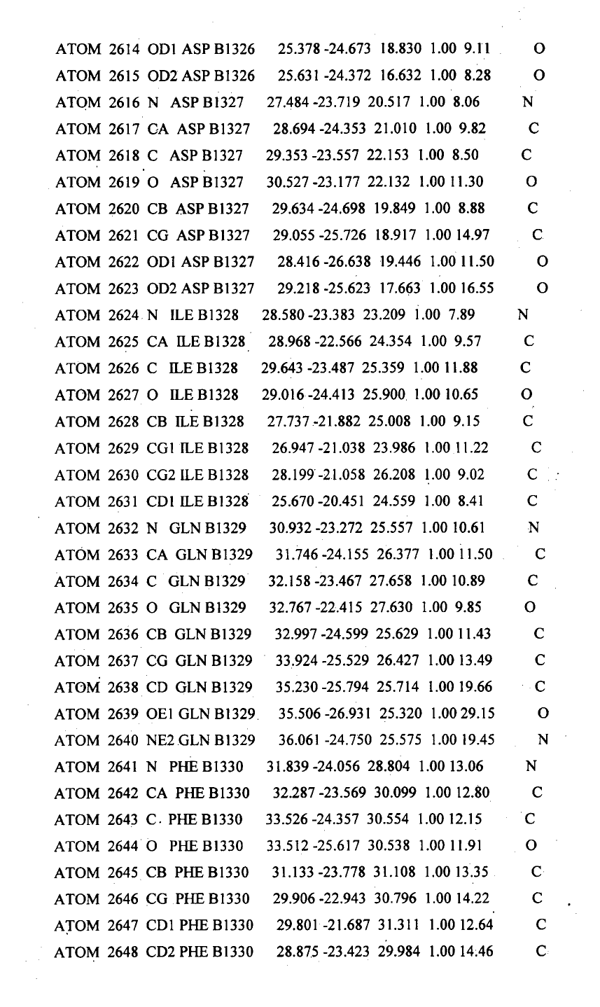

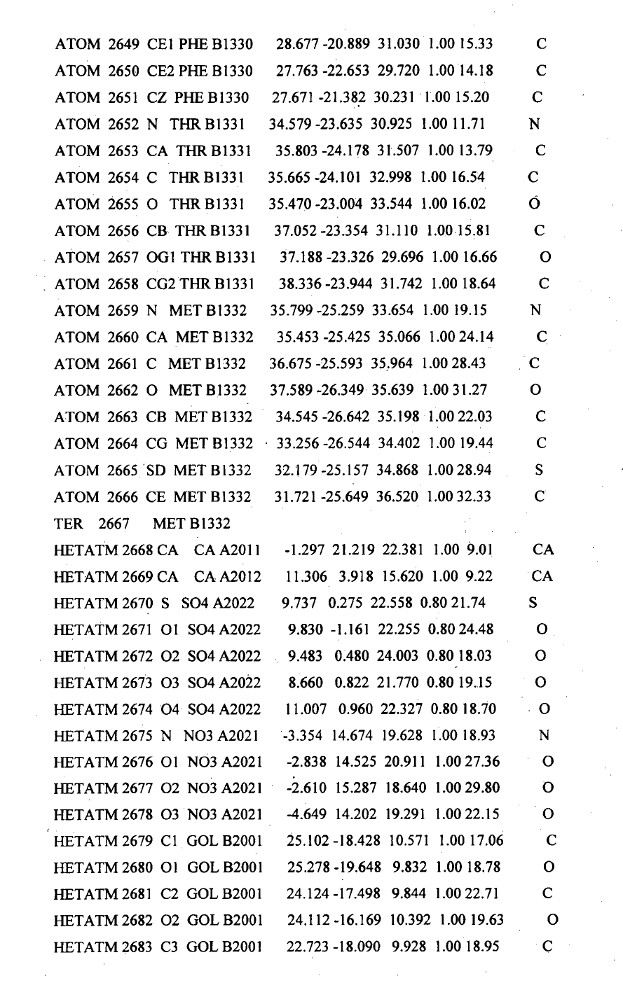

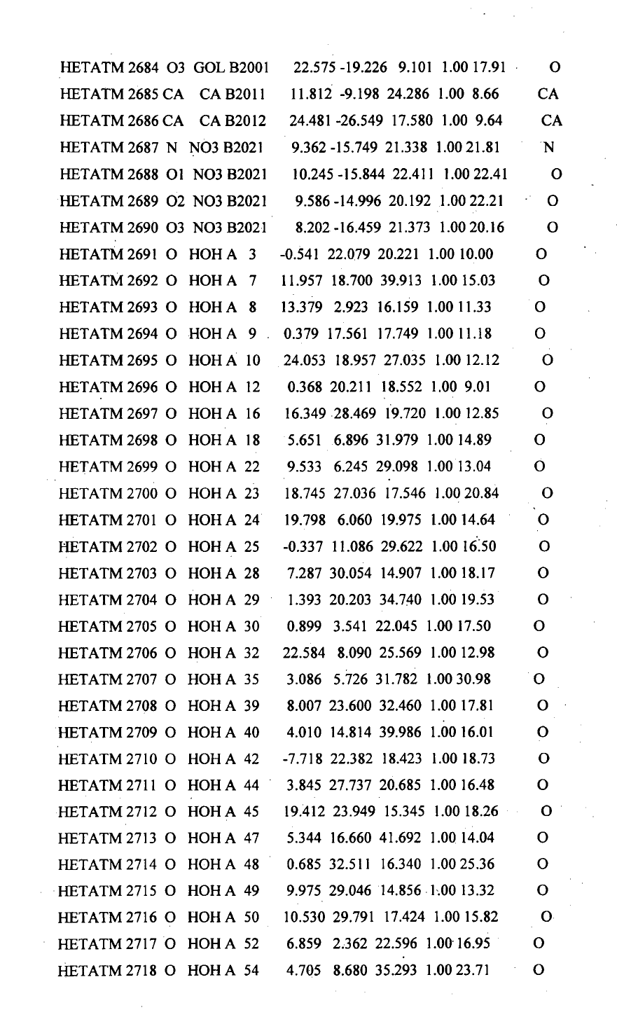

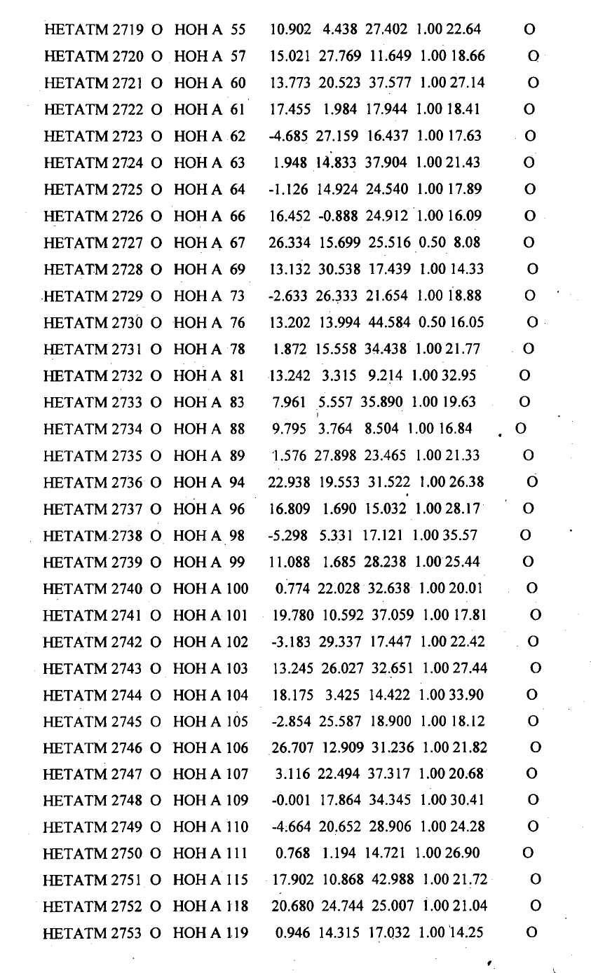

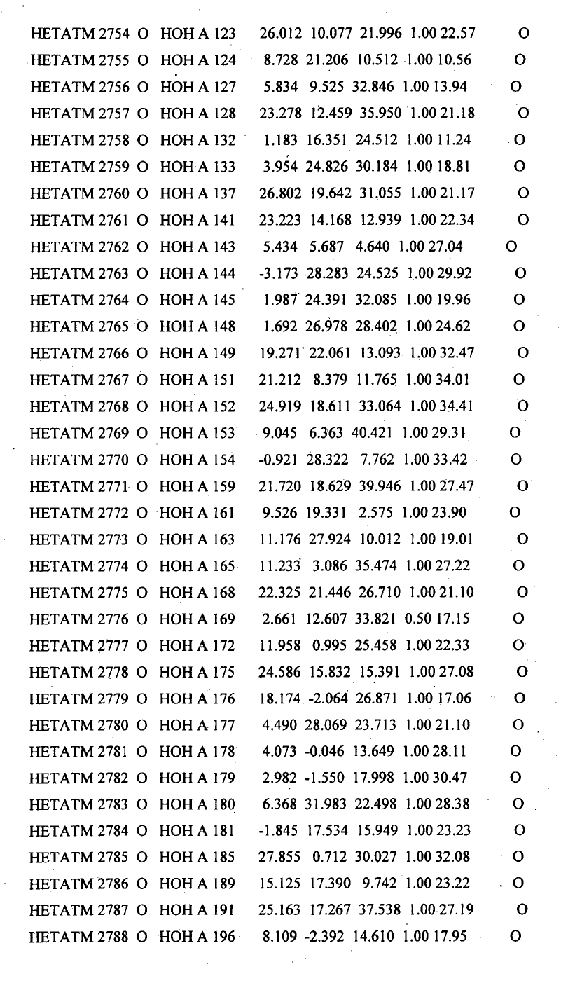

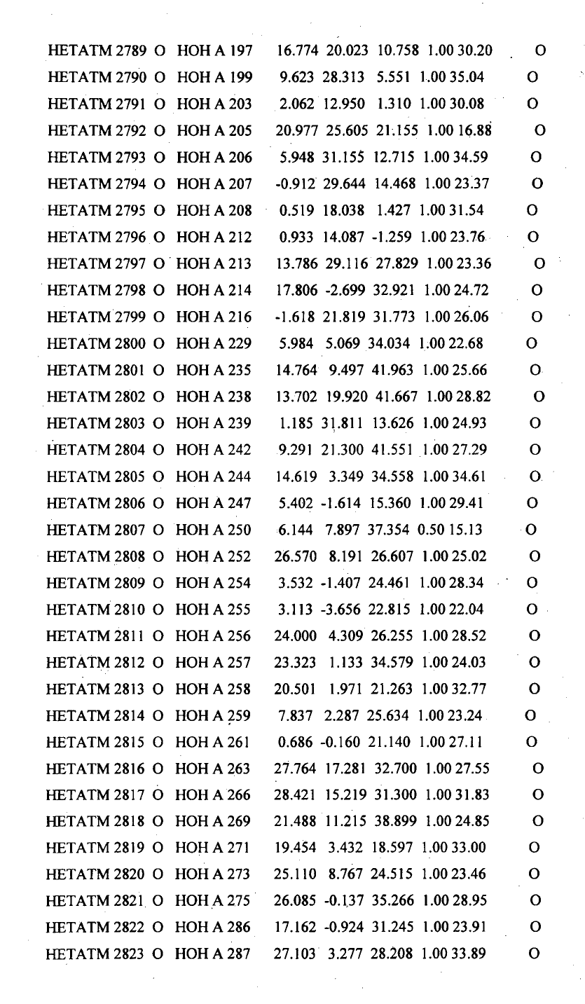

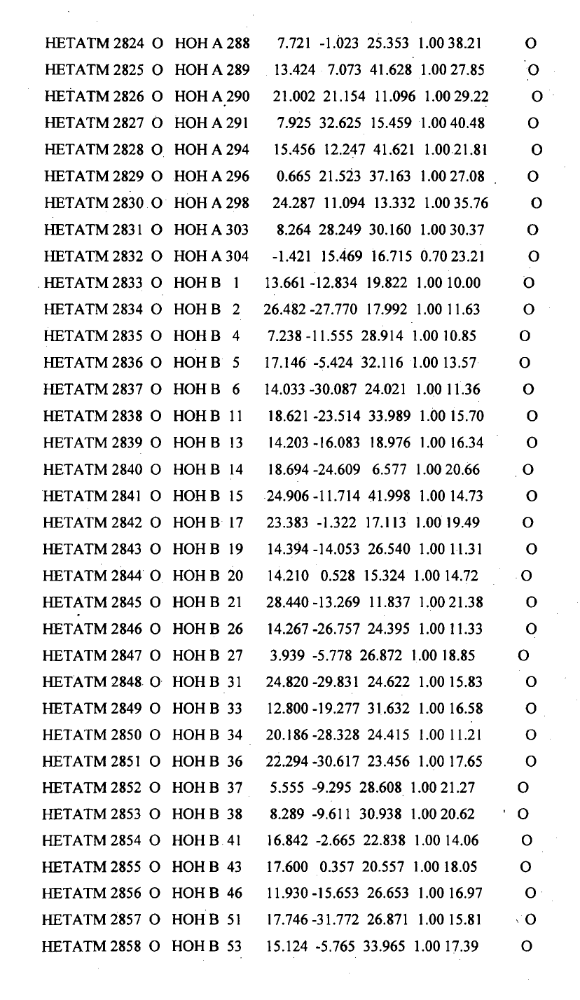

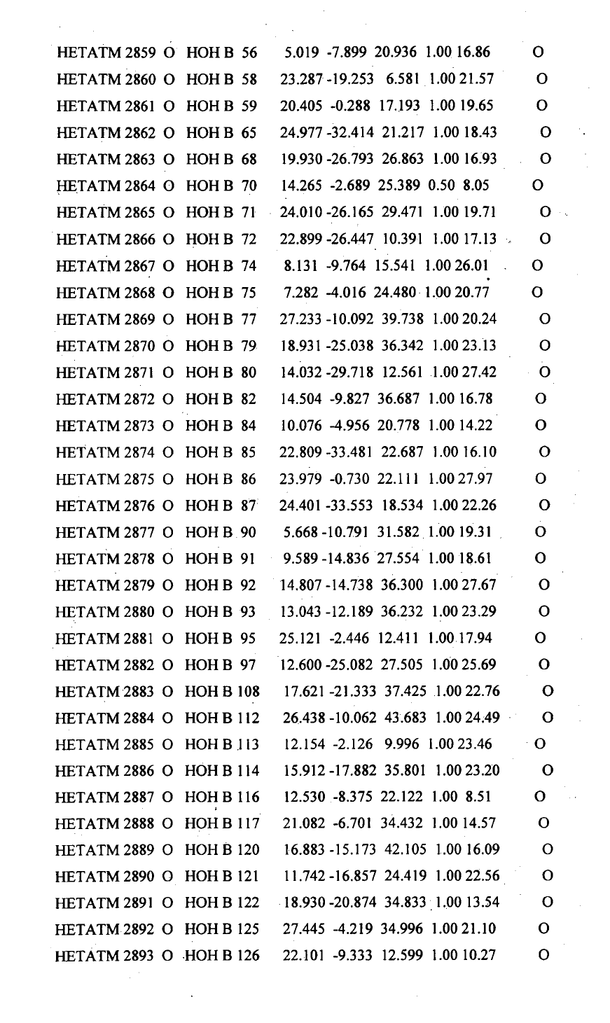

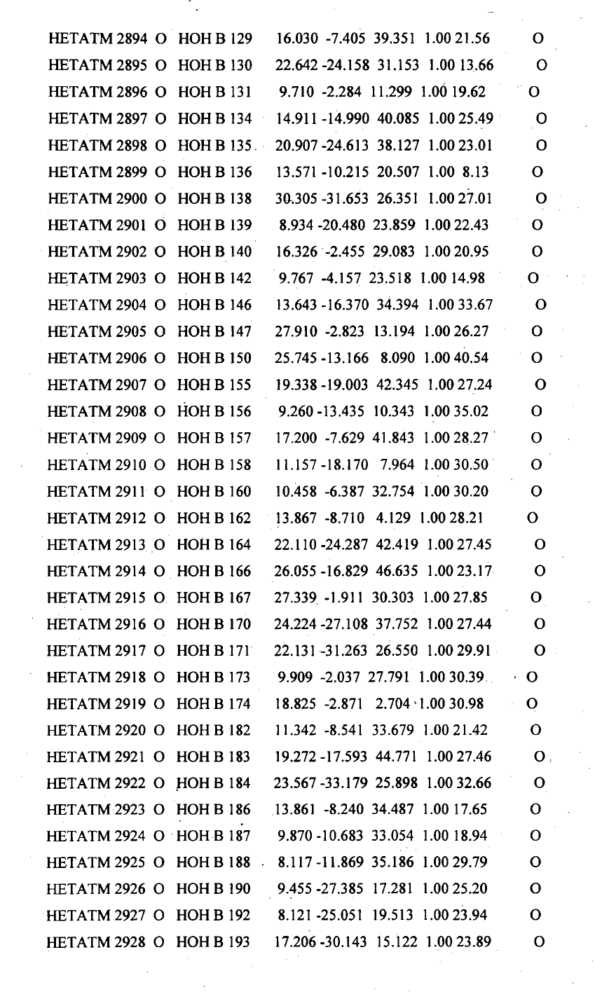

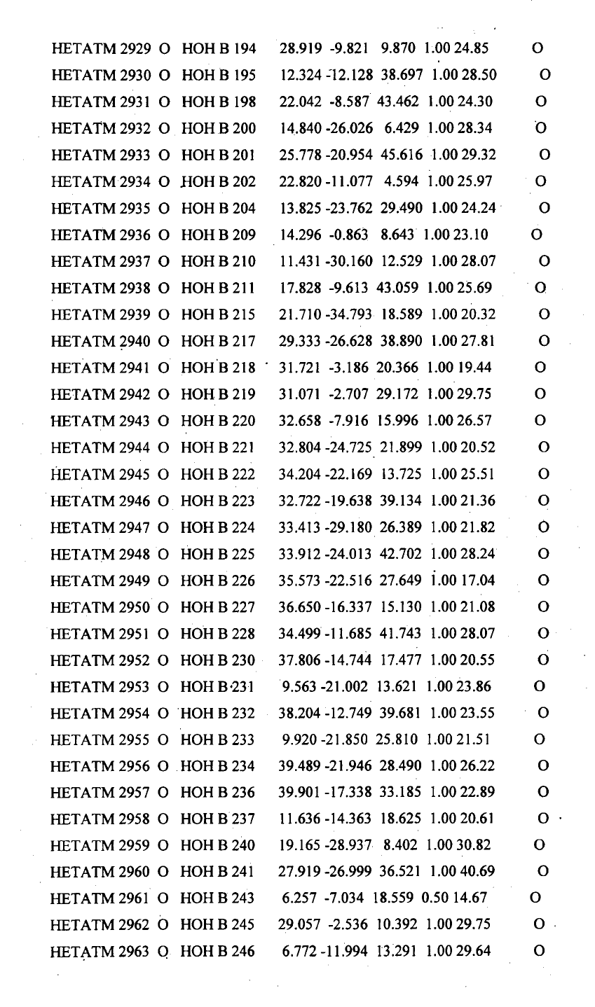

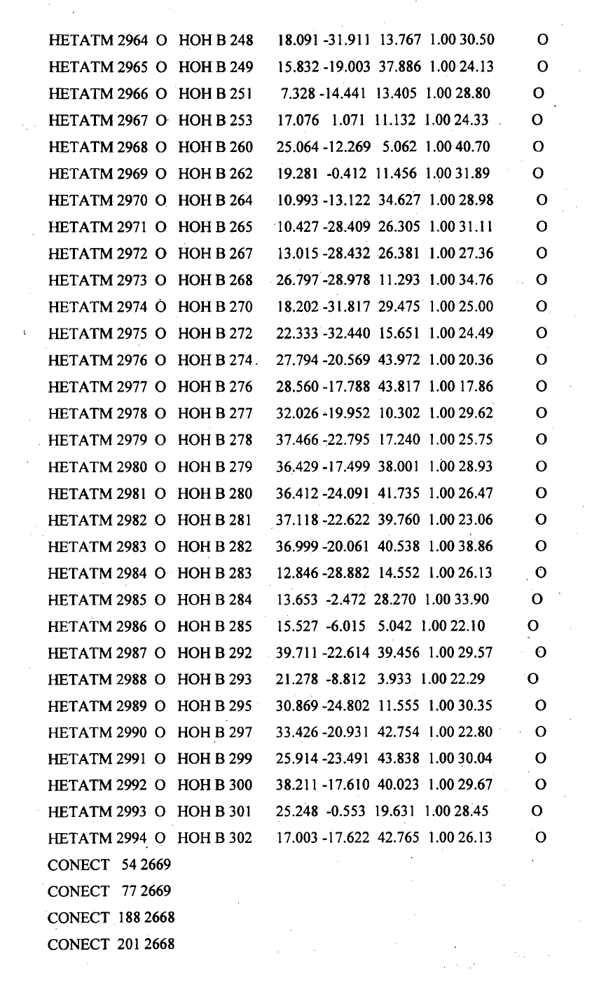

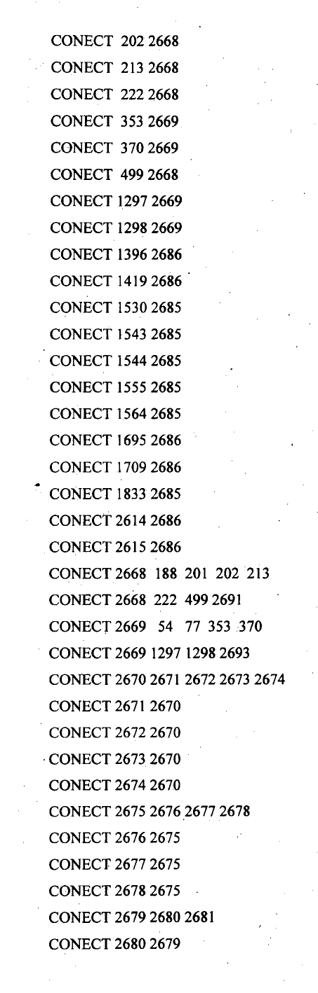

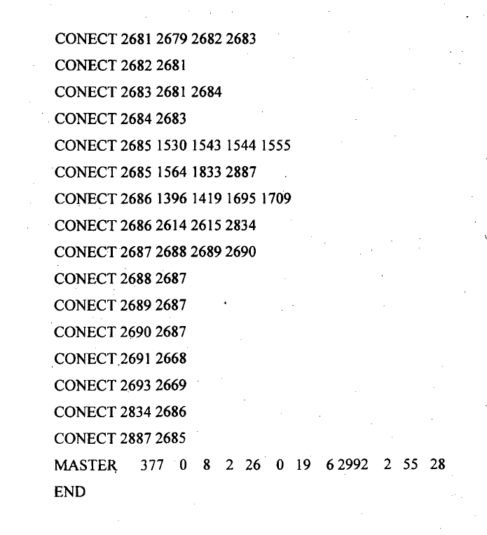

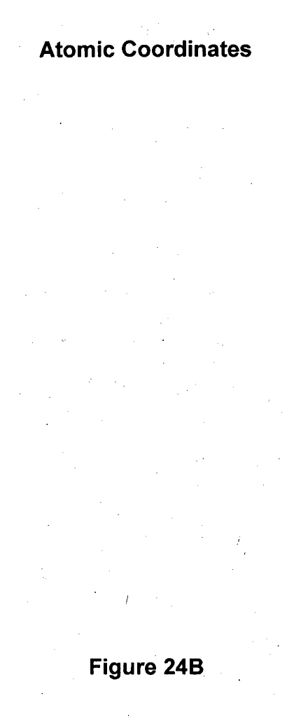

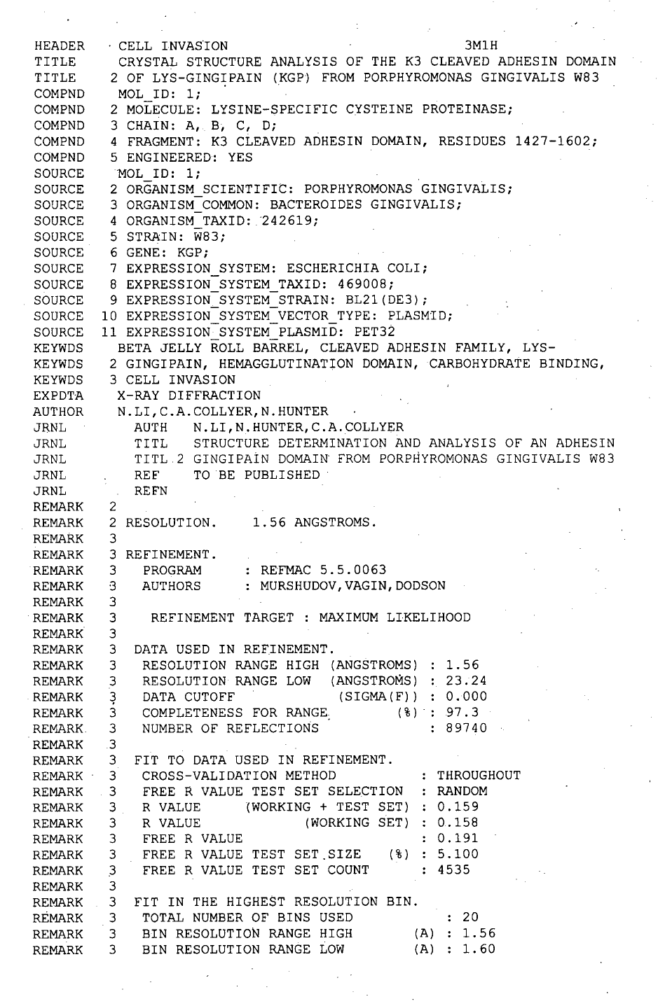

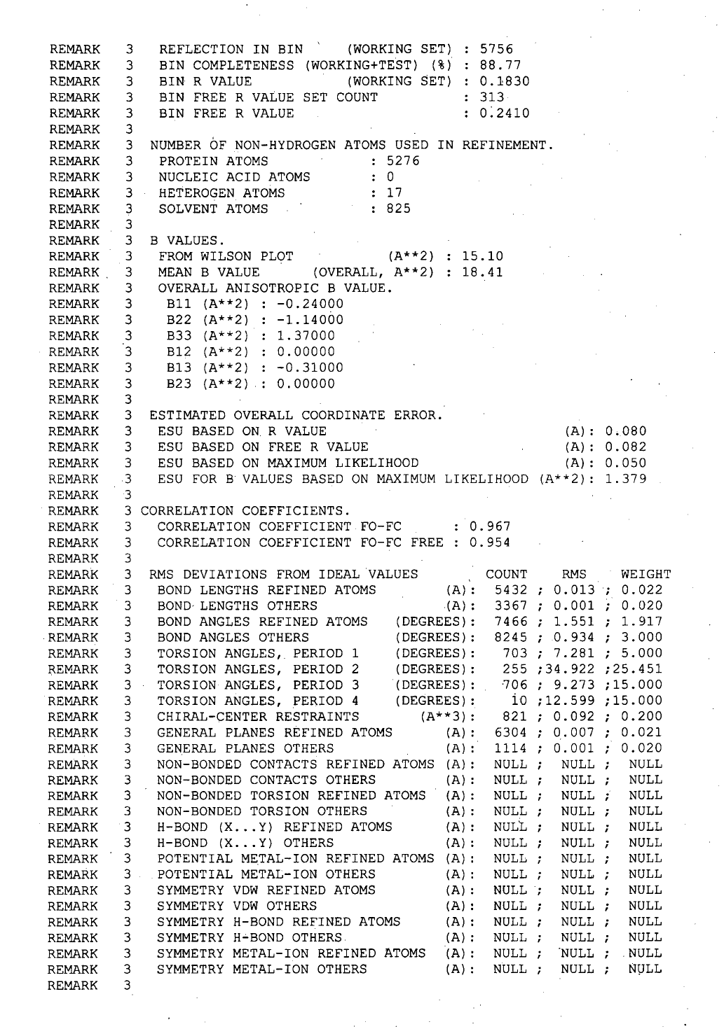

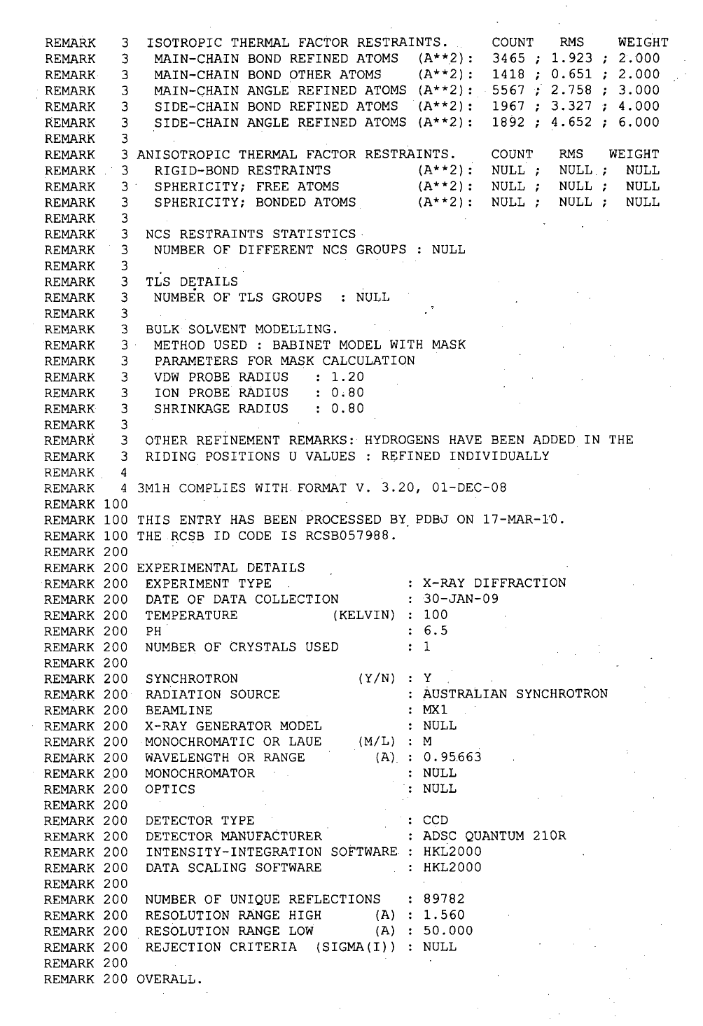

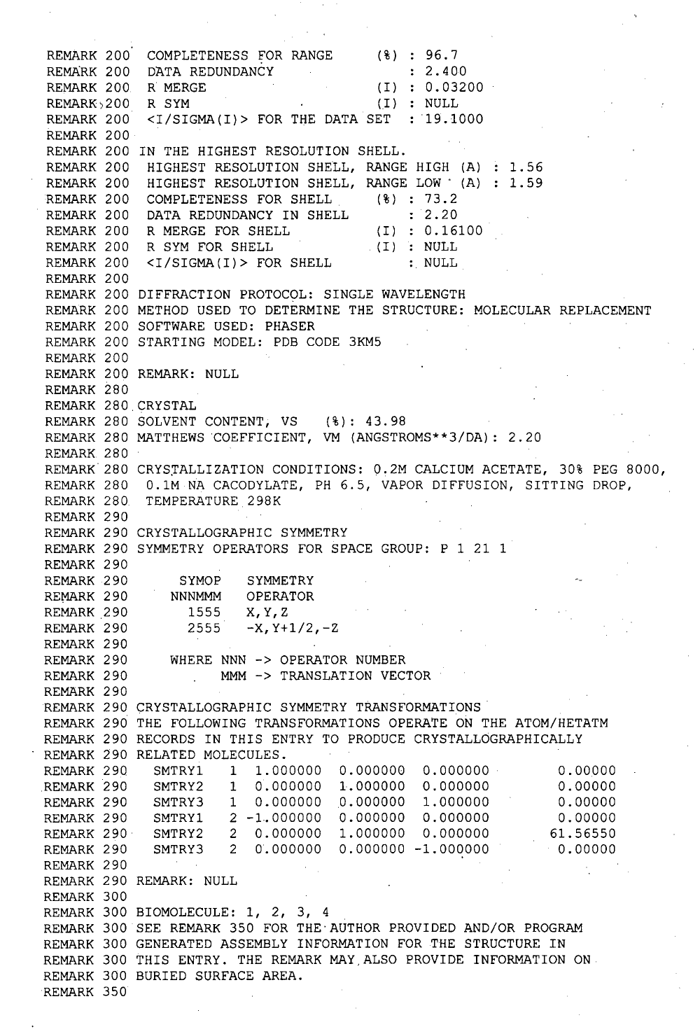

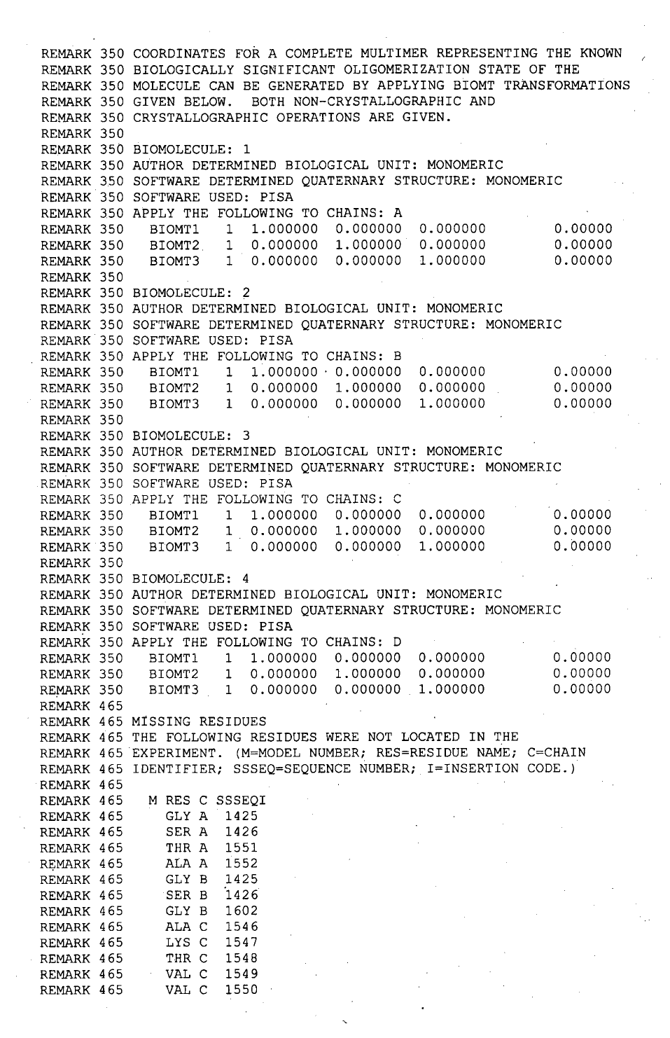

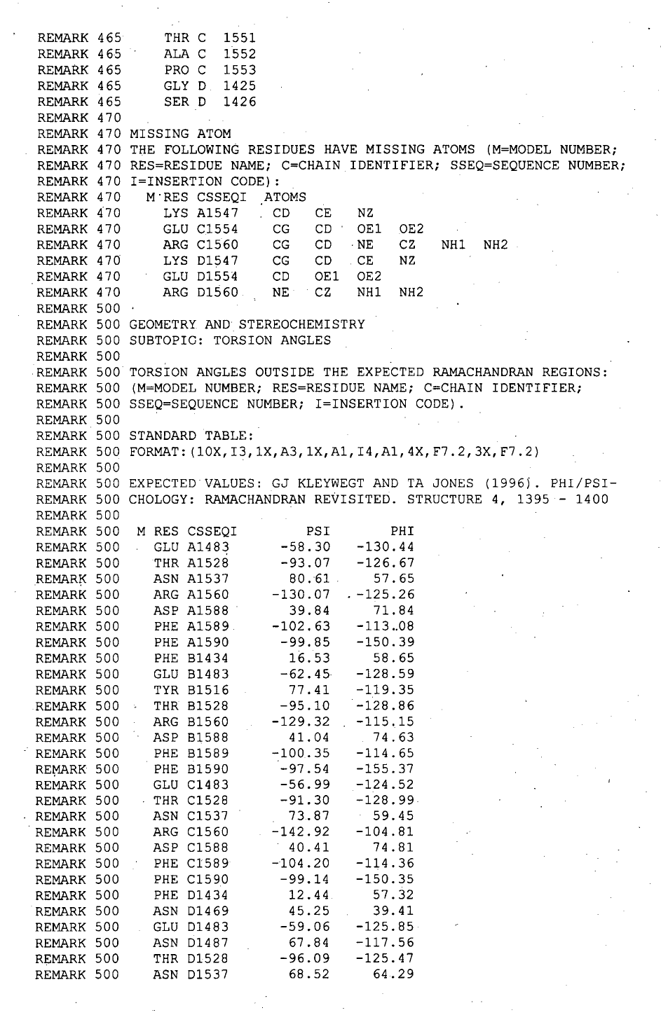

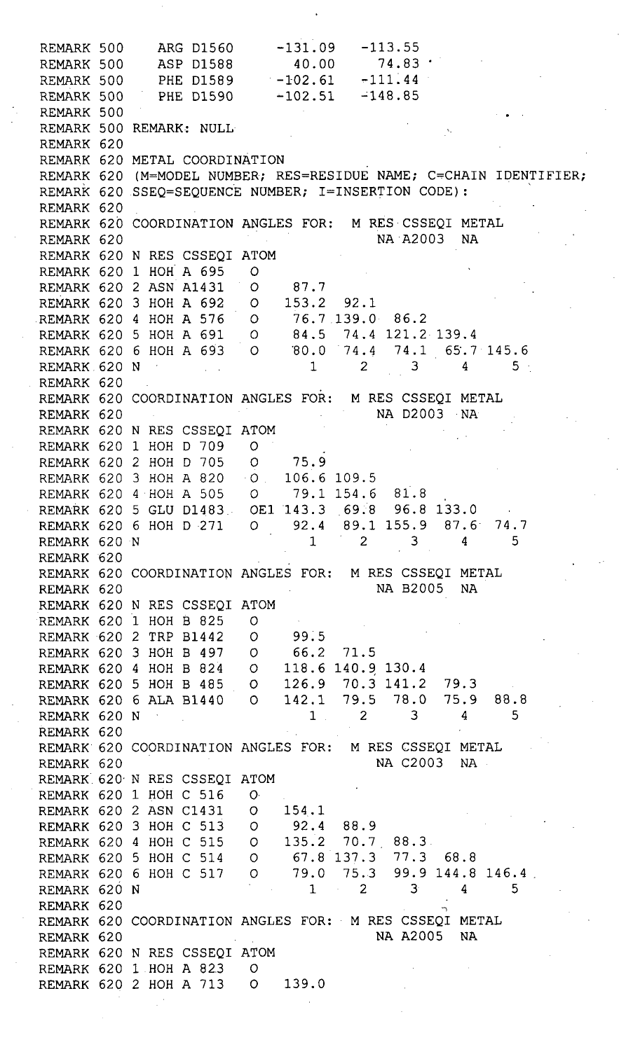

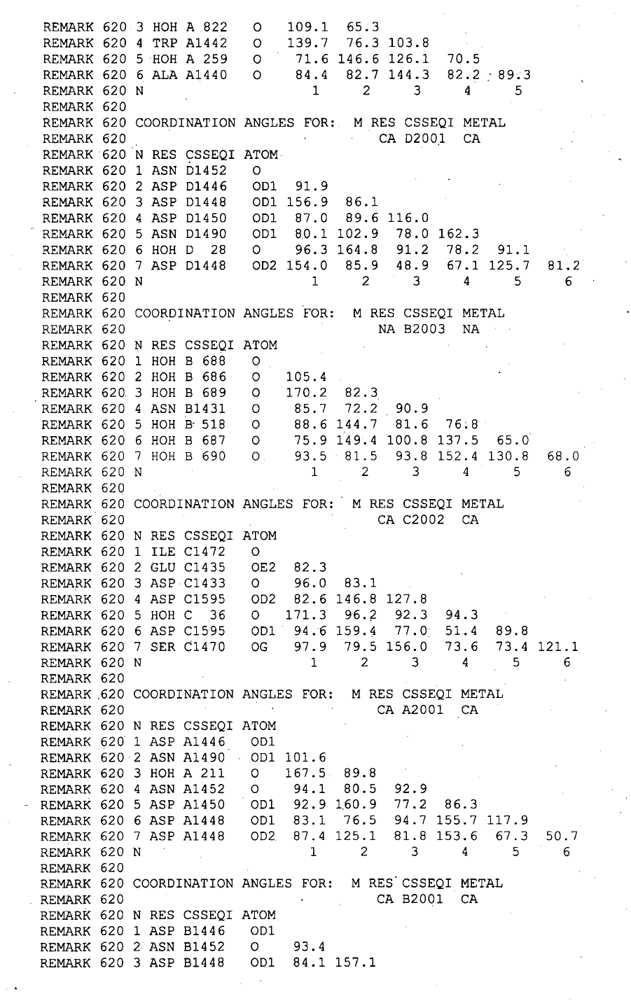

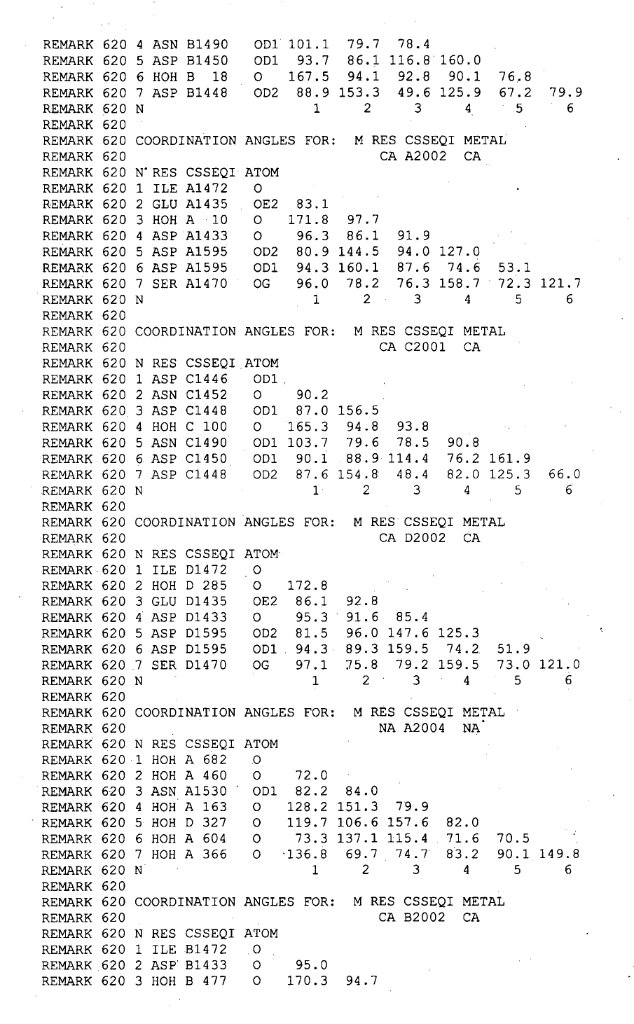

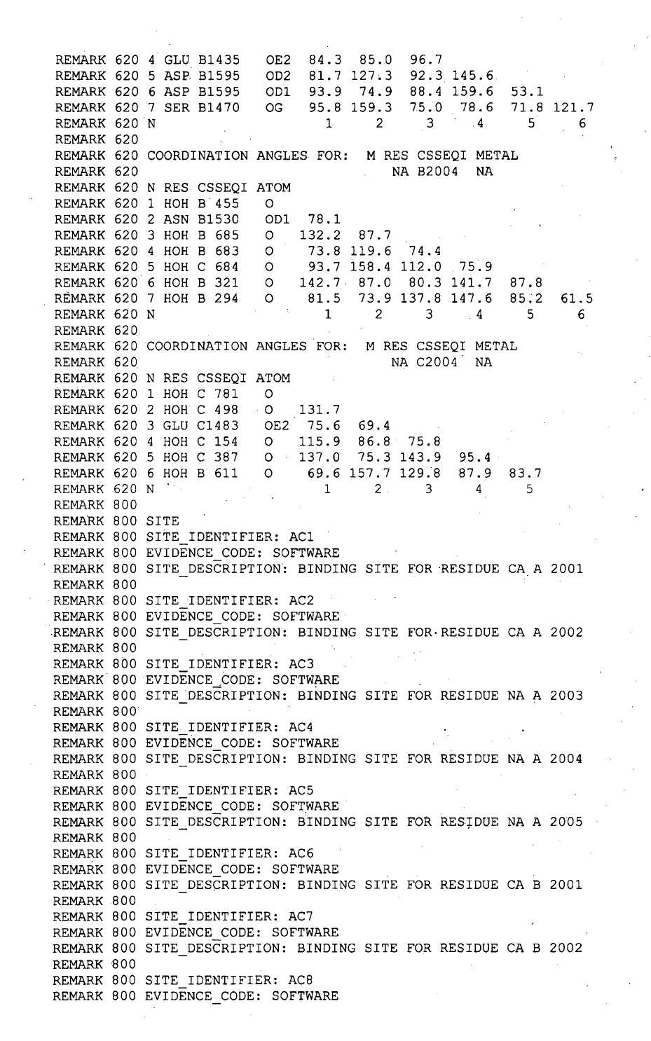

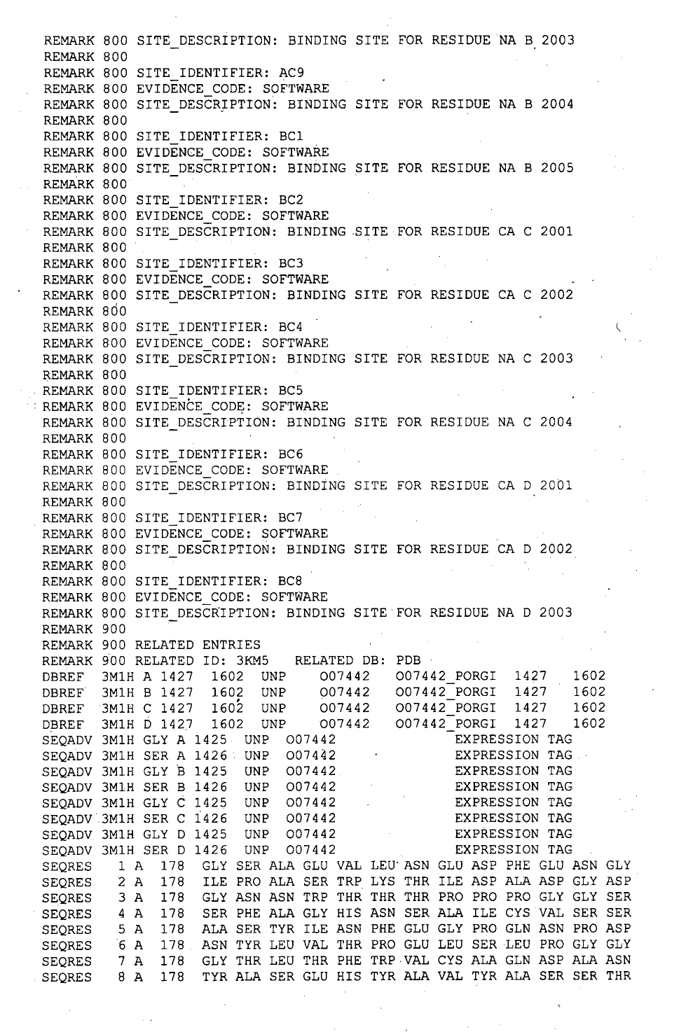

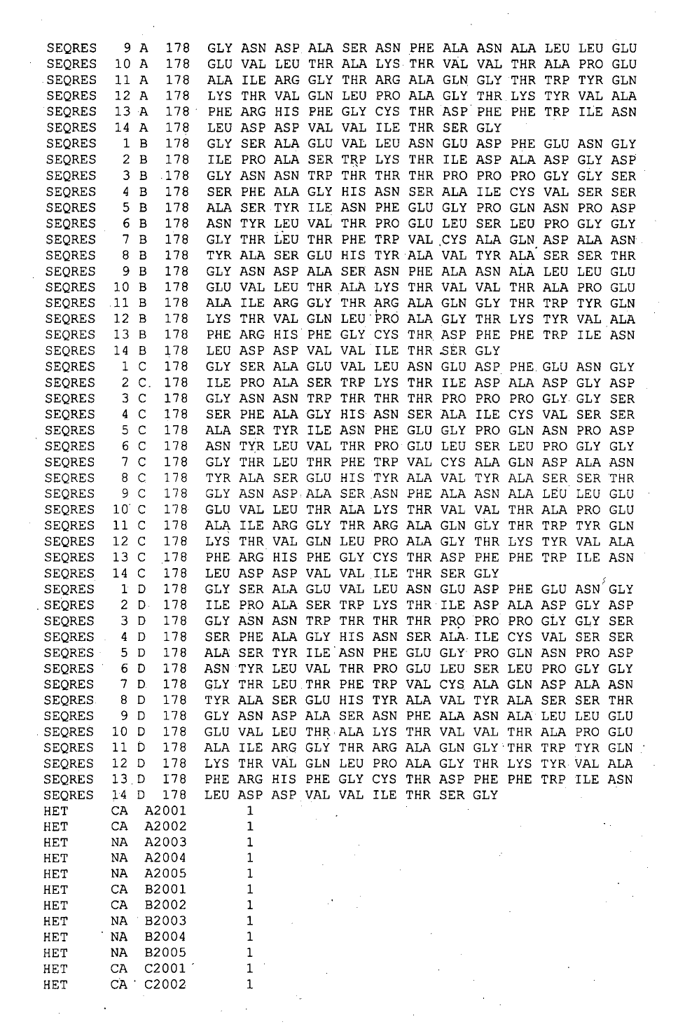

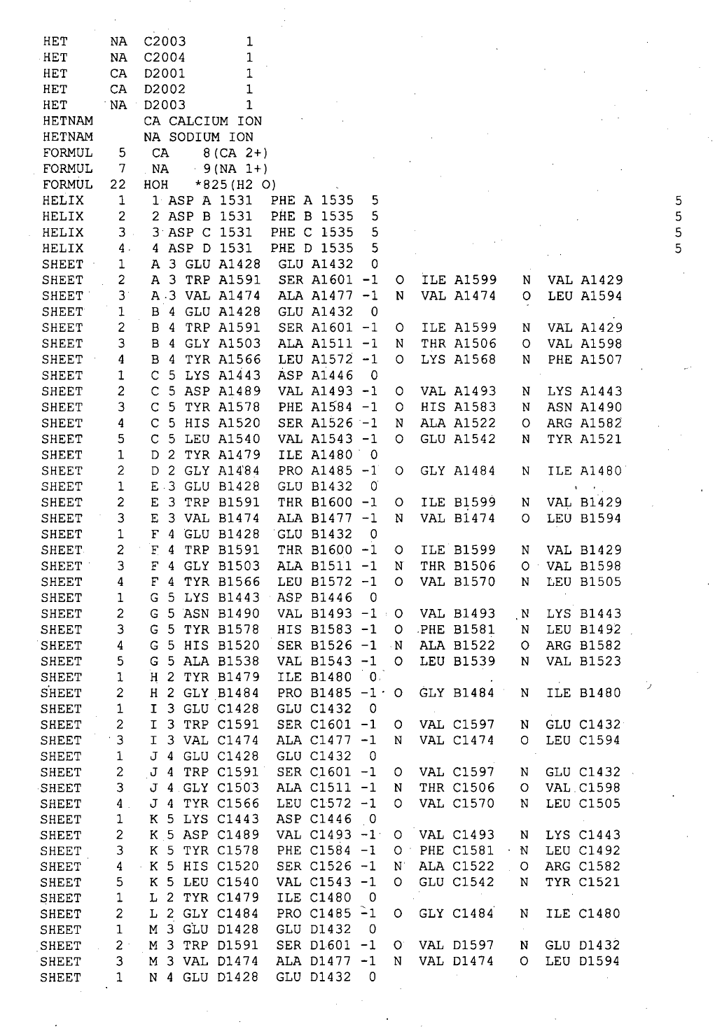

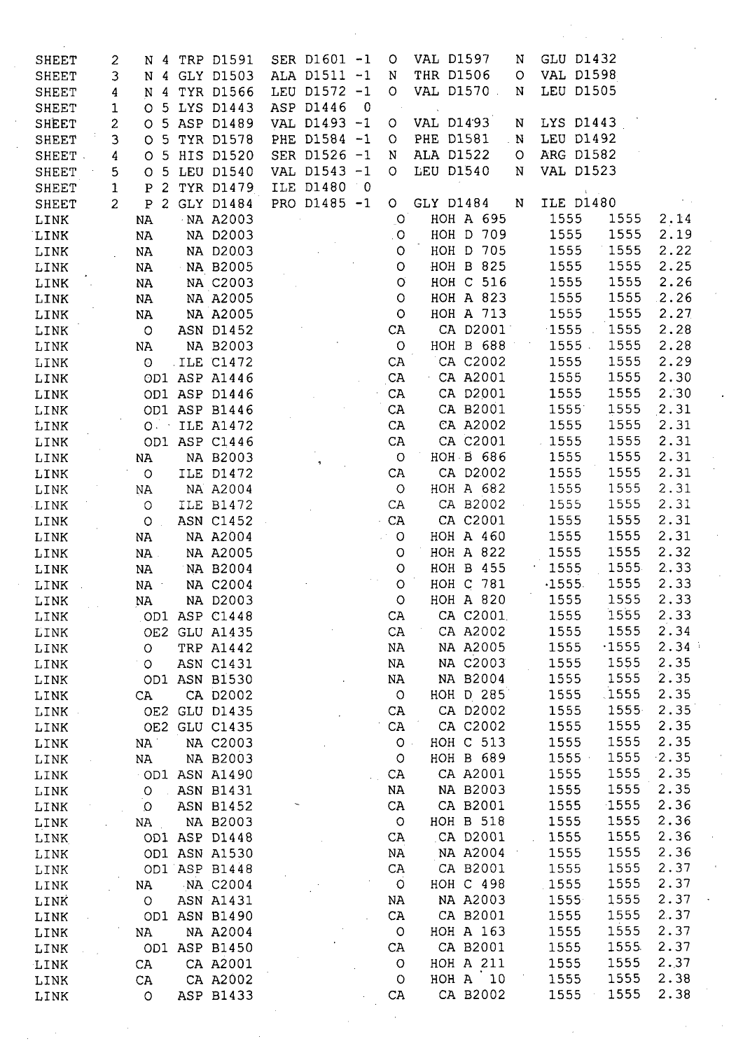

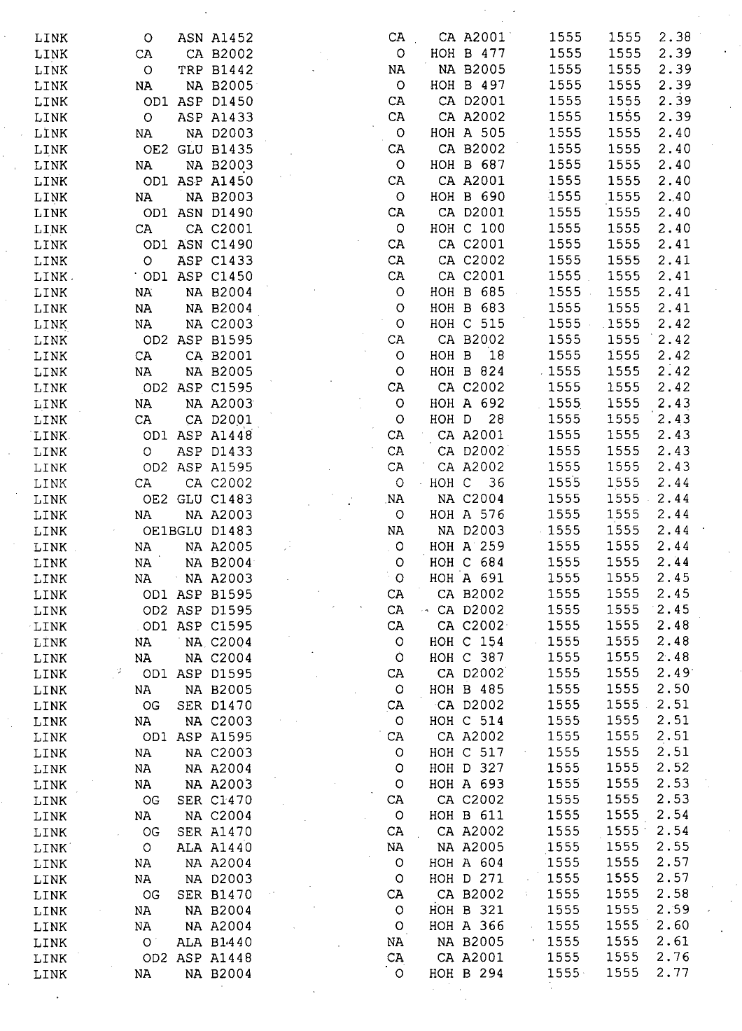

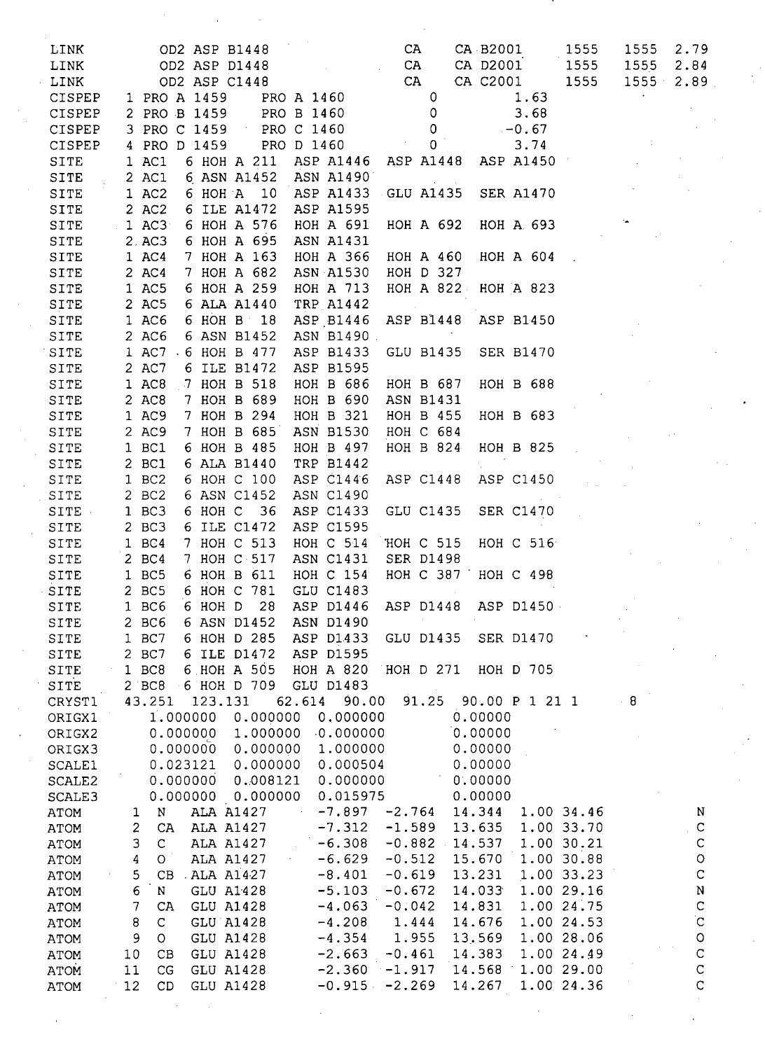

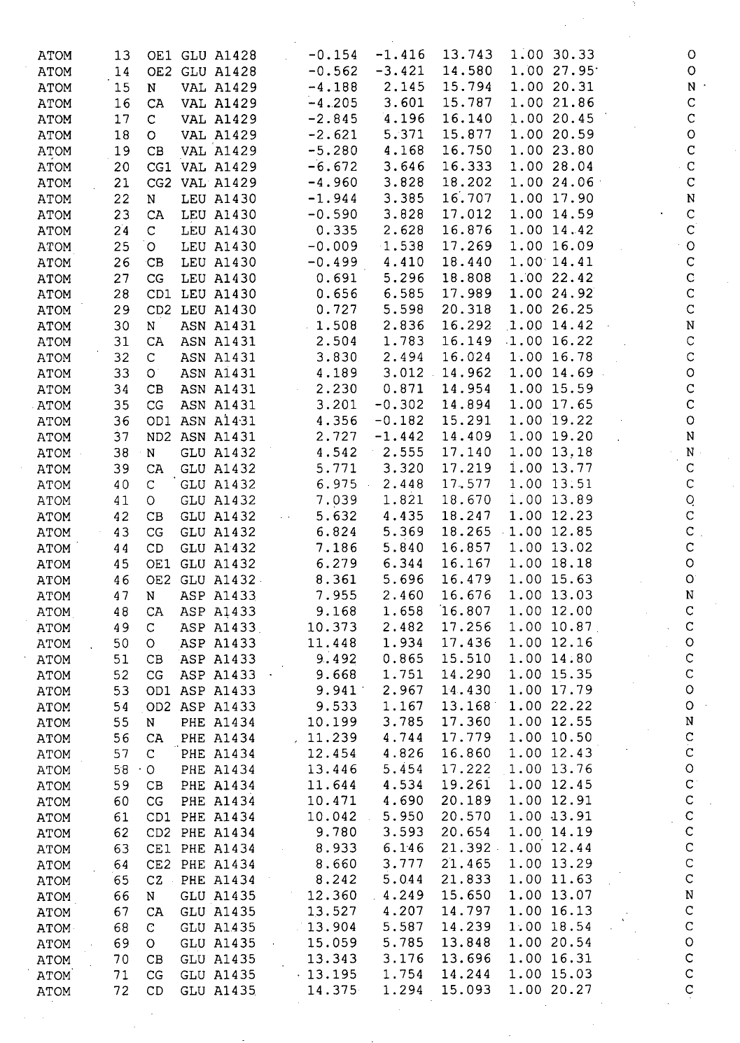

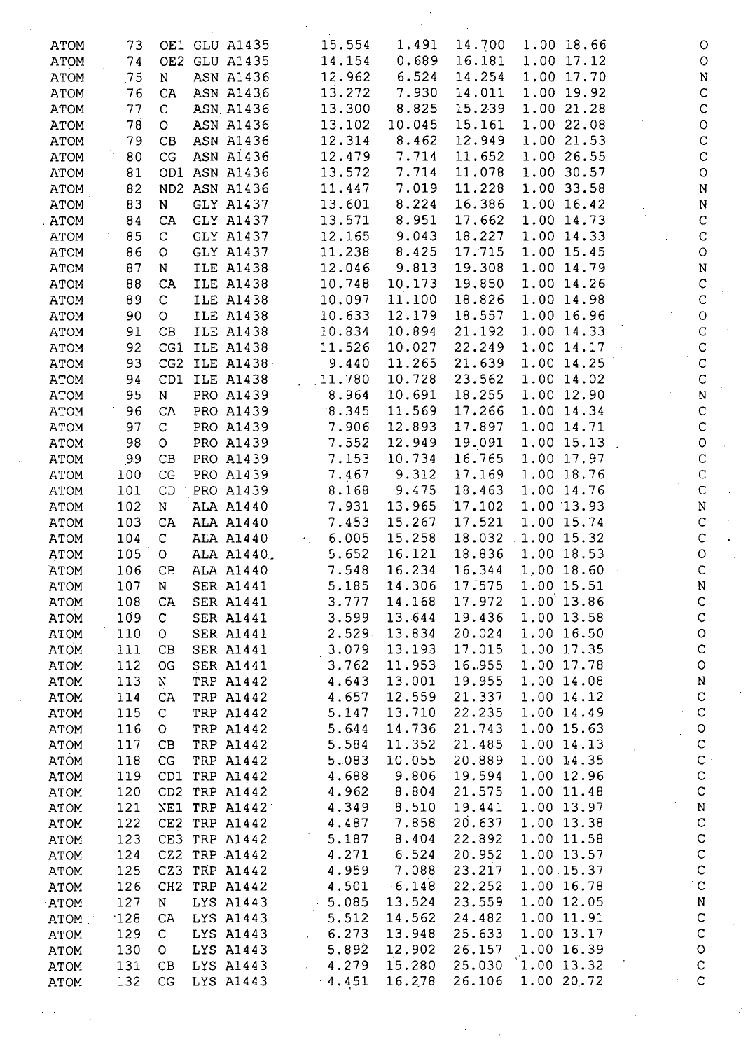

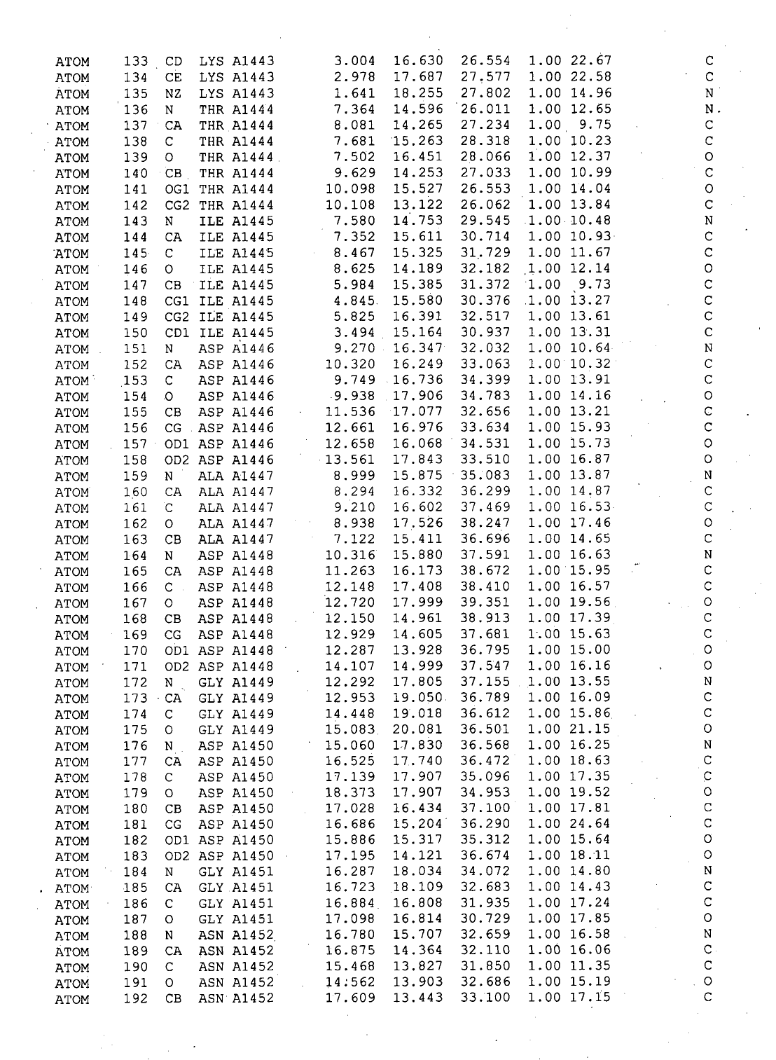

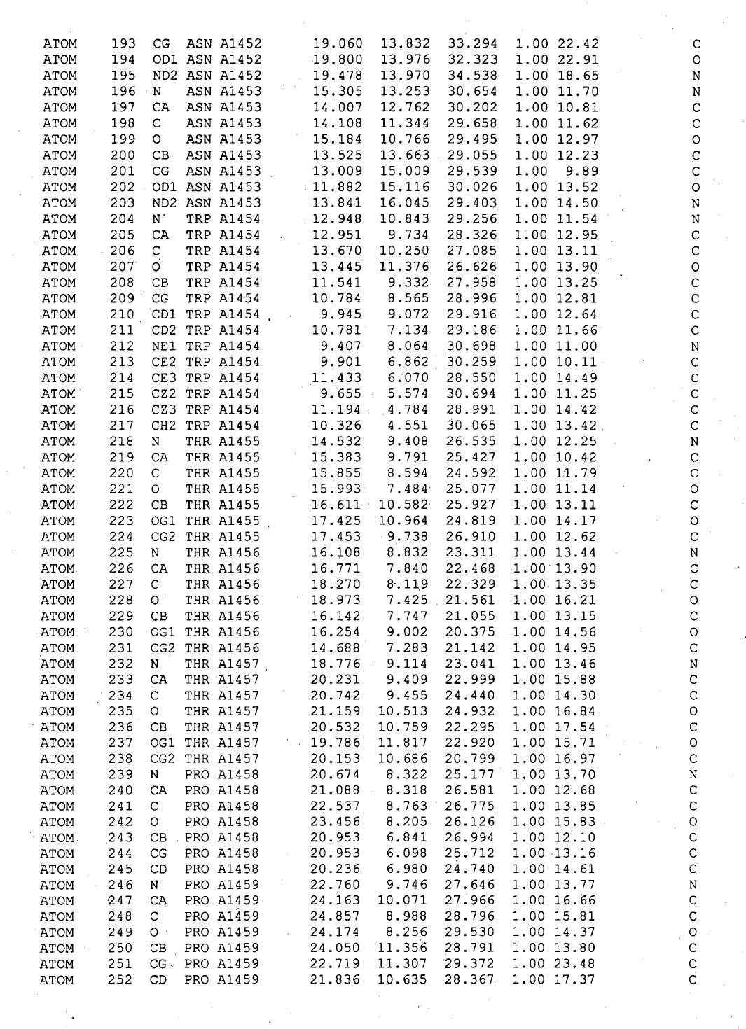

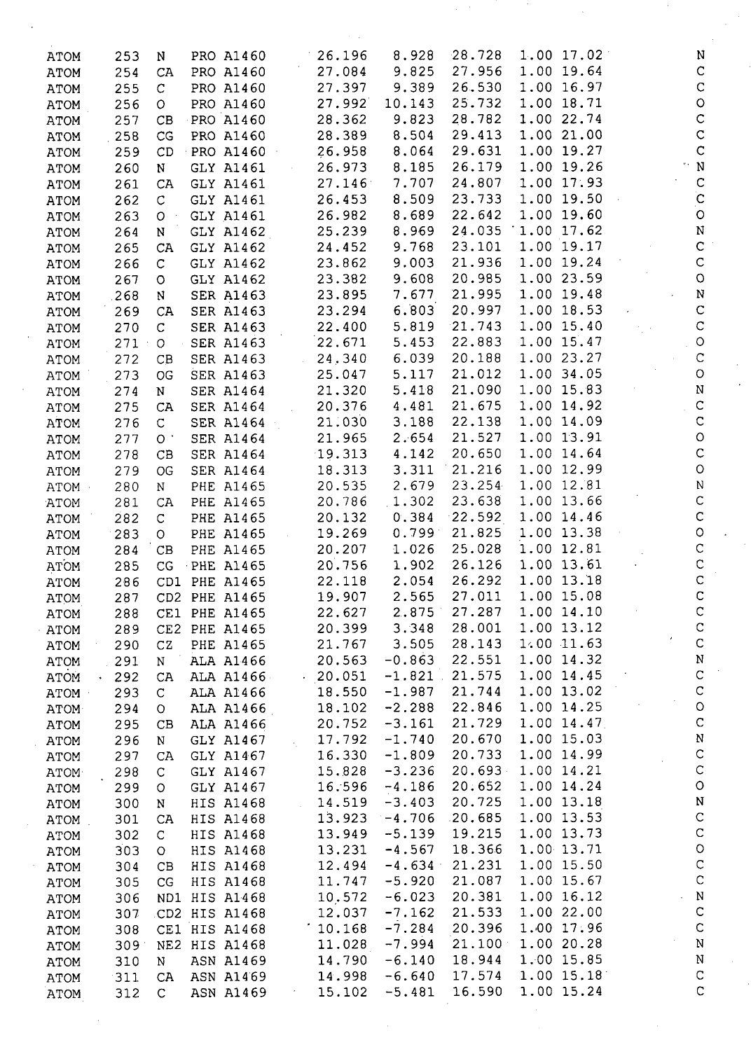

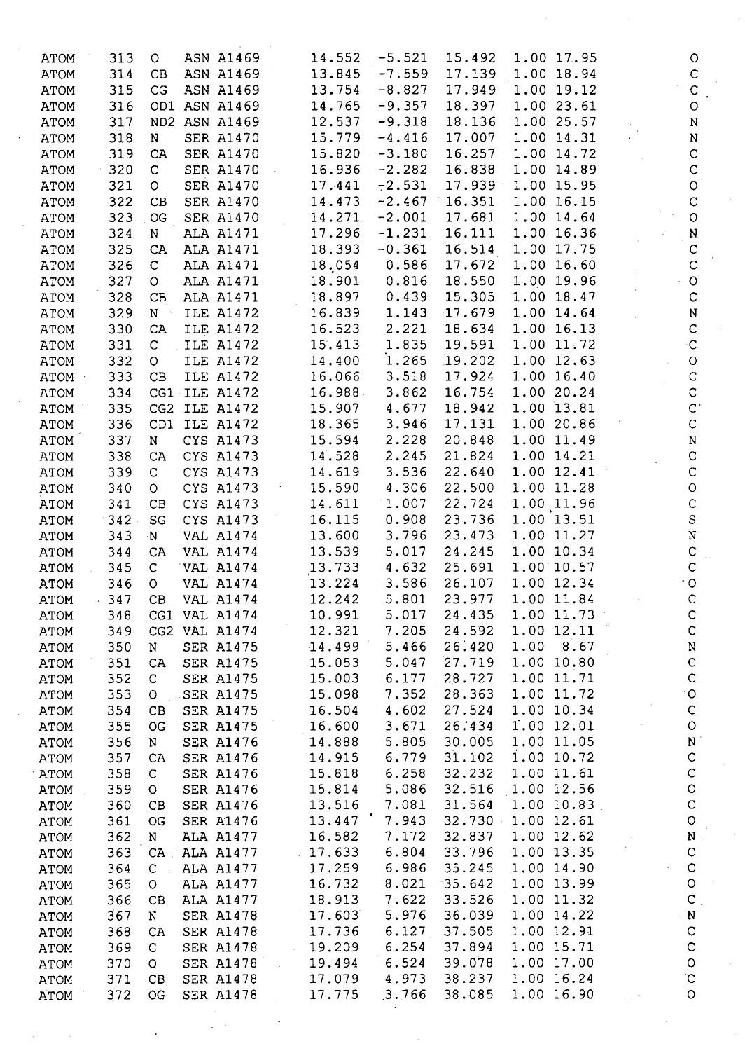

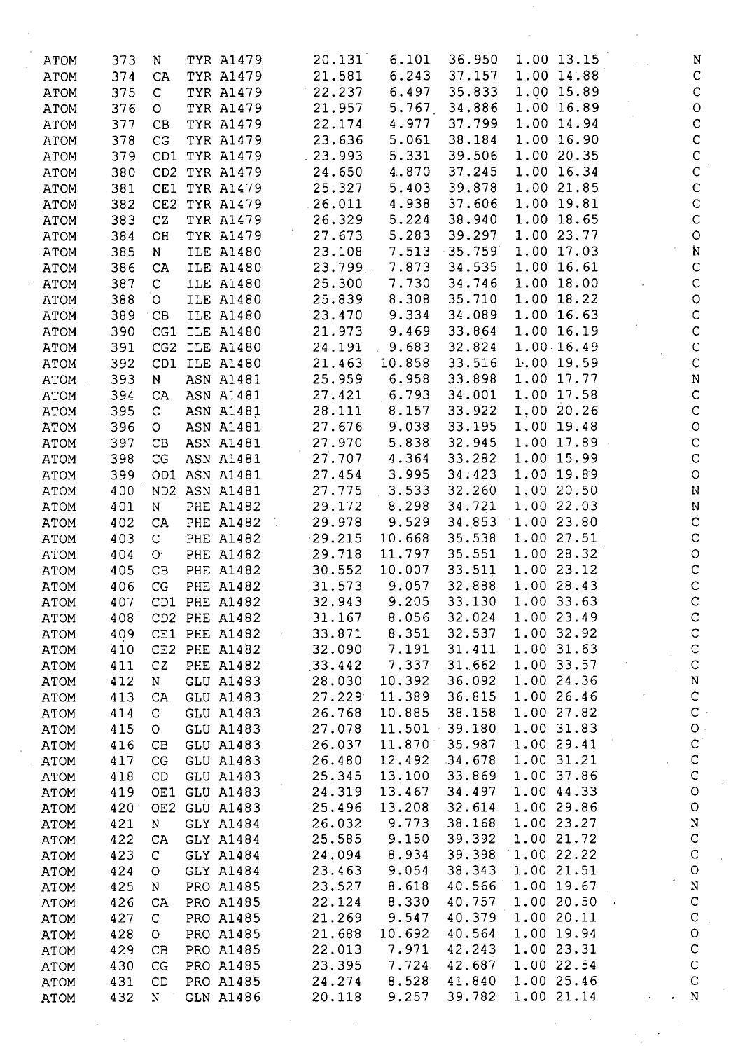

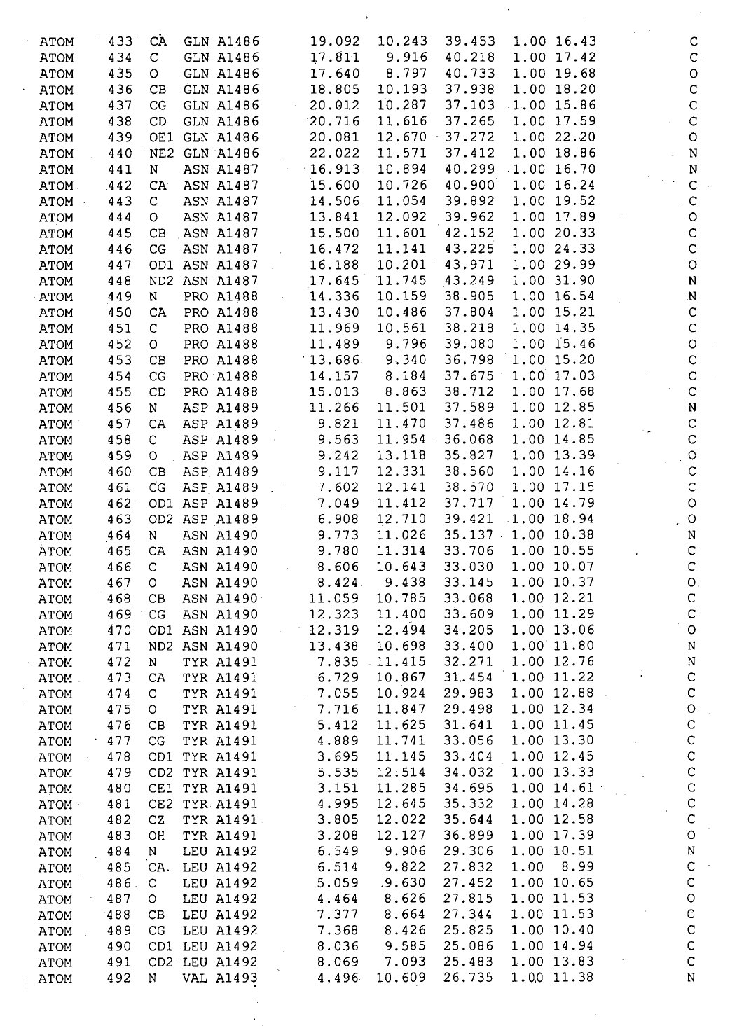

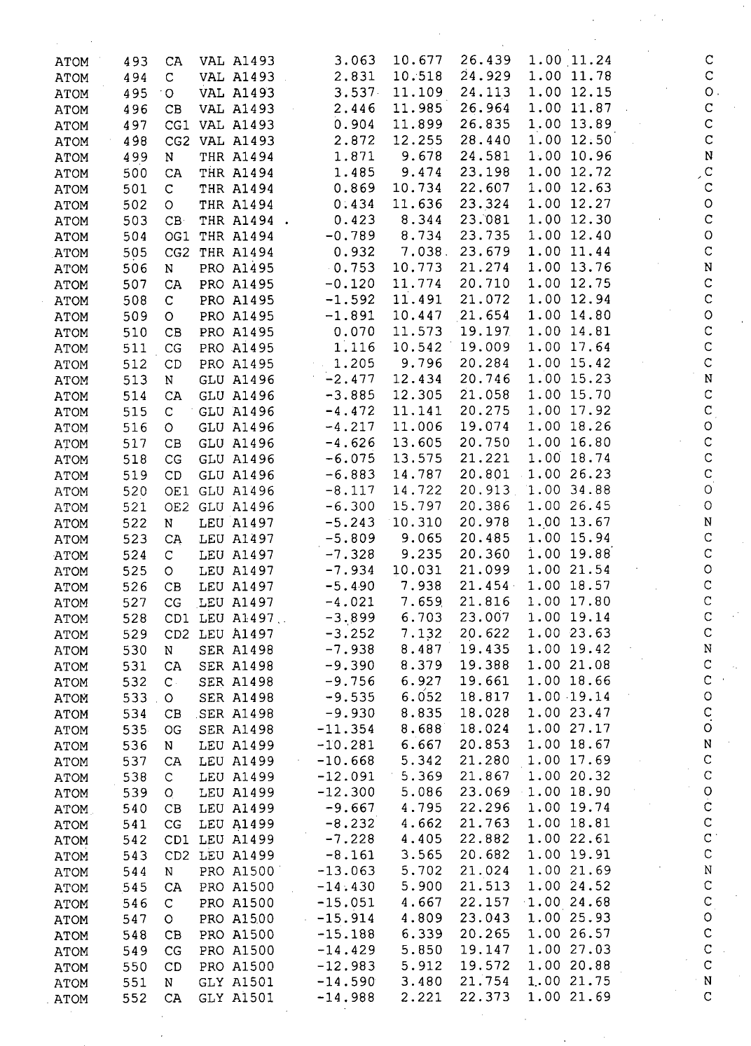

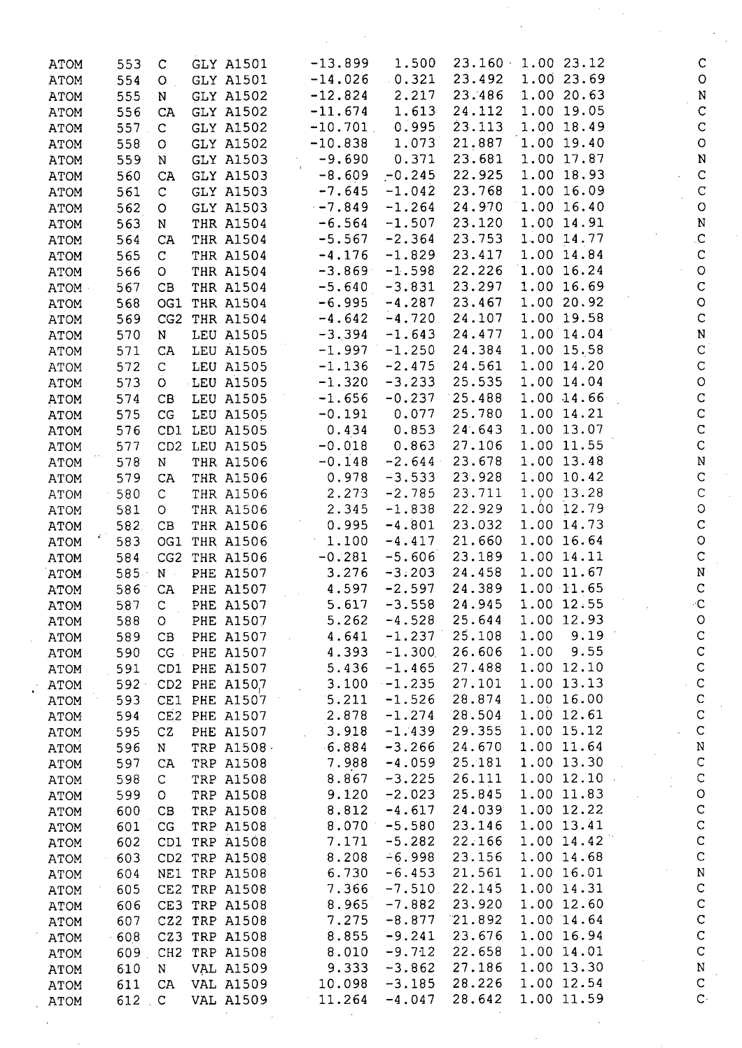

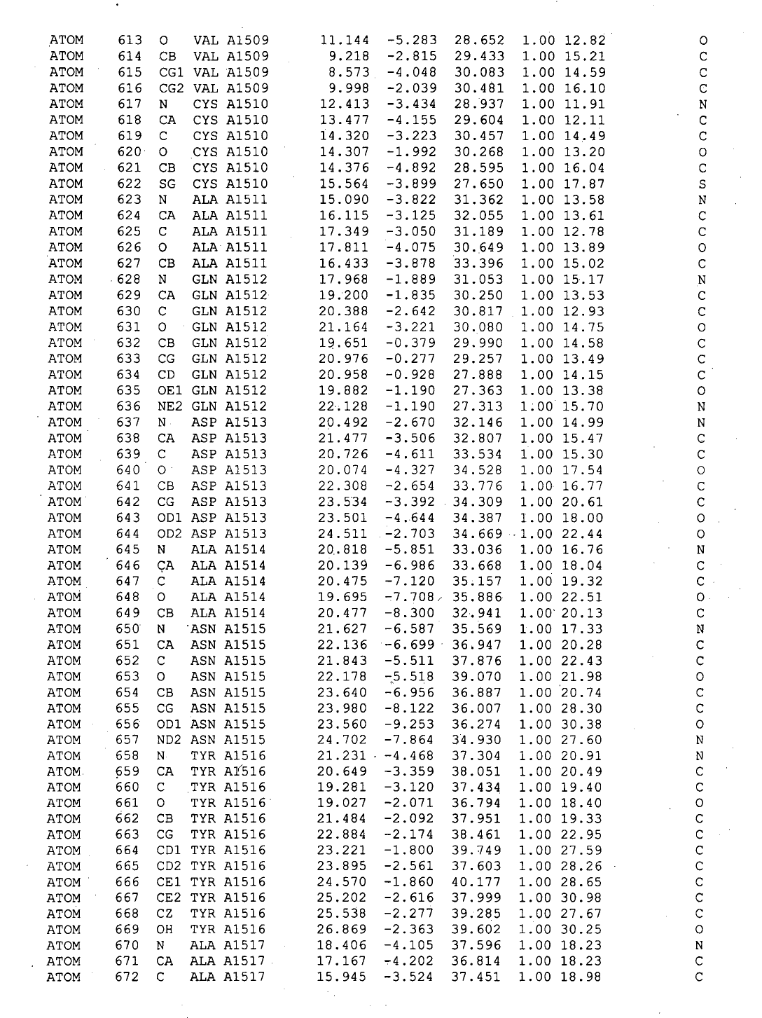

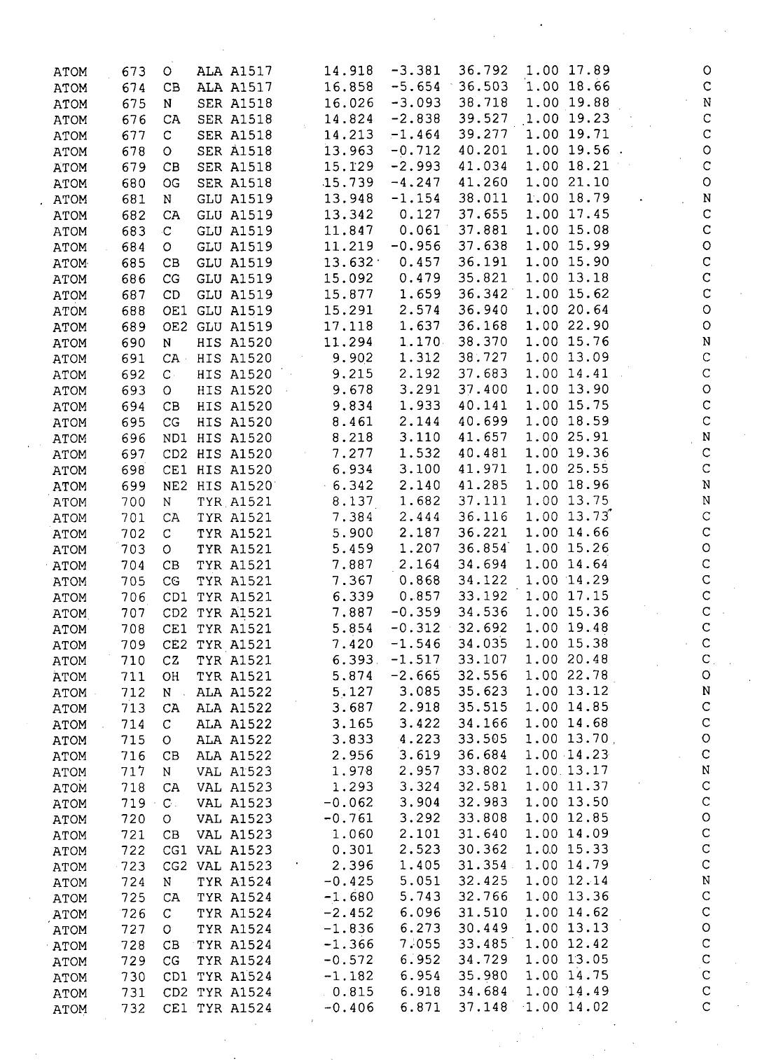

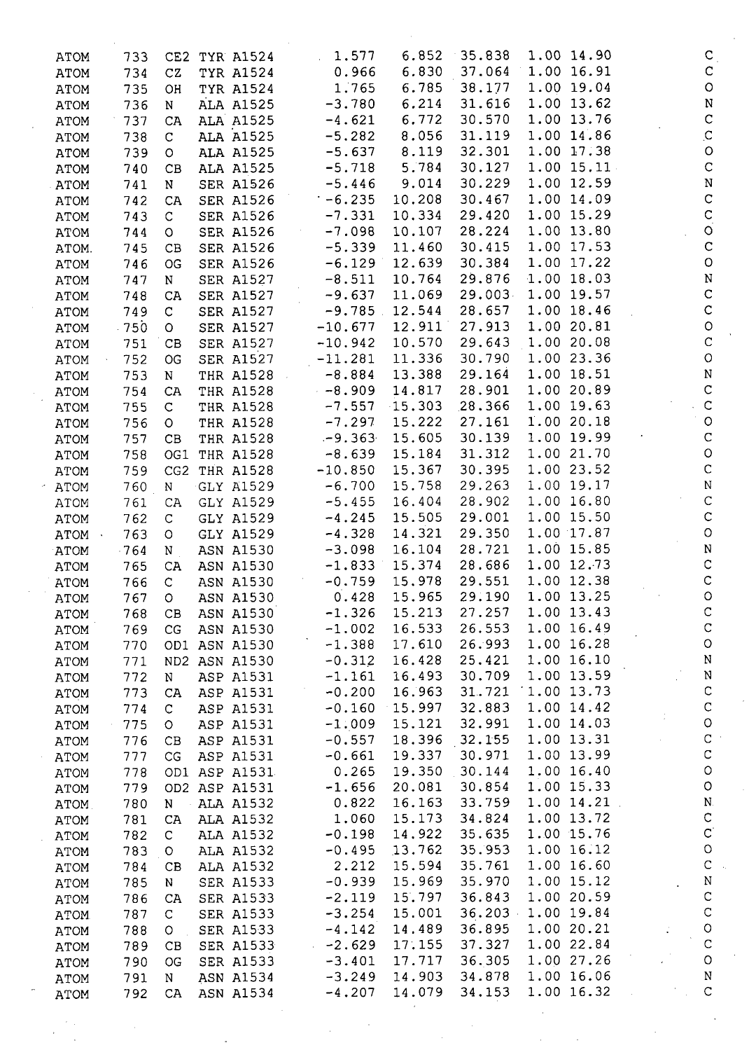

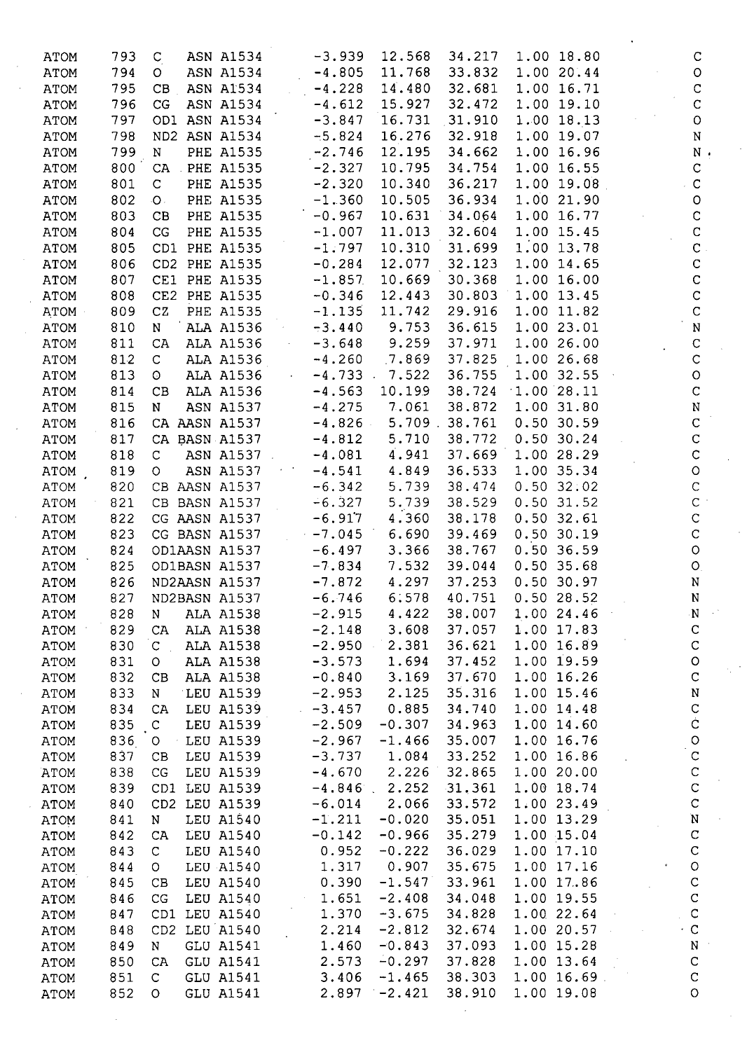

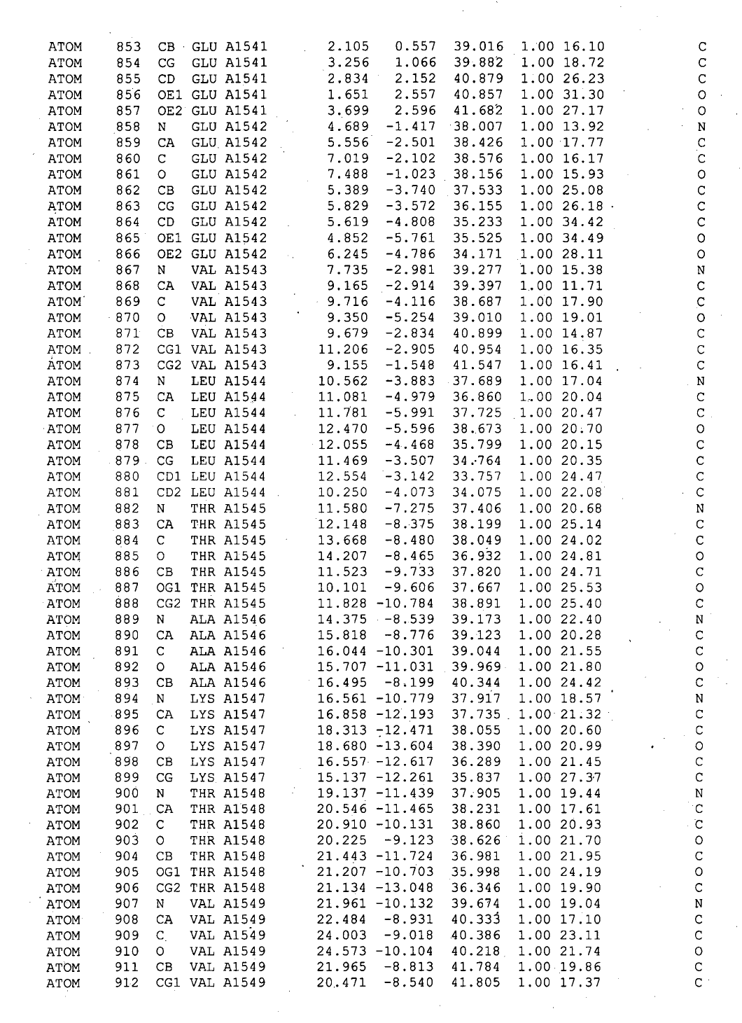

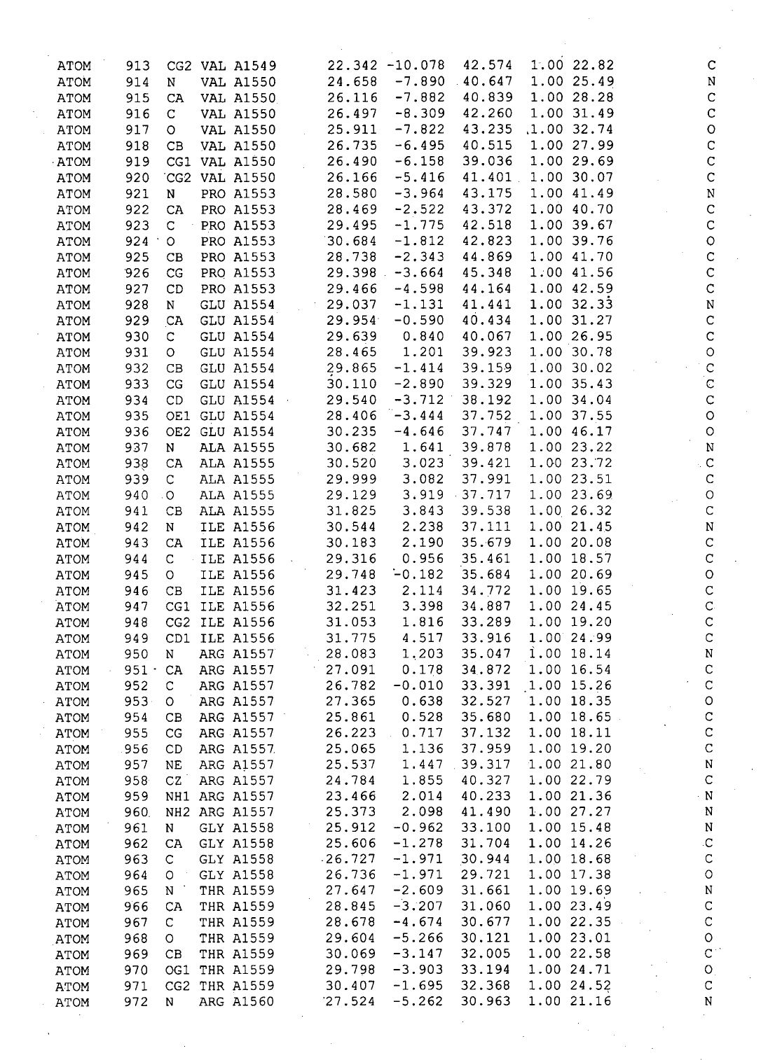

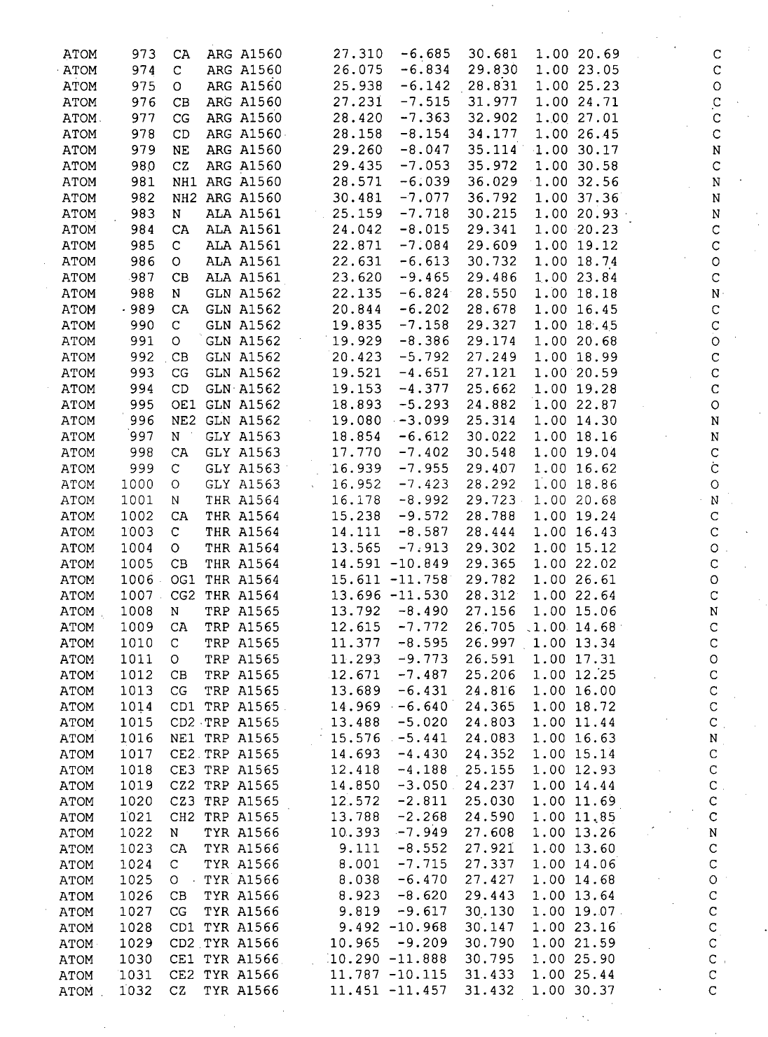

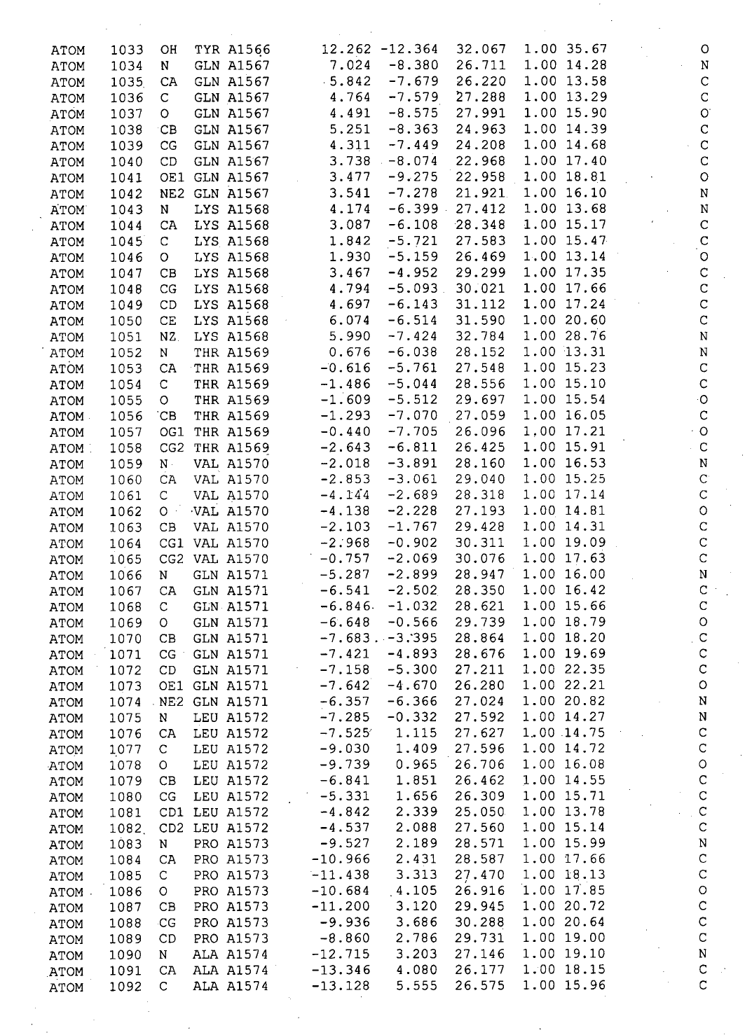

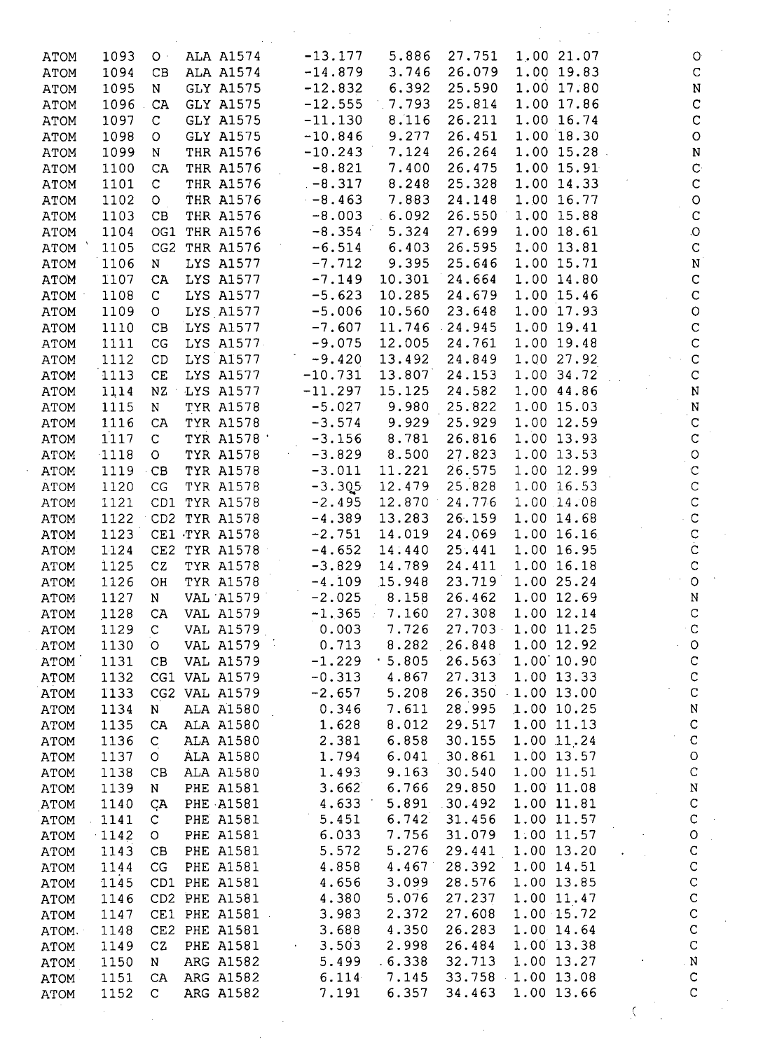

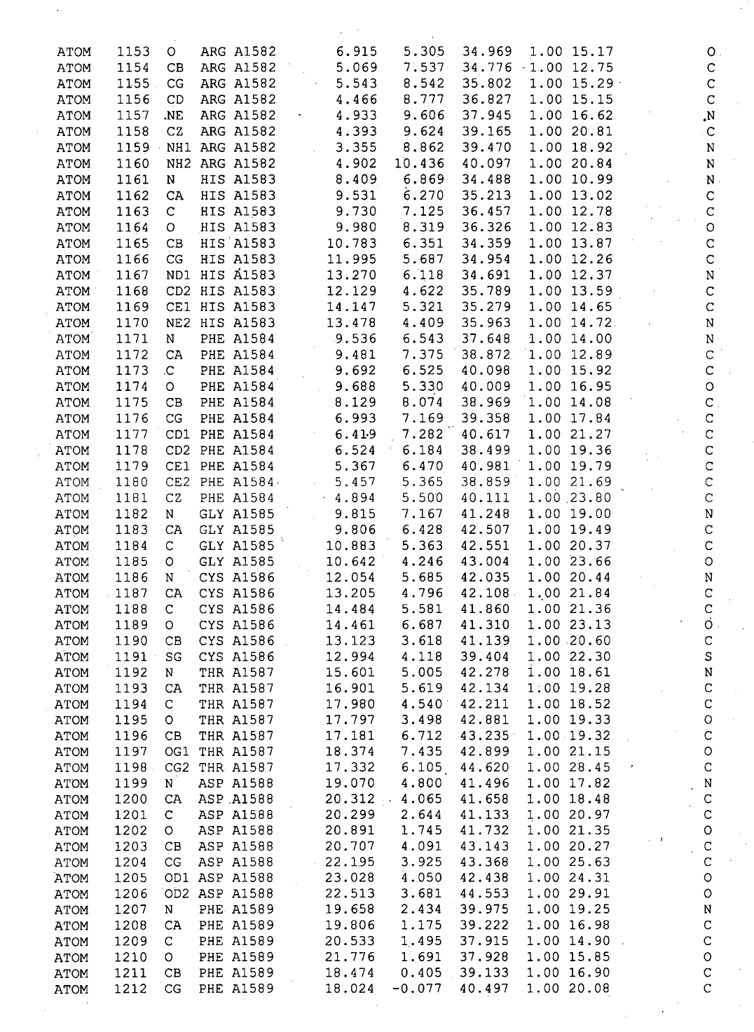

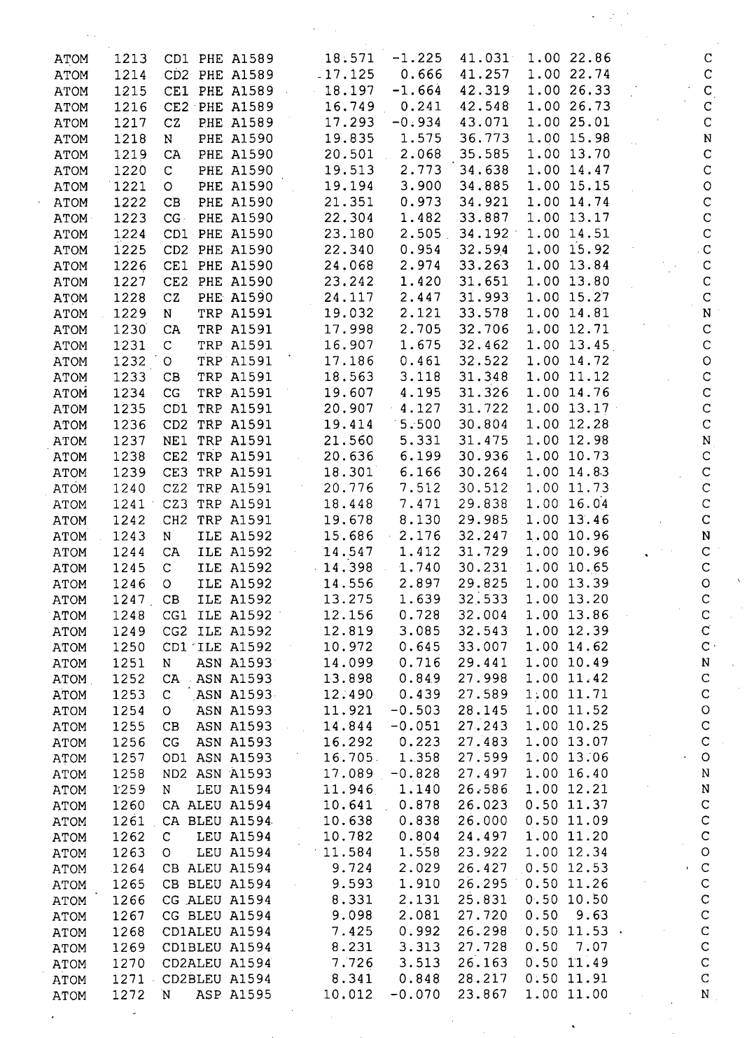

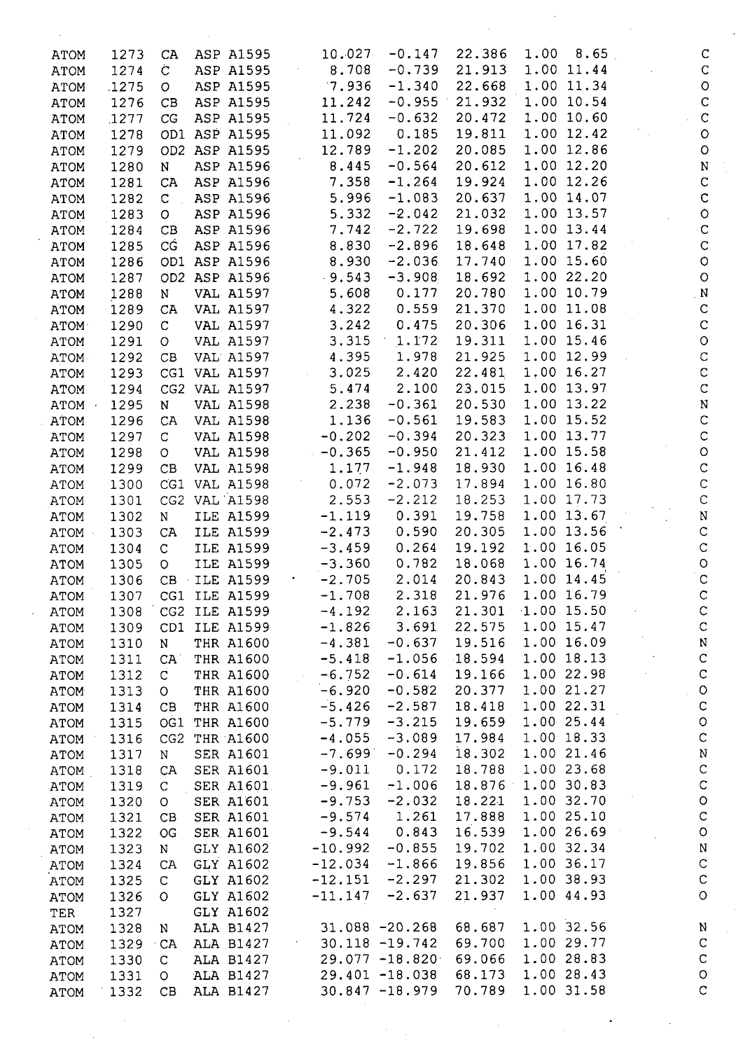

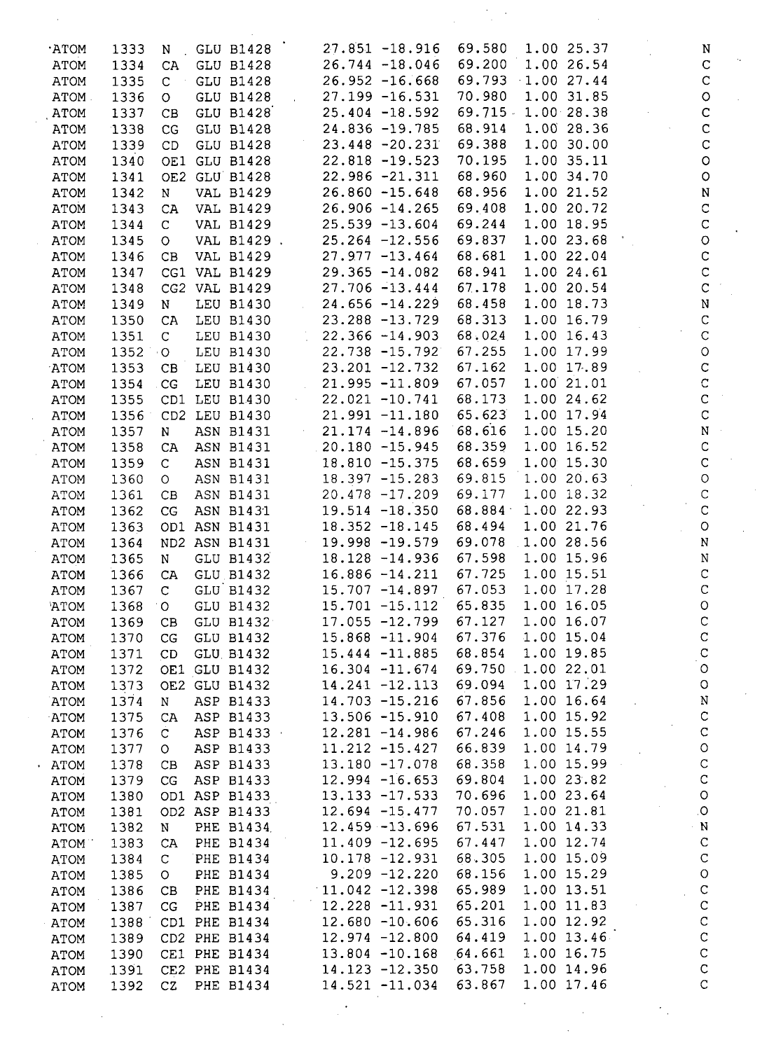

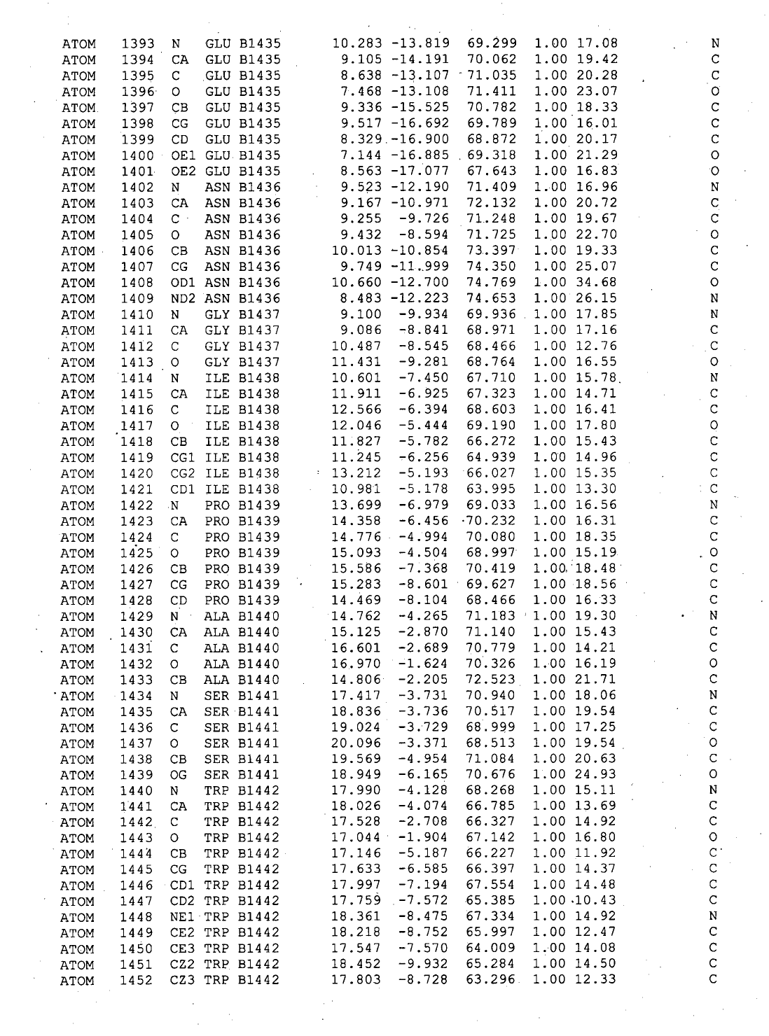

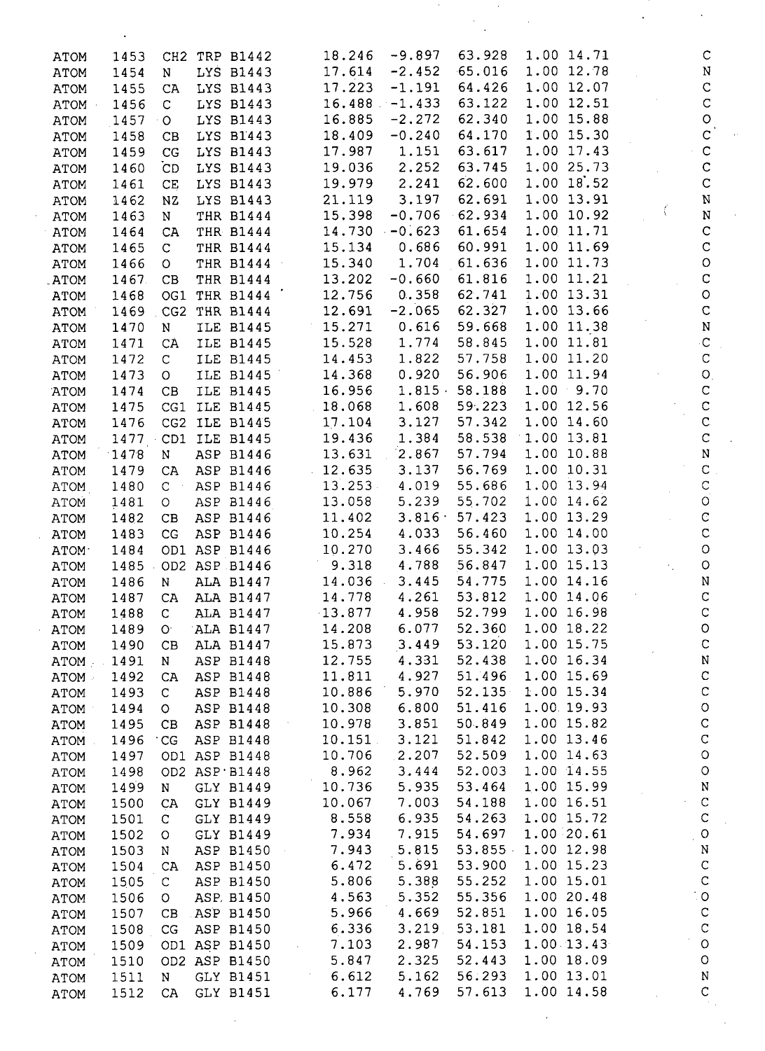

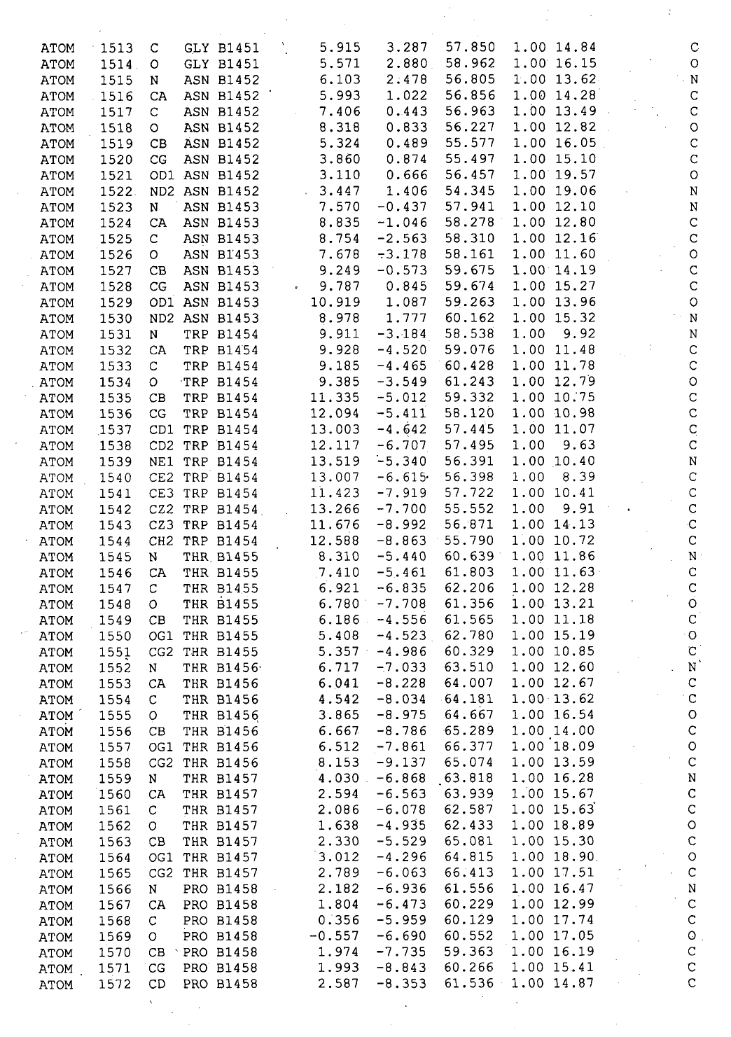

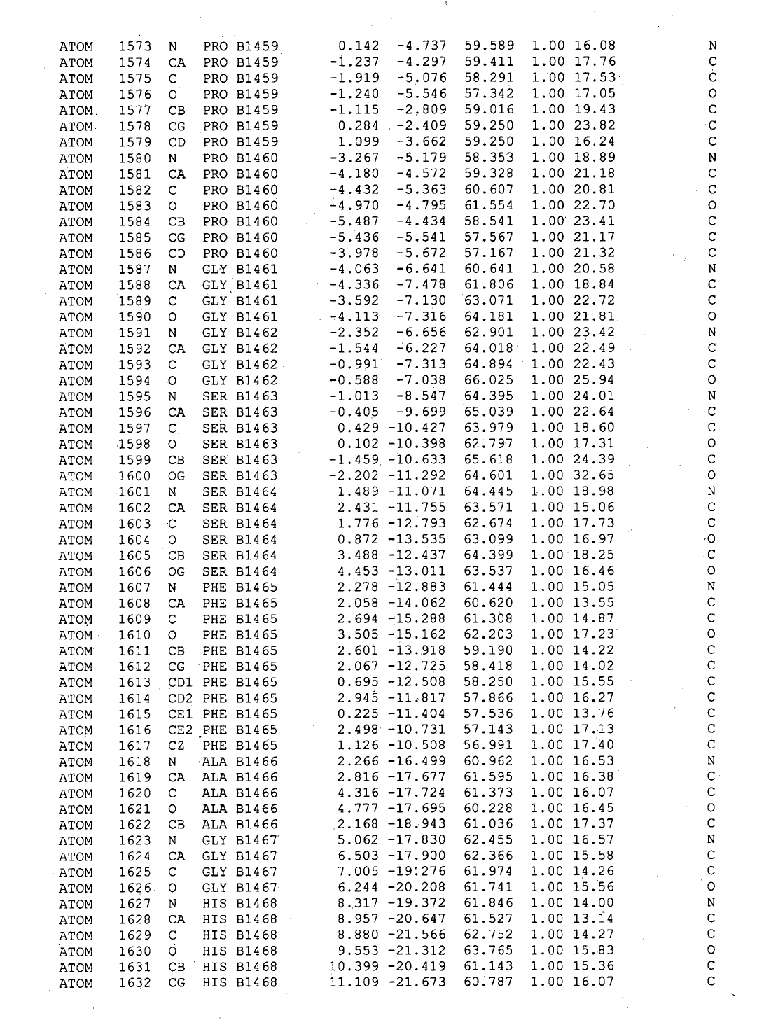

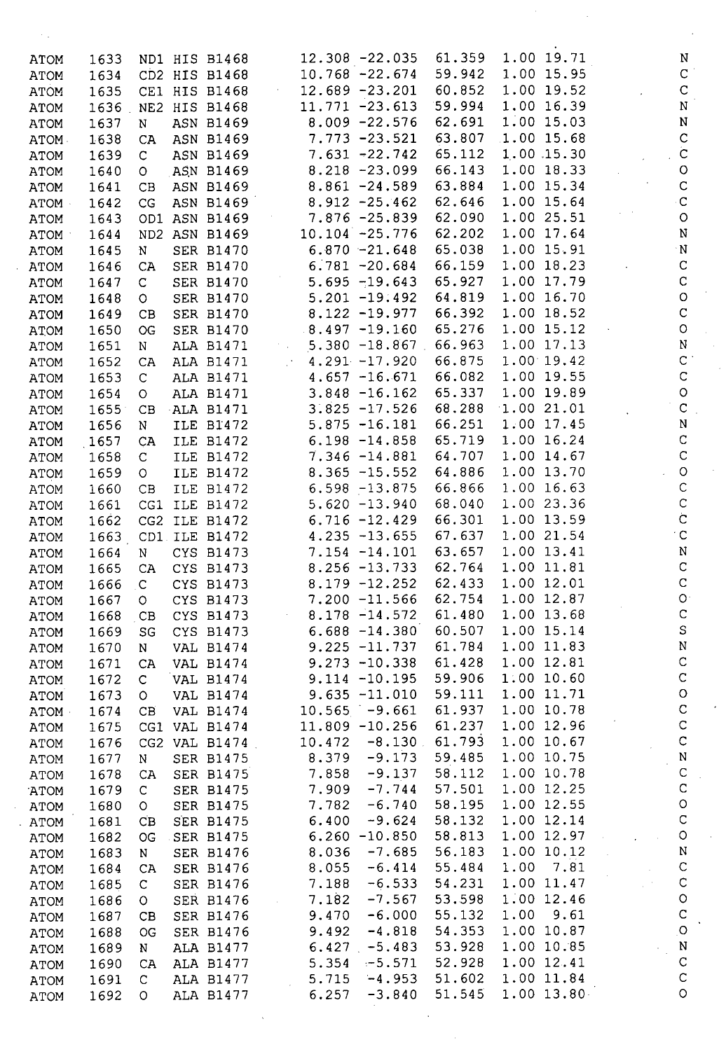

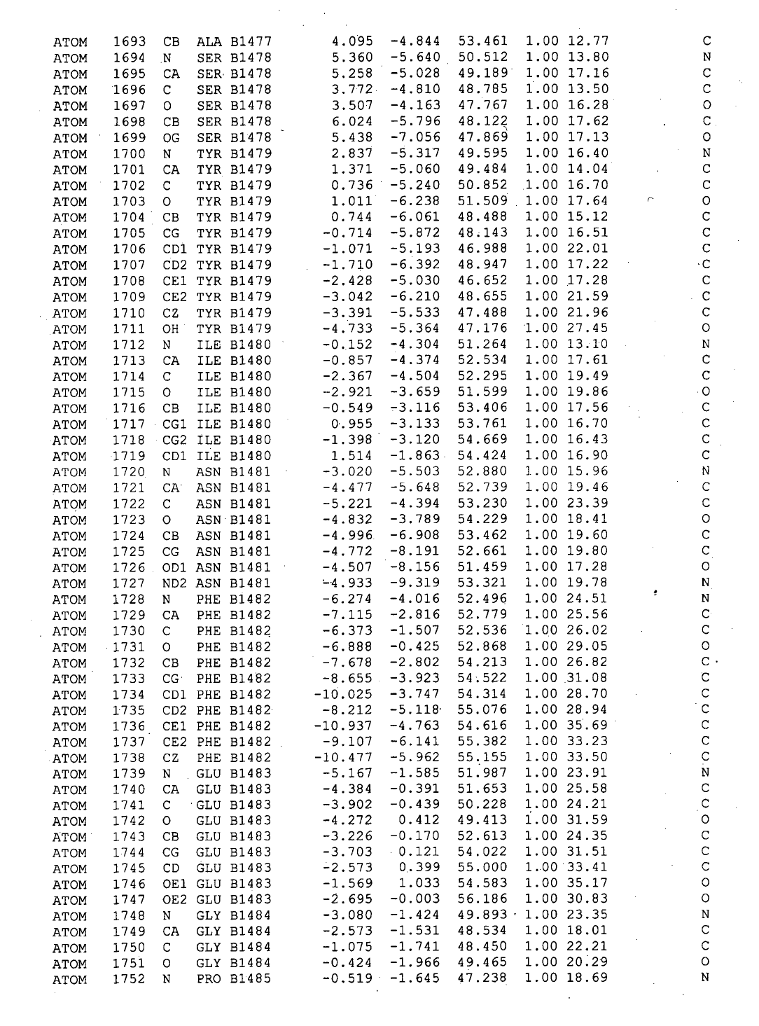

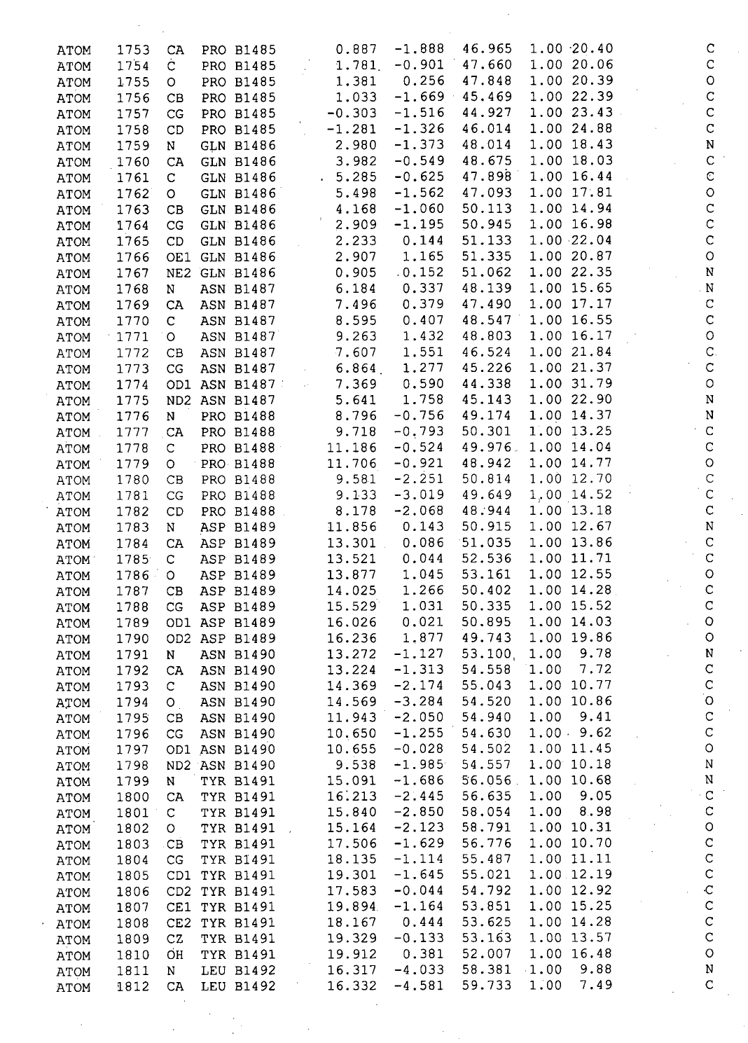

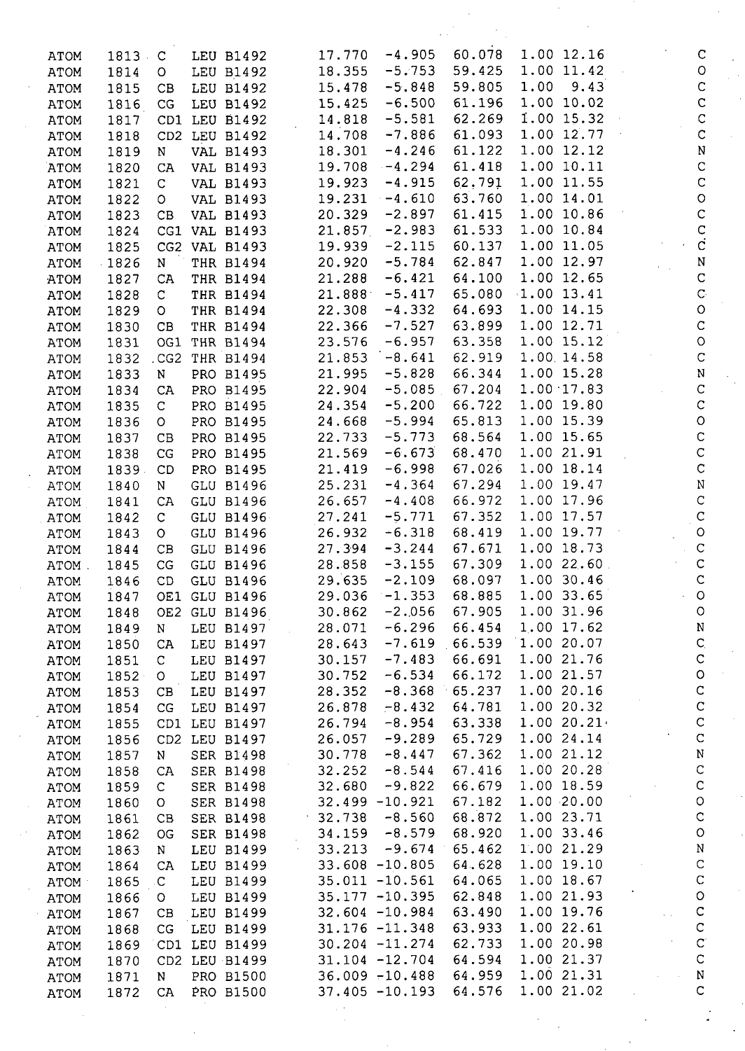

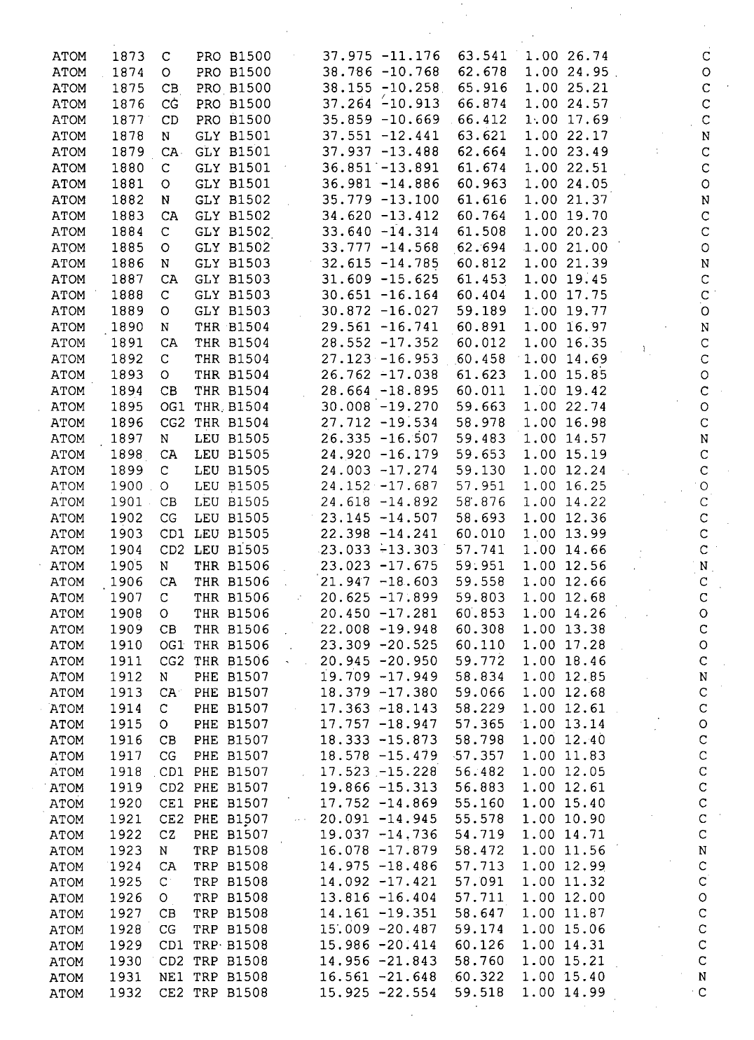

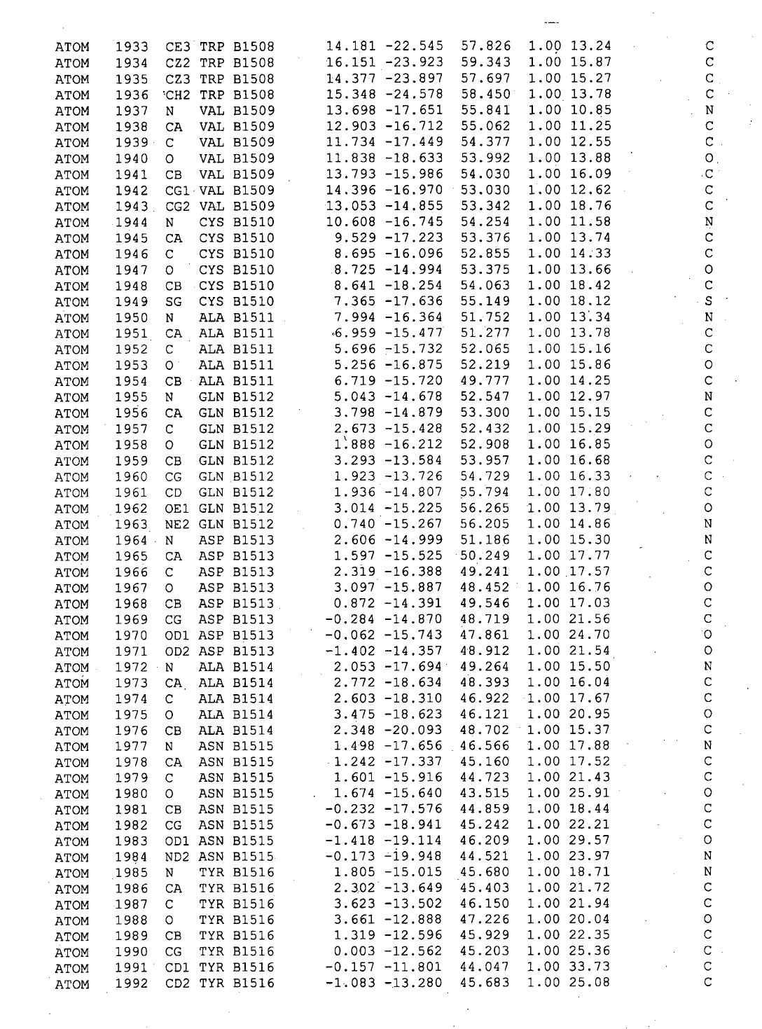

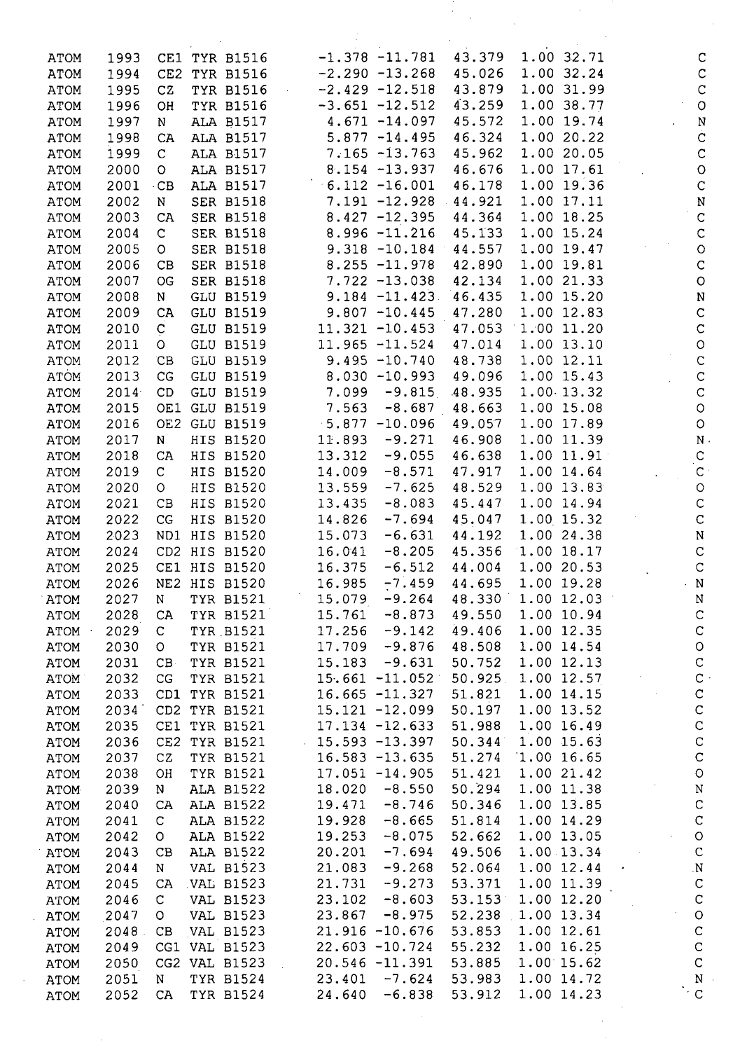

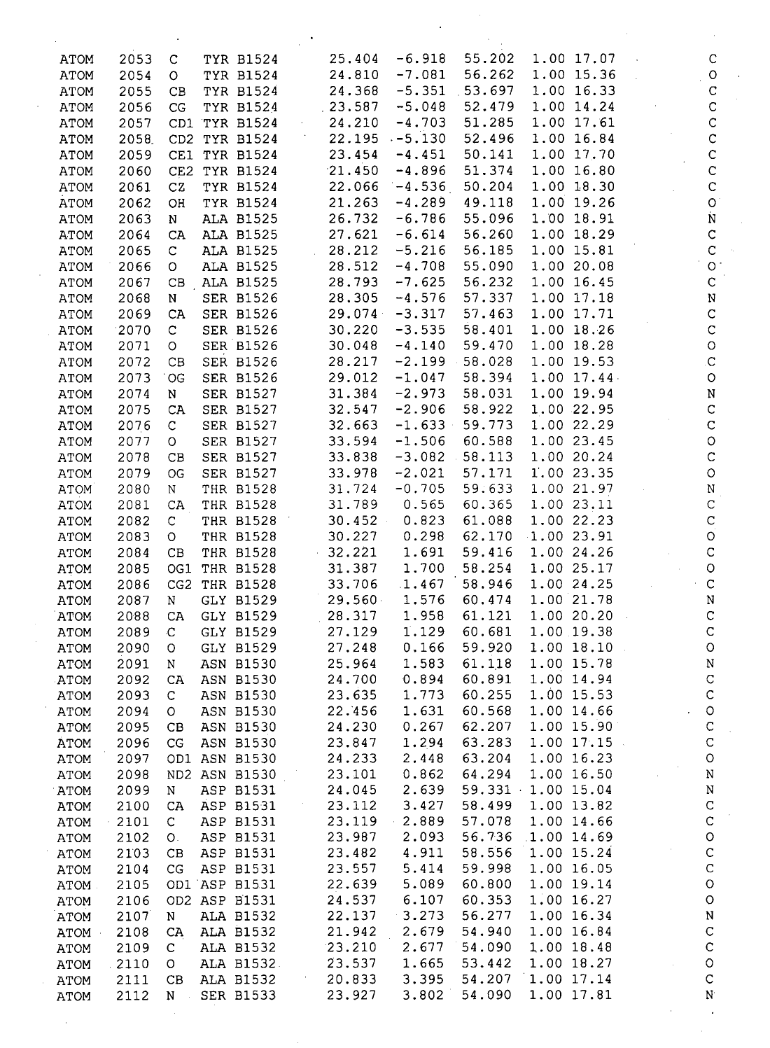

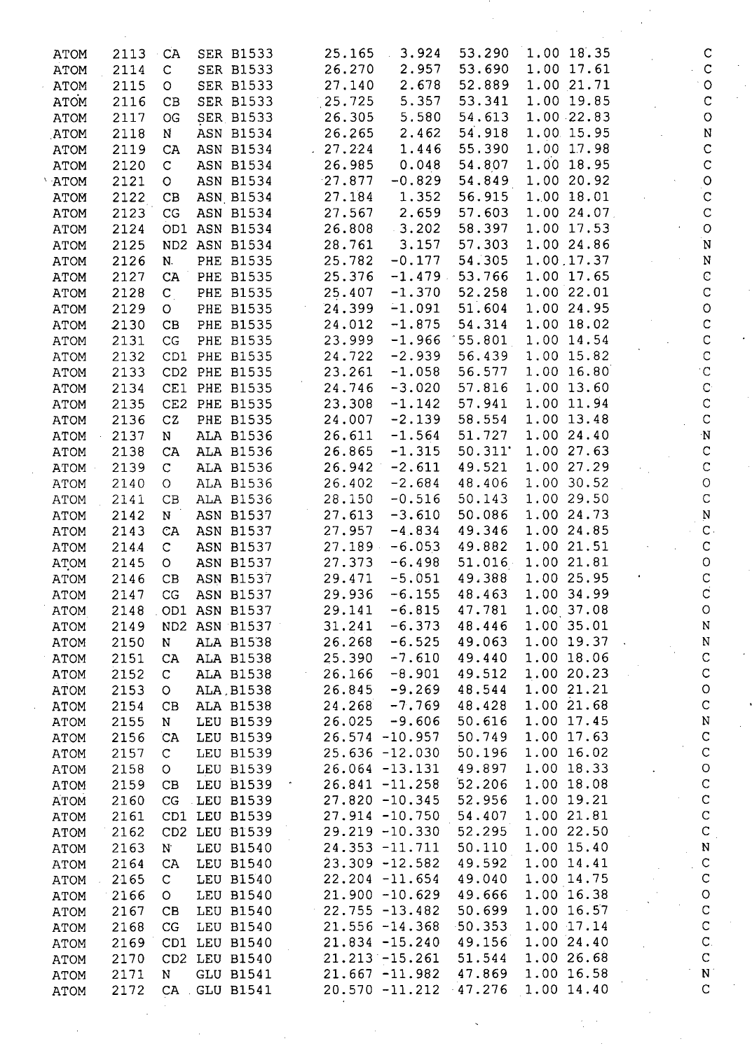

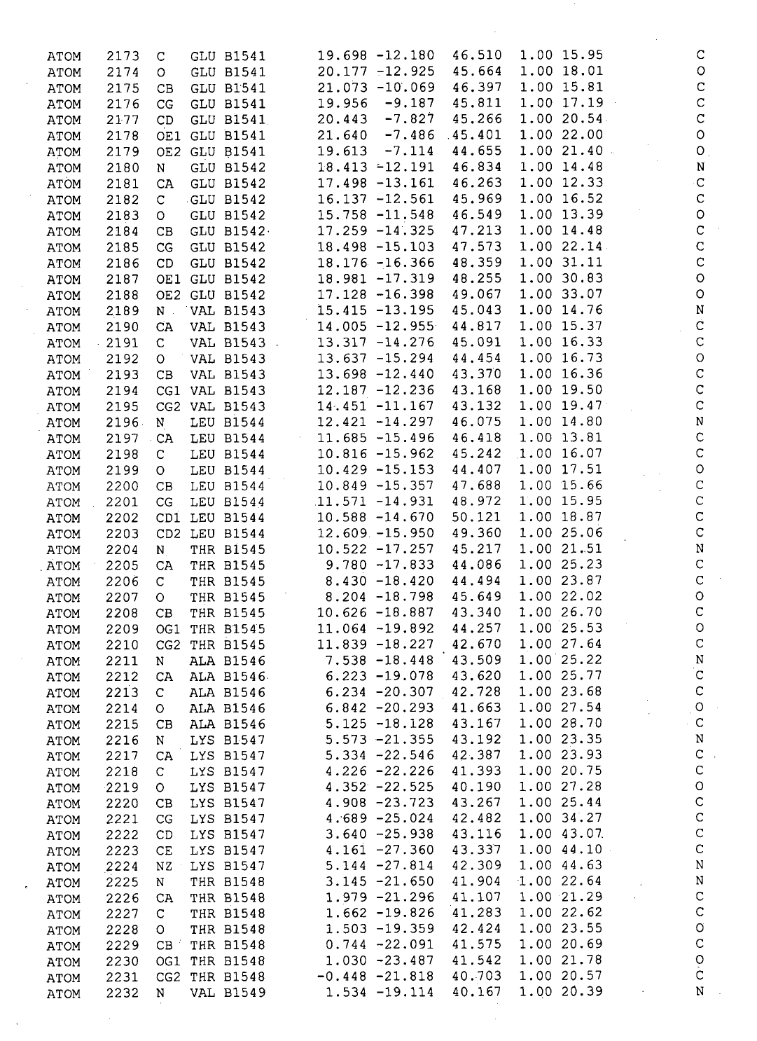

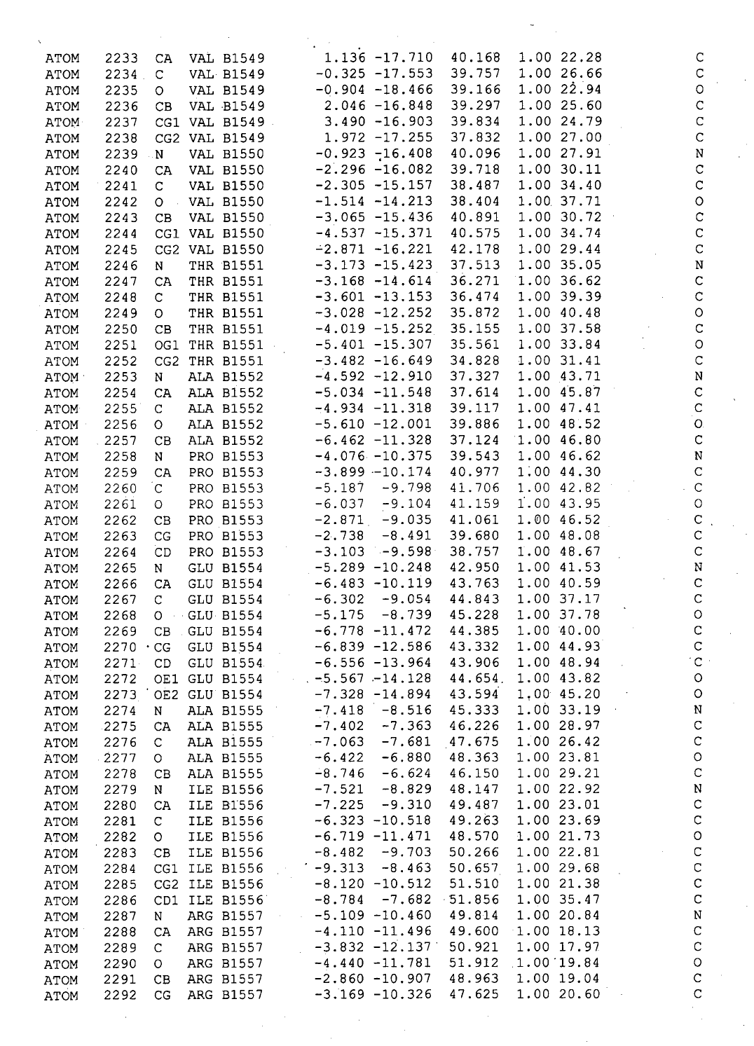

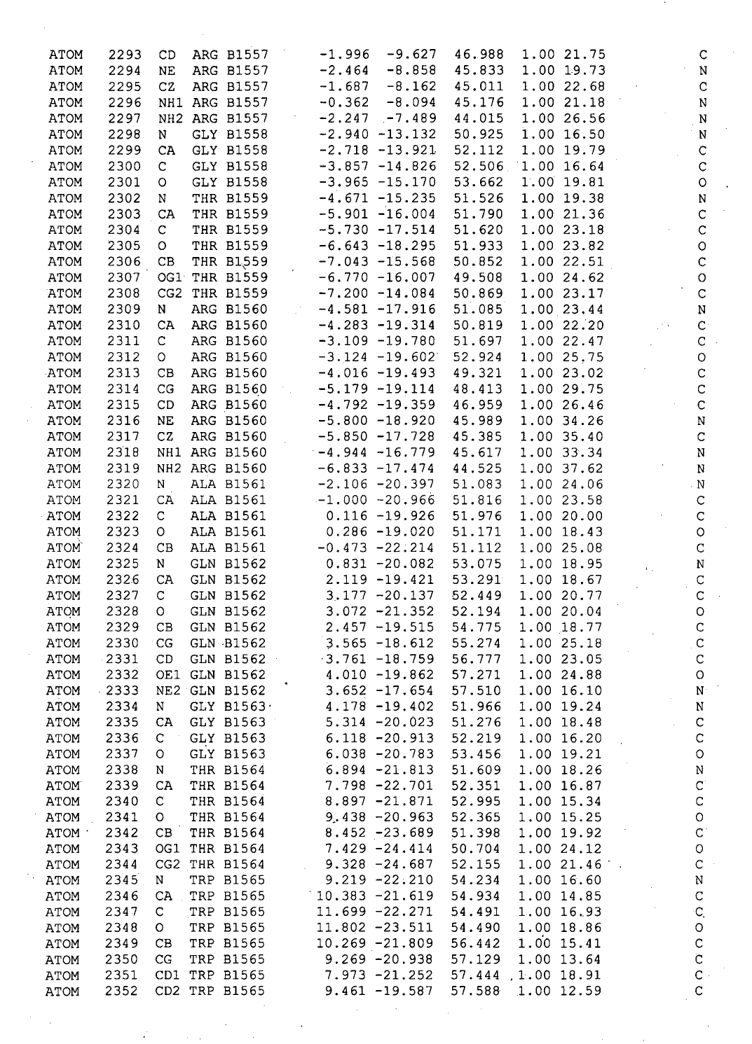

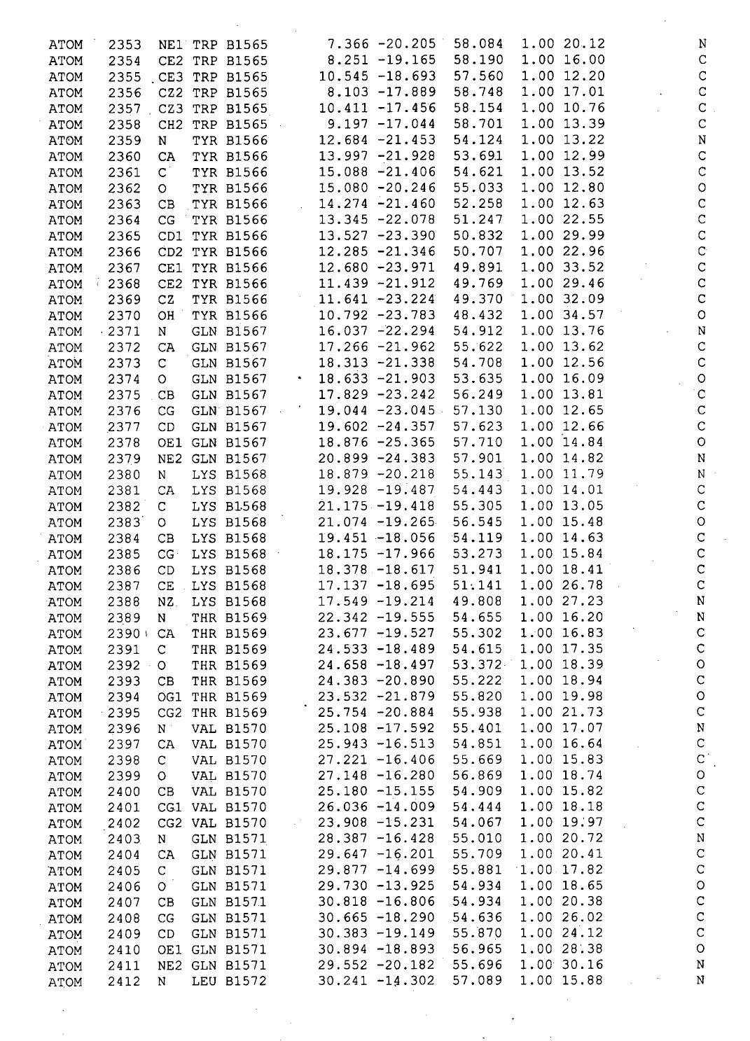

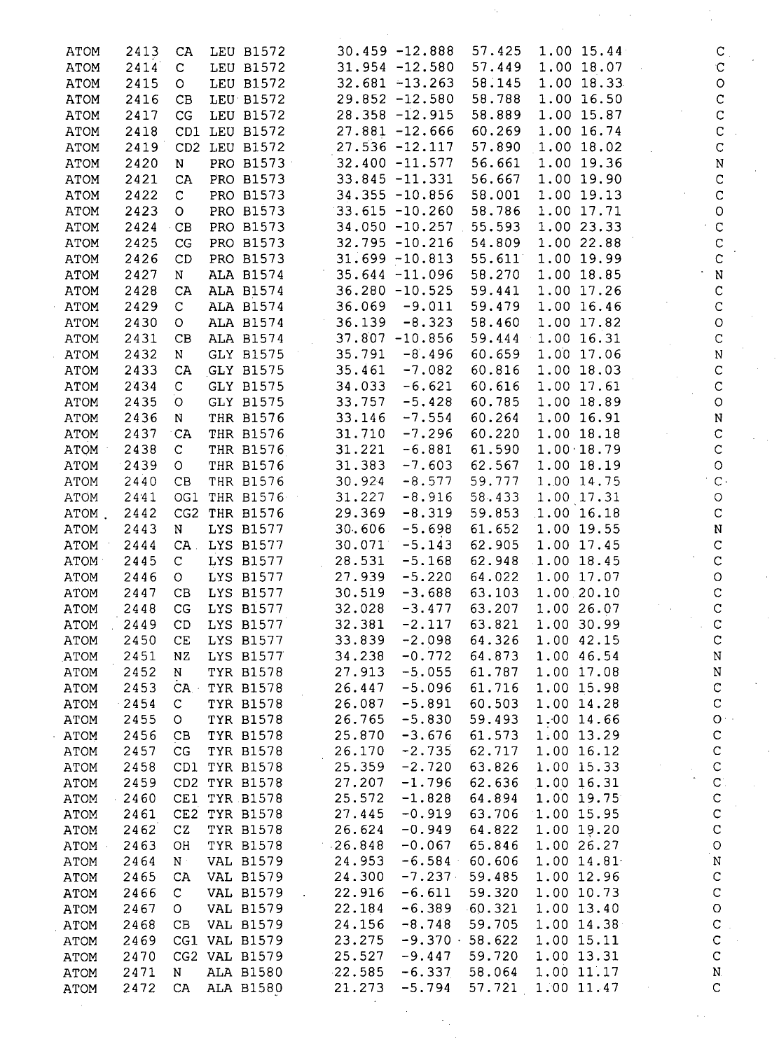

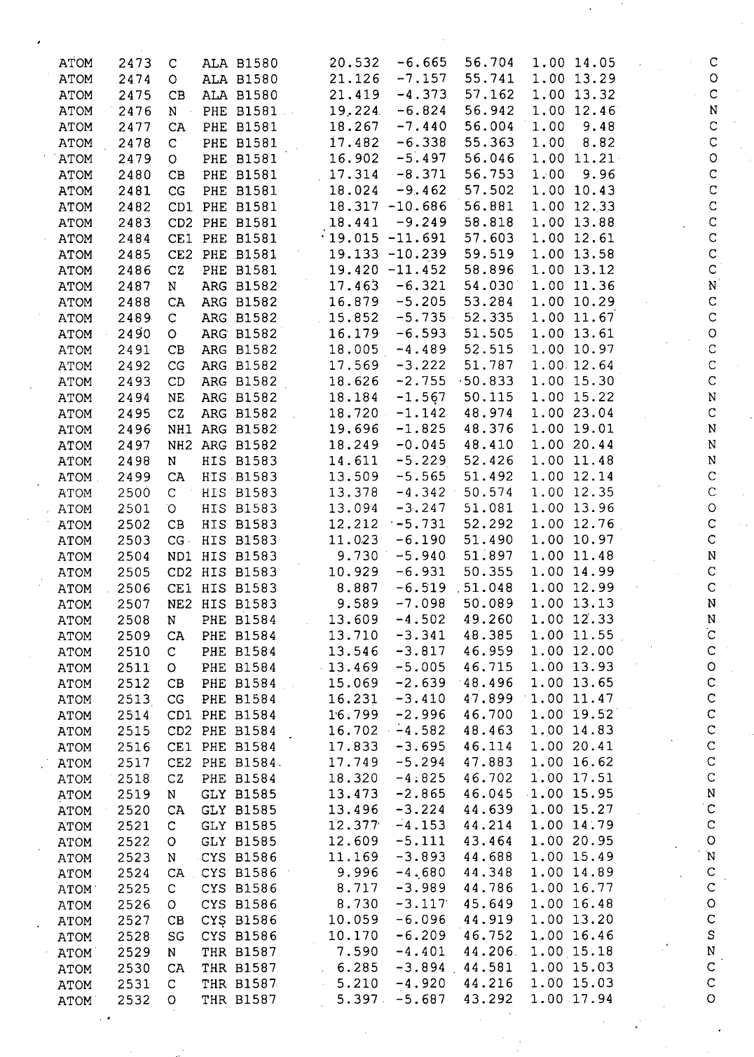

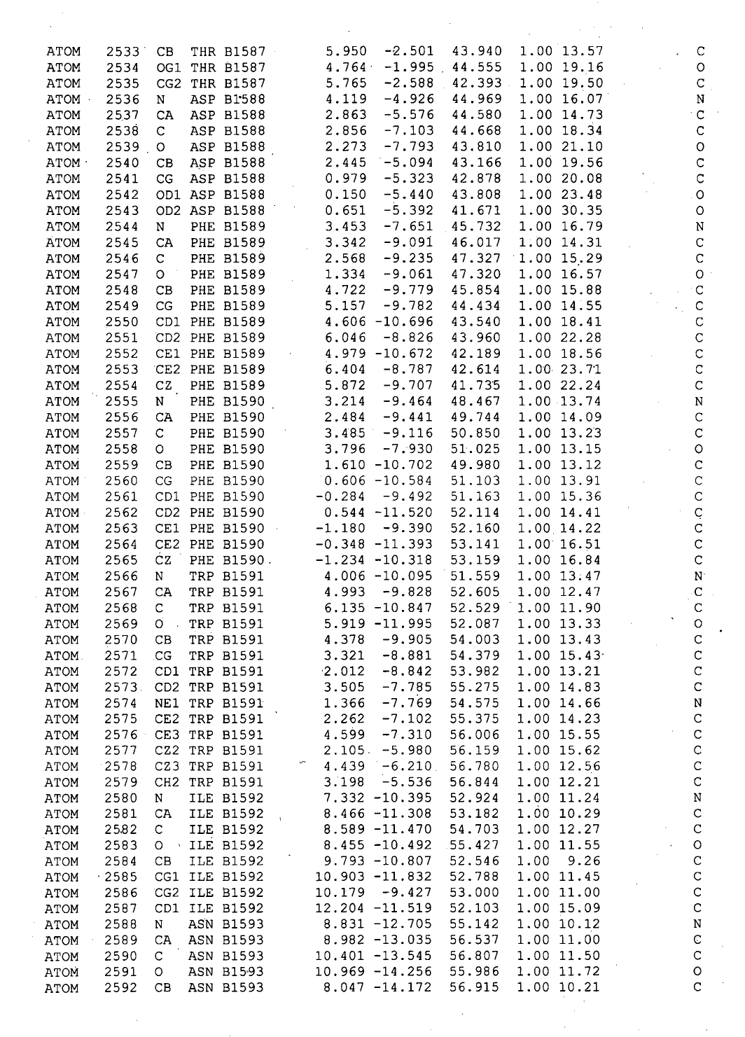

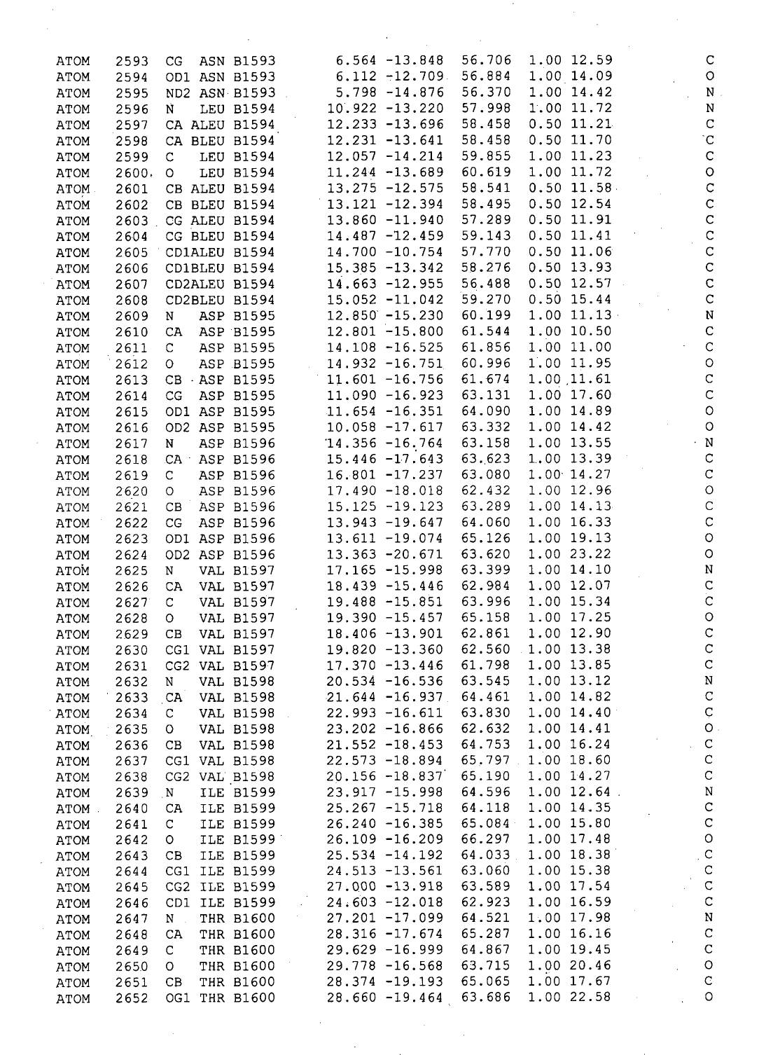

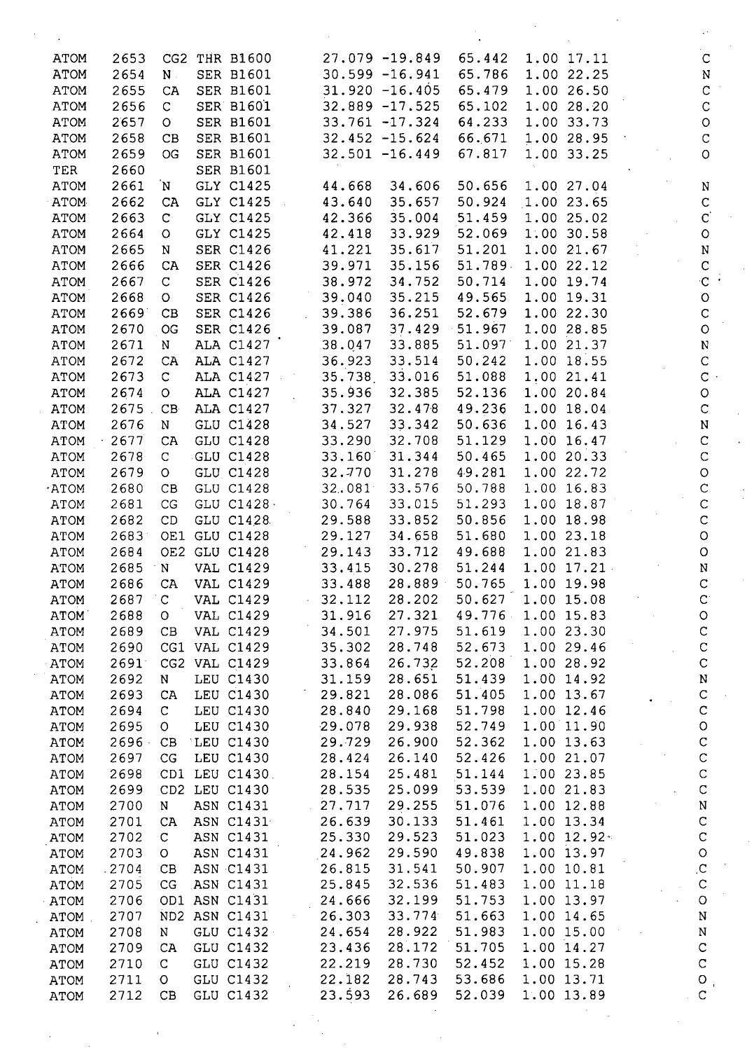

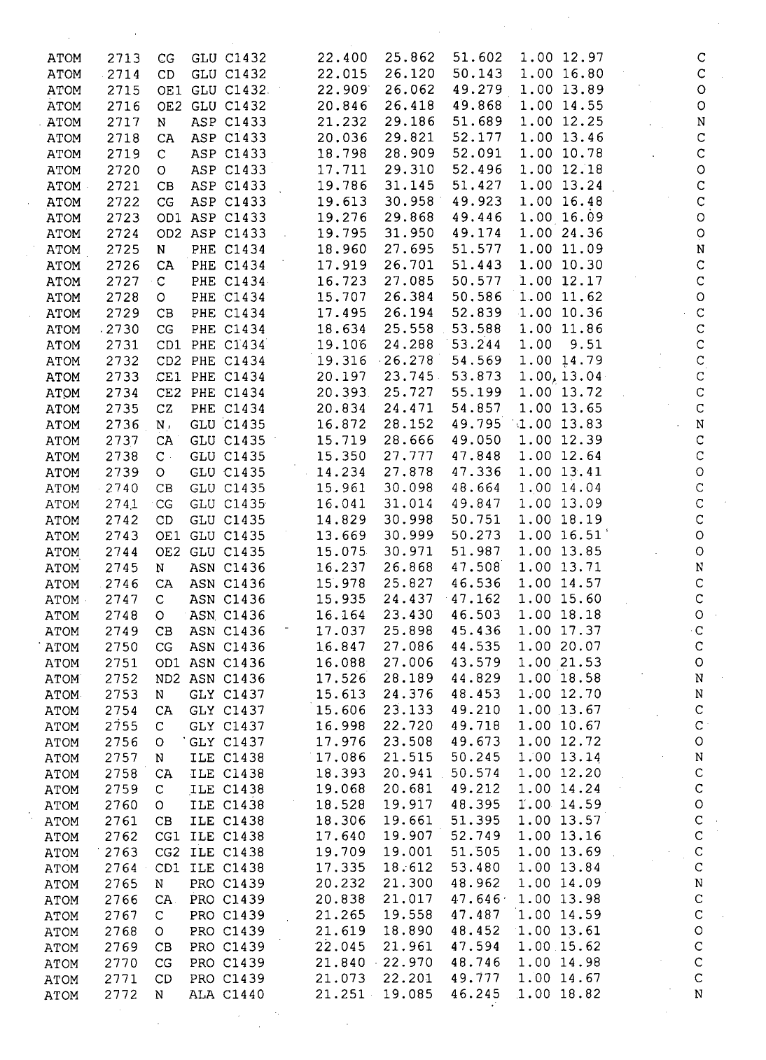

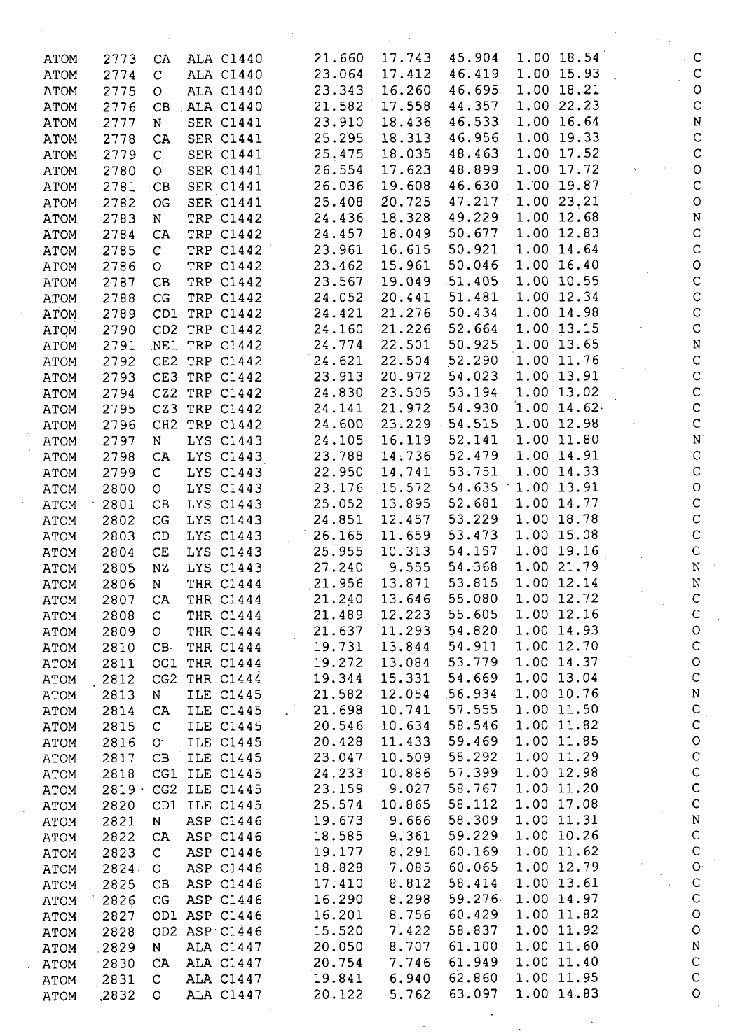

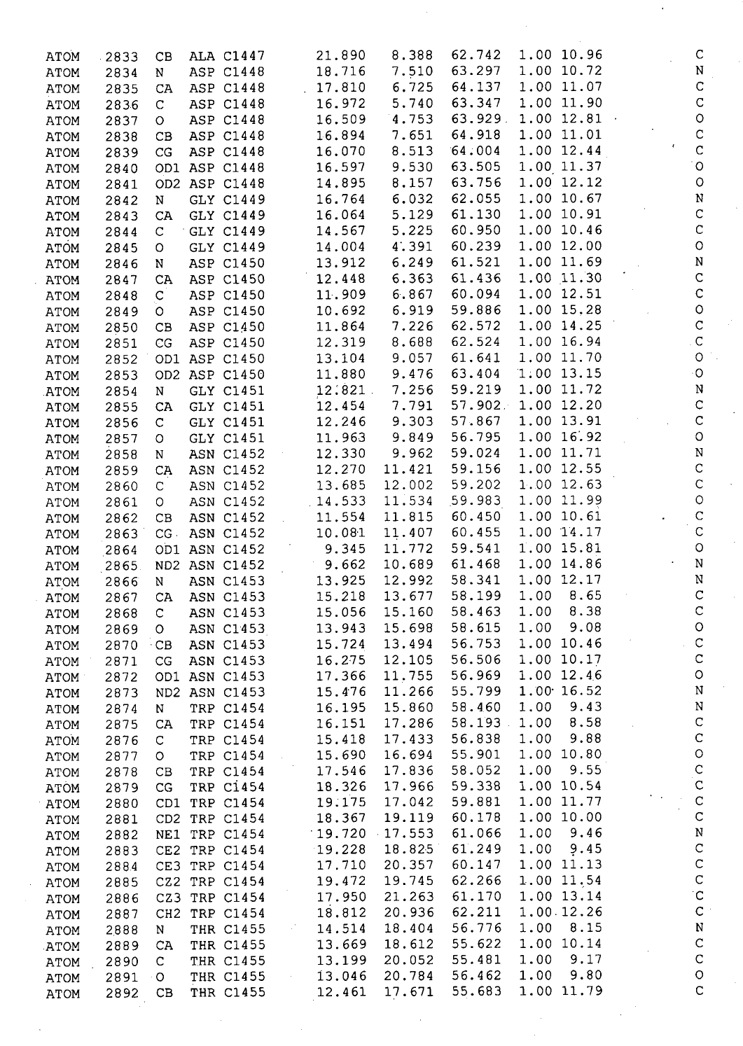

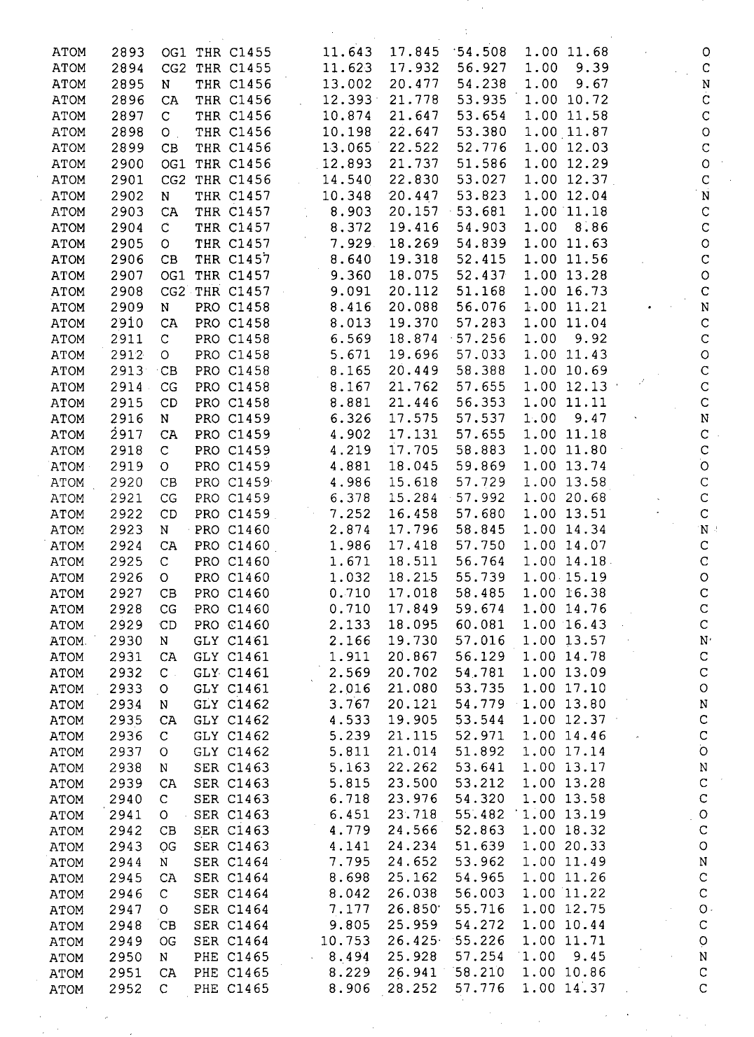

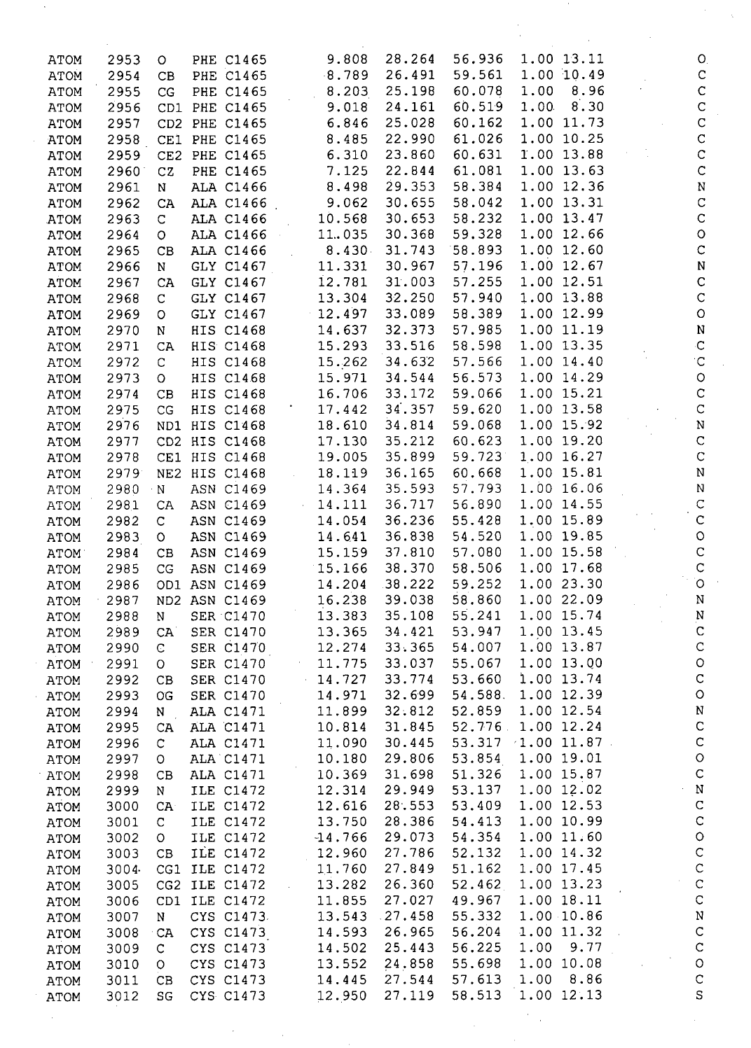

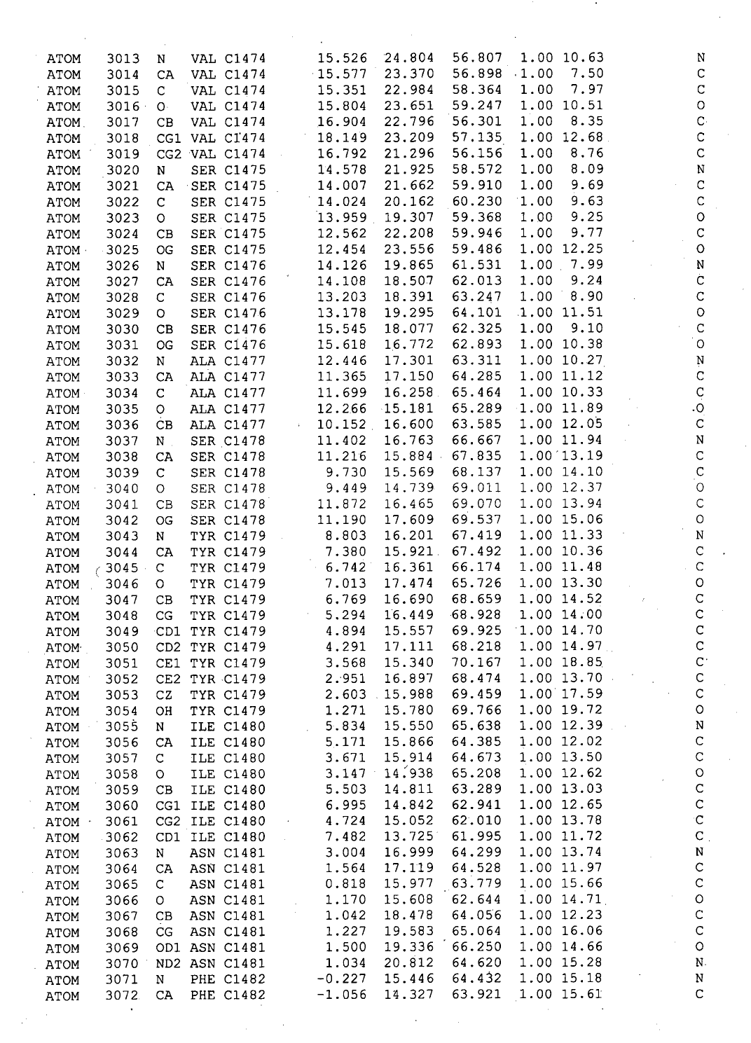

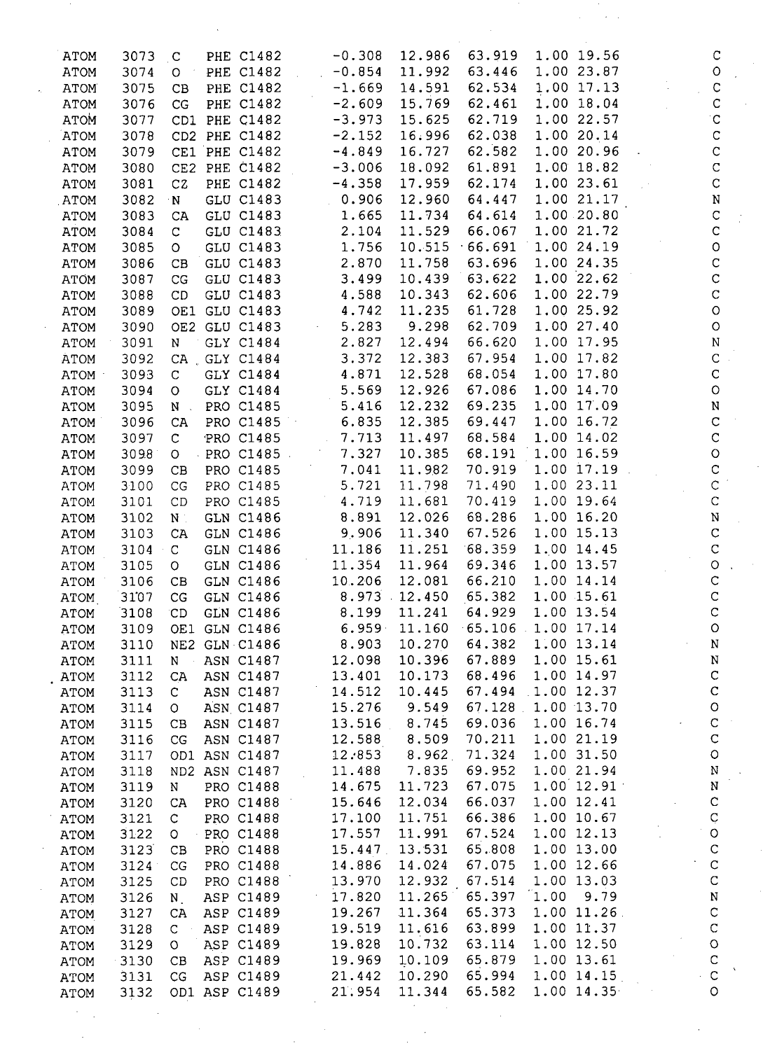

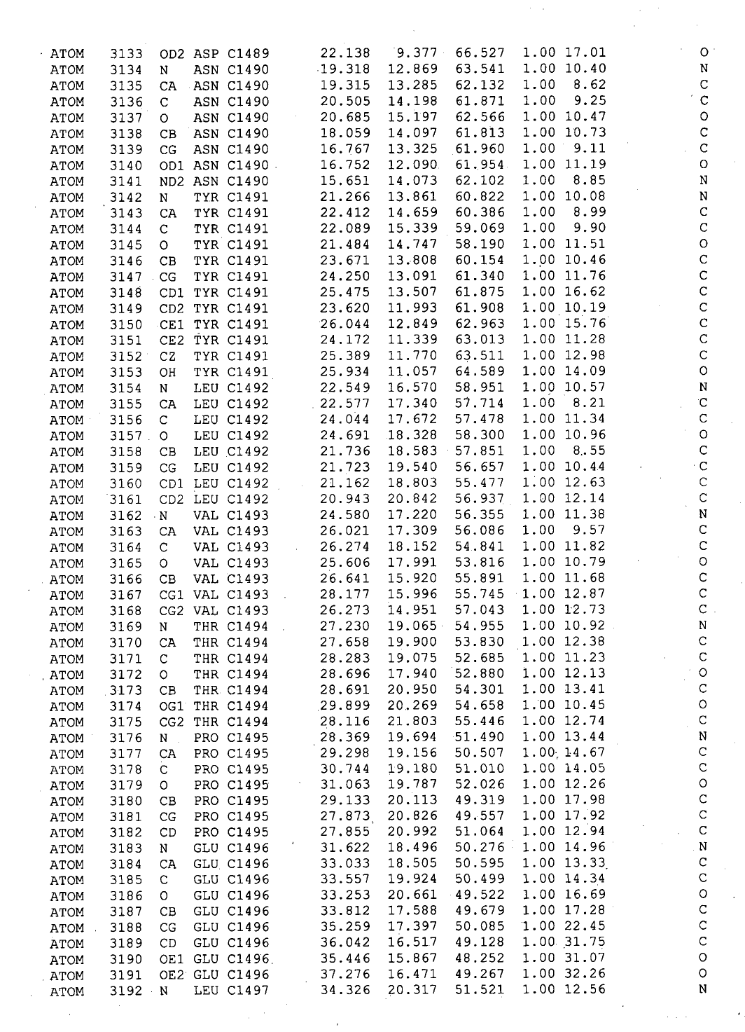

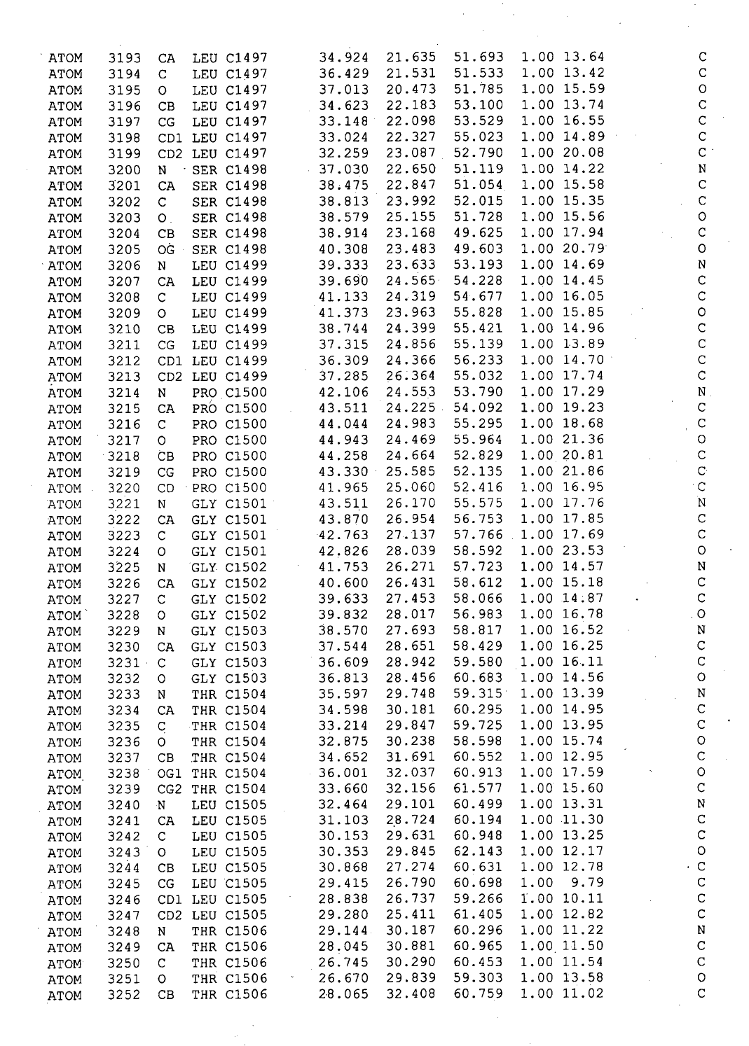

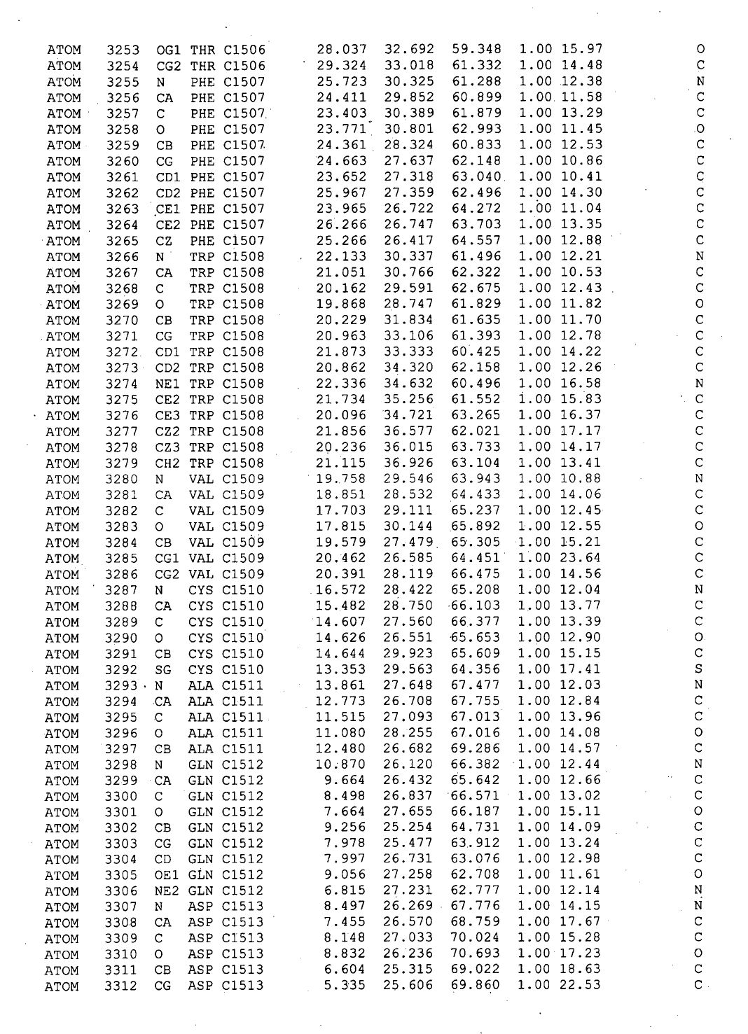

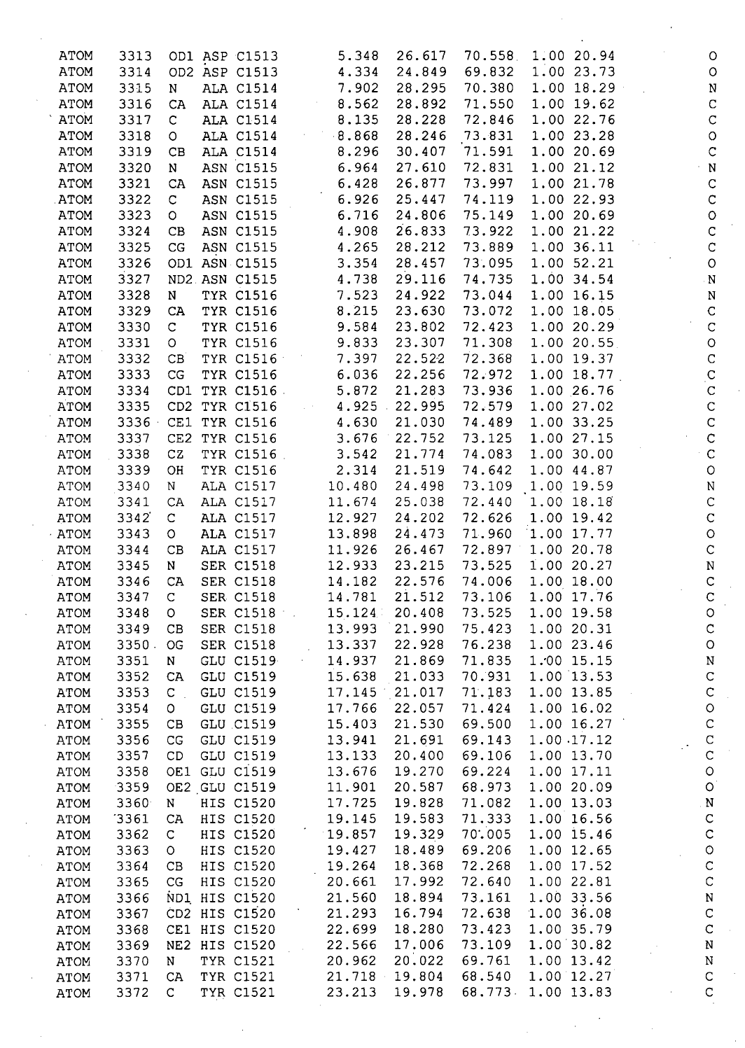

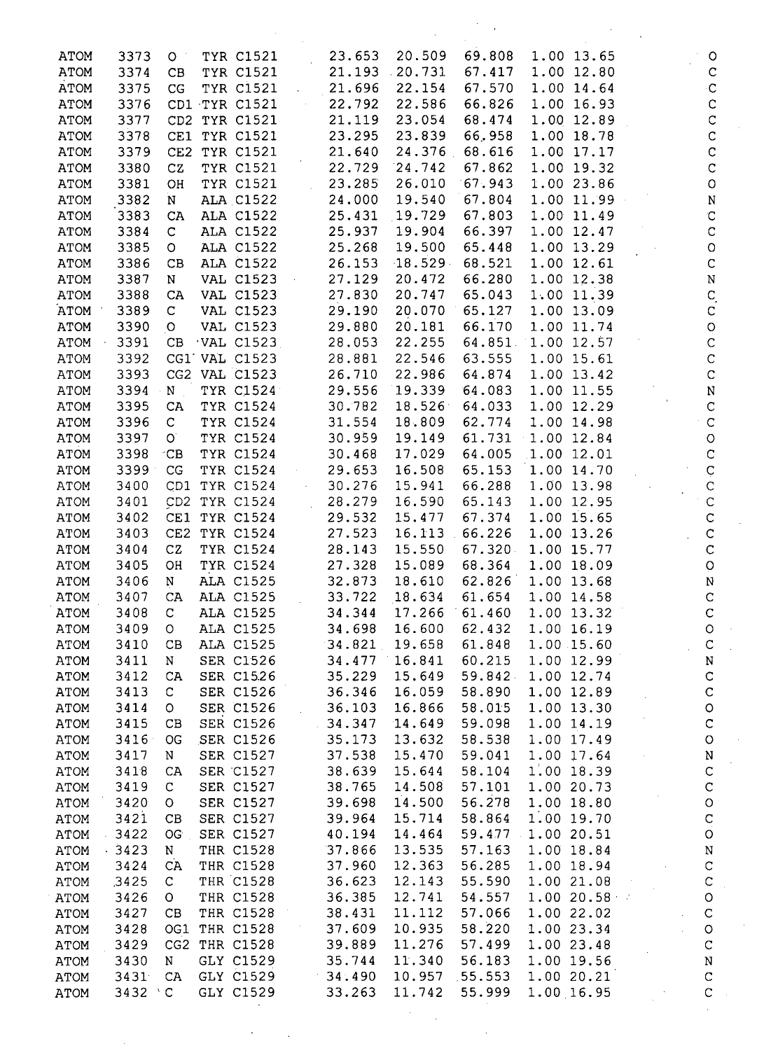

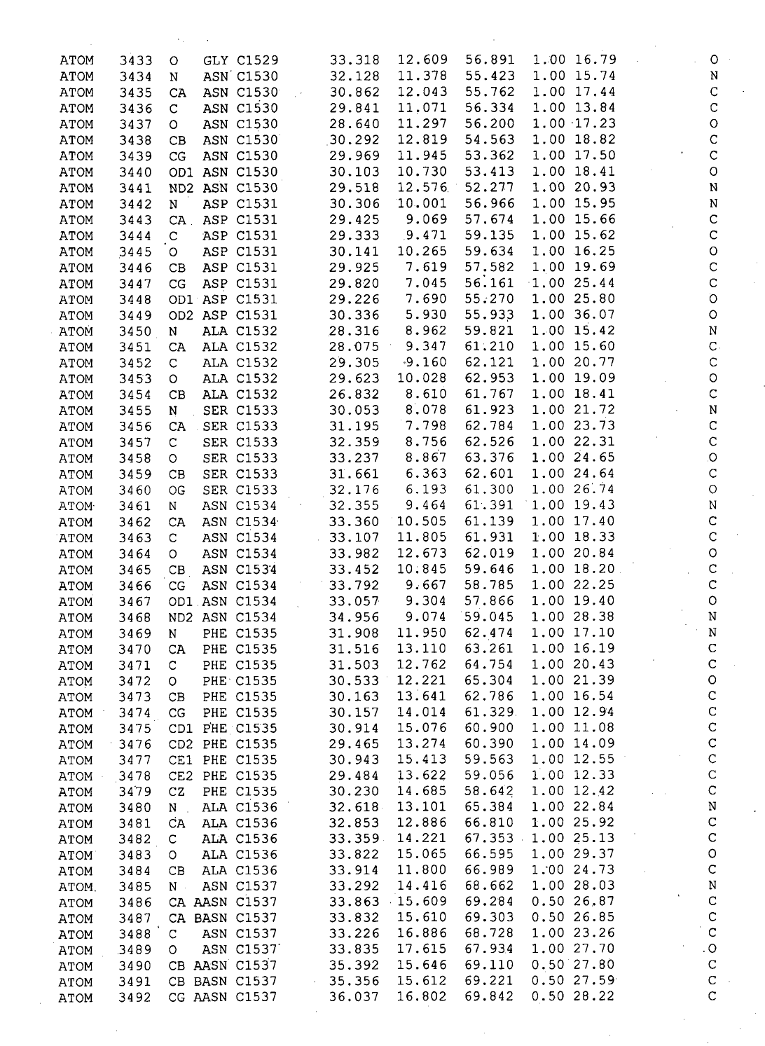

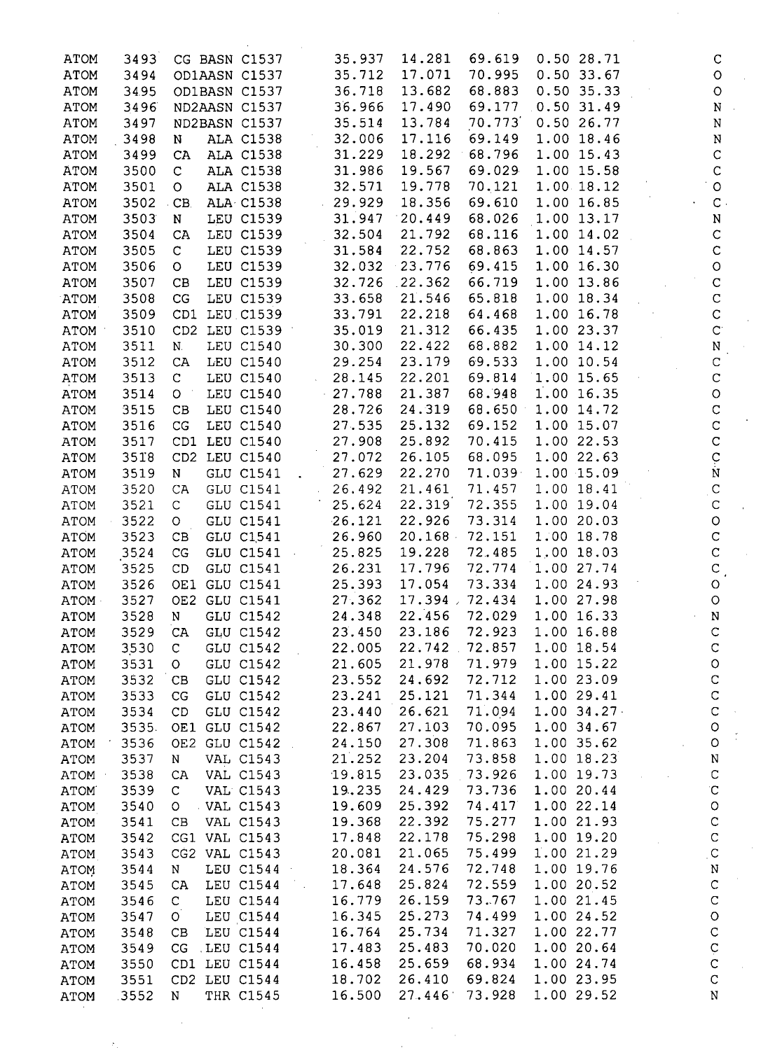

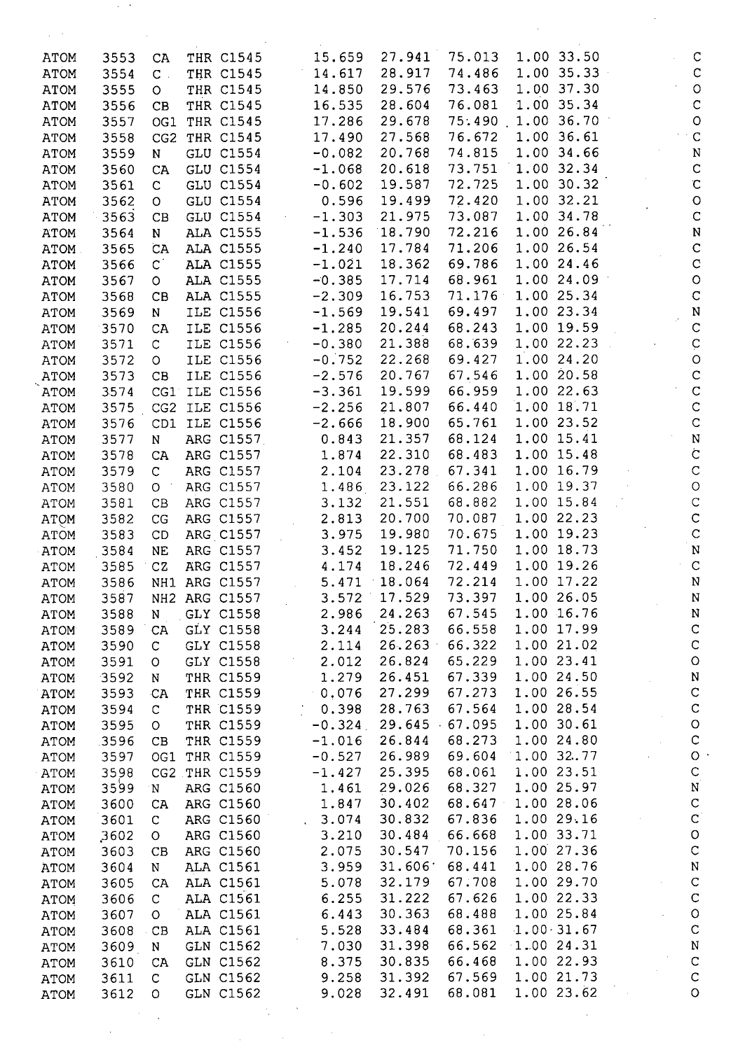

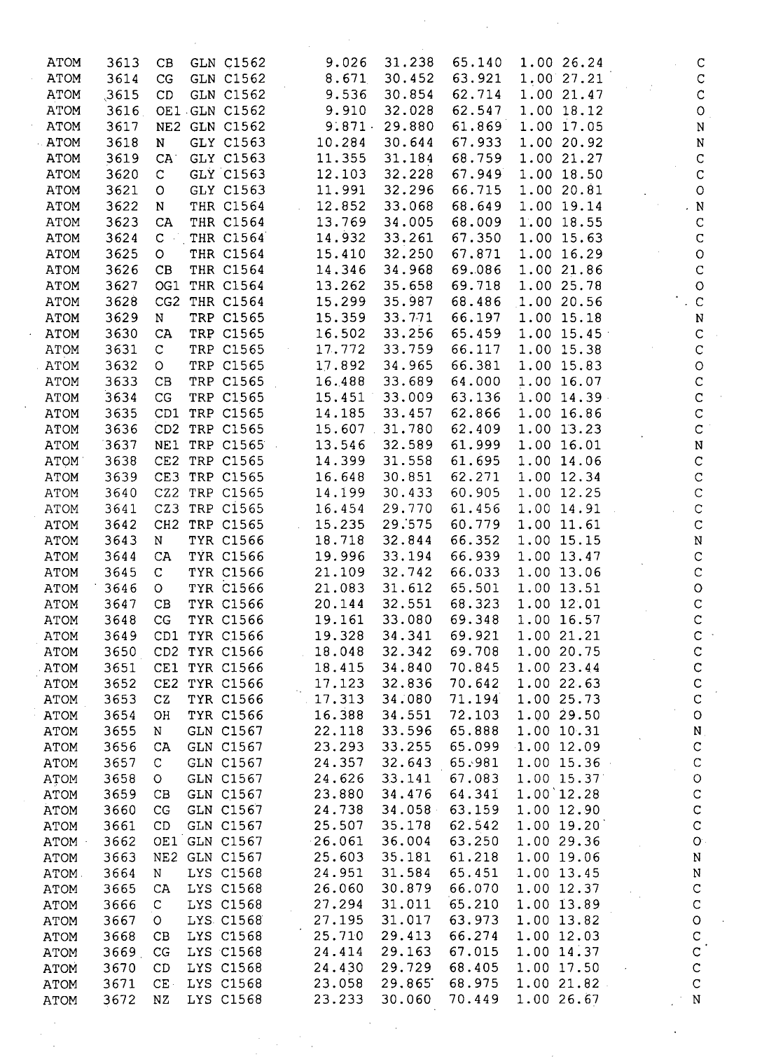

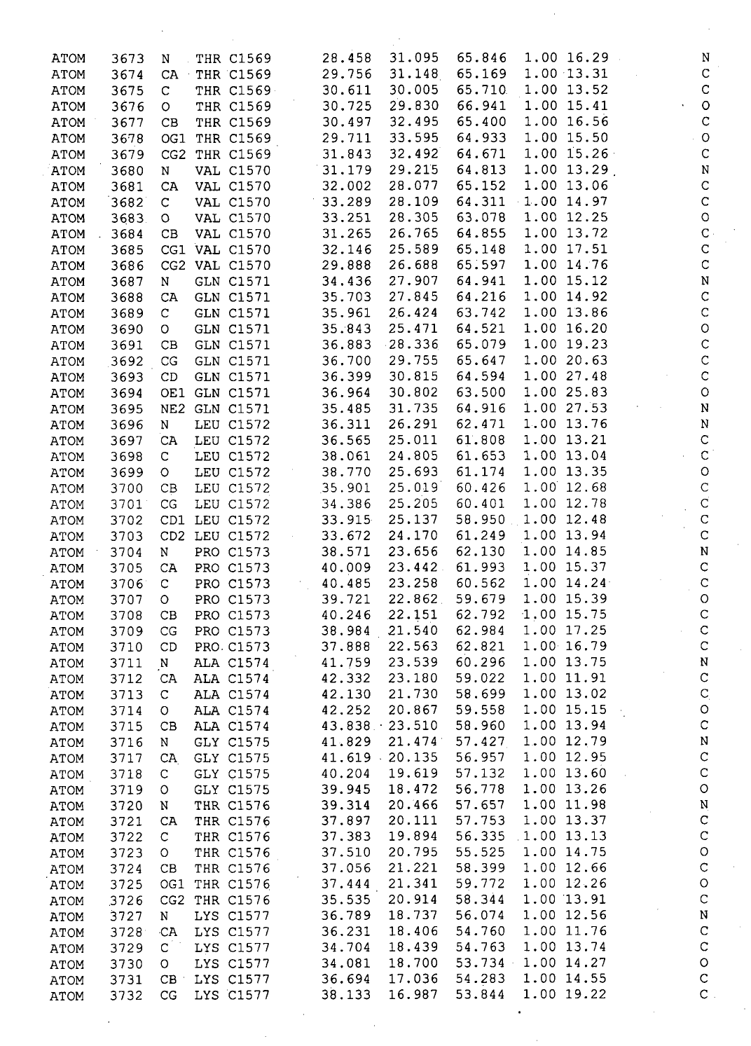

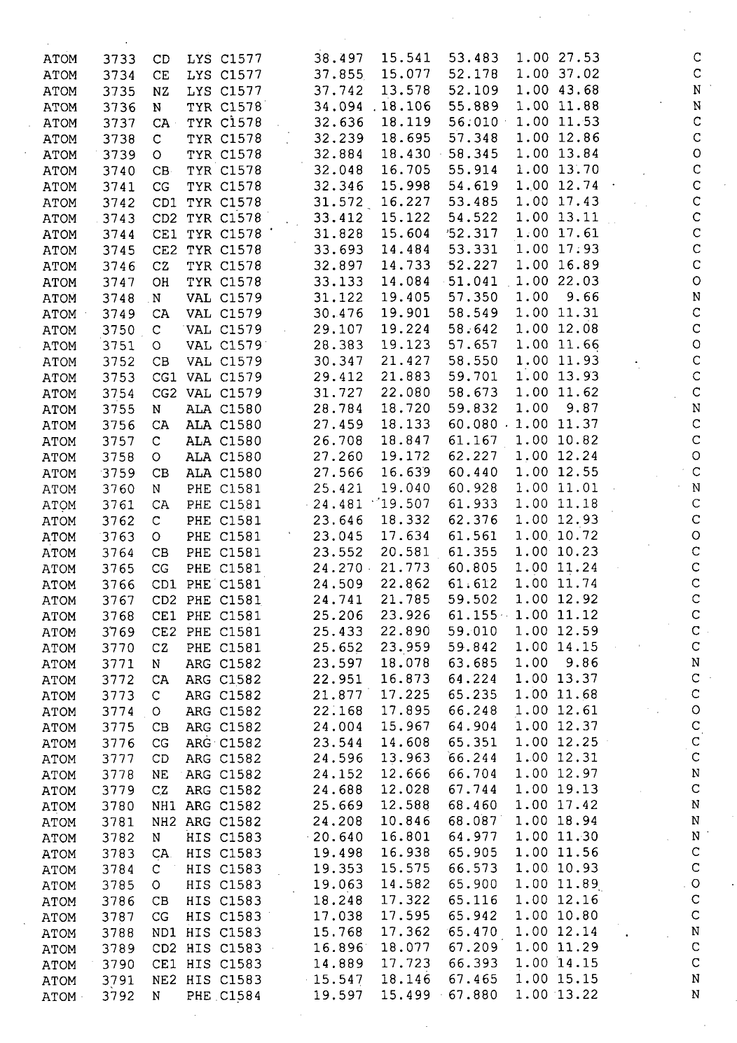

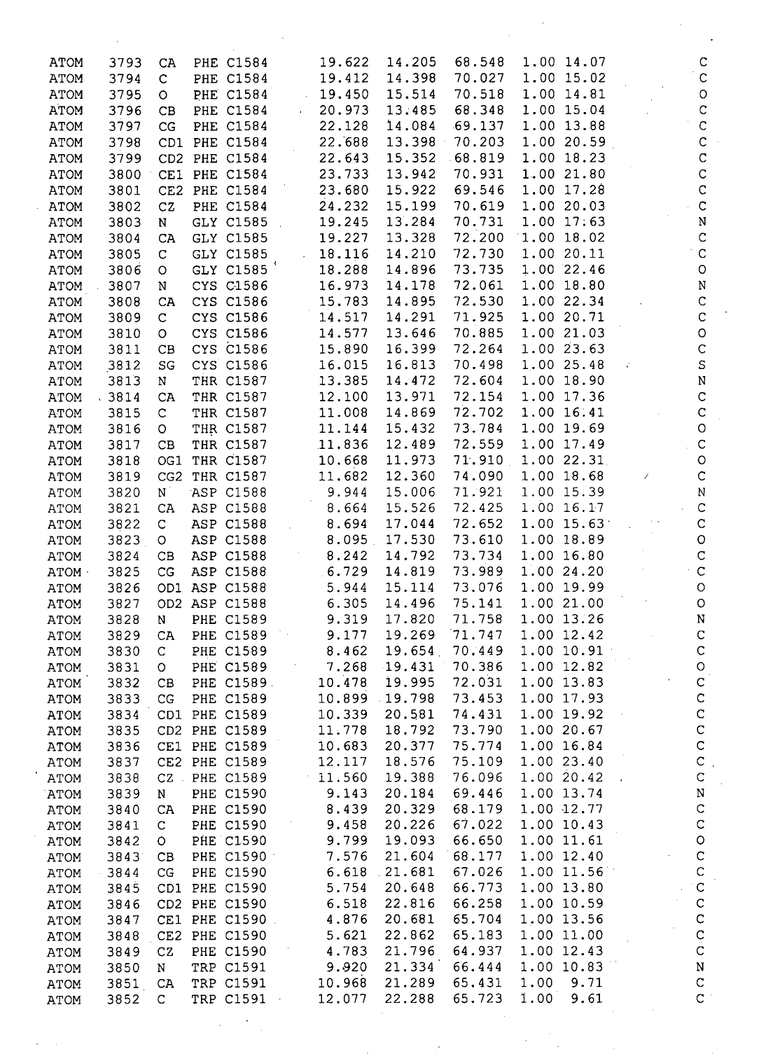

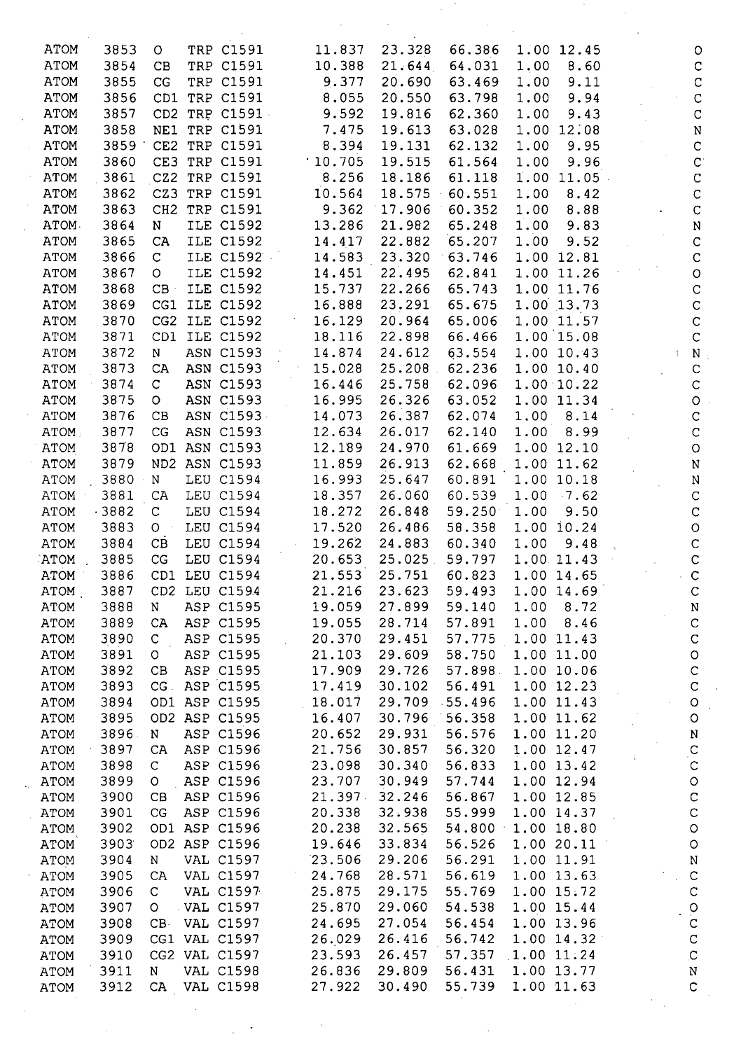

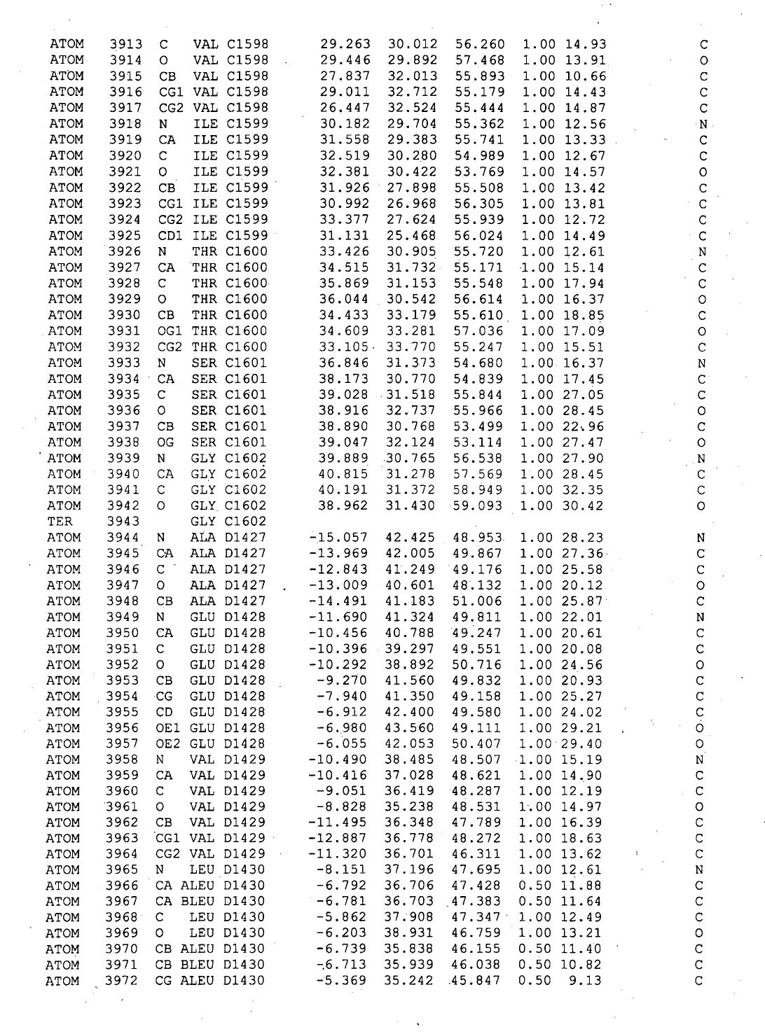

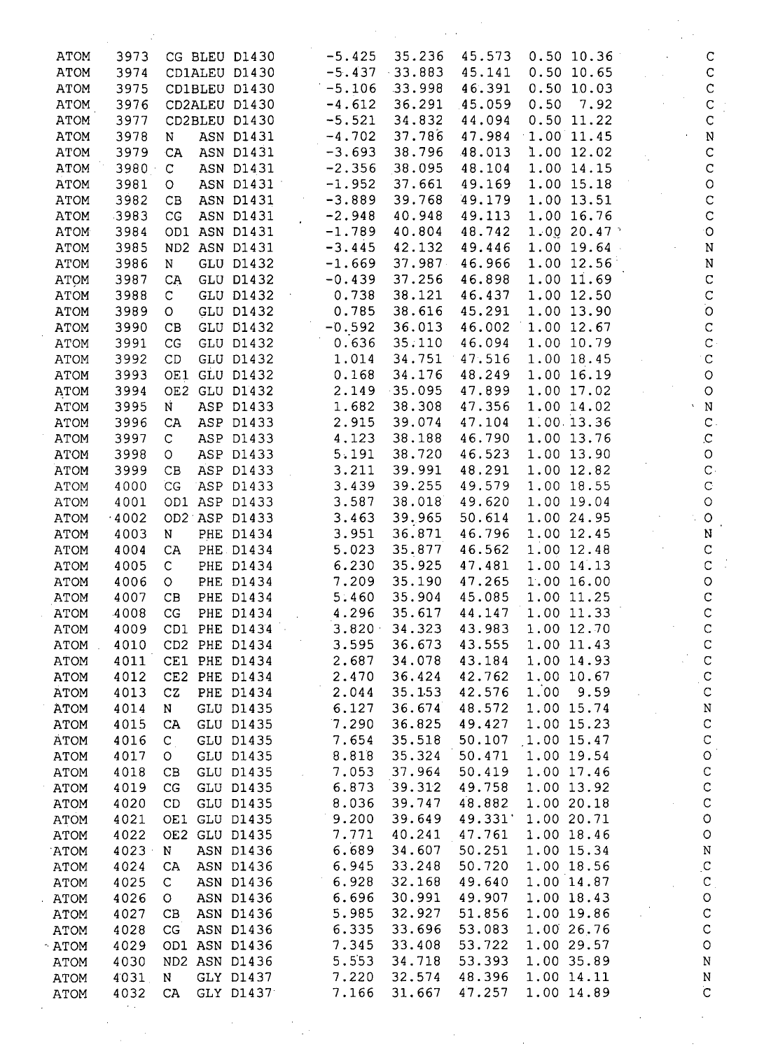

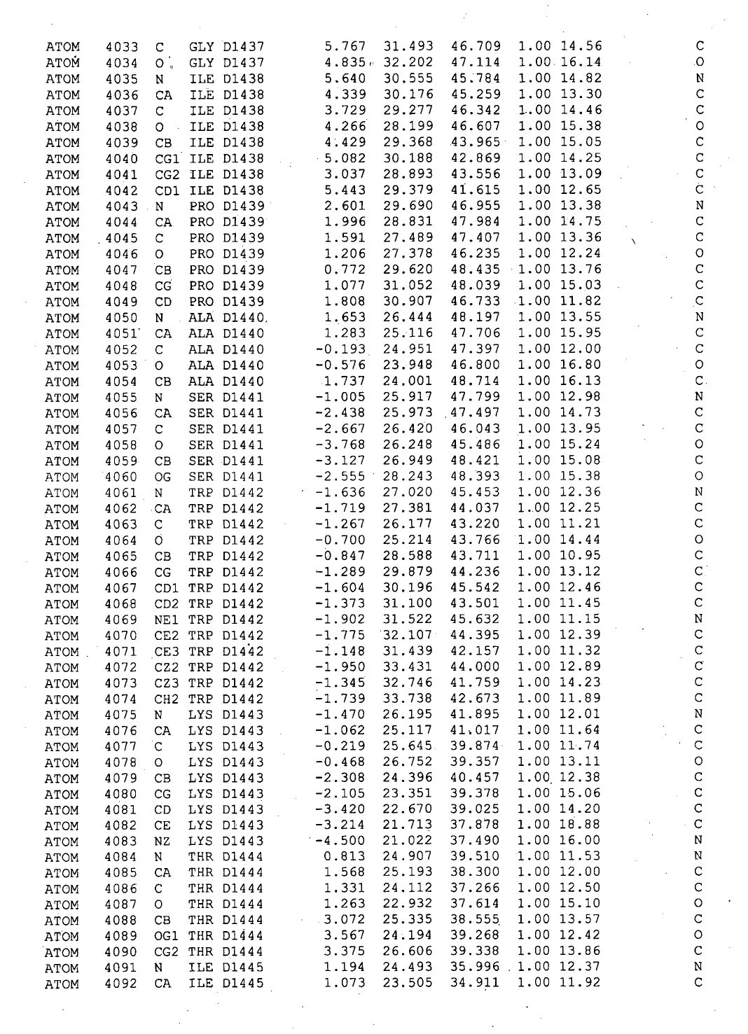

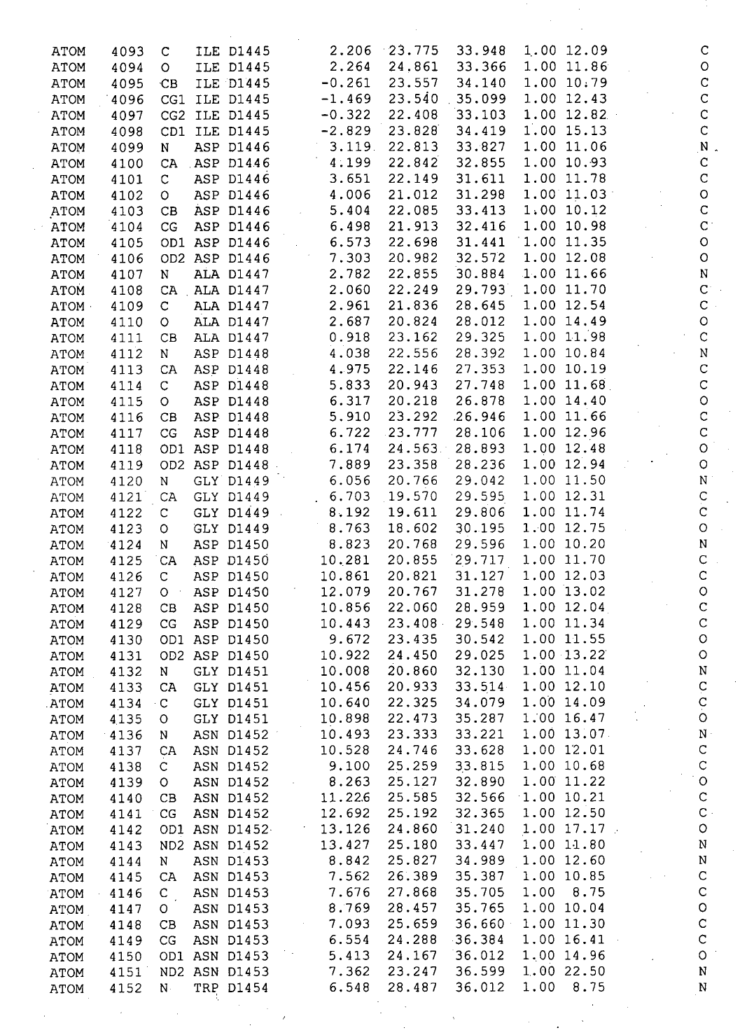

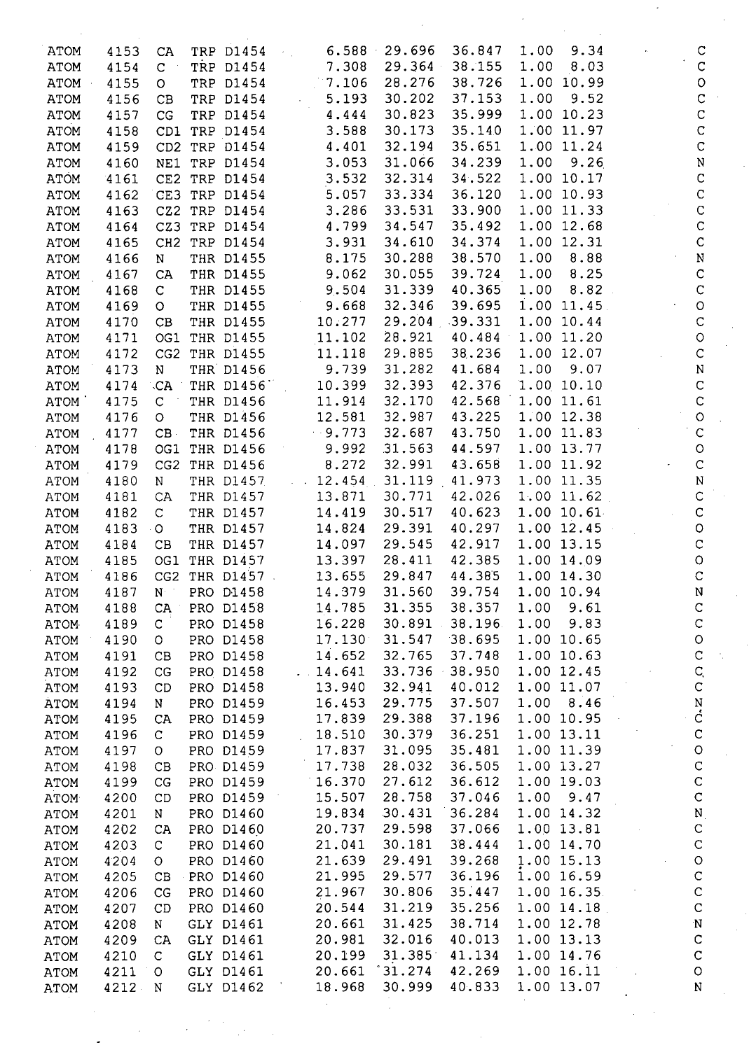

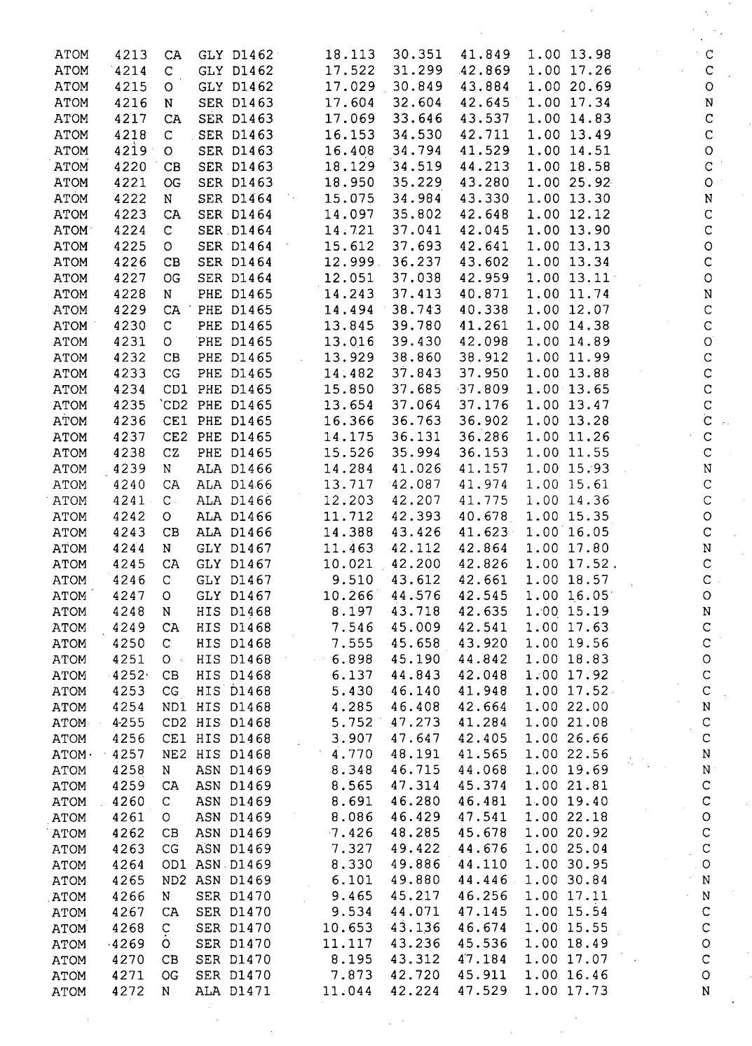

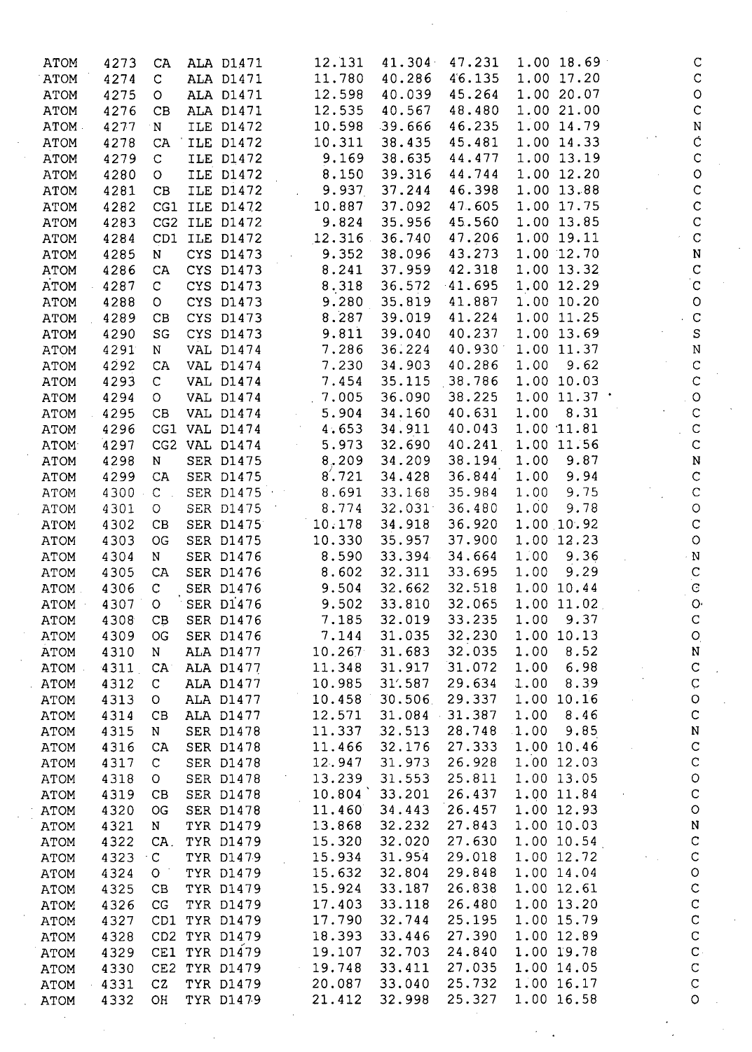

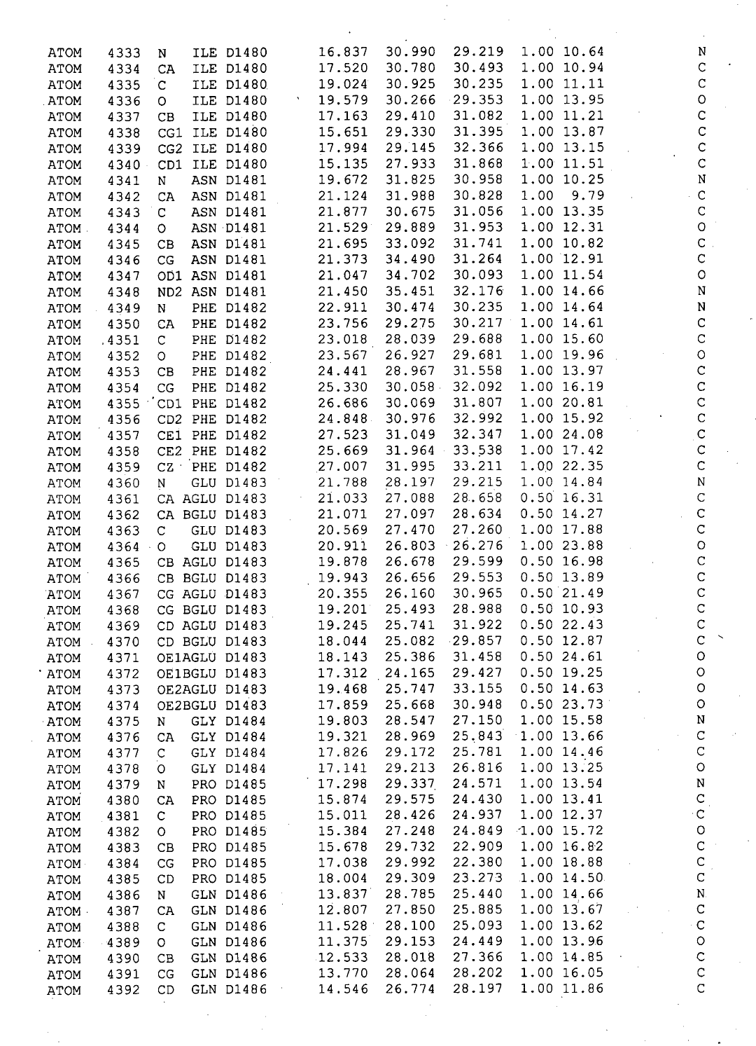

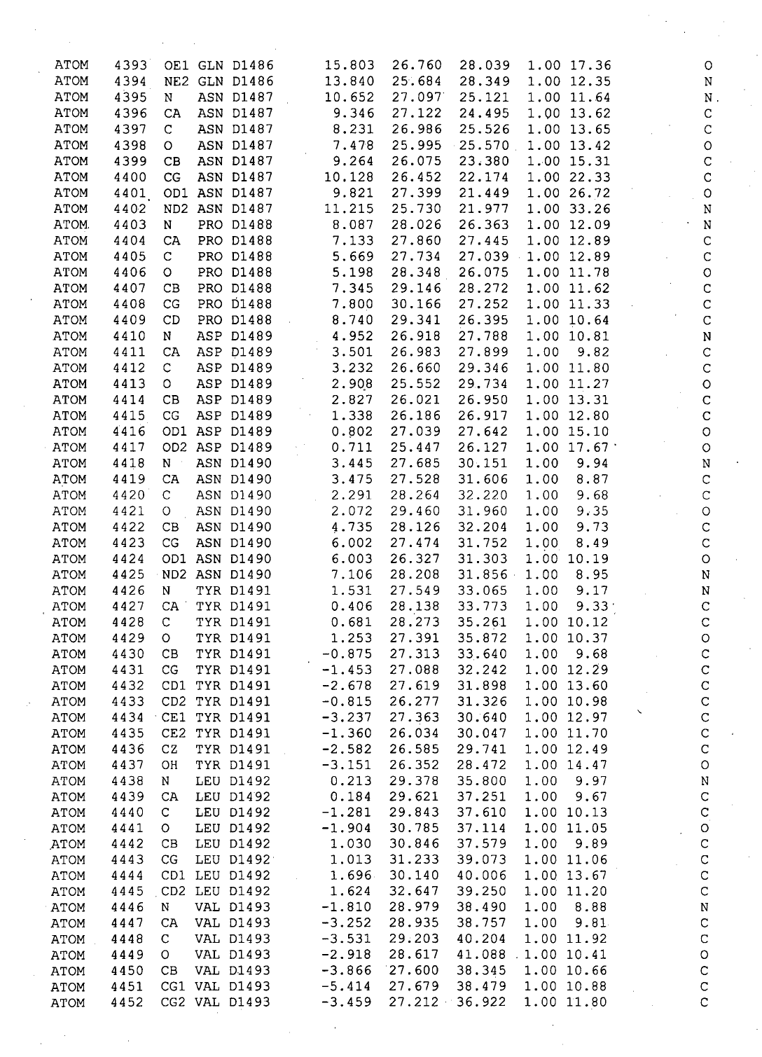

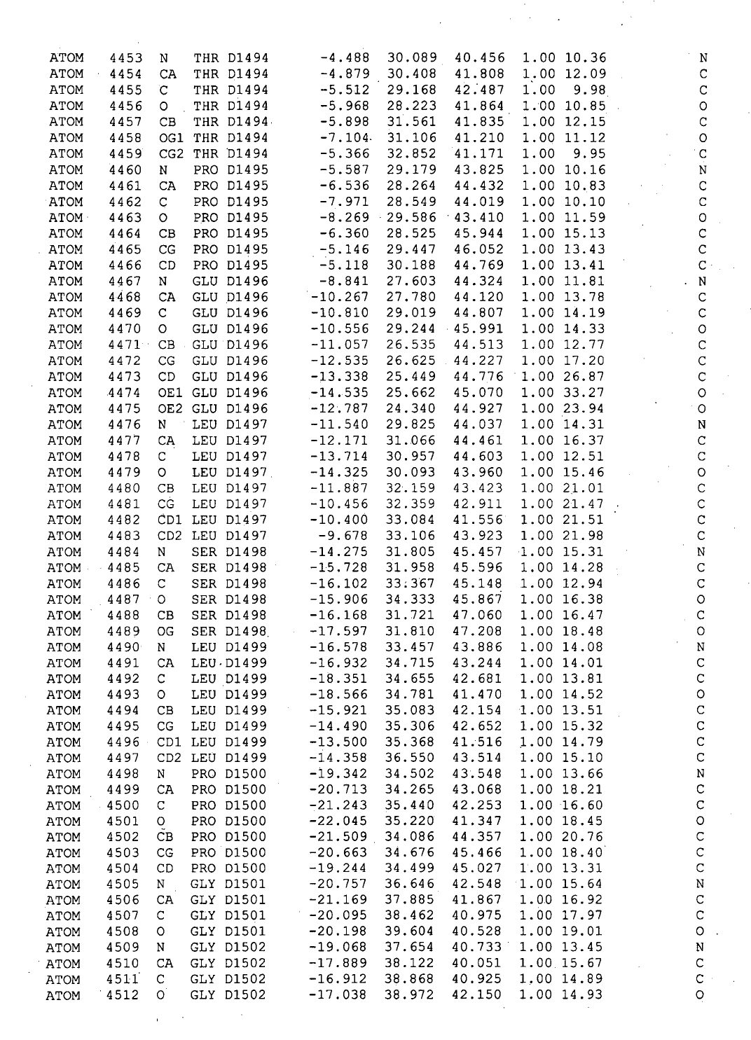

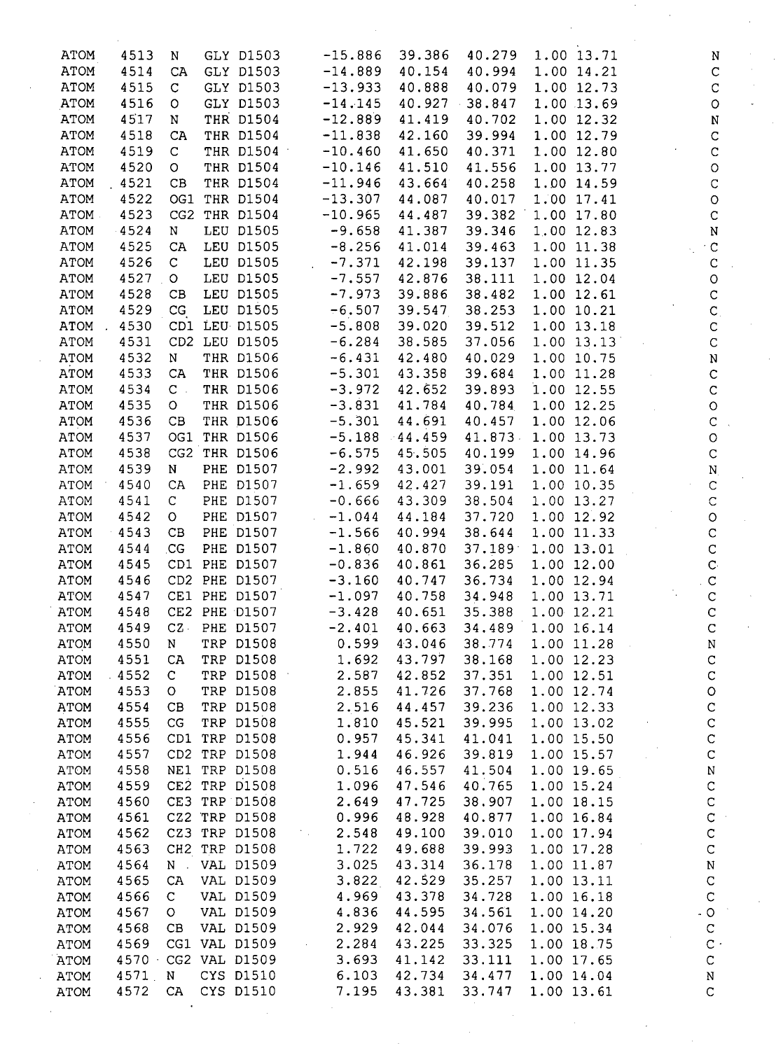

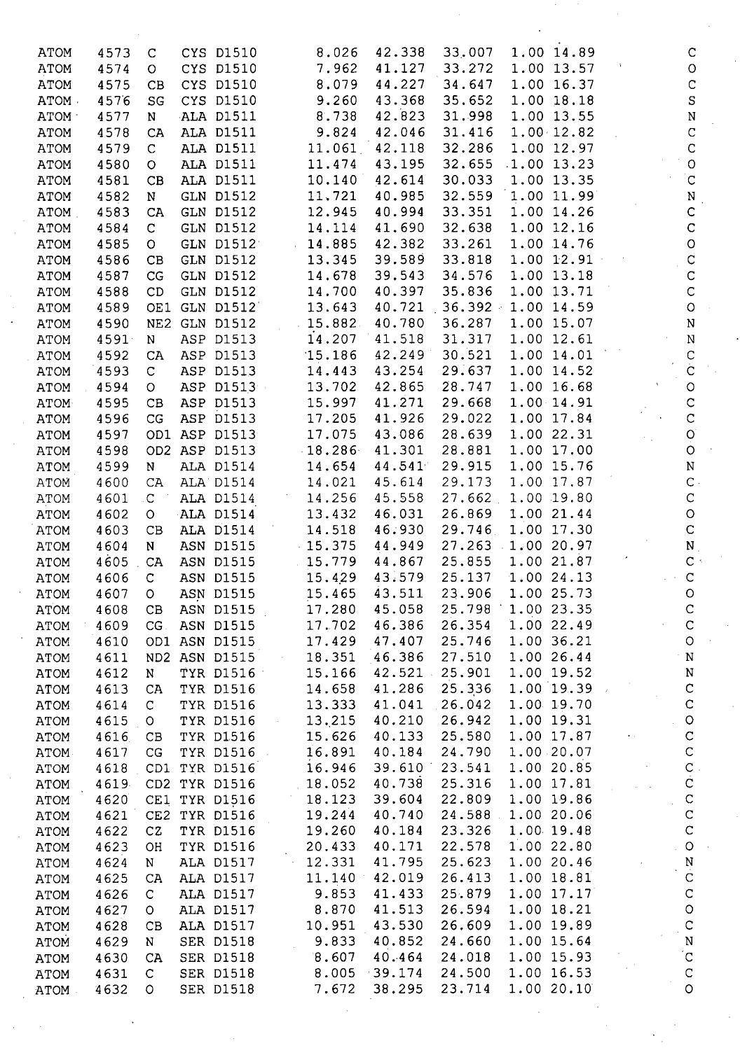

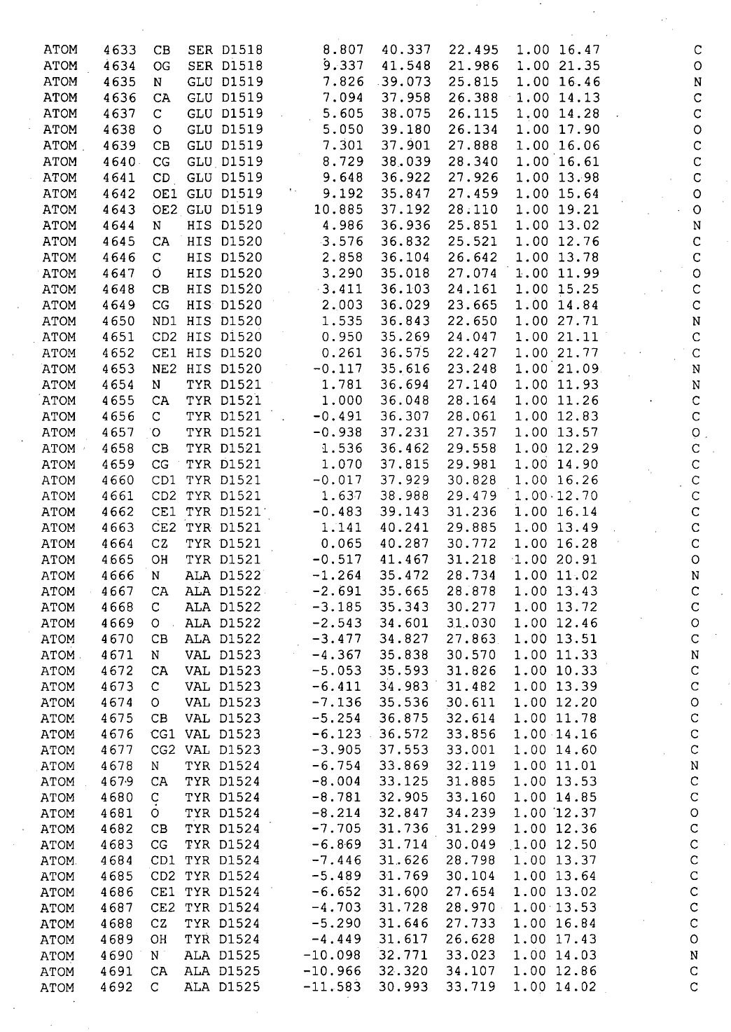

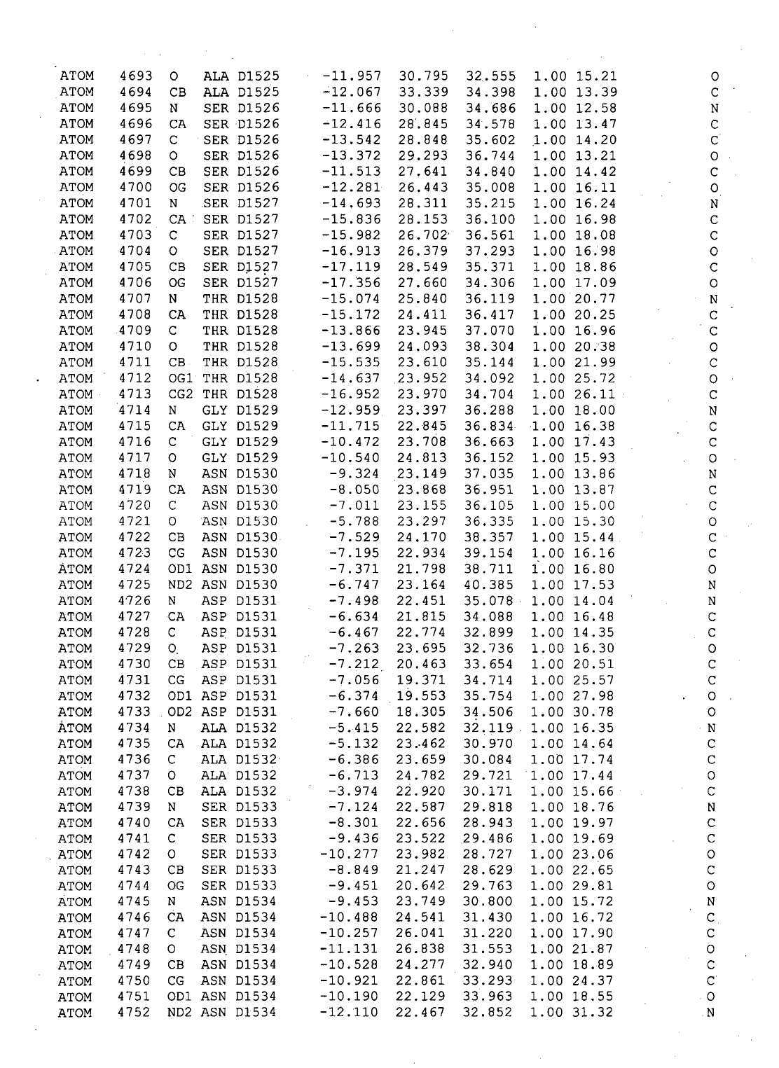

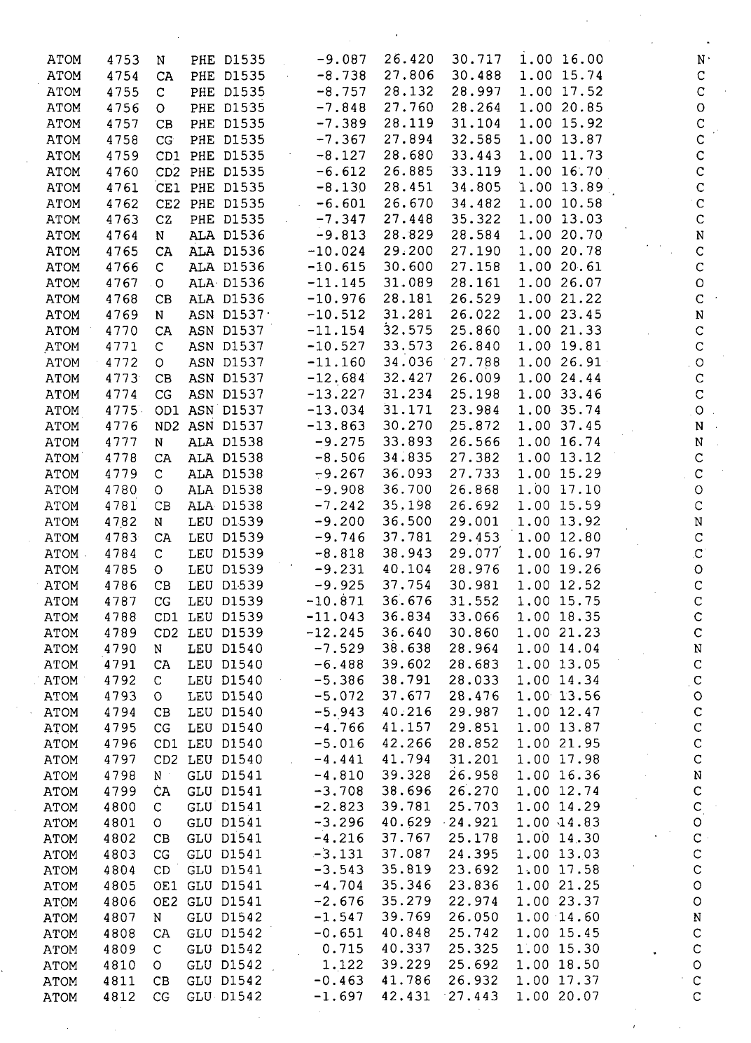

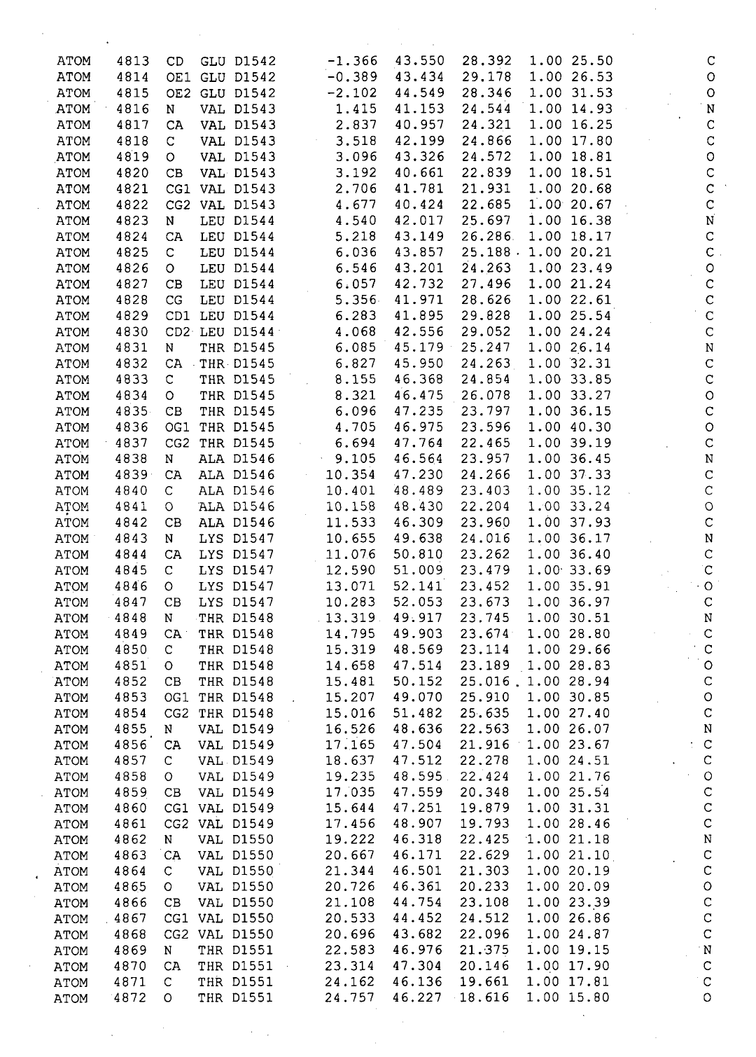

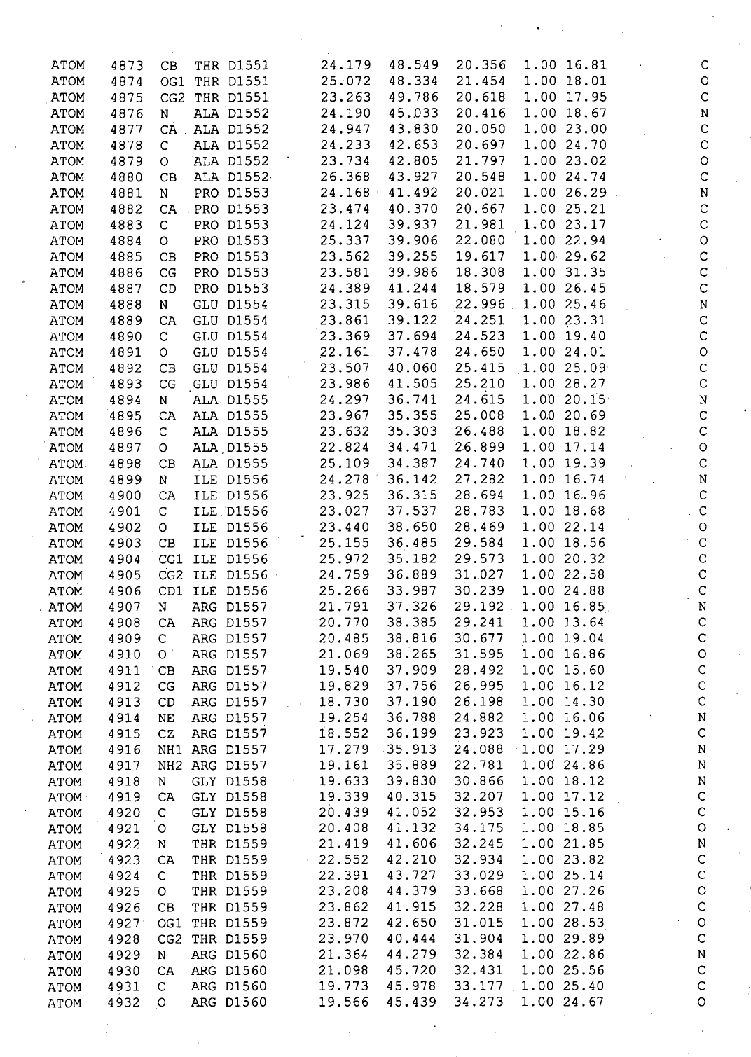

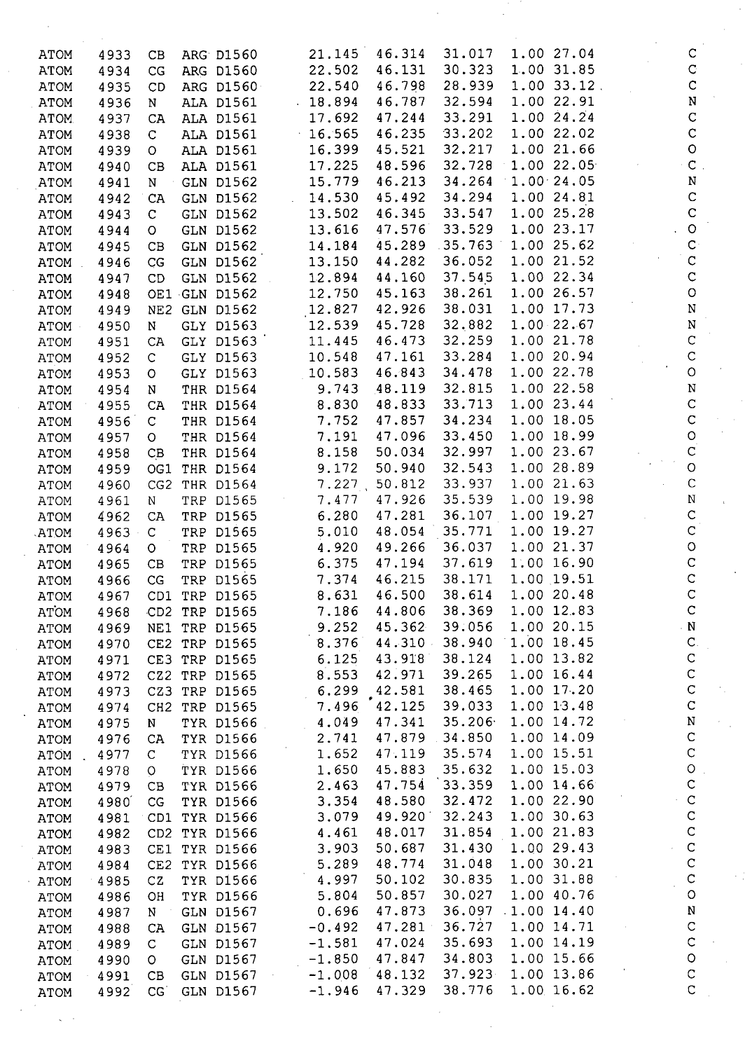

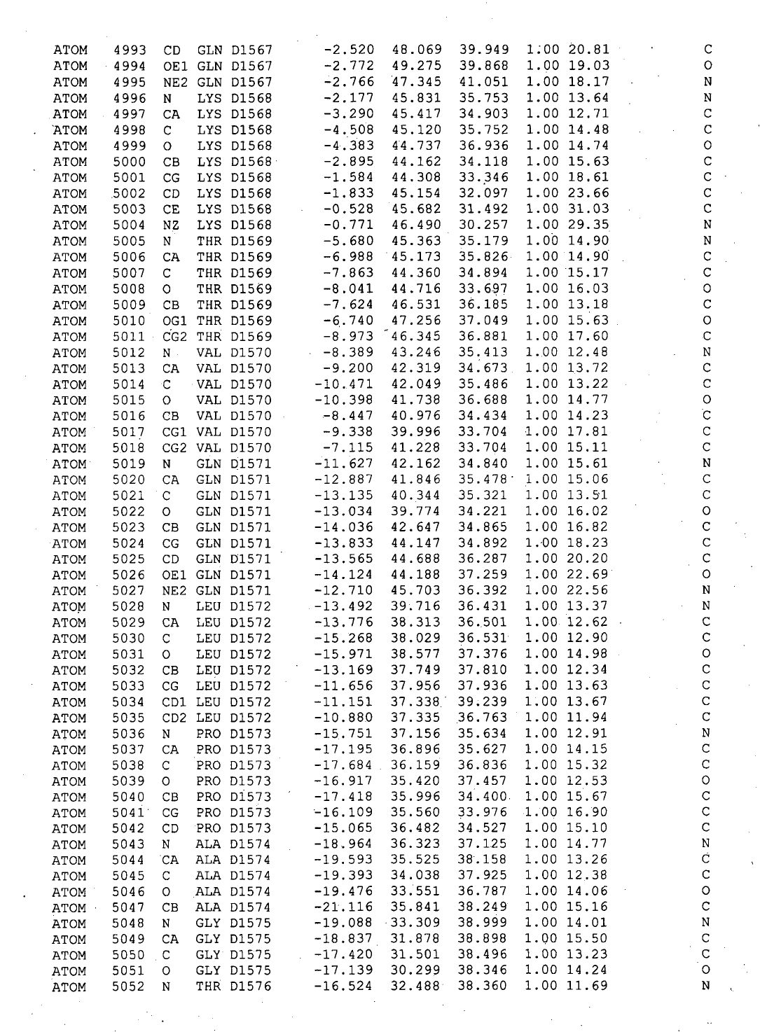

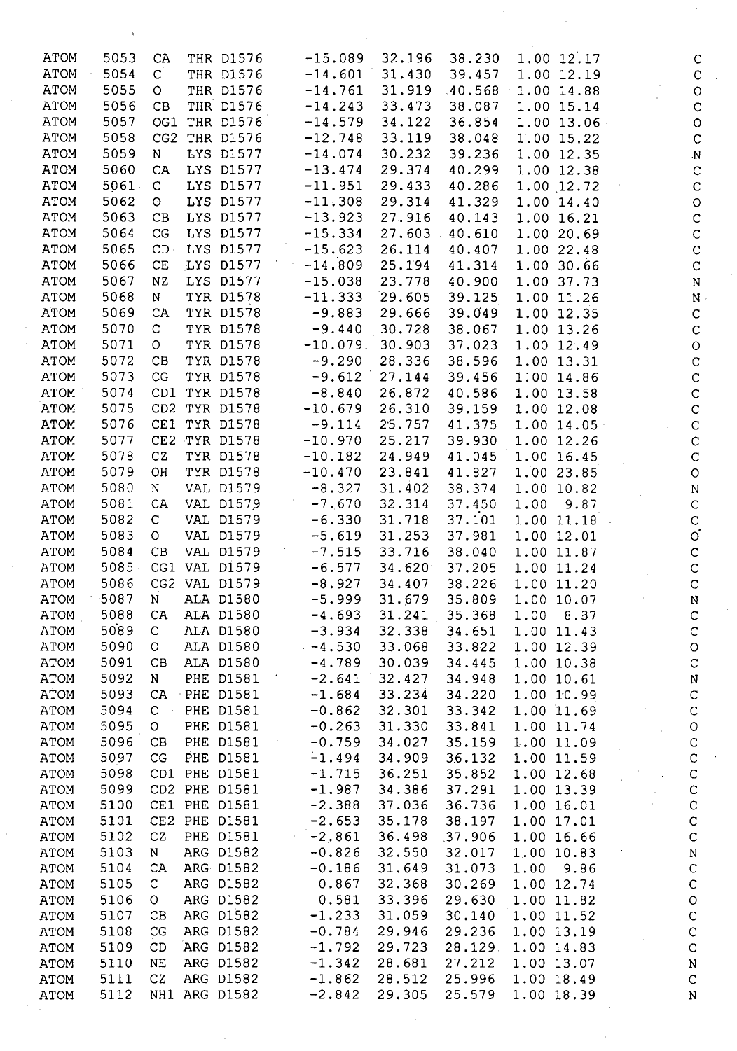

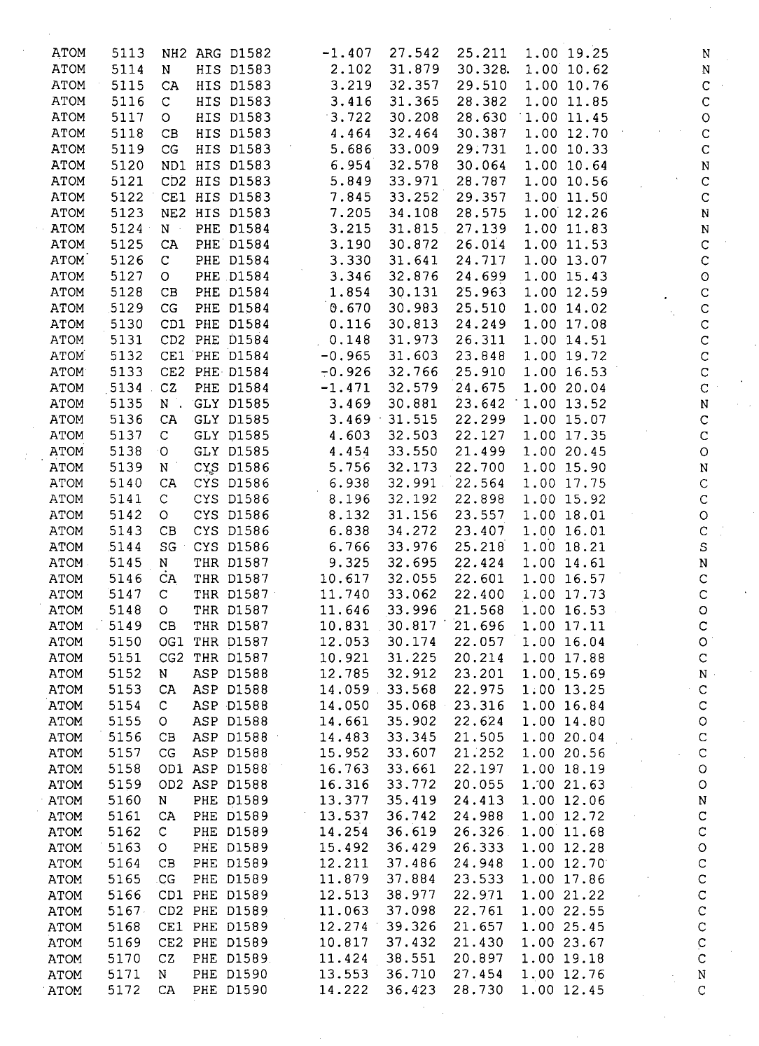

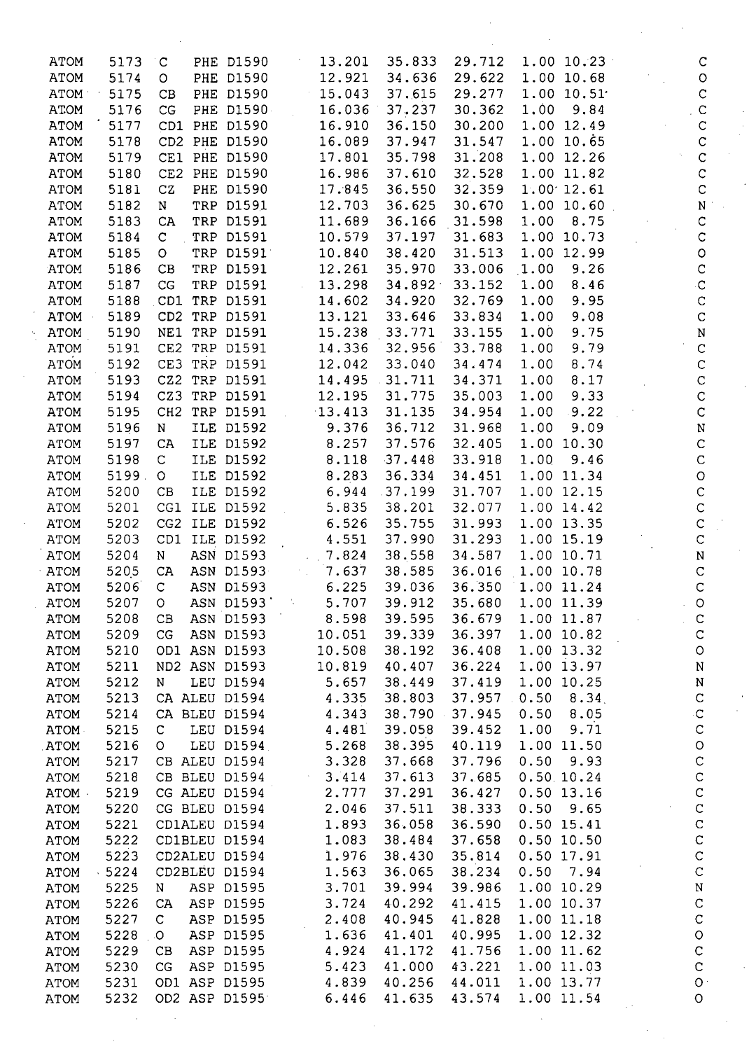

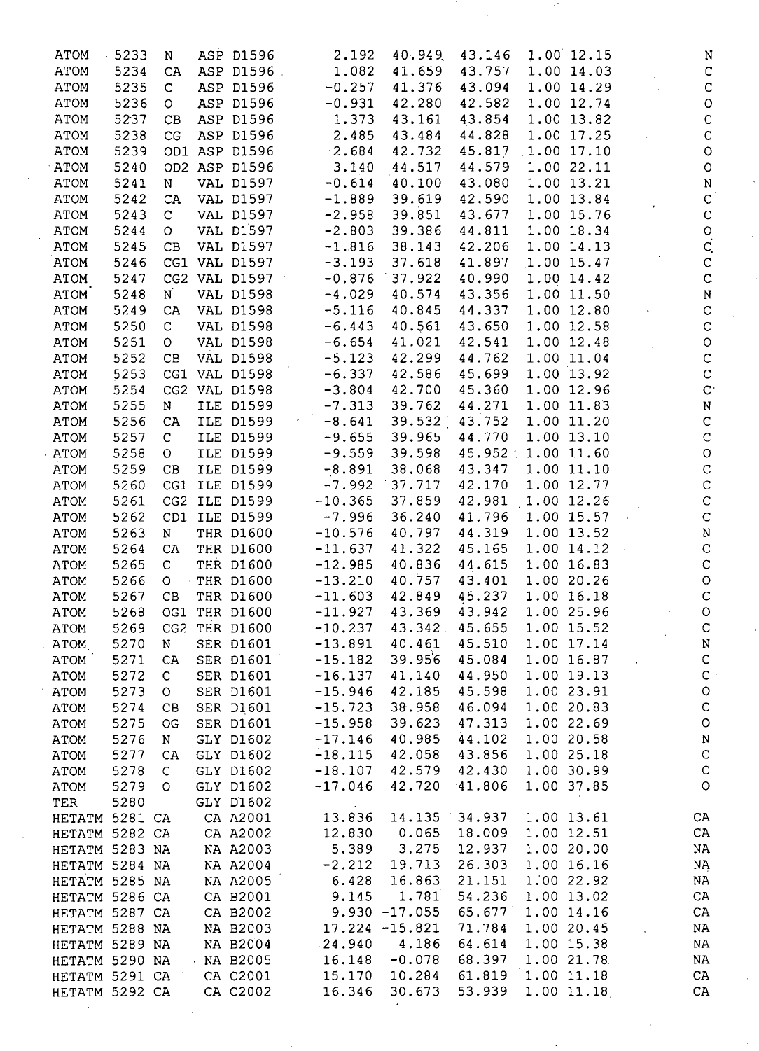

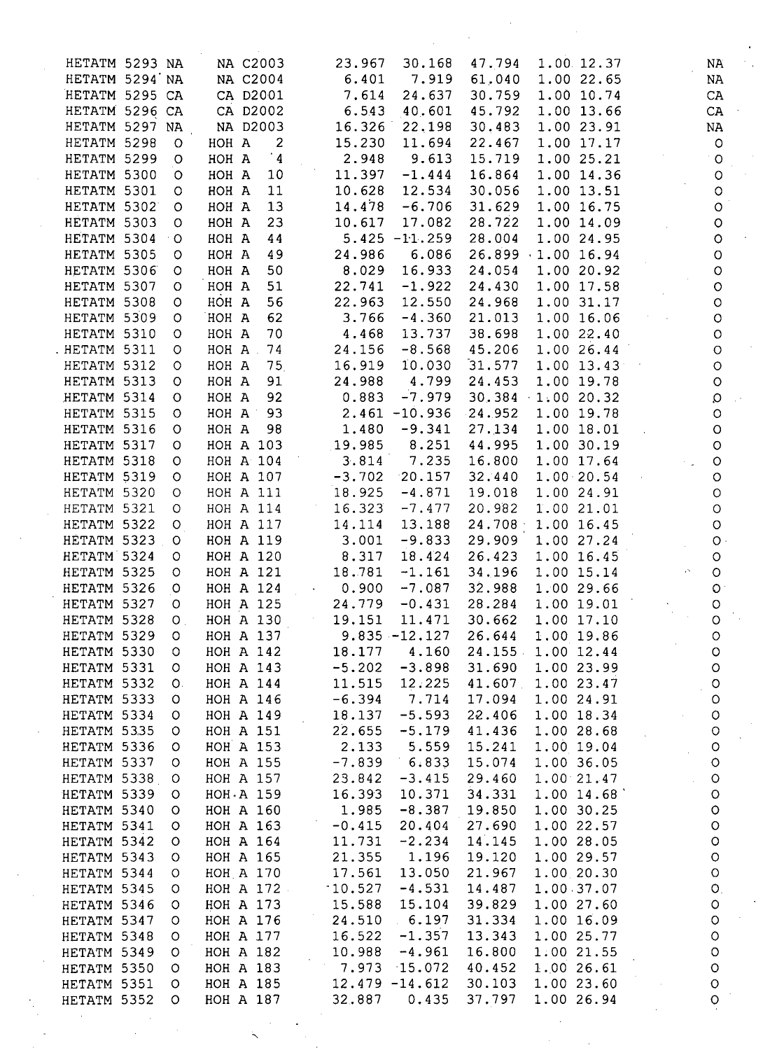

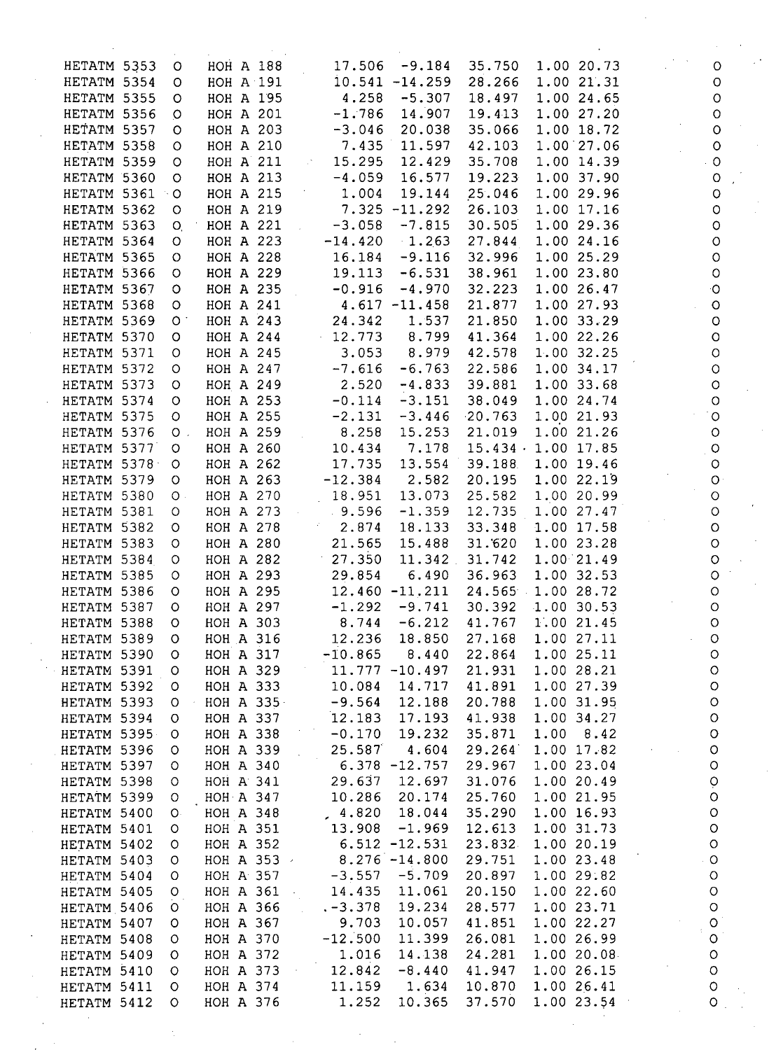

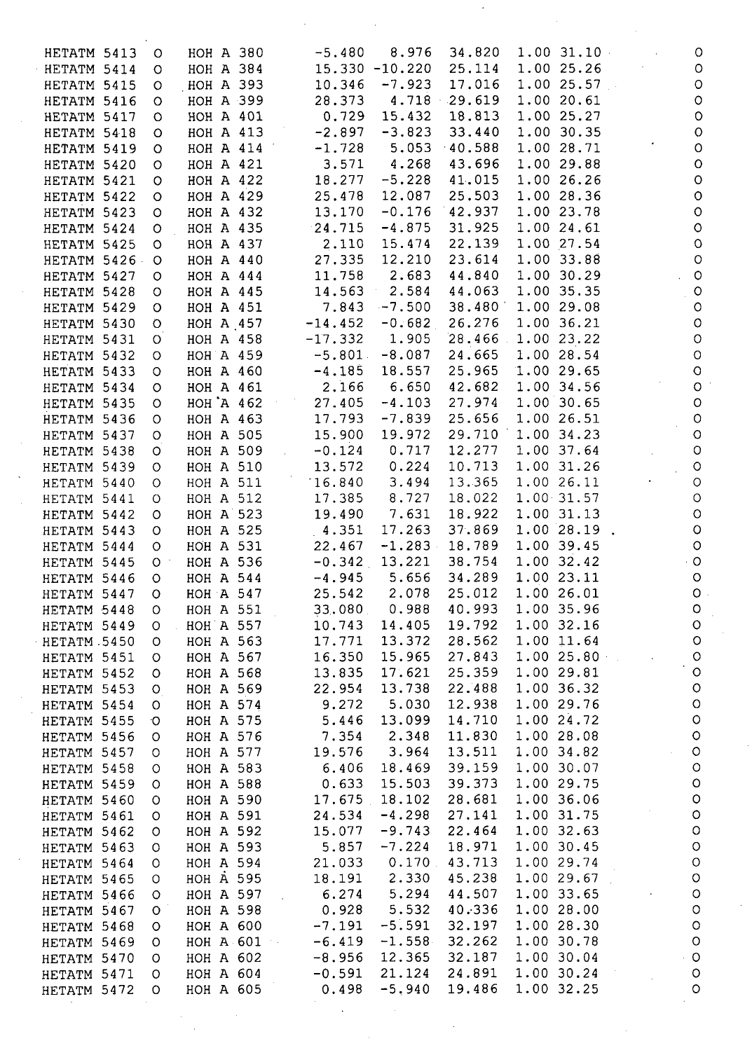

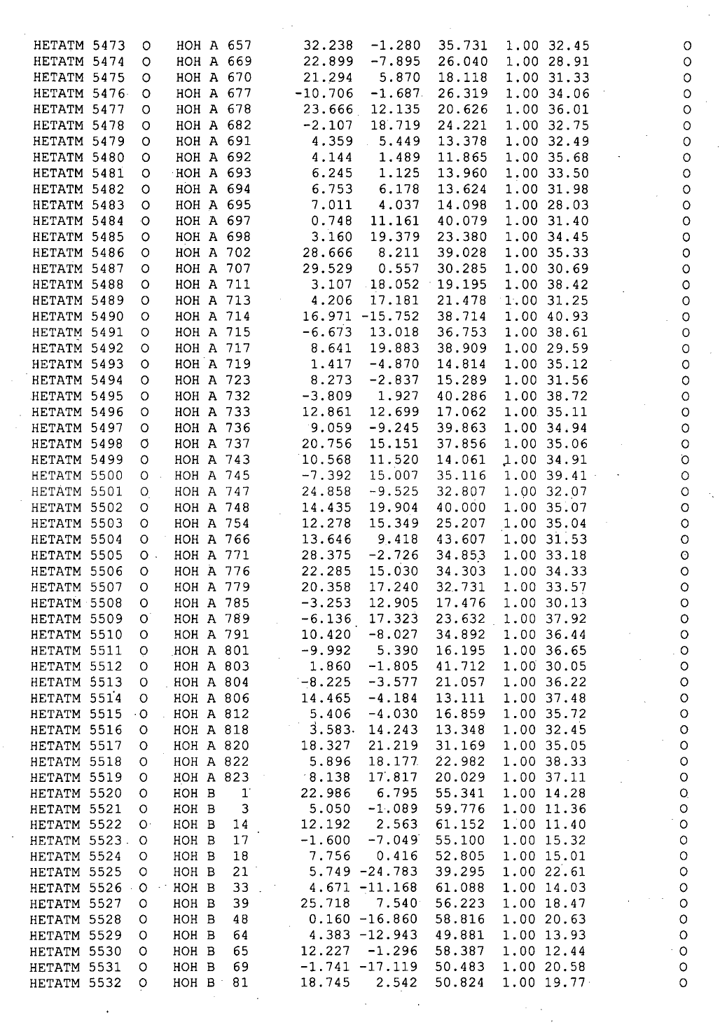

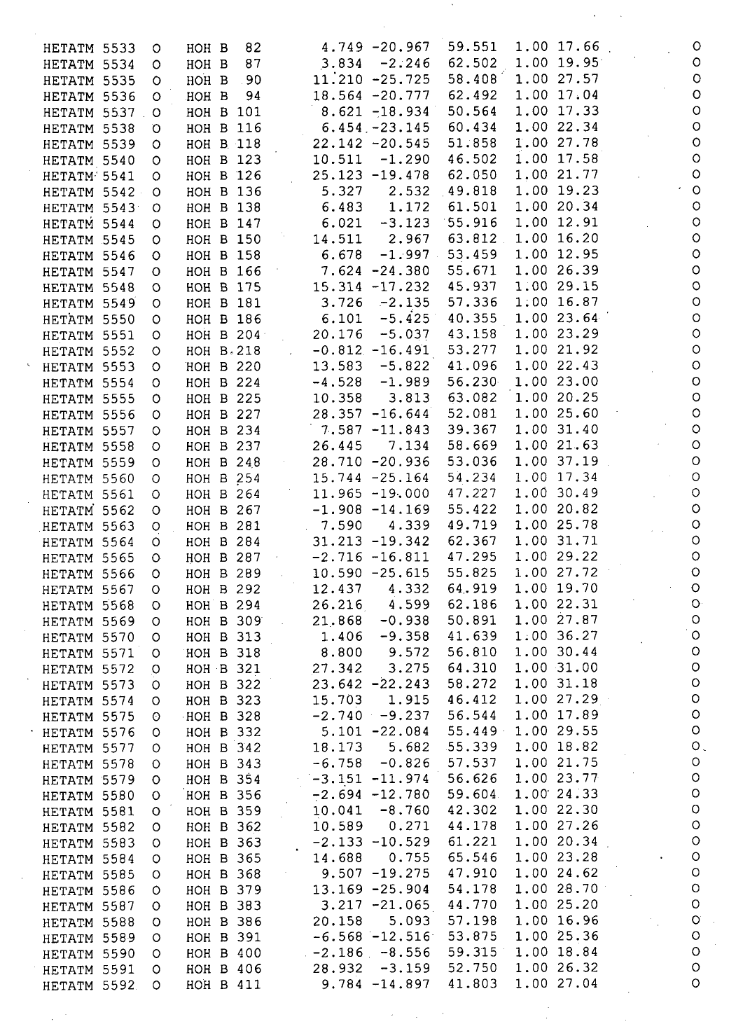

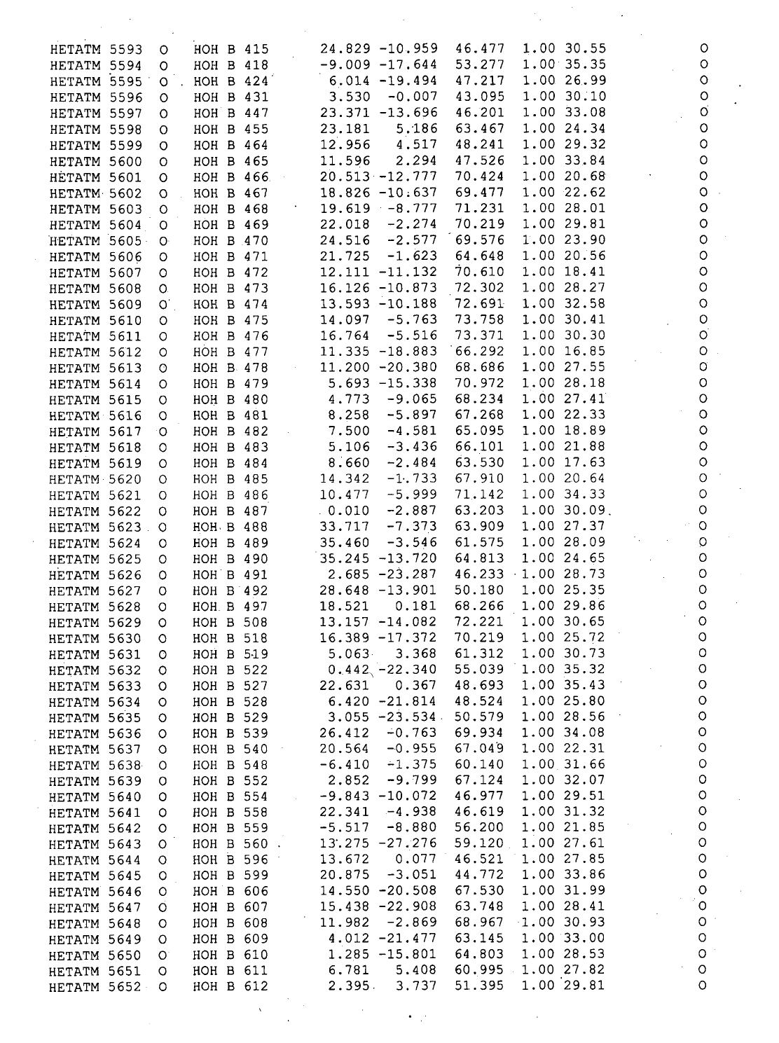

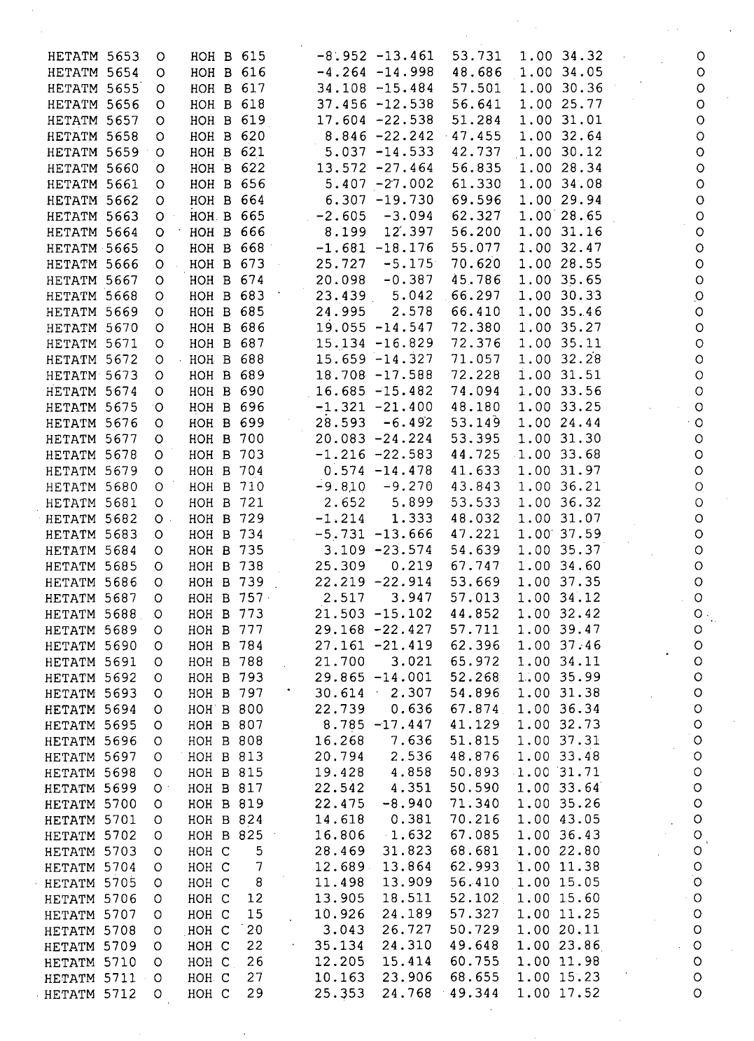

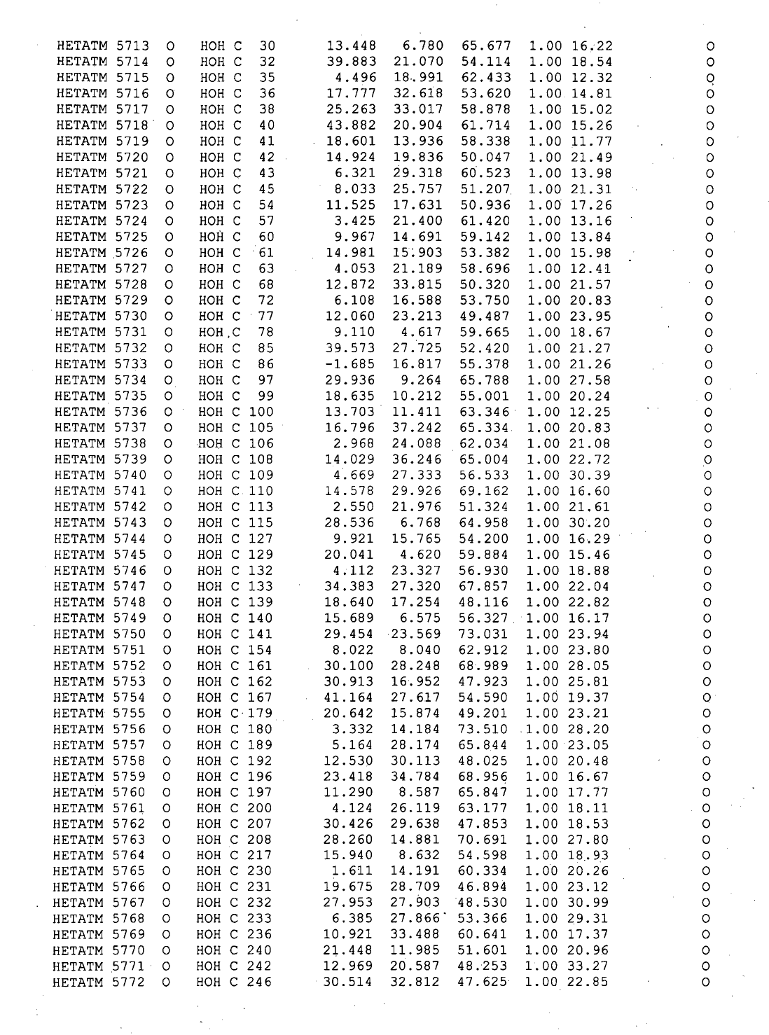

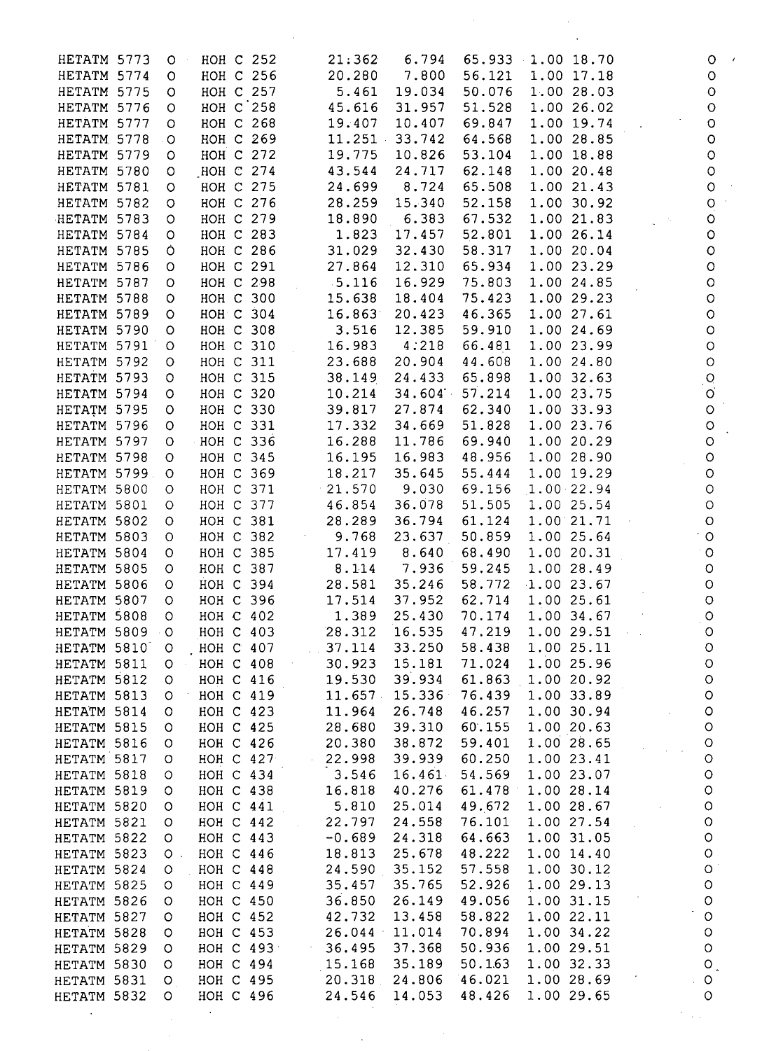

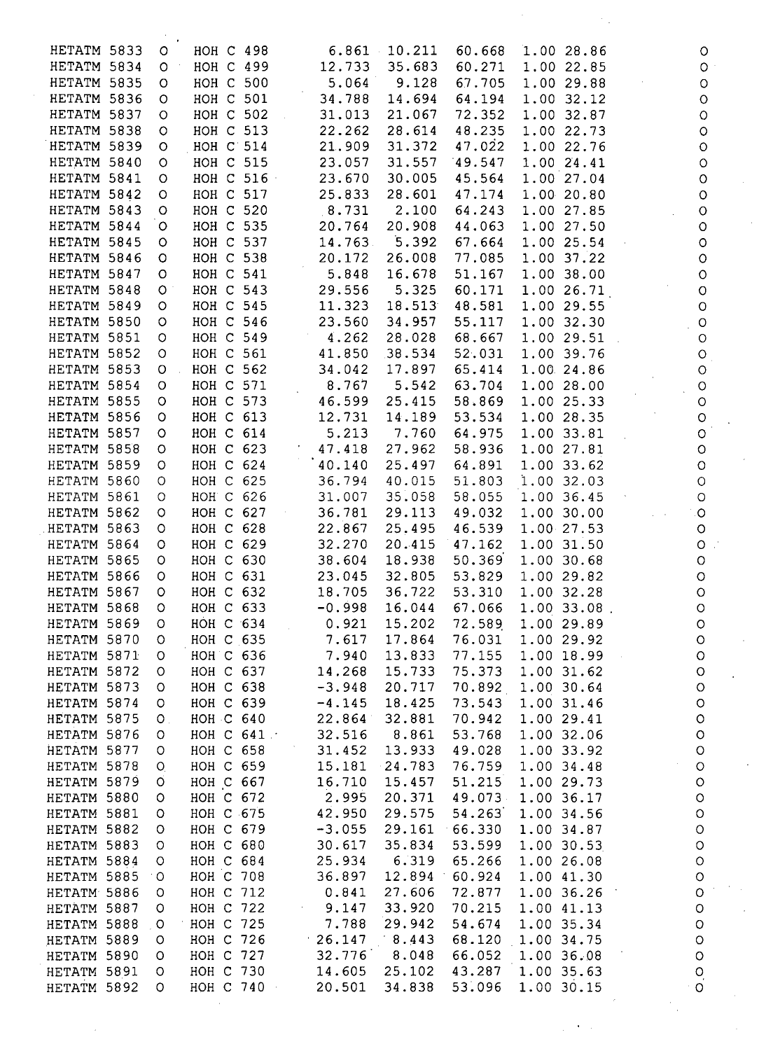

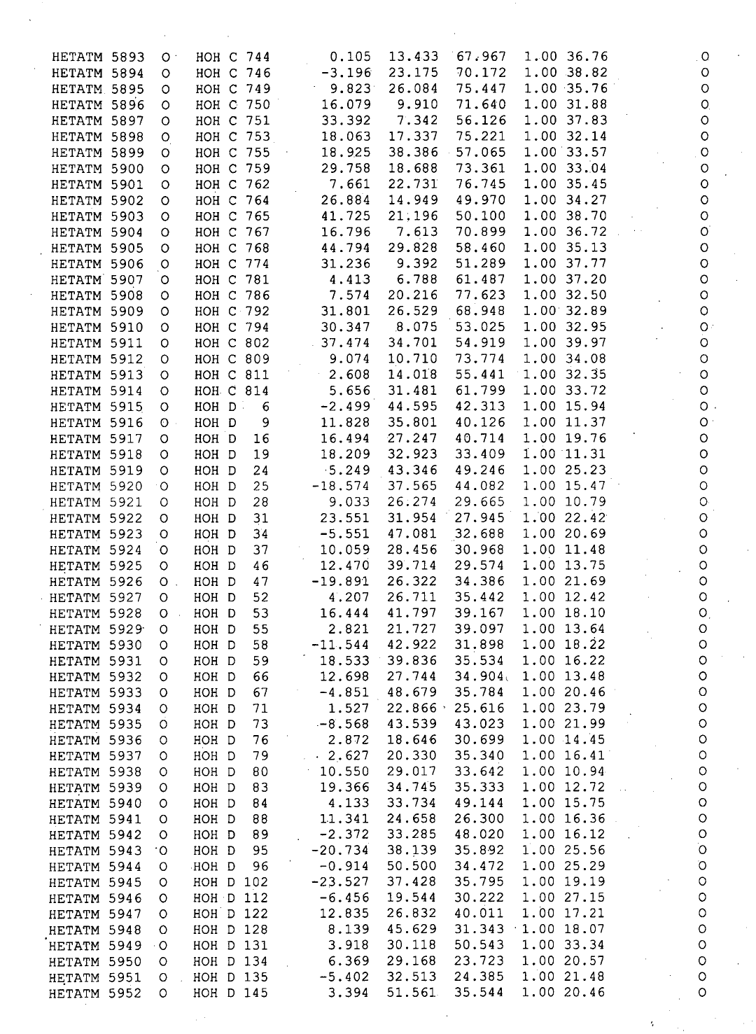

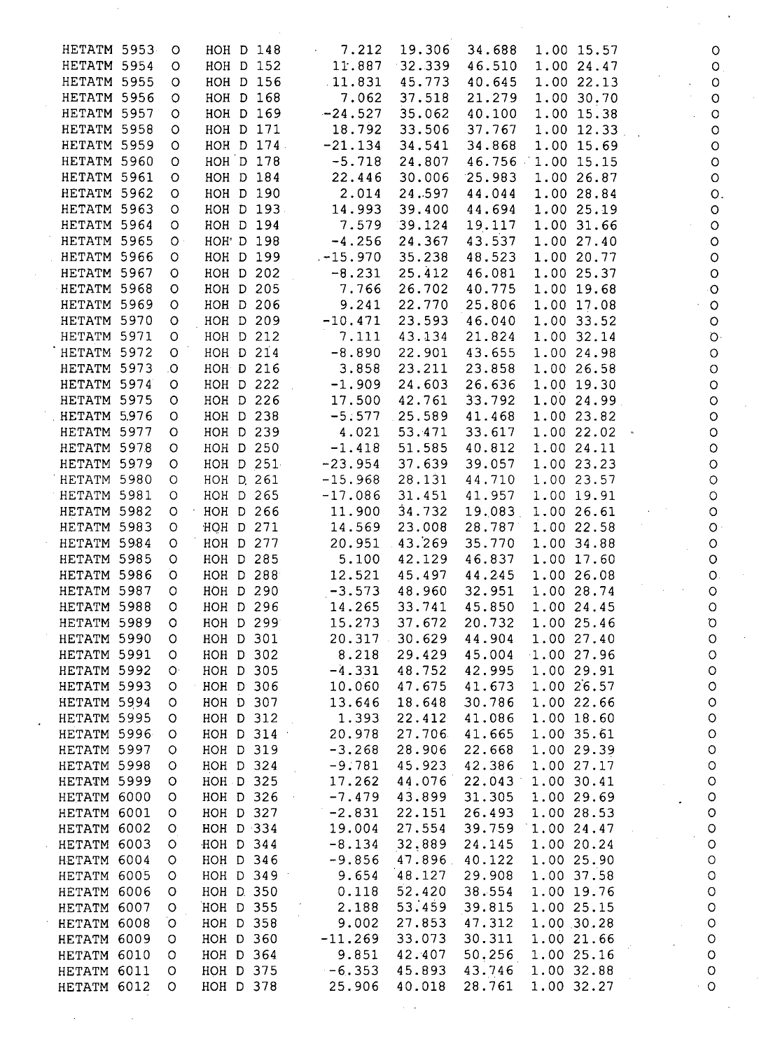

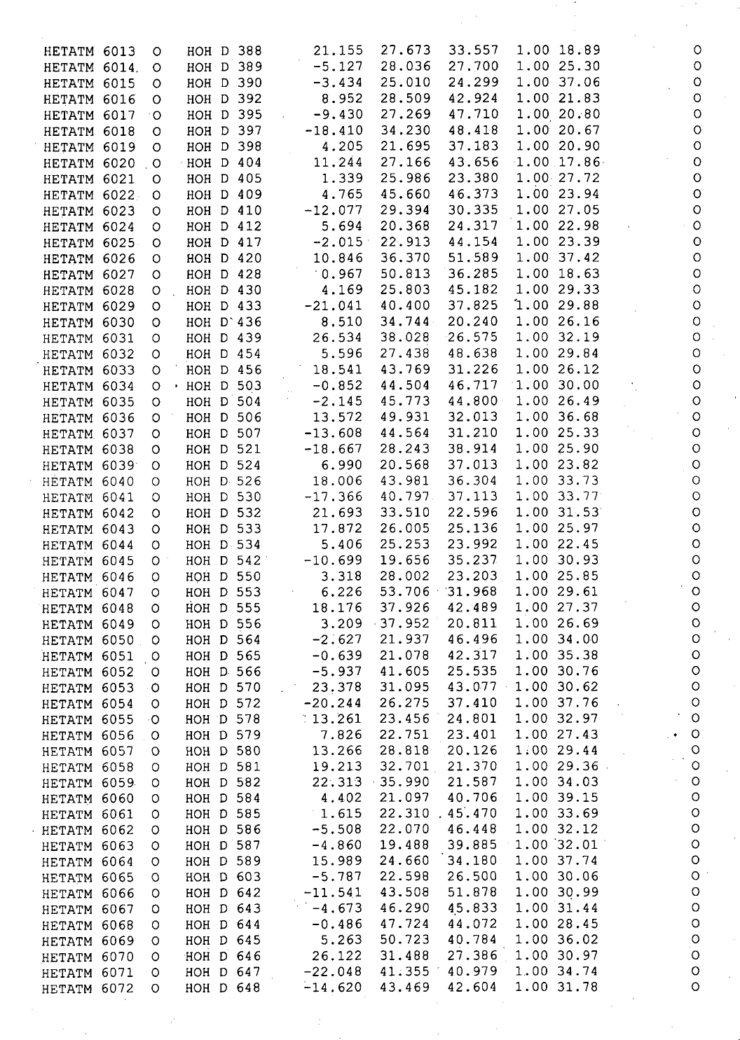

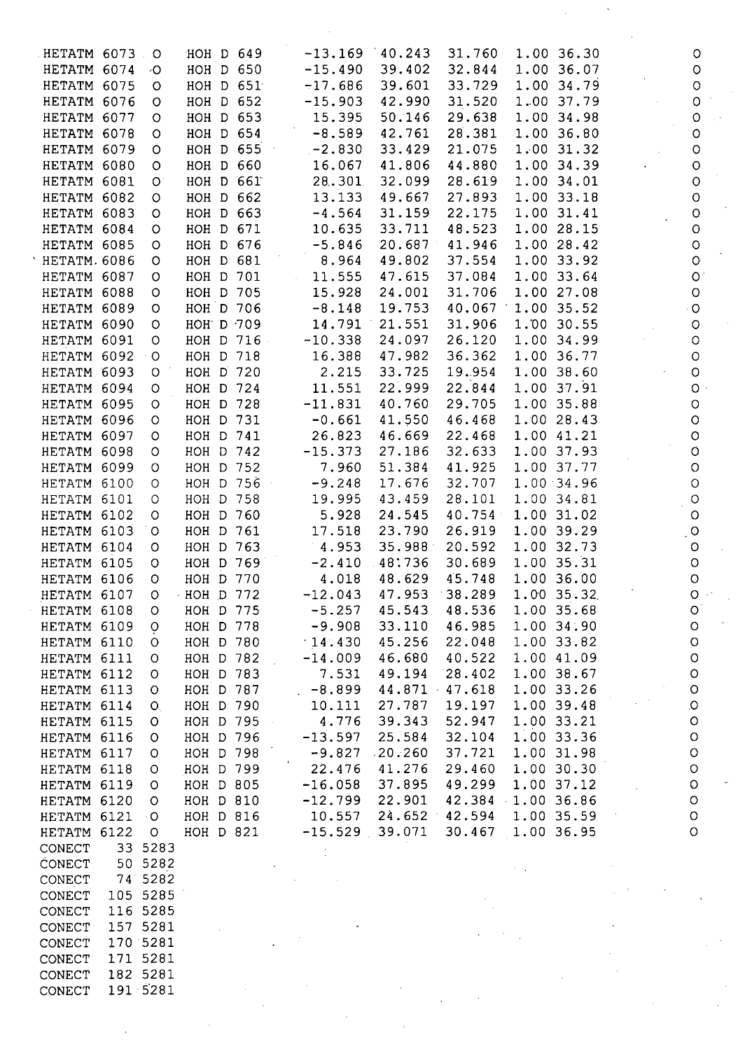

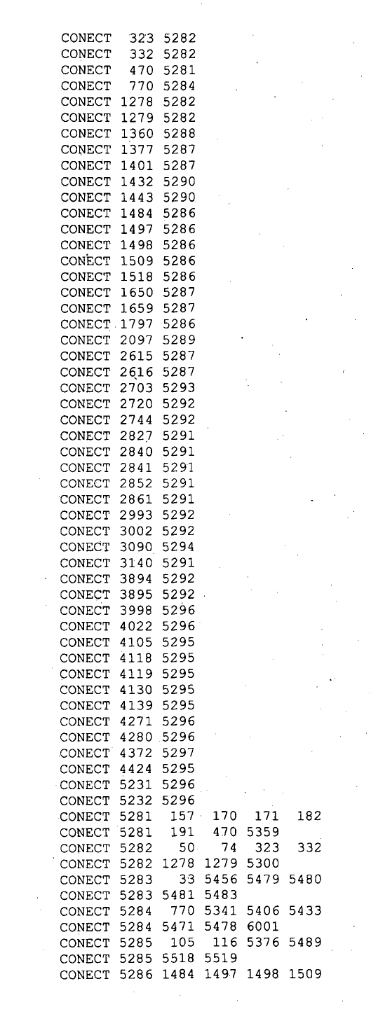

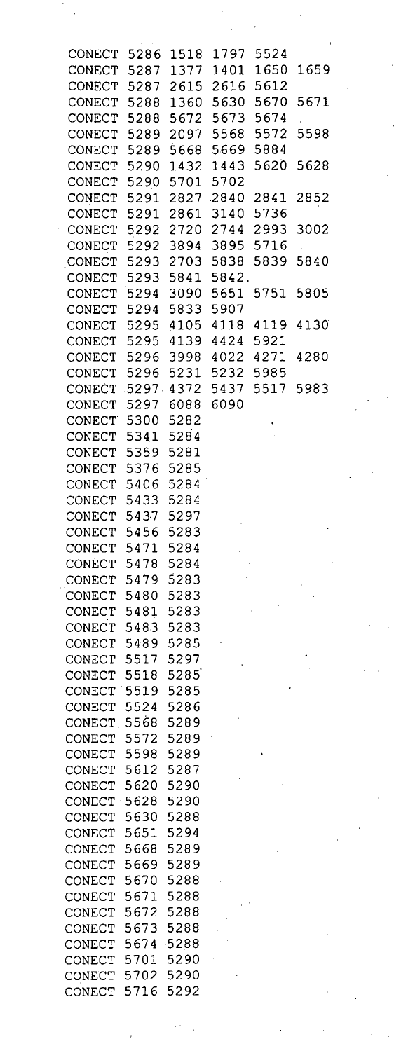

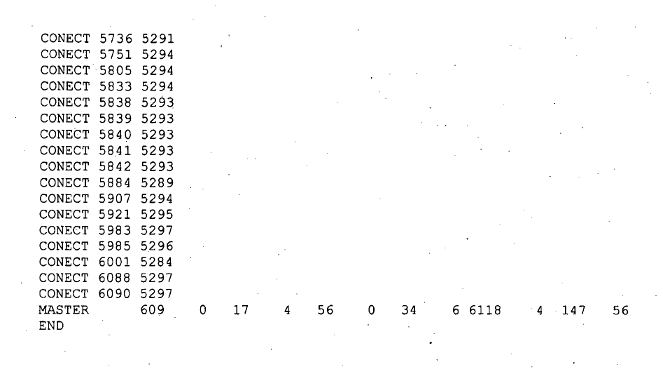

In another aspect, a method is provided for identifying a protein or part thereof which comprises a Cleaved_Adhesin domain, the method comprising subjecting amino acid sequences of proteins to Cleaved_Adhesin domain modeling based on the amino acid sequences of one or more of K1, K2, K3, R1, R2 and/or A1 through A10 and selecting amino acid sequences having homology thereto wherein such identified amino acid sequences are regarded as defining a Cleaved_Adhesin domain. By “homology” is meant an amino acid sequence identified by multiple sequence alignment of known Cleaved_Adhesin domains such as K1, K2, K3, R1, R2 and two or more of A1 through A10. In an embodiment, multiple sequence alignment modeling is used to identify homologous Cleaved_Adhesin domains in other proteins. In particular, the K1 sequence is aligned with the sequence of the K3 domain (FIG. 14G) and the K3 crystal structure is used as a template for homology modeling. Homologous sequences include amino acid sequences having at least 10% overall similarity such as 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%. The sequence and the template is used as an input for the comparative protein modeling software MODELLER (Sali and Blundell, Mol. Biol. 234:779-815, 1993), using the graphical user interface of Discovery Studio (DS) [v1.7, Accelrys, San Diego, Calif., USA]. The structural model is described by the atomic coordinates provided in FIGS. 24A and B as well as in the priority applications, Australian Provisional Patent Application No. 2010900571, filed on 12 Feb. 2010, entitled “Protein domains and uses therefor” and Australian Provisional Patent Application No. 2010900887, filed on 23 Feb. 2010, entitled “Protein domains and uses therefor-II” and in Protein Data Bank unhjder identifiers 3KM5 and 3M1H, respectively.

The present disclosure further provides an isolated protein or fragment thereof comprising a Cleaved_Adhesin domain identified by the method of subjecting amino acid sequences of proteins to Cleaved_Adhesin domain modeling based on the amino acid sequences of one or more of K1, K2, K3, R1, R2 and/or A1 through A10 and selecting amino acid sequences having homology thereto wherein such identified amino acid sequences are regarded as defining a Cleaved_Adhesin domain.

Another aspect herein is directed to a method for the treatment or prophylaxis of infection by Porphyromonas gingivalis or a related microorganism in a mammal, the method comprising administering to the mammal an antagonizing effective amount of an agent which antagonizes function of one or more of Cleaved_Adhesin domains K1, K2 and/or K3 on Kgp and/or R1 and/or R2 on Rgp and/or A 1 through A10.

The term “infection” is used in its most general sense and includes the presence or growth of P. gingivalis or related microorganism resulting in a disease condition or having the capacity to result in a disease condition. The term “infection” further encompasses P. gingivalis or related microorganism when present as part of the normal flora. Such bacteria may, under certain circumstances, be responsible for disease development. Prophylaxis is contemplated herein to reduce the levels of P. gingivalis or related microorganism or to reduce the likelihood of a disease condition developing resulting from infection by P. gingivalis or astructurally related organism.

The present disclosure teaches the treatment of P. gingivalis or a related microorganism in humans. The disclosure extends to the prophylaxis or treatment of P. gingivalis or related microorganisms in other mammals such as primates, livestock animals (e.g. sheep, cows, goats, pigs, horses, donkeys), companion animals (e.g. dogs, cats), laboratory test animals (e.g. mice, rats, guinea pigs, rabbits, hamsters) and captured wild animals. The disclosure also teaches the prophylaxis or treatment of animals such as reptiles, amphibians, fish and avian species. All recipients of treatment of prophylaxis are included by the terms “subject”.

Infection by P. gingivalis or related microorganism in accordance with this aspect of the present disclosure is one leading to or having the potential to lead to an infection of a mucosal or vascular region such in the oral cavity, nasopharynx, oropharynx, vagina or urethra as well as the hooves of farm animals.

The term “antagonize” means and includes reducing, inhibiting or otherwise adversely affecting a Cleaved_Adhesin domain on the microbial surface molecule to the extent to reduce or inhibit hemolysis or hemolytic-like activity. The functional result of such antagonism is the inability or at least reduced capacity of P. gingivalis or related microorganism from acquiring iron, heme or porphyrin for use in, for example, metabolic pathways. Antagonism may be complete, i.e. from about 90-100% or partial, i.e. from about 30 to about 90% as determined by hemolytic assays or inhibition of P. gingivalis growth or maintenance.

The sequence identifiers defining K1, K2, K3, R1, R2 and A1 through A10 are summarized in Table 1. The sequences were determined from different strains of P. gingivalis.

Accordingly, the present disclosure teaches a method for the treatment or prophylaxis of infection of a subject by Porphyromonas gingivalis or related microorganism, the method comprising administering to the mammal an effective amount of an agent which antagonizes the function of an amino acid sequence selected from K1, K2 and/or K3 on Kgp or an amino acid sequence selected from R1 and/or R2 on Rgp or an amino acid sequence selected from A1 through A10 or HagA or a homolog thereof having at least 10% amino acid sequence similarity thereto after optimal alignment, the function antagonized including hemolytic function of erythrocytes. The subject may be a mammal such as a human or a non-mammalian animal.

This aspect extends to the use of a Cleaved_Adhesin domain-interacting molecule directed to K1, K2, K3, R1, R2 and/or one or more of A1 through A10 or another protein or a homolog or similog thereof in the manufacture of a medicament or diagnostic agent.

This aspect also extends to antibodies to Cleaved_Adhesin domain or an epitope therein. Antibodies may be monoclonal or polyclonal or synthetic or derivatized forms thereof.

The terms “similarity” and “homology” as well as “homologs” and “similogs” as used herein include exact identity between compared sequences at the or amino acid level. Where there is non-identity at the amino acid level, “similarity” includes amino acids that are nevertheless related to each other at the structural, functional, biochemical and/or conformational levels.

Terms used to describe sequence relationships between two or more polypeptides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 amino acid units. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as for example disclosed by Altschul et al., Nucl. Acids Res. 25:3389, 1997. A detailed discussion of sequence analysis can be found in Unit 19.3 of Ausubel et al., Current Protocols in Molecular Biology, John Wiley and Sons Inc, 1994-1998, Chapter 15.

The terms “sequence, similarity” and “sequence identity” as used herein refers to the extent that sequences are identical or functionally or structurally similar on a an amino acid-by-amino acid basis over a window of comparison. Thus, a “percentage of sequence identity”, for example, is calculated by comparing two optimally aligned sequences over the window of comparison, determining the number of positions at which the identical amino acid residue (e.g. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present disclosure, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

Reference to “at least 10% similarity” includes from about 10 to 100% similarity such as at least 10, 11, 12, 13, 14, 15, 16, 17, 18, 19, 20, 21, 22, 23, 24, 25, 26, 27, 28, 29, 30, 31, 32, 33; 34, 35, 36, 37, 38, 19, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% similarity. The term “homology” may also be used.

The identification of the Cleaved_Adhesin domains within the hemagglutinin region of Porphyromonas gingivalis gingipains and hemagglutininA provides a means for screening for antagonists of the function of these domains. Such antagonists are useful, for example, in the development of vaccines and therapeutic compositions for preventing or treating infection by P. gingivalis or related microorganisms. The domains also provide diagnostic targets. The present disclosure teaches the use of the K1, K2 and K3 and/or R1 and R2 and/or A1 through A 10 or their equivalent domains to produce a vaccine based on a recombinant protein, a vaccine based on a 3D epitope within the domain or an agent such as a carbohydrate which inhibits hemolytic- and/or adhesin-mediated activity.

Hence, the present disclosure teaches:

(i) a target on HA-comprising molecules from Porphyromonas gingivalis wherein the target comprises a Cleaved_Adhesin domain for antagonists and diagnostic agents;

(ii) recombinant polypeptide vaccines comprising Cleaved_Adhesin domains from the Porphyromonas gingivalis HA-comprising molecules as well as homologous domains from other proteins;

(iii) antagonists and diagnostic agents designed using the atomic coordinates surrounding or defining the Cleaved_Adhesin domains of HA-comprising molecules of Porphyromonas gingivalis, K1, K2, K3, R1, R2 and/or A1 through 10.

Another aspect taught herein is an agent capable of functionally antagonizing a Cleaved_Adhesin domain on a gingipain or hemagglutinin-binding protein or Porphyromonas gingivalis or related organism.

In an embodiment, the agent antagonizes hemolytic and/or adhesin activity of Kgp, Rgp and/or HagA by targeting domain selected from K1, K2, K3, R1 and R2 and A1 through A10 or their equivalents.

Yet another aspect taught herein is an agent capable of functionally antagonizing a Cleaved_Adhesin domain which is homologous to a Cleaved_Adhesin domain selected from K1, K2, K3, R1, R2 and one or more of A1 through A10. These agents may also be useful as antagonists or diagnostic agents for Porphyromonas gingivalis infection or antagonists, agonists or diagnostic agents for the treatment or diagnosis of conditions including infection associated with proteins comprising the homologous Cleaved_Adhesin domains.

The agent may be a derivative of the gingipain or Hag protein or the agent may be a vaccine or formulation which targets the domain or is an agent identified from screening of a chemical library or following natural product screening. The latter includes screening of environments such as aquatic environments, coral, seabeds, microorganisms, plants and Antarctic environments for naturally occurring molecules capable of acting as antagonists. The agents also include antibodies such as monoclonal or polyclonal antibodies, synthetic antibody derivatives, humanized or mammalianized antibodies and the like. Alternatively, the agent may be identified by modeling of the crystal structure of the domain. In one particular embodiment, the K2 or K3 crystal structure is determined and, hence, this may be used to identify potentially interacting molecules.

The identified domains alone or as part of a carrier molecule may be used as vaccine components to generate antibodies to the domain or their immunological relatives. Alternatively, the antagonist may be an antibody to the domain or an antibody to another region resulting in reduced function of the domain. Yet in another alternative, the antagonists form part of a therapeutic or prophylactic composition or formulation. The term “vaccine” is used to cover formulations which are designed to induce an immune response as well as formulations comprising antagonists of the Cleaved_Adhesin domains.

The antagonists, therefore, may be peptides, polypeptides, proteins, antibodies, small or large chemical entities or combinations thereof and may be in an isolated, naturally occurring form or may be in recombinant or chemically synthetic form.

Screening for antagonists may be accomplished in any number of ways. In one method, preparations of gingipains or hemagglutinin-binding molecules or parts thereof are incubated with potential antagonists and then subjected to chromatography or gel electrophoresis or immunoassay to screen for the formation of a complex. In another embodiment, 3D modeling or epitope screening is employed. In yet another embodiment, recombinant vaccines are prepared comprising peptides, polypeptides or proteins which comprise a Cleaved_Adhesin domain from a gingipain or Hag protein from P. gingivalis or a homologous domain from another protein whether related to gingipain/Hag protein or not.

In addition to screening for suitable antagonists, the present disclosure enables the chemical synthesis and/or rational design for developing Cleaved_Adhesin domain. In particular, data presented herein show that the K2 domain is a “jelly-roll” fold with two anti-parallel β-sheets. Hence, one approach is to target the fold or an epitope formed within the domain.

Accordingly, another aspect of the disclosure provides an agent capable of binding or interacting with a domain selected from K1, K2, K3, R1 and R2 and A1 through A10 or an equivalent thereof or an epitope or sub-region therein, the agent antagonizing the function of the domain. Similar agents are also contemplated for use as diagnostic agents.

When the Cleaved_Adhesin domain-containing molecules or derivatives, analogs or homologs thereof are used in a vaccine composition, they are generally used as an immunogenic component to stimulate an immune response against the domain. They may also generate an immune response to other domains since this may cause conformational changes preventing protein function.

Accordingly, another aspect enabled by the present disclosure is a composition such as therapeutic or vaccine composition comprising an agent as hereinbefore described and one or more pharmaceutically acceptable carriers and/or diluents.

The immunogenic component of a vaccine composition as contemplated herein exhibits therapeutic activity, for example, in the prophylaxis and/or treatment of P. gingivalis infection when administered in an amount which depends on the particular case. For example, for recombinant peptide, polypeptide or protein molecules, from about 0.5 μg to about 20 mg, may be administered, particularly from about 1 μg to about 10 mg, particularly from about 10 μg to about 5 mg, particularly from about 50 μg to about 1 mg equivalent of the immunogenic component in a volume of about 0.01 ml to about 5 ml or from about 0.1 ml to about 5 ml. A feature is to administer sufficient immunogen to induce a protective immune response. The above amounts can be administered as stated or calculated per kilogram of body weight. Dosage regime can be adjusted to provide the optimum therapeutic response. For example, several divided doses can be administered or the dose can be proportionally reduced as indicated by the exigencies of the therapeutic situation. Booster administration may also be required.

The vaccine or other therapeutic composition taught by the present disclosure can further comprise one or more additional immunomodulatory components such as, for example, an adjuvant or cytokine molecule, amongst others, which is capable of increasing the immune response against the immunogenic component. Non-limiting examples of adjuvants that can be used in the vaccine of the present disclosure include the RIBI adjuvant system (Ribi Inc., Hamilton, Mont., USA), alum, mineral gels such as aluminium hydroxide gel, oil-in-water emulsions, water-in-oil emulsions such as, for example, Block co-polymer (CytRx, Atlanta Ga., USA), QS-21 (Cambridge Biotech Inc., Cambridge Mass., USA), SAF-M (Chiron, Emeryville Calif., USA), AMPHIGEN adjuvant, Freund's complete adjuvant; Freund's incomplete adjuvant; and Saponin, QuilA or other saponin fraction, monophosphoryl lipid A, and Avridine lipid-amine adjuvant. Other immunomodulatory agents that can be included in the vaccine include, for example, one or more cytokines, such as interferon and/or interleukin, or other known cytokines. Non-ionic surfactants such as, for example, polyoxyethylene oleyl ether and n-hexadecyl polyethylene ether may also be included in the vaccines taught herein.

The vaccine or other composition can be administered in any convenient manner such as by oral, intravenous (where water soluble), intramuscular, subcutaneous, intranasal, intradermal or suppository routes or by implantation (e.g. using slow release technology). Depending on the route of administration, the immunogenic component may be required to be coated in a material to protect it from the action of enzymes, acids and other natural conditions which may inactivate it, such as those in the digestive tract.

The vaccine or other composition may also be administered parenterally or intraperitoneally. Dispersions can also be prepared in glycerol, liquid polyethylene glycols, and mixtures thereof, or in oils. Under ordinary conditions of storage and use, these preparations can contain a preservative to prevent the growth of microorganisms. Alternatively, the vaccine composition can be stored in lyophilized form to be rehydrated with an appropriate vehicle or carrier prior to use.

The vaccine or other composition may also be within form of a mouthwash, toothpaste and the like.

Pharmaceutical forms suitable for injectable use include sterile aqueous solutions (where water soluble) or dispersions and sterile powders for the extemporaneous preparation of sterile injectable solutions or dispersions. In all cases the form must be fluid to the extent that easy syringeability exists, unless the pharmaceutical form is a solid or semi-solid such as when slow release technology is employed or it may be deliverable by spray, inhalation, nasal drip or microdroplets. In any event, it must be stable under the conditions of manufacture and storage and must be preserved against the contaminating action of microorganisms.

The carrier may be a solvent or dispersion medium containing, for example, water, ethanol, polyol (for example, glycerol, propylene glycol and liquid polyethylene glycol, and the like), suitable mixtures thereof and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents such as, for example, parabens, chlorobutanol, phenol, sorbic acid, thimerosal and the like. In many cases, it will be preferable to include isotonic agents such as, for example, sugars or sodium chloride. Prolonged absorption of the injectable compositions can be brought about by the use in the compositions of agents delaying absorption such as, for example, aluminum monostearate and gelatin.

Sterile injectable solutions are prepared by incorporating the active compound in the required amount in the appropriate solvent with various of the other ingredients enumerated above, as required, followed by filter-sterilization. Generally, dispersions are prepared by incorporating the sterilized active ingredient into a sterile vehicle which contains the basic dispersion medium and the required other ingredients selected from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

The present disclosure further provides vaccine compositions which confer protection against infection by one or more isolates or sub-types of P. gingivalis including those that belong to the same serovar or serogroup as P. gingivalis. The vaccine composition may also confer protection against infection by other species of the genus Prophyromonas or other microorganisms related thereto as determined at the nucleotide, biochemical, structural, physiological and/or immunointeractive level; the only requirement being that said other species or other microorganism produce a peptide, polypeptide or protein which is immunologically cross-reactive to the Cleaved_Adhesin domain containing molecule of P. gingivalis. For example, such related microorganisms may comprise genomic DNA which is at least about 70% similar overall to the genomic DNA of P. gingivalis as determined using standard genomic DNA hybridization and analysis techniques. By “at least 70%” means from about 70 to 100% such as 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%.

The present disclosure teaches serogroup and serovar variants of P. gingivalis and its related microorganisms. The terms “serogroup” and “serovar” relate to a classification of microorganisms which is based upon serological typing data, in particular data obtained using agglutination assays such as the microscopic agglutination test (MAT). Those skilled in the art will be aware that serovar and serogroup antigens are a mosaic on the cell surface and, as a consequence there will be no strict delineation between bacteria belonging to a serovar and/or serogroup. Moreover, organisms which belong to different species may be classified into the same serovar or serogroup because they are indistinguishable by antigenic determination. As used herein, the term “serovar” means one or more P. gingivalis strains which is/are antigenically-identical with respect to antigenic determinants produced by one or more loci. Quantitatively, serovars may be differentiated from one another by cross-agglutination absorption techniques. As used herein, the term “serogroup” refers to a group of Porphyromonas spp. whose members cross-agglutinate with shared group antigens and do not cross-agglutinate with the members of other groups and, as a consequence, the members of a serogroup have more or less close antigenic relations with one another by simple cross-agglutination.

The present disclosure teaches therapeutic and/or prophylactic compositions capable of conferring protection against a “genetic variant” of P. gingivalis, the only requirement being that such a variant produce a peptide, polypeptide or protein having a Cleaved_Adhesin domain equivalent or similar to K1, K2 and/or K3 of Kgp and/or R1 and/or R2 of Rgp.

The present disclosure also teaches combination formulations comprising an effective amount of an immunogenic component comprising or within the Cleaved_Adhesin domain combined with an effective amount of one or more other antigens or other therapeutic molecules capable of protecting the subject against other pathogens or disease conditions.

Also taught is the use of a Cleaved_Adhesin domain on a gingipain or a hemagglutinin-binding molecule in the manufacture of a medicament for the prevention or treatment of infection by P. gingivalis or related microorganism.

In a related aspect, there is provided a use of an antagonist of interaction between a HA2-containing molecule from P. gingivalis or related microorganism and a porphyrin-containing molecule such as but not limited to hemoglobin or a precursor form thereof or part thereof such as heme in the manufacture of a medicament for the prophylaxis or treatment of P. gingivalis infection.

The use of the Cleaved_Adhesin domains such as K1, K2, K3, R1, R2 and one or more of A1 through A10 and their homologs and equivalents is also provided for diagnostic targets to detect infection by P. gingivalis and its related organisms including monitoring the efficacy of a therapeutic protocol and/or to identify potential relapses in infection. The diagnostic assay can also be used to determine minimal disease resistance (MDR). The diagnostic assay may take any form such as but not limited to an antibody based assay such as a ELISA, Western blots, dip-stick assays, protein microarrays and the like. The present disclosure teaches antibodies and other reagents specific for the Cleaved_Adhesin domains as herein described and their use in the manufacture of diagnostic kits to detect and/or monitor infection.

further provided is the use of the amino acid sequence set forth in K1, K2, K3, R1, R2 and one or more of A1 through A10 in the identification of a Cleaved_Adhesin domain in a protein or to identify a protein comprising a Cleaved_Adhesin domain.

The present disclosure further contemplates the use of the atomic coorindates for K2 or K3 or the model for K1 to design or identify a range of mimetic antagonists, agonists or other interacting compounds.

The term “atomic structural coordinates” as used herein refers to a data set that defines the three dimensional structure of a Cleaved_Adhesin domain and in particular define K2 and K3 of Kgp and further define a model for K3. Structural coordinates can be slightly modified and still render nearly identical three dimensional structures. A measure of a unique set of structural coordinates is the root-mean-square deviation of the resulting structure. Structural coordinates that render three dimensional structures that deviate from one another by a root-mean-square deviation of less than about 1.5 Å may be viewed by a person of ordinary skill in the art as identical.

X-ray crystallography is used to elucidate the three dimensional structure of crystalline forms of K2 and K3 of the present disclosure. Typically, the first characterization of crystalline forms by X-ray crystallography can determine the unit cell shape and its orientation in the crystal. The term “unit cell” refers to the smallest and simplest volume element of a crystal that is completely representative of the unit of pattern of the crystal. The dimensions of the unit cell are defined by six numbers: dimensions a, b and c and angles α, β and γ. A crystal can be viewed as an efficiently packed array of multiple unit cells. Detailed description of crystallographic terms are described in Hahn, The International Tables for Crystallography, volume A, Fourth Edition, Kluwer Academic Publishers 1996 and Shmueli, The International Tables for Crystallography, Volume B, First Edition, Kluwer Academic Publishers.

The present disclosure enables the identification of potential modulators of proteins having a Cleaved_Adhesin domain homologous to or comprising a domain selected from K1, K2, K3, R1, R2 and one or more of A1 through A10. In relation to a modulator of Kgp, Rgp or HagA, the modulator includes an antagonist or is a binding protein useful as a diagnostic agent. For other proteins, the modulators in the form of antagonists, agonists and diagnostic agents may be useful. By using the atomic coordinates of K2 or K3 and the model for K1, to identify potential modulators from a larger group, it is possible to reduce the total number of molecules which need to be tested.

The modulators may be identified by a range of means including docking a three dimensional representation of a potential modulator with the three dimensional structure of K1, K2 and/or K3. The computer representation of K2 and K3 is defined by atomic structural coordinates, similarly, the K1 model. In an embodiment, one or more modulators are docked into the Cleaved_Adhesin domain structure of K1, K2 and/or K3. The method includes: (a) providing a three dimensional representation of the atomic coordinates of a Cleaved_Adhesin domain comprising or homologous to one or more of K1, K2 and K3 of Kgp and docking a three dimensional representation of a compound from a computer database with the three dimensional representation of K1, K2 and/or K3; (b) determining a conformation of the resulting complex having a favorable geometric fit and favorable complementary interactions; and (c) identifying compounds that best fit K1, K2 and/or K3 as potential modulators of K1, K2 and/or K3 function and/or as potential diagnostic agents of K1, K2 and/or K3 and/or potential antagonists, agonists or diagnostic agents for protein comprising a homologous Cleaved_Adhesin domain.

Conveniently, the atomic coordinates for K2 and K3 are shown in FIGS. 24A and B, respectively and are available from the Protein Data Bank under identifiers 3KM5 and 3M1H, respectively.

The term “docking” refers to the process of placing a three dimensional representation of the compound in close proximity with the three dimensional representation of K1, K2 and/or K3. In an embodiment, the docking process refers to finding low energy conformations of the resulting compound/K1, K2 and/or K3 complex.

The term “favorable geometric fit” refers to a conformation of the compound/K1, K2 and/or K3 complex where the surface area of the compound is in close proximity with the surface of K1, K2 and/or K3 without unfavourable interactions (i.e. steric hindrances, etc).

Yet another aspect taught herein includes is a method of identifying potential modulation of the function of a protein which comprises a Cleaved_Adhesin domain comprising of homolgous to K1, K2, K3, R1, R2 and one or more of A1 to A10 by operating modulator construction or modulator searching computer programs on the compounds complexed with K1, K2 and/or K3. The method comprises the steps of: (a) providing a three-dimensional representation of one or more compounds, complexed with K1, K2 and/or K3, where the computer representation of the compounds and K 1, K2 and/or K3 are defined by atomic structural coordinates; and (b) searching a database for compounds similar to the compounds, using a compound searching computer program or replacing portions of the compounds complexed with K1, K2 and/or K3 with similar chemical structures from a database using a compound construction computer program, where the representations of the compounds are defined by structural coordinates. The skilled artisan will recognize that a number of suitable computer programs are available for compound searching and construction, including UNITY (Trade Mark) [Tripos, Inc.] and CATALYST (Registered) [MSI, Inc.].

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The present disclosure illustratively described herein suitably may be practiced in the absence of any element or elements, limitation or limitations which is not specifically disclosed herein.

When a group of substituents is disclosed herein, it is understood that all individual members of those groups and all subgroups, including any isomers and enantiomers of the group members, and classes of compounds that can be formed using the substituents are disclosed separately. When a compound is claimed, it should be understood that compounds known in the art including the compounds disclosed in the references disclosed herein are not intended to be included. When a Markush group or other grouping is used herein, all individual members of the group and all combinations and subcombinations possible of the group are intended to be individually included in the disclosure.

When a range is recited herein, it is intended that all subranges within the stated range, and all integer values within the stated range, are intended, as if each subrange and integer value was recited.

Aspects of the disclosure are further described by the following non-limiting Examples. In these Examples, materials and methods as outlined below are employed. Since the filing of the priority applications on which the present disclosure is based, aspects were published in Li et al., 2010 supra the entire contents of which are incorporated herein by reference.

### Materials and Methods

### Sequence Analysis

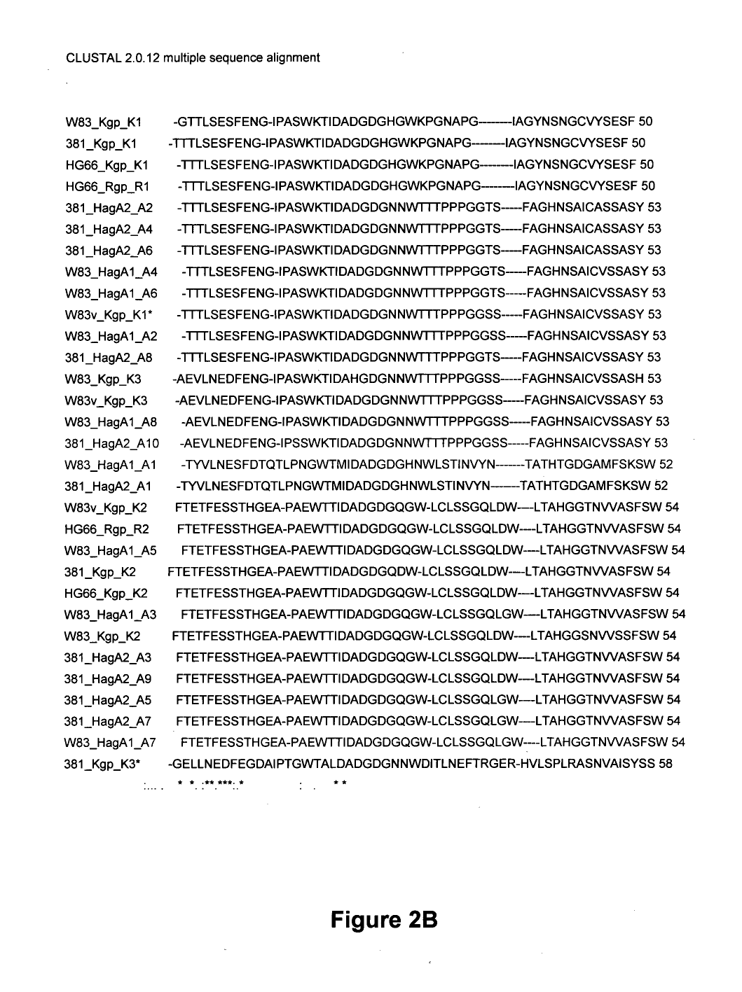

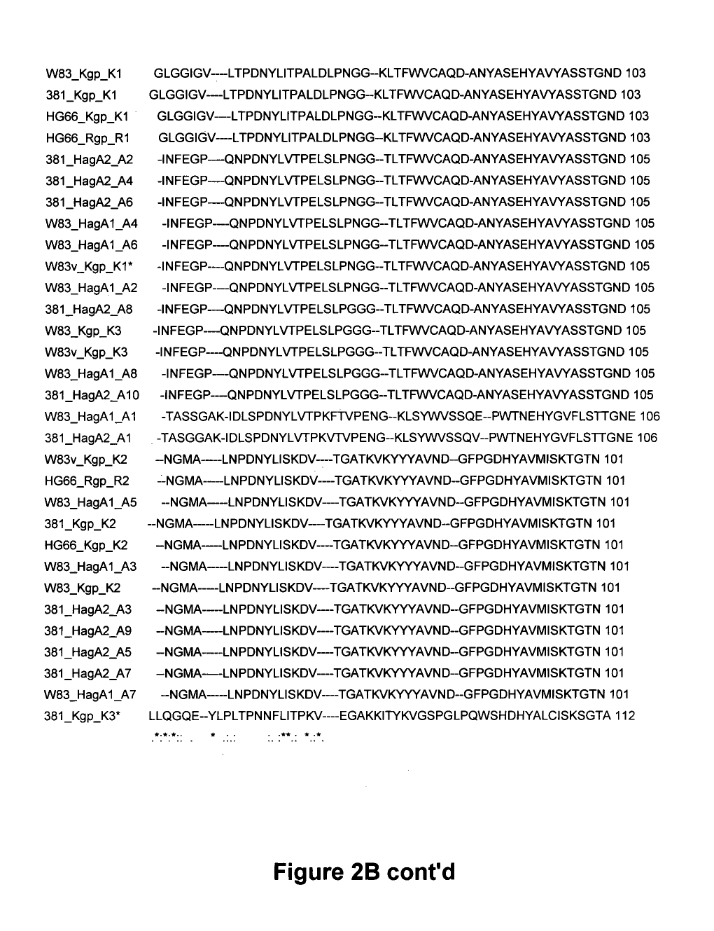

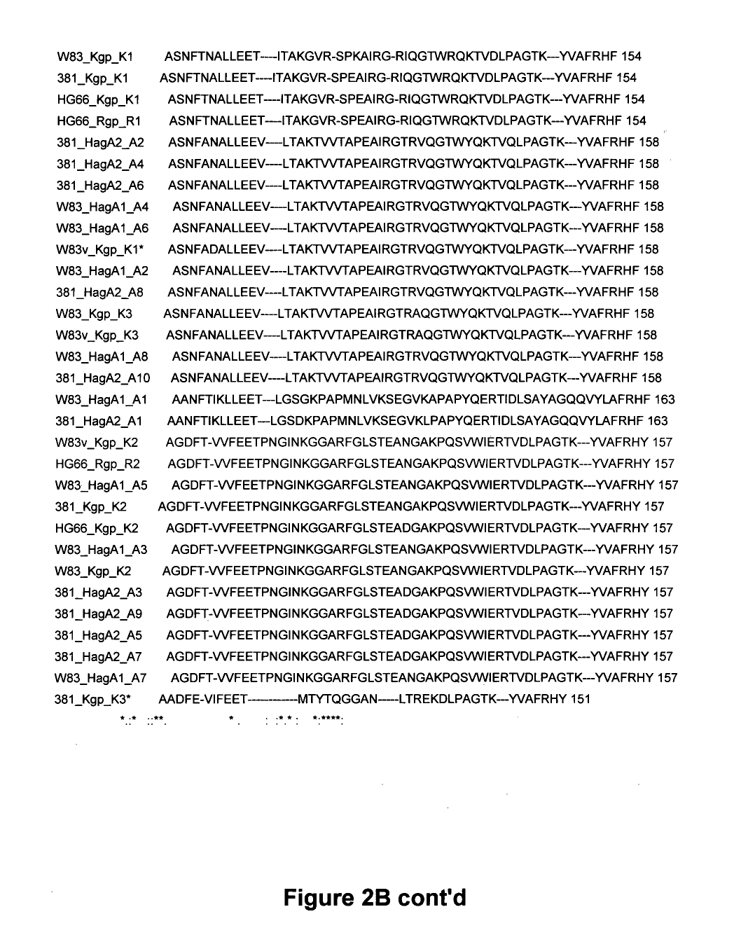

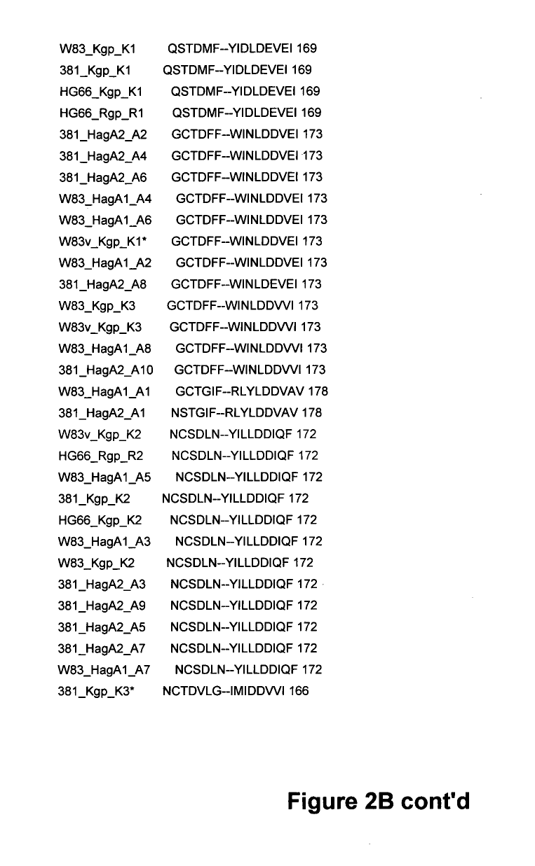

Sequences of the HagA proteins and the HA regions of Kgp and RgpA were analyzed to identify possible homologous domains. A series of multiple sequence alignments were conducted using the program ClustalW (Laikin et al., Bioinformatics 23:2947-2948, 2007). Fragments of sequences with similarities were aligned and the putative domain boundaries of each of the fragments extended until a minimum of 30% pairwise sequence identity was present. The putative homologous regions of the proteins identified by this procedure are shown in FIG. 2 and correspond to those predicted by multiple sequence alignments and sensitive sequence database searching using hidden Markov models as statistical descriptions of a sequence family's consensus (see the Cleaved_{—}Adhesin Family PF07675 in The Pfam protein families database, Finn et al., 2008 supra). A summary of related domains can be viewed at http://pfam/sanger.ac.uk/family?acc=PF07675.

### Plasmid Construction, Protein Expression and Purification

The gene fragment encoding the K2 domain (Ala1157-Gly1334) of Kgp from P. gingivalis W83 was cloned into vector pETM-21 at cloning sites of BamHI/XhoI. The construct was sequenced to confirm that there was no mutation present. The constructed plasmid was transformed into E. coli BL21(DE3) competent cells for protein expression. Cells were grown in LB medium with ampicillin (100 g/ml) at 37° C. until OD₆₀₀ reached 0.6 and the temperature was reduced to 20° C. IPTG was added to a final concentration of 0.1 mM to induce protein expression. The recombinant K2 protein with six histidines at the N-terminus was purified using Ni-NTA (Novagen) affinity chromatography. After removal of the N-terminal 6×His-tag by thrombin cleavage at room temperature, non-tagged recombinant K2 was further purified by size exclusion chromatography using a Superdex 75 16/60 column (Amersham). Approximately 20 mg protein was obtained from a 1 L culture.

Selenomethionine-substituted protein was expressed using the Overnight Express (Trade Mark) Autoinduction System (Novagen) and purified using the same methods as for native protein. They were both concentrated to 15 mg/mL in buffer comprising 10 mM Tris pH 7.6 and 150 mM NaCl. Protein concentration was determined by UV absorbance at 280 nm with a molar extinction coefficient for K2 of 39545 M⁻¹ cm⁻¹. The dispersity of purified protein samples with concentrations were monitored by dynamic light scattering using a Protein Solutions Dynapro instrument at 20° C.

Circular dichroism spectra were recorded on a Jasco 720 circular dichroism spectropolarimeter over a wavelength range of 184-260 nm with 0.5 nm resolution using a quartz cell with 1.00 mm pathlength. The K2 and the Kgp treated K2 purified proteins were prepared for analysis in 10 mM Na borate at a concentration of 7.7 μM.

The gene fragment encoding the K3 domain (Ala1427-Gly1602) of Kgp from P. gingivalis W83 (gene code AF017059 and protein ID O52050) were cloned as previously described for K2 (Li et al., 2010 supra). The gene encoding the K1 domain (Gly982-Gly1154) and the constructs for K1K2 (Gly982-Gly1334) and K1K2K3 (Gly982-Leu1661) derived from cDNA were cloned into pGEX-6P-1 vector (Amersham) at the cloning sites of BamHI/XhoI. DNA sequencing of the cloned gene fragments revealed five differences with the published sequence (gene code AF017059) which have mutated 1351Asn to Lys, 1364Tyr to Asp, 1390Asp to Asn, 1448His to Asp and 1479His to Tyr. Each of these mutations was confirmed by sequencing to be present in the cDNA of kgp used as the PCR template. Each of the five specific mutations is found in another kgp entry for P. gingivalis W83 with a gene code of AE015924 and protein code of PG1844. Based on this information, it was concluded that these mutations probably occur spontaneously in nature. Nucleotides coding for two additional residues, Gly-Ser were added to the N-terminus in the K3 construct following the 6×His-tag and the thrombin cleavage site. Five additional residues Gly-Pro-Leu-Gly-Ser were added to the N-terminus in the K1, K1K2 and K1K2K3 constructs following the GST fusion partner and the PreScission protease cleavage site.

The constructed plasmids were transformed to E. coli BL21 (DE3) competent cells for protein expression. The expression and purification of recombinant K3 protein with 6×His-tag at the N-terminus followed the same procedure as previously described for the K2 domain (Li et al., 2010 supra). After removing the N-terminal 6×His-tag by thrombin cleavage, the K3 protein was further purified by size exclusion chromatography in a buffer containing 10 mM Tris pH7.6 and 150 mM NaCl.

Similarly, the recombinant proteins, K1, K1K2 and K1K2K3 each with a GST fusion partner at the N-terminus were purified using Glutathione Sepharose 4B affinity chromatography. The beads bound with protein were washed with PreScission protease cleavage buffer containing 50 mM Tris pH 7.0, 150 mM NaCl and 1 mM DTT, followed by protease cleavage. The eluted proteins were further purified by gel filtration chromatography using Superdex 75 Hiload 16/60 or Superdex 200 Hiload 16/60 columns (Amersham) according to the size of the proteins with a running buffer of 10 mM Tris pH 7.6, 150 mM NaCl and 0.5 mM CaCl2. The protein buffer solutions were exchanged to 10 mM Tris pH 7.6 and 150 mM NaCl, leaving additional non-bound Ca²⁺ at <0.5 mM post-dialysis to avoid the formation of calcium salt crystals during crystallization screening. The purified proteins were concentrated to 10-15 mg/ml determined by UV absorbance at 280 nm.

### Crystallization, Data Collection and Structure Determination

Crystallization screenings of the K2 domain were performed by hanging-drop vapour-diffusion method using Mosquito (TTP LabTech), a Nano-drop crystallization robot, and 96-well screening kits (Qiagen). Equal volumes (0.2 μl) of protein (15 mg/mL) and reservoir solution were mixed together and incubated. Initially clusters of needle-shaped crystals were observed under the condition of 0.2 M NH₄NO₃ and 2.2 M (NH4)₂SO₄. To improve the quality of crystals, micro-seeding was performed using cat's whiskers. The larger needle shaped crystals used for X-ray diffraction were grown in the refined conditions of 0.2M NH₄NO₃, 0.1 M Na citrate pH 5.6 and 2.0 M (NH₄)₂SO₄ after 7 days of incubation at 23° C. Selenomethionine-substituted crystals were obtained under the same conditions. The crystals were cryo-protected by adding 5-20% glycerol to the mother liquor and then flash-cooled in a N₂ stream at 100° K for data collection.

Selenomethionine MAD data were collected at three wavelengths near the selenium K_{α} absorption edge: λ₁=0.97949 Å (peak); λ₂=0.97962 Å (inflection); λ₃=0.94947 Å (a high energy remote) on the beam line 23ID-B at the Advanced Photon Source, Argonne National Laboratory, using a Mar 300 CCD detector. A total of 180 successive frames in each set were collected with a 1° oscillation for each frame. A 1.4 Å native data set was collected later on the beam line 3BM1, Australian Synchrotron, Melbourne, using an ADSC Quantum 210r CCD detector. Intensities were integrated and processed using the HKL2000 program (Otwinowski and Minor, Macromolecular Crystallography, Pt A276:307-326, 1997). Both the native and Se-Met crystals belong to P2₁ space group with similar unit cell dimensions (a=30, b=60, c=86 Å and β=94°). Solvent-content analysis indicated that the asymmetric unit contained two molecules with a Matthews coefficient (VM) of 2.0 Å³/Da (38% solvent content). The data collection statistics are summarized in Table 3.

Although the nominal resolution for the three data sets recorded on a selenomethionine-substituted crystal was 1.63 Å, data extending to only 1.8 Å were employed for searching for Se sites using the program SHELX D (Sheldrick, Acta Cryst. A 64:112-122, 2008) Two fully occupied Se sites were located along with four partially occupied sites. The initial phases to 1.63 Å resolution were calculated and the density modifications were performed with the program SHELX E (Sheldrick, 2008 supra). A readily traceable electron density map resulted from the modification procedure. Automated model building was carried out using Arp/Warp (Perrakis, Nature Struct. Biol. 6:458-463, 1999), which traced 80% of main-chain structure. The resulting phases were combined with the high-resolution native data set (1.4 Å) and further manual modeling building was performed using COOT (Emsley and Cowtan, Acta Crystallographica Section D-Bilogical Crystallography 60:2126-2132, 2004) between the cycles of refinement using REFMAC5 (Murshudov et al., Acta Crystallographica Section D-Biological Crystallography 53:240-255, 1997). Analysis and validation of the structure were carried out with the assistance of the program MOLPROBITY (Lovell et al., Proteins-Structure Function and Genetics 50:437-450, 2003). The refinement statistics are summarized in Table 3:

Crystallization of K3 protein was carried out at room temperature. Crystallization screens were performed by using Mosquito (TTP LabTech), a Nano-drop crystallization robot, and 96-well screening kits (Qiagen). K1 could be crystallized but the crystals were not suitable for data collection. Bunches of tiny needle/plate crystals were observed for K3 from several conditions in the Classic and PEG suites (Qiagen) soon after the trays were set up (i.e. within ˜20 min). The conditions were optimized to 0.2 M calcium acetate, 26-28% PEG8000 and 0.1 M sodium cacodylate pH 6.5 to grow bigger crystals using the sitting drop vapor diffusion crystallization method. 2 μL of protein with a concentration of 15 mg/mL was mixed with 2 μL of solution from the reservoir. Crystals appeared after three days as bunches of plates. A suitable part of a plate with a single-crystal appearance was sectioned off and mounted on a loop. The crystal was subsequently flash-cooled in an N₂ stream at 100 K using the mother liquor as cryo-protectant. Diffraction data were initially collected using an in-house X-ray source with a rotating anode x-ray generator (Rigaku RU200) with an image plate detector (Marresearch scanner 345 mm plate). Data were also measured on the beam line 3BM1, Australian Synchrotron, Melbourne, using an ADSC Quantum 210r CCD detector. Intensities were integrated and processed using the HKL2000 program (Otwinowski and Minor, 1997 supra). Solvent-content analysis indicated that the asymmetric unit contained four molecules with a Matthews coefficient of 2.22 Å3/Da (44.58% solvent content). The structure was solved using the molecular replacement method and the program Phaser (McCoy et al., J Appl Crystallogr 40:658-674, 2007) with the K2 structure (PDB entry 3KM5) as the search model. The program COOT (Emsley and Cowtan, 2004 supra) was used for further manual model building and cycles of refinement were performed by using REFMAC5 (Murshadov et al., 1997 supra).

The data reduction and refinement statistics are summarized in Table 3.

TABLE 3
Comparison of the Ca²⁺ binding sites observed in the K2 and
K3 crystal structrues and the observed bond interaction distances

| Ca²⁺ | K2 | | K3 | |
|---|---|---|---|---|
| binding | | Distance | | Distance |
| site | Ligands | (Å)^{a} | Ligands | (Å)_{b} |
| Site-I | Thr 1162 O | 2.42/2.41 | Asp 1433 O | 2.400.02 |
| | Glu 1164 O 2 | 2.36/2.35 | Glu 1435 O2 | 2.360.02 |
| | Gly 1202 O | 2.46/2.45 | Ser 1470 Oγ | 2.540.03 |
| | Asn 1205 O | 2.36/2.40 | Ile 1472 O | 2.310.01 |
| | Asp 1326 O 1 | 2.48/2/43 | Asp 1595 O 1 | 2.480.02 |
| | Asp 1326 O 2 | 2.61/2.64 | Asp 1595 O 2 | 2.430.02 |
| | H₂O | 2.36/2.38 | H₂O | 2.390.03 |
| Site-II | Asp 1179 O 1 | 2.28/2.27 | Asp 1446 O 1 | 2.310.01 |
| | Asp 1181 O 1 | 2.42/2.44 | Asp 1448 O 1 | 2.370.04 |
| | Asp 1181 O 2 | 2.81/2.74 | Asp 1448 O 2 | 2.820.05 |
| | Asp 1183 O 1 | 2.40/2.37 | Asp 1450 O 1 | 2.390.02 |
| | Gln 1185 O | 2.27/2.24 | Asn 1452 O | 2.330.04 |
| | Asn 1221 O 1 | 2.42/2.43 | Asn 1490 O 1 | 2.280.02 |
| | H₂O | 2.44/2.42 | H₂O | 2.410.02 |

### Kgp Cleavage of K2 Protein and Purification of Kgp-Cleaved K2

Kgp and RgpB were extracted from strain HG66 P. gingivalis and purified according to established protocols as previously reported (Potempa and Nguyen, Purification and characterization of gingipains In: Current protocols in Protein Science, Chapter 21:Unit 21.20, 2007). 250 μL Ni-NTA beads bound with 2.5 mg 6×His-K2 was equilibrated in buffer containing 200 mM Tris pH 7.6, 100 mM NaCl, 5 mM CaCl₂, 0.02% NaN₃ and 10 mM L-cysteine. 20 μL of Kgp (2.13 mg/ml) containing 0.1 mM leupeptin (an Rgp-specific inhibitor) was added to 250 μL of above buffer and subsequently mixed with the beads in a 1.5 mL microfuge tube. The tube was rotated at 37° C. for 6 hours. Near complete cleavage was confirmed by SDS-PAGE.

After cleavage, beads were washed with 1 mL of buffer containing 20 mM Tris pH 8.0 and 100 mM NaCl for 10 times to remove the gingipain and 6×His-tag was removed by incubating with 45 μL thrombin (1 μg/μL, Sigma) in 250 μL buffer of 20 mM Tris pH 8.0 and 100 mM NaCl overnight at room temperature. The de-tagged cleaved K2 was further purified by exclusion chromatography using a Superdex 75HR, 10/30 column (Amersham). The purified cleaved K2 was analyzed by electrophoresis on 16% w/v Tricine-SDS-PAGE to resolve the low molecular weight fragments. These were identified by N-terminal sequence analysis at the Australian Proteome Analysis Facility, Macquarie University, Sydney.

Preparation of Other Recombinant Domains within the Hemagglutinin of Kgp

The HA2 domain corresponding to the N-terminus of K2 (see FIG. 1A) was expressed as a His-tagged product as described previously (Paramaesvaran et al., 2003 supra) and de-tagged by thrombin cleavage. The purified preparation was a predominant disulfide-linked homo-dimer. KPAD corresponding to the entire hemagglutinin of Kgp of P. gingivalis W83 (FIG. 1A) was expressed using the Impact T7 expression system (New England Biolabs Inc., USA) as described previously (Nguyen et al., Infect Immun 72:1374-1382, 2004) and purified by size exclusion chromatography to yield a relatively stable unprocessed monomer.

Analysis of protein fold stability in response to temperature was performed using the “Thermofluor” technique, which has been used as a drug discovery tool (Pantoliano et al., J Biomol Screen 6:429-440, 2001) and as a means of optimizing crystallization propensity (Ericsson et al., Anal Biochem 357:2890-298, 2006; Malawski et al., Protein Sci 15:2718-2728, 2006). Briefly, the protein of interest is incubated in the presence of SyproOrange (Invitrogen), a fluorescent dye which exhibits enhanced fluorescence when exposed to the hydrophobic residues normally buried in the interior of most natively folded proteins. Fluorescence is monitored as the temperature of the sample is increased. During a typical melting experiment fluorescence increases as the protein unfolds, then decreases as the unfolded protein molecules fall out of solution.

Melting experiments were performed in a 96-well plate format using a 7500 Fast RealTime PCR System (Applied Biosystems) in TAMRA filter mode. Protein (K1, K2 or K3) was diluted in 50 mM Tris (pH 7.5) and spiked with fluorescent dye such that a 20 μL aliquot contained 10 μg of protein and SyproOrange at a final concentration of 20×. An additional 5 μL of water or CaCl₂ stock solution was added such that final CaCl₂ concentrations of 0.00, 0.02, 0.20, 2.00, and 20.0 mM CaCl₂ were achieved. Melting curves commenced at 25° C. and progressed to 95° C. at a rate of 45 sec/° C., with fluorescence readings taken every degree. Data were normalized for basal fluorescence after which each melt curve was expressed as a percentage of maximum fluorescence.

Sequence alignment of K1 and K3 reveals that they share 71% sequence identity. Homology modeling of K1 was performed using the K3 crystal structure as a template. The aligned K1 and K3 sequences and the K3 template was used as input for the comparative protein modeling software MODELLER (Sali and Blundell, 1993 supra) using the graphical user interface of Discovery Studio (DS v1.7, Accelrys, San Diego, Calif., USA). Variations in loop conformations at loops L2, L4 and L10 suggested by sequence alignments were incorporated into the model. The arginine from L10 was manually located at the conserved anchoring site and the calcium binding sites-I and -II incorporated into the model. This homology model for K1 was subsequently used in rigid body refinements of the K1 component in the models of K1K2 and K1K2K3 derived from SAXS data.

A complex of rHSA (Prospec, Rehovot, Israel) and hemin (Sigma) was prepared as previously described (Fanali et al., FEBS J 274:4491-4502, 2007). A stock solutions of hemin at 12 mM was prepared in 100 mM NaOH. A solution of rHSA with a concentration of 0.1 mM was prepared in 0.1 M sodium phosphate buffer pH 7.0. A solution mixture with a molar ratio of rHSA:hemin=1:1.2 of the complex had a light brown color and was stable at room temperature for 20 min. It was centrifuged for 10 min and a dark precipitate discarded. The resulting supernatant was used to purify the complex by gel filtration using a Superdex 200 HR 10/30 column in 1×PBS buffer pH 7.4.

### Hemolysis

Chemicals and Reagents.

Bovine serum albumin (BSA), L-cysteine, sodium dodecyl sulfate (SDS), N-α-tosyl-L-lysyl chloromethyl ketone (TLCK), tosyl-Gly-L-Pro-L-Arg p-nitroanilide (GPR-pNA), Trizma base, Tris-hydrochloride (Tris-HCl), trypsin, and Tween 20 were purchased from Sigma (St. Louis, Mo.). Fetal calf serum (FCS) and RPMI medium were obtained from ICN Biochemicals (Irvine, Calif.). Phosphate buffered saline (PBS) and Trypticase Soy Broth were purchased from Oxoid (Basingstoke, United Kingdom). All reagents for electrophoresis and Western blotting were from Bio-Rad (Richmond, Calif.).

Assay of Hemolytic Activity.

Blood was drawn from human donors into 0.1 M citrate anticoagulant. Erythrocytes were separated from platelet-rich plasma and the buffy coat by differential centrifugation at 150×g for 15 minutes. The erythrocytes were pelleted by centrifugation at 350×g and washed twice in PBS (pH 7.4) and resuspended to 1% (v/v) in PBS. Various concentrations of K2 up to 10,000 nM were added to the erythrocytes in a total volume of 200 μL and incubated at 25° C. or 37° C. After periods of incubation, the microtiter plate was centrifuged at 1000×g for 10 min and the supernatants (100 μL) transferred into a new microtiter plate. Hemoglobin release was determined spectrophotometrically using a microtitre plate reader (absorbance at 405 nm).

Inhibition of Anion Transport.

Washed erythrocytes were treated with 0.1 mM of the anion transport blocker 4-Acetamido-4′-isothiocyanato-2,2′-stilbenedisulfonic acid disodium salt hydrate (SITS; Sigma Pharmaceuticals) at 37° C. for 1 hour. After incubation, cells were washed several times with PBS to remove the inhibitor, and K2 polypeptide was then added to the erythrocytes at different concentrations and incubated at room temperature for 24 hours.

Proteolytic Treatment of Erythrocytes.

The capacity of RgpB to sensitize erythrocytes to the hemolytic effect of K2 was assessed by pre-incubating erythrocytes with 5 mM L-cysteine-activated RgpB at 4 or 20 nM for 30 minutes at 37° C., followed by a wash step to remove excess RgpB. Cell-bound RgpB were inhibited with the protease inhibitor TLCK prior to addition of K2. K2 polypeptide or control buffer were added to the pre-treated erythrocytes and incubated at room temperature for 48 hours. Hemoglobin released was then measured as described above.

Enzyme Activity Assays.

The amidolytic activity of the purified RgpB was confirmed with the chromogenic substrate GPR-pNA (1 mM final concentration). RgpB was pre-incubated in 50 mM Tris, 1 mM CaCl₂, pH 7.5 (Tris buffer), containing 5 mM cysteine for 5 min at room temp. Enzyme and substrate were combined in a total volume of 200 μL Tris buffer and the rate of hydrolysis was measured at 37° C. within 1 hour on the basis of the increase in optical density at 405 nm, using a Bio-Rad Benchmark microplate reader.

Glycophorin A and Immunoblot Analysis.

Human glycophorin from blood type B negative which is predominantly glycophorin A, was purchased from Sigma (St. Louis, Mo.). Mouse monoclonal antibody specific for human glycophorin A (clone: GA-R2) was purchased from Becton Dickinson Inc. (Heidelberg, Germany).

Pre-activated gingipain was incubated with glycophorin A at a final enzyme to substrate (E/S) ratio of 1:100 (10 nM RgpA or Kgp with 1 mM glycophorin A) in the absence of serum. The reaction was then incubated at 37° C. for a time-course study. Hydrolysis was terminated at the indicated time with TLCK (2 mM final conc.). Aliquots were then resolved by 12% w/v SDS-PAGE under reducing and denaturing conditions and subjected to Immunoblot analysis. Immunoblot detection was performed using the primary mouse anti-human glycophorin A mAb (1:500 dilution) and the corresponding AP-conjugated rabbit anti-mouse mAb (1:1000). Membranes were washed five times in Tris-buffered saline-0.1% v/v Tween 20 between each step. Color was developed in a solution containing nitroblue tetrazolium chloride (1.65 mg) and 5-bromo-4-chloro-3-indolylphosphate p-toluidine salt (0.8 mg) in 10 mL of 100 mM Tris-HCl (pH 9.5).

Analysis of K2 (Li et al., 2010 supra) indicated the functional importance of the conformational state of the adhesin domains. For instance, poor binding of K2 to immobilized targets was observed. In the present analysis binding studies were performed using immobilized gingipain domains. Ninety six well ELISA plates were coated with K3, K2 or K1 polypeptide (0.4 g/well in PBS) and incubated overnight at 4 C. The wells were blocked with 100 L of 1% w/v skim milk in PBS for 1 hr. Haemoglobin (Hb) [Sigma] was added to the plates in various concentrations. Thereafter, anti-human Hb rabbit polyclonal antibody was added, followed by alkaline phosphatase conjugated goat anti-rabbit IgG. Alternatively, fibrinogen from human plasma (Sigma) was added to the coated plates at different levels. Thereafter, anti-fibrinogen mAb (FG-21, Sigma) was added, followed by alkaline phosphatase-conjugated rabbit anti-mouse IgG. The binding affinity between the polypeptides and rHSA or rHSA-heme was also detected by adding different levels of rHSA in PBS buffer or rHSA-heme in buffer containing 0.1 M heme to the coated plates. Thereafter, anti-HSA mAb (15C7, AbCam) was added, followed by alkaline phosphatase-conjugated rabbit anti-mouse IgG. The plates were washed with 0.05% v/v Tween 20 in PBS solution except for the rHSA-heme solution in the wells were washed with 0.1 M heme in Tween solution three times between each step. Color development was detected with phosphatase substrate. Data were fitted by non-linear regression using GraphPad Prism 4.0 software (GraphPad Inc., La Jolla, Calif., USA). Apparent Kd values were calculated from the fitted curves.

Loop 1 of K2 represents a unique characteristic for this particular adhesin domain. To probe the contribution of this loop to binding of mammalian proteins customized affinity purified rabbit antibodies to the antigenic sequence ETFESSTHGEAPAEC (SEQ ID NO:32) were prepared by GenScript Corp. This preparation was evaluated by pre-incubation of K3 or K2 at 10 μM/well in PBS with or without rabbit anti-HA2 polyclonal antibody at 5 μg/ml overnight at 4° C. The wells were then blocked with 100 μl of 1% w/v skim milk in PBS for 1 h. rHSA at various levels (1 to 100 μM) was added to the plates. Thereafter, anti-HSA mAb pre-absorbed with normal rabbit serum at a ratio of 1:1 was added, followed by alkaline phosphatase-conjugated rabbit anti-mouse IgG. Color development was detected with phosphatase substrate.

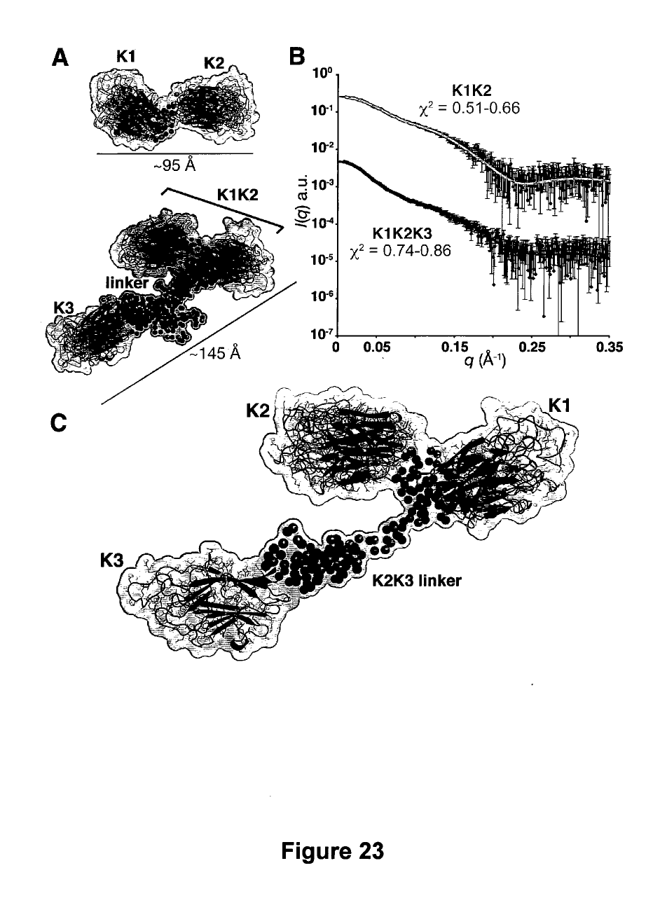

K1K2 and K1K2K3 were buffer-exchanged using a Superdex 75 (10/300) size-exclusion column in 150 mM NaCl, 10 mM β-mercaptoethanol, 10 mM Tris, pH 7.6. Both individual protein samples eluted as a single peak and the pooled peak fractions from the column were analyzed immediately using SAXS. A protein free fraction was used as an exact solvent blank for the SAXS experiments. SAXS data of I(q) vs q (q=(4π sin θ)/λ), 2θ is the scattering angle and the λ wavelength of the radiation; CuKα, 1.54 Å) were measured as essentially described in (Jeffries et al., Journal of Molecular Biology 377:1186-1199, 2008) at 15° C. over a period of 3 hr using a SAXSess (Anton Paar, Austria) line collimation instrument equipped with a CCD detector over a q-range of 0.010-0.37 Å⁻¹ (K1K2) or 0.008-0.4 Å⁻¹ (K1K2K3) and 10 mm integration width. The program SAXSquant 2.0 (Anton Paar, Austria) was used to subtract the scattering of the solvent blank from the proteins in solution to yield the scattering profiles from the protein molecules alone, while also including corrections for sample absorbance and detector sensitivity. The program GIFT (Bergmann et al., Journal of Applied Crystallography 33:1212-1216, 2000) was used to calculate the probable distribution of distances between atom pairs in real space (P(r) profiles) using an indirect Fourier transformation, that included a correction for beam geometry, from which the maximum dimension (Dₘₐₓ), radius of gyration (R_{g}) and forward scattering intensity at zero angle (I(0)) of both K1K2 and K1K2K3 were determined. The smoothed I(q) vs q profile output from GIFT was used to apply the beam-geometry correction to the experimental data and all subsequent structural parameters and modeling as quoted in the text are derived from the K1K2 and K1K2K3 beam-geometry corrected datasets. This includes Guinier analysis, (analyzed in PRIMUS [Konarev et al., Journal of Applied Crystallography 36:1277-1282, 2003]), ab initio shape restoration using the program DAMMIF (Franke and Svergun, Journal of Applied Crystallography 42:342-346, 2009) and rigid-body modeling using BUNCH (Petoukhov and Svergun, Biophysical Journal 89:1237-1250, 2005). It must be noted that ab initio shape restoration was performed 10 independent times using the K1K2 and K1K2K3 GIFT outputs and the final solutions (average K1K2 fit=0.61; K1K2K3, χ=0.82) were aligned, averaged and volume corrected (Volkov and Svergun, Journal of Applied Crystallography 36:860-864, 2003) to produce the restored shape models. The normalized spatial discrepancy values of K1K2 and K1K2K3 were 0.66+/−0.01 and 1.0+/−0.02, respectively (Volkov and Svergun, 2003 supra). BUNCH refinement was repeated five independent times on both K1K2 and K1K2K3 datasets to derive the consensus ensembles that fit that data shown in FIG. 23A. The goodness-of-fit of the final BUNCH-refined rigid-body models of K1K2 and K1K2K3 were evaluated using the χ² parameter from CRYSOL (Svergun et al., Journal of Applied Crystallography 28:768-773, 1995). CRYSOL was also used to estimate the molecular volumes of K2 and K3. The experimental molecular mass (Mᵣ) of K1K2 or K1K2K3 was evaluated from the I(0) of each SAXS dataset (on an absolute scale) using the method of Orthaber et al., Journal of Applied Crystallography 33:218-225, 2000. The necessary parameters required for this method; namely the contrast, Δp, (or mean scattering/electron density difference between K1K2 or K21K2K3 against the solvent, in cm⁻²) and partial specific volume (in cm³·g⁻¹) were calculated using the programs MULCH (Whitten et al., Journal of Applied Crystallography 41:222-226, 2008). and NucProt (Voss and Gerstein, 2005), respectively. Protein concentrations (g·cm⁻³) were determined at by UV absorbance measurements A₂₈₀ₙₘ using theoretical extinction coefficients.

PDB Deposition Coordinates of K2 have been deposited in the Protein Data Bank (PDB ID Code: 3KM5). PDB deposition Coordinates of K3 have been deposited in the Protein Data Bank (PDB ID CODE: 3M1H). The atomic coordinates are also shown in FIGS. 24A and B, respectively.

### Example 1

### Model of the Domain Structures of HA Regions

A bioinformatic analysis of the sequences of HA regions expressed on the surface of P. gingivalis supported the proposal that an alternative domain structure existed in the gingipains (see the Cleaved_{—}Adhesin Family PF07675 in The Pfam protein families database, Finn et al., 2008 supra). The Cleaved_Adhesin 19 kDa domains K1, K2, and K3 or R1 and R2 defined herein are similar in sequence and share more than 30% sequence identity indicating structural homology (FIG. 2d). The Cleaved_Adhesin domains is designated in RgpA as R1 and R2 and those in Kgp as K1, K2 and K3 (FIG. 1b). R1 and K1 are contained at the C-terminus of the previously defined Rgp44 and Kgp39 regions respectively while K3 lies at the N-terminus of the Kgp44 region. R2 and K2 span the conserved Rgp15/Kgp15 region (also referred to as HA2 or HGP15 or HbR in previously defined domain models) and continue across the region boundary for a further 44 residues of the RgpA17 and Kgp44 regions (FIG. 1). In the same way, homologous domains found in HagA can be designated A1-A10 (FIG. 2). This alternative domain model for the HA regions of gingipains also presents one possible reason for the observed stability of these proteins after proteolysis. Since the catalytic and adhesin domains of the gingipains are stable as non-covalently bound multi-domain complexes (Takii et al., Infect Immun 73:883-893, 2005, Pike et al., 1994 supra), the integration of the 44-residue extension beyond the proteolysis site into a stable K2/R2 globular domain could serve as an anchoring mechanism of the HA2 region to the rest of the gingipain complex.

In addition to these particular P. gingivalis proteins, genomic analyses indicate that homologous protein modules associated with the Cleaved_Adhesin domain family may be expressed in at least 7 other bacterial phyla including Flavobacterium and Proteobacterium species (see the Cleaved_{—}Adhesin Family PF07675 in The Pfam protein families database, Finn et al., 2008 supra). A number of these hypothetical proteins have sequences that suggest a more complex domain structure that includes extra domains such as fibronectin type III (FNIII) and/or Meprin, A5, μ (MAM) domains. Such complex domain structures are consistent with putative roles in cell adhesin. Sequence features of the Cleaved_Adhesin domain family imply structural similarities exist with other related protein domain families and that it forms part of the galactose-binding domain-like superfamily (GBD CL0202: The Pfam protein families database, Finn et al., 2008 supra). The GBD superfamily is defined by a beta sandwich fold with a distinct β-barrel topology different from that found in immunoglobulin (Ig)-like and FNIII domains. Representatives of the superfamily include the MAM domain (Aricescu et al., Science 317:1217-1220, 2007), a number of the carbohydrate binding modules (CBMs) [Jamal-Talabani et al., 2004 supra, Bae et al., 2008 supra], and the ephrin receptor ligand binding domains (Ephrin_lbd) [Himanen et al., 2001 supra]. Many of the protein modules in these structurally related families are involved in cell adhesin that is mediated by specific protein-protein interactions, or by carbohydrate binding.

### Example 2

### The K2 Protein Domain

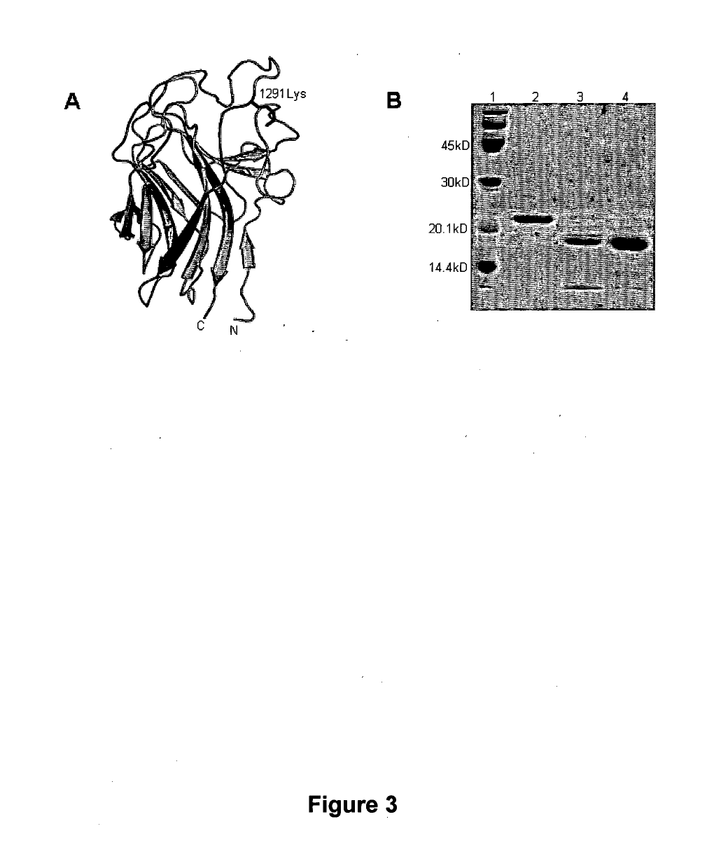

Recombinant 6×His-tagged K2 protein was expressed at a high level in E. coli and purified to a purity of more than 95% (as estimated on SDS-PAGE) by affinity and gel filtration chromatography. The protein migrates as a 23 kDa species on SDS-PAGE although the calculated molecular weight is 19.3 kDa (FIG. 3). Thrombin treated non-tagged recombinant K2 can be readily concentrated to 30 mg/ml and remains monodisperse as observed by Dynamic Light Scattering. The purified protein is very stable and no proteolysis was detected by SDS-PAGE after storage at 4° C. for 3 months.

### Example 3

### K2 Structure Analysis

### (1) Structure Overview

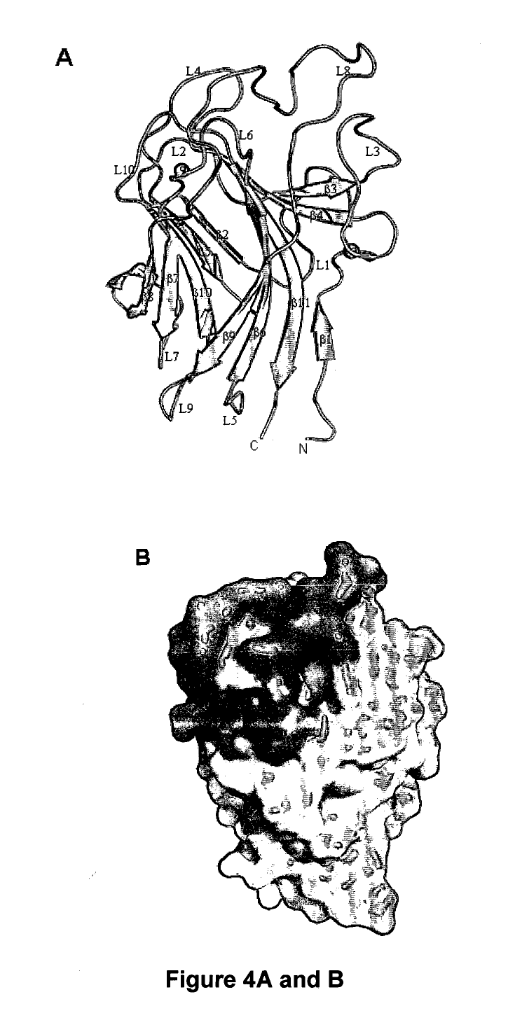

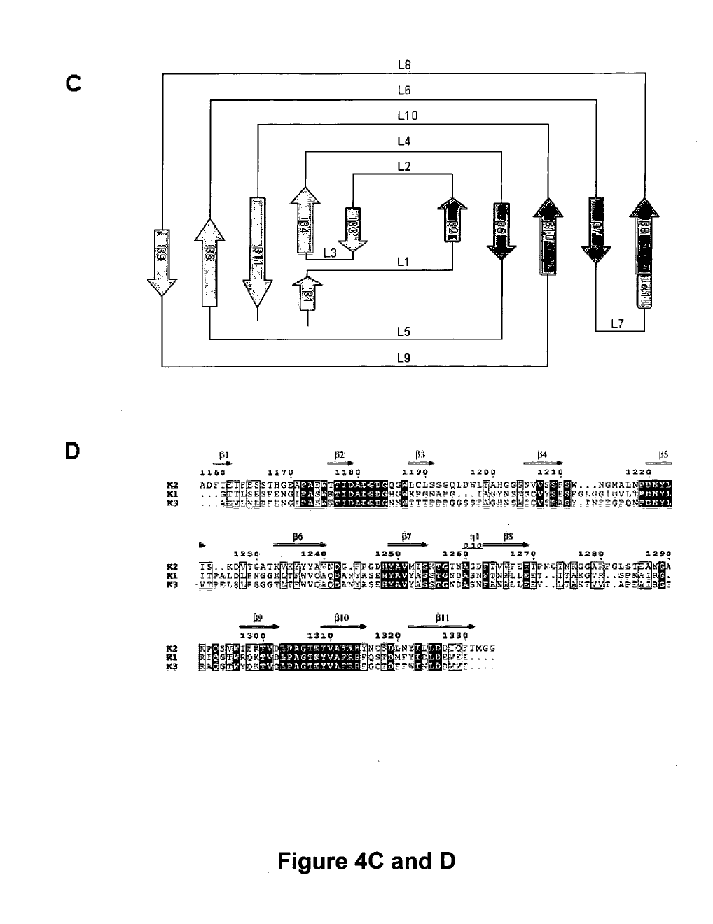

Overall, the K2 domain has a ‘jelly-roll’ fold with eleven β-strands forming two anti-parallel β-sheets (FIG. 4). These eleven β-strands are linked by ten loops and a one-turn helix which is formed by residues Asn1259-Asp1262. The β-barrel is formed by two β-sheets comprising of β1-β3-β4-β11-β6-β9 and β2-β5-β10-β7-β8 respectively (FIG. 4d) and is the core of the whole structure. β-bulges are observed at Val1266 and Asp1327 found in β8 and β11 respectively distorting both β-strands. All but one of the loops, L1, is located at either end of the β-barrel. While three short turns L5, L7 and L9 which connect strands β5-β6, β7-β8 and β9-β10 respectively are at one end of the β-barrel, the other six relatively longer loops L2, L3, L4, L6, L8 and L10 stretch out from the other end of the β-barrel and constitute the ‘head end’ with loop L8 connecting β8 and β9 being the longest. Consisting of 30 residues (Glu1269-Glu1298), L8 extends across one end of the β-barrel and then turns around and comes back to the same side to connect to β9. From the side view of the β-barrel, L8 almost covers one end of the β-barrel (FIG. 4b). Three fragments with weak or missing electron densities (see below) are all part of the long L8 loop. An analysis of the surface electrostatic potential shows that one side of the molecular ‘head end’ containing Lys1276, Arg1280 and Lys1291 from L8 is highly positive charged while the flanking regions of this end of the β-barrel, formed by L1-β2-L2, L3 and L4 are highly negatively charged as contributed by acidic residues Glu1170, Asp1179, Asp1181, Asp1183, Asp1196 and Asp1220 (FIG. 4c).

Two independent observations of the molecular structure of K2 are observed in the crystal (designated chains A and B) and they vary significantly only in their crystal packing arrangements and in interactions with additives such as glycerol. A number of intermolecular interactions result from the crystal packing, but there is no significant protein-protein interaction surfaces suggested by these arrangements. They involve residues in 6 β strands (β1, β2, β6, β9, β10, β11) and 7 loops, (L1, L2, L3, L4, L7, L8, L10) in chain A. Alternative loop conformations may be populated in solution or ligand complexes.

Gly1333 in the C-terminus is only present in chain A and Gly1334 is absent in both chain A and chain B. Two calcium (FIG. 4a) and one NO₃⁻ ions were modeled in each of the two molecules and one SO₄²⁻ ion binds at the molecular interface within the asymmetric unit but interacting more closely with the chain A molecule. In each molecule, a Ca²⁺ coordinates to Thr1162, Glu1164, Gly1202, Asn1205, Asp1326 and one H₂O molecule binding L2 tightly to the β-barrel. Since there is no Ca²⁺ related function known for K2, this ion might act primarily to stabilize the conformation of the loop which surrounds it. Another Ca²⁺ coordinates to Asp1179, Asp1181, Asp1183, Gln1185, Asn1221 and one H₂O molecule interacting with L3 and binding N-terminal β1 to C-terminal β11. The binding of this ion may act to stabilize the overall β-barrel structure of the domain. As the Ca²⁺ ions are not present in the crystallization and cryo-protectant solutions and appear to be tightly bound, they were most likely present when the protein was expressed and folded.

With a diffraction resolution of 1.4 Å, the overall electron densities are very clear and strong except those for residues Gly1273-Lys1276 and Ser1284-Gly1289 in L8. Electron densities in these two fragments are so weak that only the main chain for these residues is able to be modeled in chain A and no mainchain model for residues Ala1287-Gly1289 in chain B is reported here. The fragment Gln1293-Val1295 in chain A and chain B are observed in different conformations. The residual electron densities for the fragment Gln1293-Val1295 in chain A indicate that there is at least one alternative conformation present in the crystal but this was not able to be modeled because of the weak residual densities, while in chain B the equivalent fragment has only one conformation with clear density. One glycerol (added as a cryo-protectant) and one water molecule were found to sit in a closed pocket formed by the fragments of Trp1197-Thr1199, Lys1291-Trp1296 and Tyr1322-Leu1324 only in chain B. With the hydrogen bond connections, these glycerol and water molecules are believed to have stabilized the conformation of fragment Gln1293-Val1295 in chain B. Observations of weaker densities in residues Gly1273-Lys1276 and Ser1284-Gly1289 may indicate that other conformers of loop L8 can be readily adopted in protein or ligand bound complexes formed by K2.

### (2) Structural Homologs of K2

K2 is the first structure solved in the Cleaved_Adhesin family. An analysis of the sequences of those known to be associated with this family suggests that the structural differences are most likely to be found primarily in the loop regions. A comparison of the sequences of the closely related K1 and K3 domains (71% sequence identity in strain W83) with K2 (36% and 33% identity respectively) indicates that loops L3 and L8 are shorter in these particular Cleaved_Adhesin domains (FIG. 4c). Variability in the ‘head end’ of the β-barrel loop conformations may present different binding surfaces to a range of possible ligands interacting with these homologues. In contrast, two highly conserved regions of sequence are observed when comparing these three domains which correspond to regions that include loops L2 and L9. These conserved loop regions may present surfaces which interact with shared common ligands.

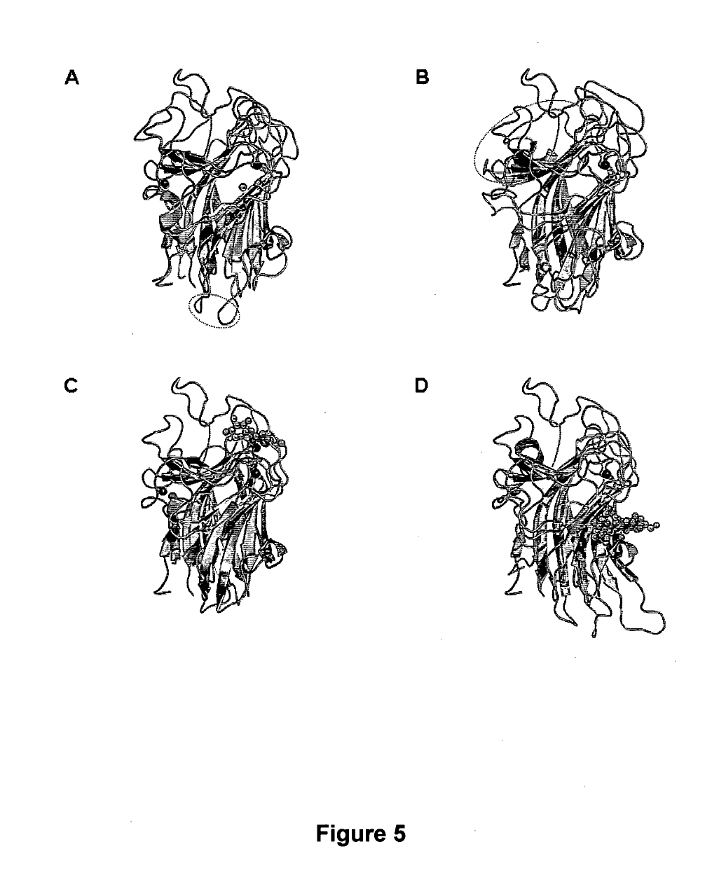

In order to understand more about the structure and its membership in the galactose-binding domain-like superfamily, a search for structural resemblances and common structural cores found in K2 was performed by the program DALI (Holm AND Park, Bioinformatics 16:566-567, 2000). The list of the closest structural homologues includes the MAM domain found in human receptor-type tyrosine-protein phosphatases (RPTPμ MAM domain), a protein adhesin, followed by ephrin type-A/B receptors EPHA2, EPHA4, EPHB2 and EPHB4 and a number of carbohydrate binding modules (sorted by the Z score, which represents the strength of structure similarity). The superimpositions of K2 with a number of these structural homologues are shown in FIG. 5. Each of these structural homologs neatly superpose with K2 in the O-barrel regions which form the cores of the structures even though they have only 6-16% sequence similarities. K2 superimposes to RPTPμ MAM domain and ephrinB2-binding domain with 139 and 133 aligned residues and rmsds of 2.5 and 2.4 respectively while K2 superimposes to CBM36 and TpolCBM16-1 with 109 and 122 aligned residues and C_{α} rmsds of 2.4 and 2.7 respectively. The smaller numbers of aligned residues between K2 and the CBM domains are due to the shorter loops at one end of the ‘jelly-roll’ β-barrels of these modules. The binding sites for calcium in K2 that stabilizes the overall β-barrel structure are mirrored in a number of the CBMs while in the MAM domain structure, a bound sodium ion is observed.

The differences between K2 and these homologs are mainly in the loop structures at the ‘head end’ of the β-barrels. Some of these loops from K2, MAM domain and ephrinB2-binding domain are partially super imposable but overall their conformations are quite different in K2 (FIG. 5), particularly loops L1, L3, L6, L7 and L8 at the ‘head end’ of the β-barrel. In the MAM domain the regions which act in protein-protein recognition are actually the two loops at the other end of the β-barrel. These two loops partially superimpose to loops L5 and L9 in K2 and are slightly longer. In comparing K2 and the ephrinB2-binding domain, all of the loops involved in the interaction between the receptor and its ligand ephrinB2 do not superpose to loop conformations observed in the K2 structure. Overall, in these comparisons the conformations of loops L8 and L3 in K2 are the most different and L8 is observed to be partially disordered indicating flexibility. Whether these differences and loop L8 conformational flexibility are related to varying functional roles is yet to be determined.

In comparing the ‘head ends’ of the β-barrels of K2 and CBM domains, the loops are shorter in CBM domains and there is no equivalent to the longest loop K2-L8 in the CBM structures (FIG. 5). Type C CBM domains have a grooved surface on the ‘head end’ of the β-barrel which has been to be shown to be a carbohydrate binding site. A typical example of this is the CBM36 structure. The equivalent spatial position of this type C binding site in the K2 structure is occupied by the longer flexible loops of the ‘head end’. The CBM16 has a type B carbohydrate binding site which is a groove formed by one bent β-sheet. The equivalent position in K2 structure is blocked by the small helix which fills the cleft and makes it inaccessible to any ligand.

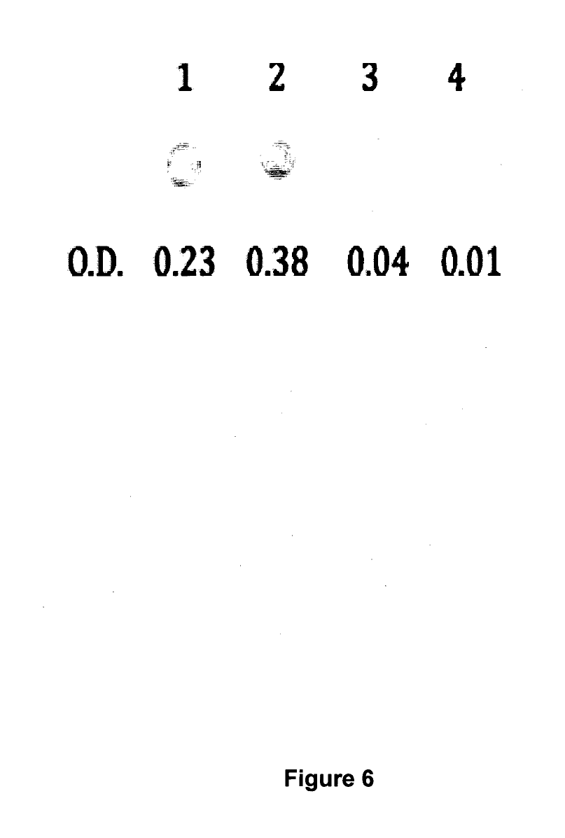

To examine carbohydrate binding by K2 a selection of glycans was absorbed to nitrocellulose membranes and probed with recombinant K2 as described in FIG. 6. Weak binding was detected for galactose and chondroitin sulfate (containing N-acetylgalactosamine). The data indicated specificity for galactose and N-acetylgalactosamine commensurate with homology to galactose binding proteins.

### Example 4

### Gingipain Cleavage of K2 Protein

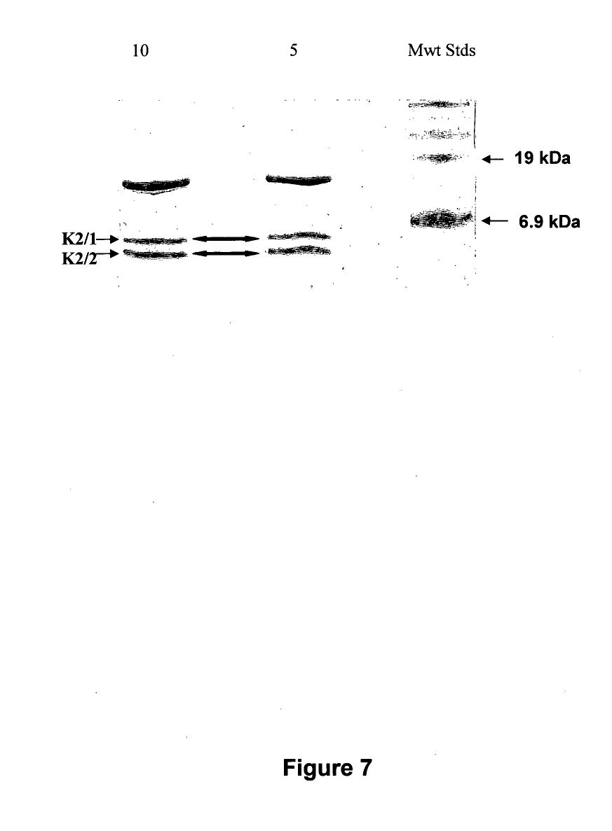

HA2 (also known as Kgp15 or Rgp15 or HbR) has been assigned as a heme binding acceptor relating to binding capacity for hemin and hemoglobin. From sequence analysis, K2 extends for 44 residues beyond the C-terminal of HA2. In the K2 structure, the 44 residues form β-strands 9, 10 and 11 which are intrinsically part of the two anti-parallel β-sheets (FIG. 4). The proposed Kgp processing site Lys1291 which produces the C-terminus of HA2 is found on the surface of L8 in K2 (FIG. 4). Lys1291-Pro1292 form a small arch suggesting suitable access for the catalytic action of Kgp (FIG. 3). To establish this, digestion of K2 was performed with a purified Kgp preparation. Kgp cleaved K2 elutes from size exclusion chromatography with a size comparable to native K2 and analysis by SDS-PAGE of these fractions (FIG. 3) confirmed that the cleaved protein is a non covalent complex of cleaved fragments. The cleaved K2 product was shown to be stable by this method prior to and after the period in which the hemolysis assays were conducted. Comparisons of native PAGE and SDS-PAGE run without prior boiling of the cleaved K2 product with standard SDS-PAGE gels (in which the prior boiling of sample is conducted) also indicate the presence of a non-covalent complex. A comparison of CD spectra for K2 and cleaved K2 indicated no observed difference in secondary structure. Attempts to grow crystals of cleaved K2 were not successful. Digestion with RgpB did not produce detectable cleavage of K2. The cleavage products were analyzed and confirmed by N-terminal sequencing. This analysis established the processing sites to occur at Lys1291 (FIG. 7) and Lys 1276.

### Example 5

### Hemolysis

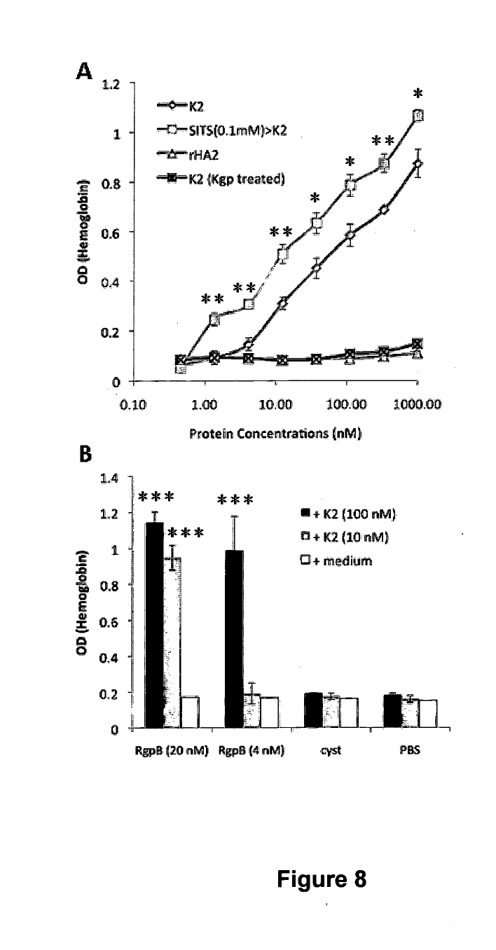

Freshly isolated human Blood Group A and O erythrocytes were studied. Hemolysis of red cells induced by K2 was concentration- and time-dependent. K2 was effective in a range of 10 to 5000 nM with 50% hemolysis observed at ˜250 nM (FIG. 4a). The absence of the catalytic domain in the K2 polypeptide indicated that the hemolysis was independent of proteolytic activity. In this regard there is no data in the literature to indicate that the hemagglutinin domain of Kgp including K2, has proteolytic activity and K2 does not contain any recognized catalytic moiety. Hemolysis induced by K2 developed over several hours. Buffer controls did not produce detectable hemolysis. The hemolytic effect of K2 was eliminated by incubating the polypeptide for 15 min at 95° C. or for 40 minutes at 60° C. K2 induced hemoglobin release at room temperature but not at 4° C. Cleaved K2 was also studied for hemolytic action and was found to be completely ineffective (FIG. 8a). Extracted Kgp, processed by proteolysis, either non-activated or cysteine-activated, was also ineffective in inducing hemolysis. This was also the case for rHA2 (FIG. 8a) and for the recombinant polyadhesin domain of Kgp.

As the Kgp cleavage sites in K2 at Lys 1291 and Lys 1276 are located within loop L8, the data provide evidence that cleavage or truncation specifically affecting this loop critically removes hemolytic capacity. For hemolytically active K2 evidence that the 30 residue surface loop L8 is flexible is not inconsistent with involvement in a specific binding function. Accordingly, proteolytic processing of the loop (at Lys 1291 and perhaps Lys 1276) would readily modify the conformational state of L8 leading to loss of capacity to engage in intermolecular associations necessary for hemolytic action.

The effect of the anion transport inhibitor SITS on hemolysis of erythrocytes induced by the K2 domain was examined (FIG. 8a). There is evidence that SITS acts as an inhibitor of erythrocyte Band 3 protein, a major mediator of anion transport (Galtieria et al., Biochimica et Biphysica Acta (BBA), Biomembranes 1564:214-218, 2002). SITS is reported to have either a protective effect or to promote erythrocyte lysis depending on the source of erythrocytes studied and the experimental conditions (Yamaguchi et al., J Biochem 118:760-764, 1995). While SITS did not induce detectable hemolysis alone, pre-incubation with this inhibitor decreased the threshold for detectable hemolysis by K2 by an order of magnitude. On this basis, it can be asserted that interference with anion transport sensitizes erythrocytes to the hemolytic effect of K2.

The potential for proteolytic activity of the gingipains acting cooperatively with K2 to enhance hemolytic activity was analyzed. This was investigated to develop the findings of Chu et al., 1991 supra indicating that proteinase inhibitors effectively blocked the hemolytic action of P. gingivalis. RgpB was selected for initial studies as this gingipain lacks the predicted domains of the region of RgpA. When 5 mM L-cysteine activated RgpB at 4 or 20 nM was incubated with erythrocytes there was only a low level of hemoglobin release (FIG. 8b). Subsequent addition of K2 (10 nM or 100 nM) to the RgpB-treated erythrocytes induced extensive lysis of erythrocytes at an earlier time point (FIG. 8b) than for K2 incubated with non-treated cells (FIG. 8a). RgpB degrades glycophorin A (Sakai et al., J Bacteriol 189:3977-3986, 2007) thereby potentially exposing the closely associated Band 3 protein on the erythrocyte surface (Auffray et al., Blood 97:2872-2878, 2001). However, treatment with SITS did not augment this effect of RgpB (FIG. 8b). The potential of activated Kgp to sensitize erythrocytes to K2 was also studied. Results shown in FIG. 8a indicate augmentation of hemolysis that was less potent than for RgpB treatment. Pre-treatment with SITS did not modify the pattern of hemolysis. The results explain the apparent dependence of hemolysis on proteolytic activity of P. gingivalis (Chu et al., 1991 supra). Published reports (Chu et al., 1991 supra and Lewis et al., 1999 supra) indicate that hemolysis by P. gingivalis is relatively slow and, therefore, demonstration of rapid hemolytic mechanisms would not be expected. The time course reported for K2 in the present study is compatible with this finding.

### Example 6

### Contribution of K2 to Other Biological Functions Reported for Gingipain Hemagglutinin Domains

Using standard ELISA binding assays (O'Brien et al., J Immunol 75:3980-3989, 2005), the binding of gingipain domains to fibrinogen and fibronectin was examined. No binding of K2 or cleaved K2 could be detected while high affinity binding of Kgp to both fibrinogen and fibronectin was detected as previously reported (O'Brien et al., 2005 supra). In this study the authors provisionally mapped fibrinogen and fibronectin binding to sites located beyond C-terminus of K2. Accordingly, the lack of recognition by K2 of these substrates is predicted.

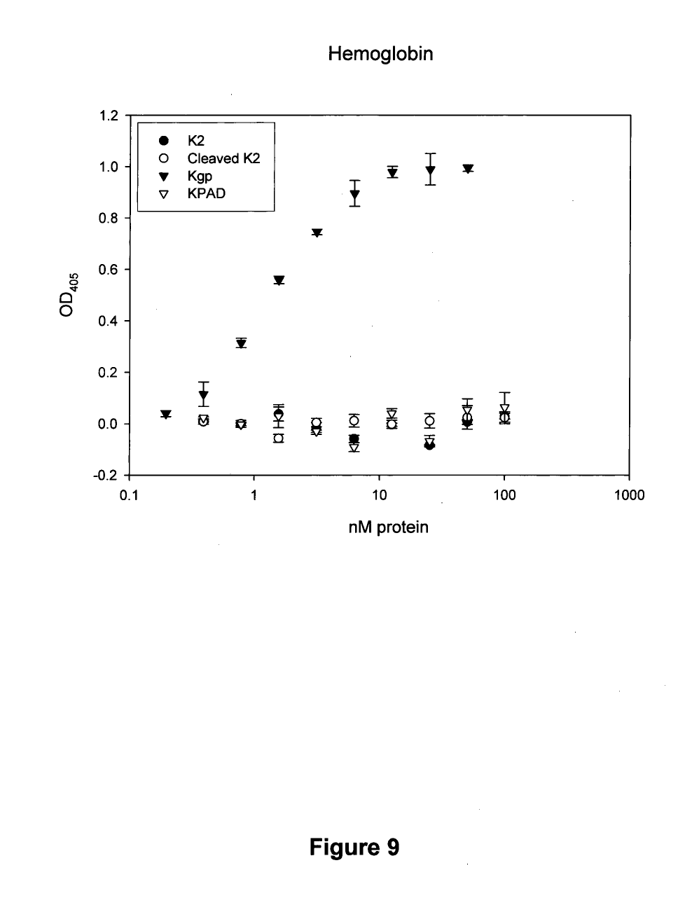

As the N-terminal fragment of cleaved K2 corresponds to the putative hemoglobin/heme receptor (HbR or HA2) (FIG. 1A) it was of interest to examine these functions. Analysis (FIG. 9) demonstrated that while Kgp recognized both hemoglobin and heme by high affinity binding as expected (DeCarlo et al., 1999 supra), K2 and cleaved K2 did not show detectable binding. Recombinant unprocessed Kgp HA polypeptide (KPAD) (FIG. 1A) bound heme but did not bind hemoglobin. The results indicate the complexity of binding that could be regulated by folding restrictions imposed by the multi-domain gingipain complex.

### Example 7

### Use of Cleaved_Adhesin Domain

The Cleaved_Adhesin Domain modeling confirms a relationship to the galactose-binding domain (GBD) superfamily. These domains are based on bacterial genomic data of a number of expressed proteins and likely to be acting in concert with other adhesin modules such as fibronectin III and MAM domains. These relationships lead to the consideration of a number of specific roles in which these proteins and their domains may be involved. The structural models of the HA protein regions expressed on the surface of P. gingivalis have previously been based on the analysis of proteolytic fragments observed after extraction from the cell. Analysis of the in-vitro biochemical activities of these fragments has suggested a number of different possible functions for the HA regions of gingipains and Hag proteins. The determination of the K2 structure and the confirmation that an integral folded state exists within a novel boundary of the polyprotein suggests that an alternative domain model represents the functional states of these proteins. In the case of K2, it is shown that the structural domain defined by these boundaries facilitates a hemolytic function consistent with that previously associated with the outer membrane gingipain complexes (Lewis et al., 1999 supra). This is an example of the use of the alternative domain model in understanding the exact biological and physiological roles of these domains in this organism. Knowing these domain boundaries and by inference, the domain boundaries of other components of HA regions in gingipains and Hag proteins, enables the identification of agents which specifically target these domains and to inhibit protein activity.

### Example 8

### Applications of the Structure and Function of Cleaved-Adhesin Domains of Gingipains in P. gingivalis

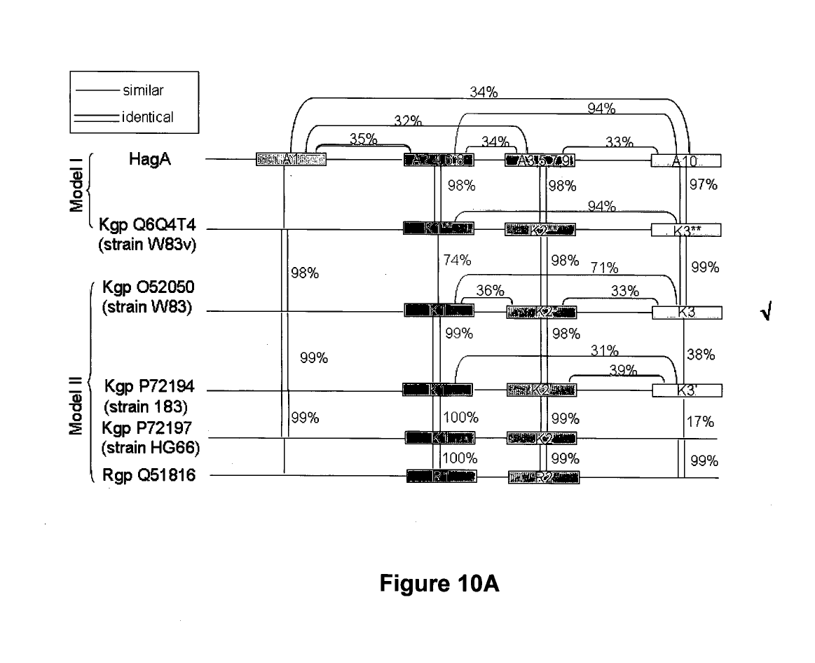

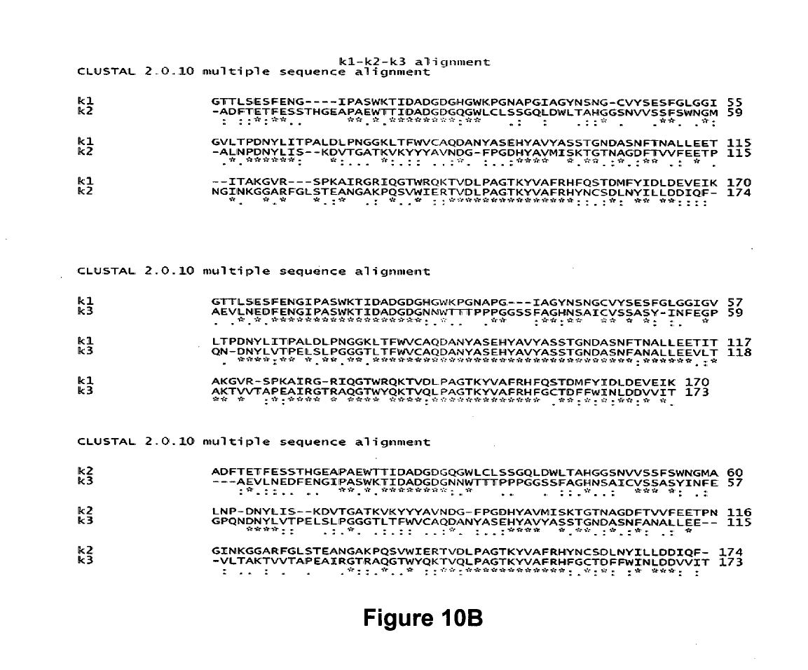

The present disclosure defines the domain sub-structure (FIG. 10) of HA containing protease-like molecule domain as comprising of three carbohydrate-binding domains linked by flexible hinge regions. Cleaved-Adhesin domains are proposed to be clan members linked to a super-family of domains (Galactose-binding domain-like superfamily), some of whom bind to carbohydrates.

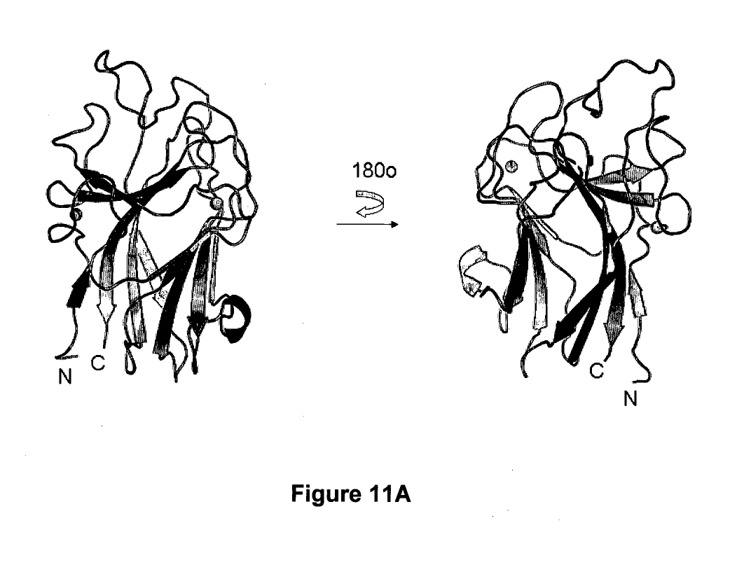

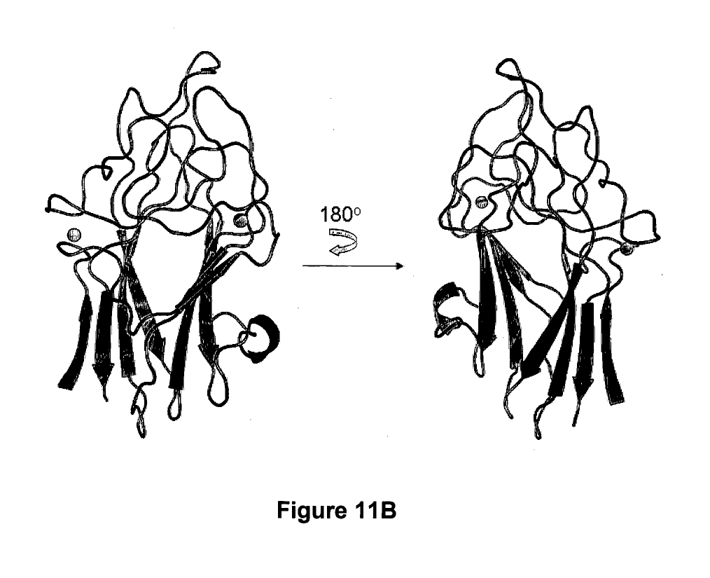

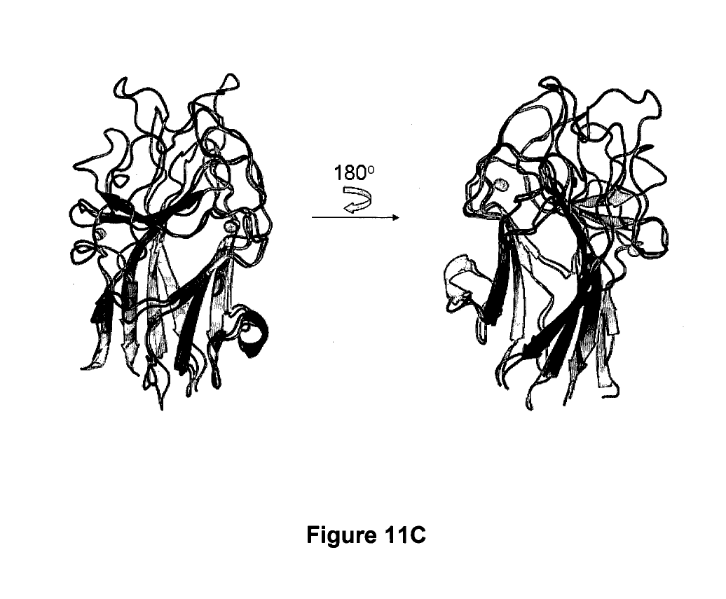

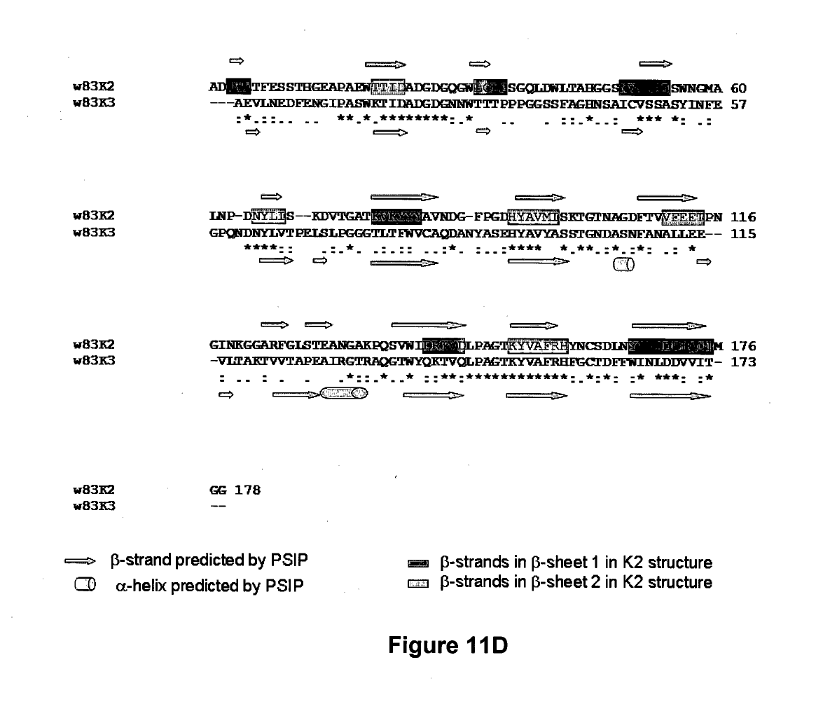

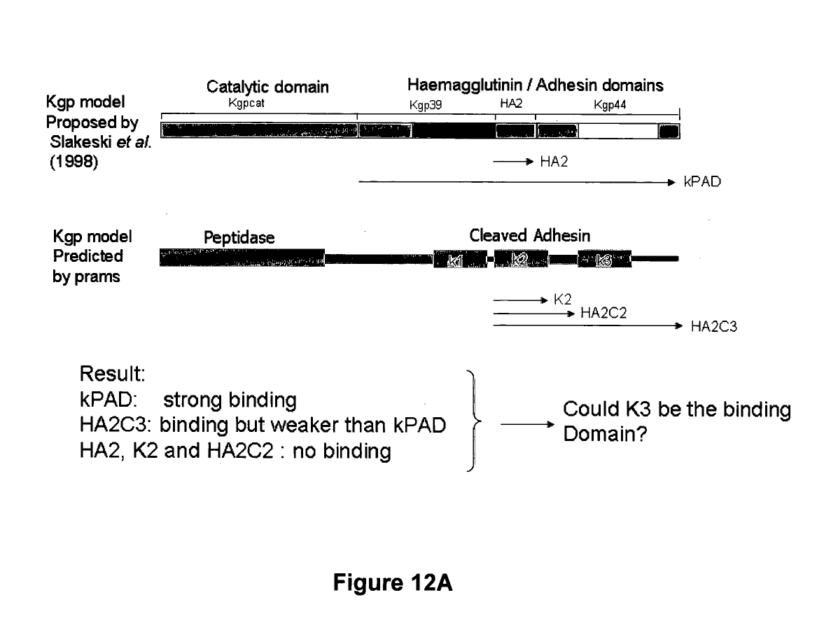

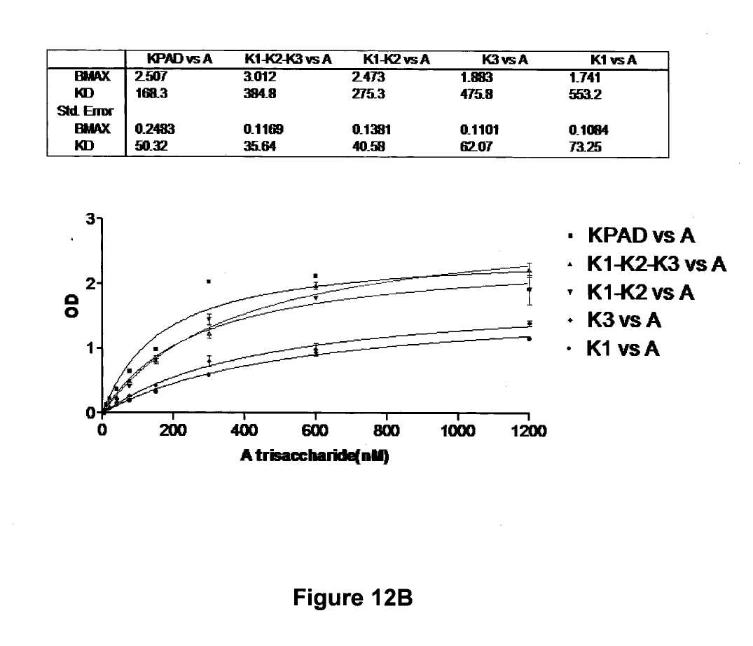

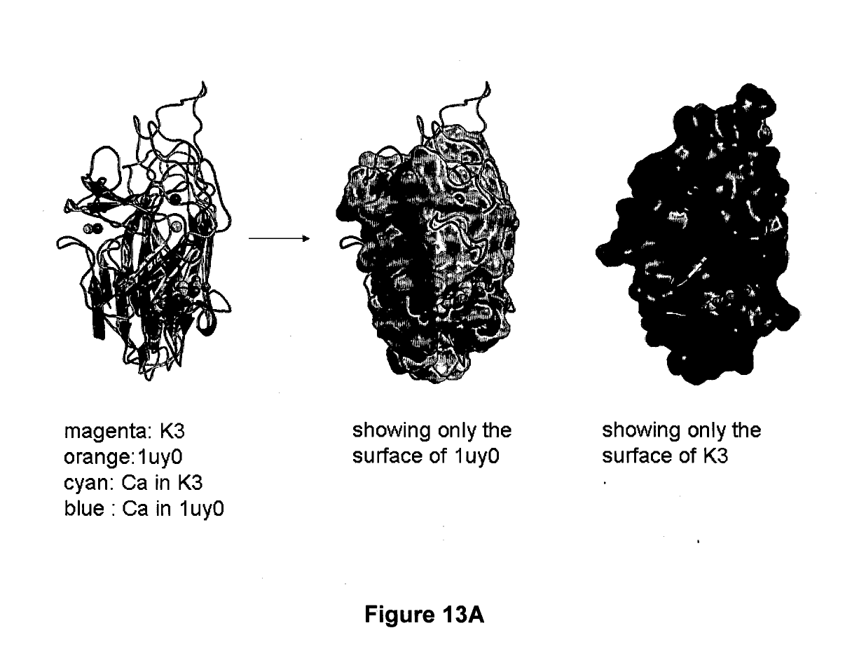

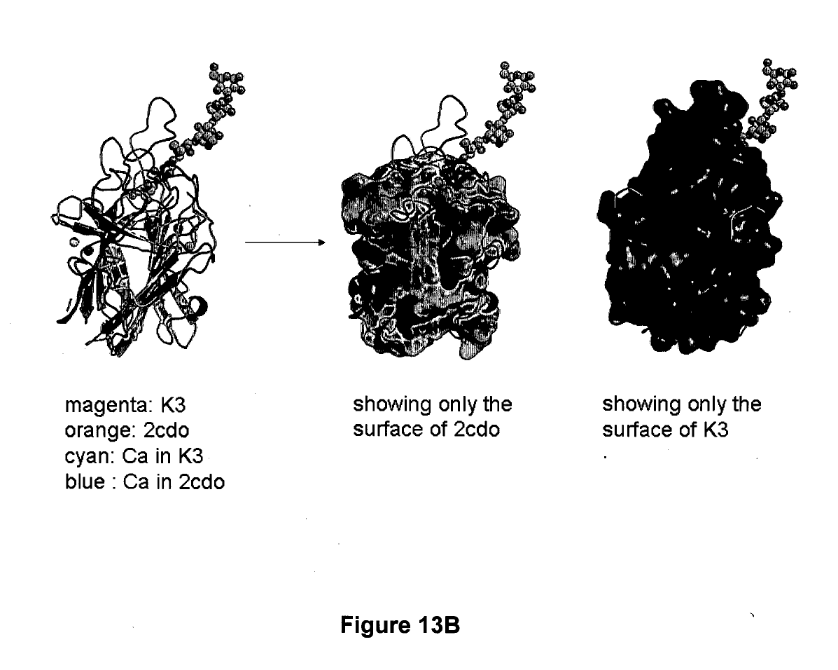

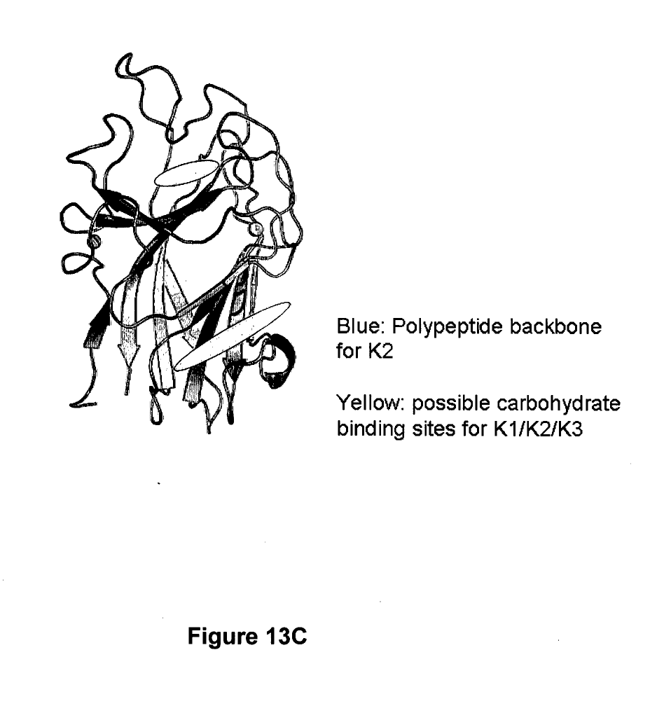

The link to function is based research involving X-ray crystallography (FIG. 11) and by carbohydrate binding assays to recombinant proteins (FIG. 12). The high resolution structures enable detailed mapping of potential 3D epitopes that have potential utility for the generation of universal vaccine immunogens and therapeutic antibodies. Carbohydrate-binding sites can now be defined which facilitate the pathogen adhesin to the host are defined (FIG. 13). Recombinant HA constructs are biologically active and these actions can be inhibited by selected oligosaccharides. On this basis two modes of targeting of these structures are proposed. In particular, this knowledge is to: (1) engineer recombinant proteins as vaccines or for use in identifying clinically active antibodies or antibody fragments or for use in a production process of antibodies for antibacterial therapies; (2) model conformational epitopes by constructing multiple loop peptides that mimic the surface configuration of the protein (i.e. a 3D epitope). This vaccine design is guided and facilitated by the exact knowledge of the critical functional sites; and (3) design oligosaccharides to inhibit the attachment of the organism to oral mucosa, thereby preventing colonization.

### Example 9

### Binding Sites for Carbohydrates in K3

The Docking program, Haddock, is used to predict the site of interaction of K3 with the trisaccharide of the blood group A antigen. It is proposed that the site of interaction is amino acid residues 34, 35, 37, 38, 39, 50, 134 and 135 which correspond to ProPro GlyGlySer Asn GlyThr, respectively. This target is used for designing sugar mimetics in a number of domains including K1 and K3 and R1 and A1, A2, A4, A6, A8 and A10. FIG. 14 A through F provide photographs of how it is envisaged that these host cell sugars bind to the protein domains (modules).

### Example 10

### Atomic Coordinates for K2 and K3 and Model for K1

FIG. 24A provides the atomic coordinates for K2 crystal refined at 1.4 Angstrom and FIG. 24B provides the K3 crystal refined at 1.6 Angstrom. The K3 structure is complexed with A-antigen trisaccharide docked into the putative carbohydrate binding site. The K1 homology model is provided based on K3's crystal structure refined at 1.6 Angstrom. See also Protein Data Bank identifiers 3KM5 and 3M1H, respectively.

### Example 11

### Generation of Antibodies

Monoclonal antibodies (Mabs) 5A1 and 2B2 were raised against a gingipain preparation. 5A1 was partially mapped to a PDNYL sequence partially buried on K2. This antibody also reacts with this sequence in HA1 of RgpA and Kgp39. 2B2 recognizes a determination, on HA1/HA3 of Kgp39. A rabbit antibody is also raised to a peptide sequence that is entirely on the surface of K2.

### Example 12

### Modeling the Structure of Homologous Cleaved_Adhesin Domains

Multiple sequence alignment modeling was used to identify homologous Cleaved_Adhesin domains in other proteins. In particular, the K1 sequence was aligned with the sequence of the K3 domain (FIG. 14G) and the K3 crystal structure was used as a template for homology modeling. The sequence and the template was used as an input for the comparative protein modeling software MODELLER (Sali and Blundell, 1993 supra), using the graphical user interface of Discovery Studio (DS) [v1.7, Accelrys, San Diego, Calif., USA]. The structural model is described by the atomic coordinates (FIGS. 24A and B) and Protein Data Bank.

### Example 13

### Crystal Structure of K3

The crystal structure of the W83 Kgp K2 module (PDB code: 3KM5) shares a highly conserved sequence with modules found in each of the Kgp, RgpA and HagA proteins expressed by strains of P. gingivalis (Li et al., 2010 supra). This recombinant protein module was shown to be haemolytic in vitro. In this disclosure provides the crystal structure of the K3 module of W83 Kgp at a resolution of 1.56 Å. K3 folds into a similar β-barrel module as K2 and it is also stabilised by two Ca²⁺ ions. This indicates that these HA region modules share some functional roles. Given the sequence identity of 71% between K3 and K1, the structure of the K1 module in Kgp can now be predicted with confidence. this disclosure shows that the HA region of Kgp W83 is composed of three tandem repeats of homologous protein modules. A recombinant construct containing these three modules was shown by small-angle X-ray scattering (SAXS) to be multi-globular and with each module being only loosely associated in solution. The variable loop regions of each of the modules are solvent accessible in the SAXS-derived molecular models. Of note, each of the HA modules presents loops which form significantly different molecular surfaces implying different possible adhesin functions, while some areas of the surface are structurally conserved and may act synergistically in common functional roles.

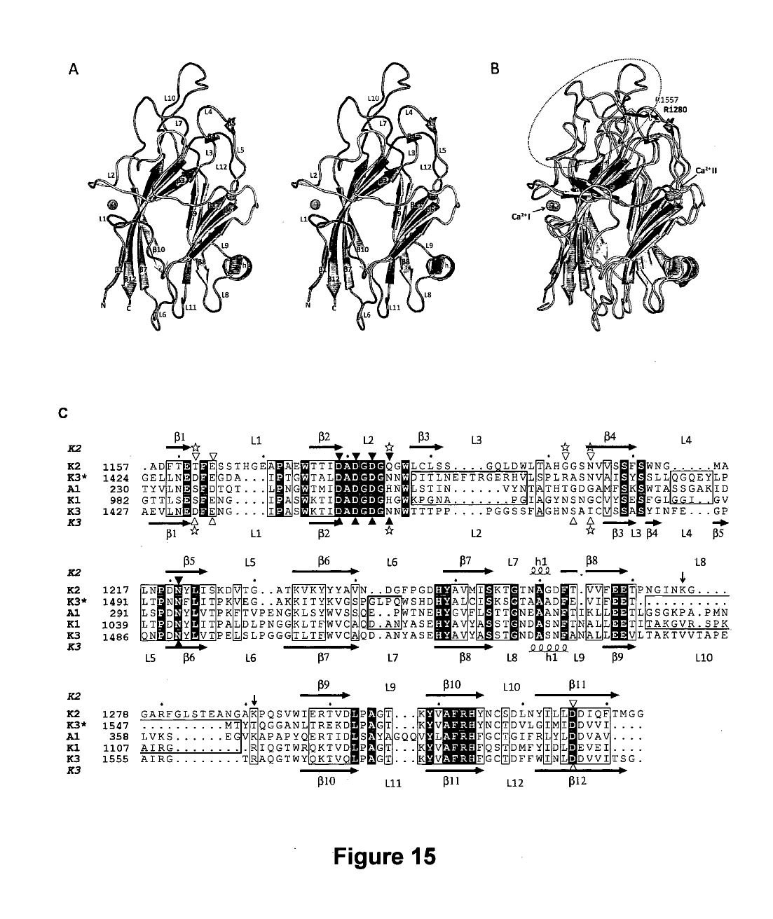

The K3 module, as crystallized, is composed of 178 residues with a molecular weight of 19 kDa (residues Ala1427-Gly1602 of Kgp W83 with glycine and serine attached to the N-terminus). While K3 has the same principal structural feature of the β-jelly roll-barrel observed in K2 (FIG. 15) minor differences complicate direct comparison. The β-barrel core of K3 is formed by two anti-parallel β-sheets each consisting of five β-strands β1-β3-β12-β7-β10 and β2-β6-β11-β8-β9, respectively (FIG. 15A). All of the β-strands are linked by long loops gathered at one end of the β-barrel and short loops on the other end. A small helix formed by residues 1531Asp-1535Phe is observed between loop8 (L8) and L9 on the short-loop end. A U-shaped feature which connects 133 and 136 has been divided into three loops designated L3, L4 and L5 by a four residue anti-parallel β-sheet formed by β-strands β4 and β5. L2 (Ala1447-Cys1473), the longest loop, consisting of 27 residues, extends around the top of the β-barrel and makes three tight turns when connecting β2 to β3 (FIG. 15A). As in the K2 structure, two Ca²⁺ ions flank the long loop end of the β-barrel in the K3 module linking the barrel core to loop residues (FIG. 15).

In this crystal structure, nine Na⁺ ions in total have been modeled on the surfaces of the four independently determined K3 molecules (designated chains A, B, C and D) and it is likely that these ion bound states are at least partly dependent upon the particular crystal packing arrangement. With a resolution of 1.56 Å, the calculated electron densities derived from the refined K3 structure are generally consistent with a single molecular model except for the N-terminal half of L10, Leu1544-Pro1553. Of the four K3 molecules in the asymmetry unit, only chain B and D have continuous observed main chain electron densities in this region with weak side chain densities for residues Leu1544-Lys1547. The electron densities are missing for residues Thr1551-Ala1552 in chain A and Ala1546-Pro1553 in chain C. The weak, variable and missing electron densities indicate that the N-terminal half of L10 is a flexible region and that the observed conformations of the residues in this loop are at least partly derived from the specific crystal packing arrangements.

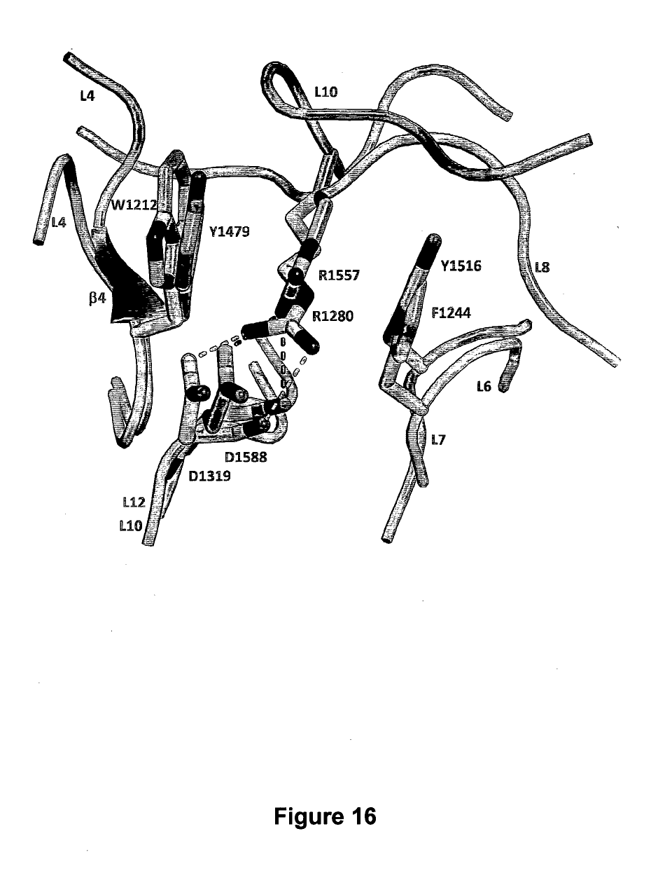

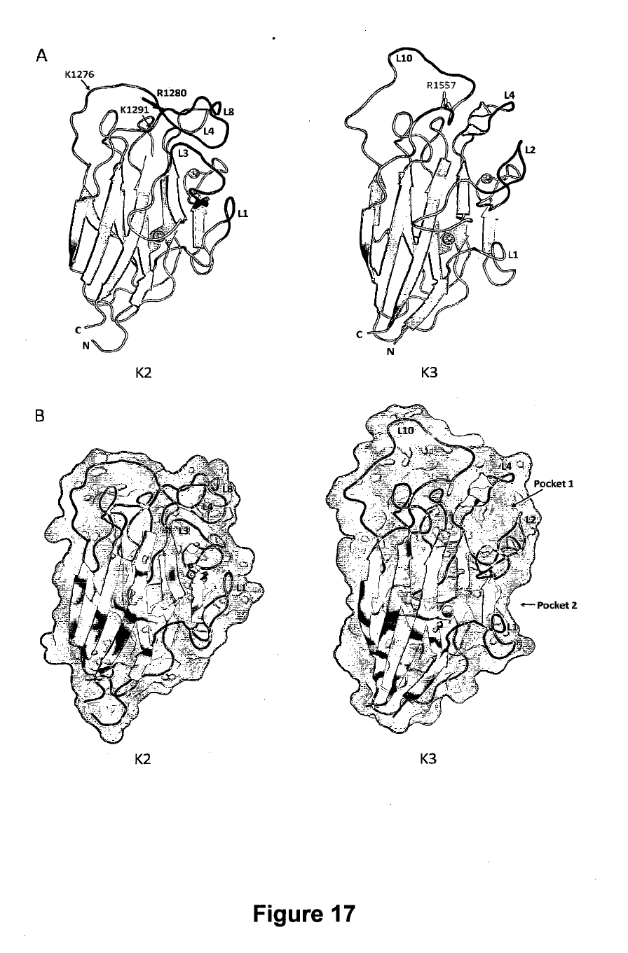

A stabilizing feature of the more ordered C-terminal fragment of L10 is noteworthy. Sequence alignments do not match the structural alignment derived from a comparison of the crystal structures of K3 and K2 at this location. When the three dimensional structures are superimposed however, Arg1557 of L10 in K3 is located in an almost identical position, conformation and interacting environment, as observed for Arg1280 of L8 in K2 (FIG. 16). This is despite the quite different observed conformations of these loops in the two structures (FIG. 17A). This conserved arginine anchoring site is located near the surface of the extensive loop region and in both these adhesin modules is formed by flanking planar aromatic residues, a salt bridge and H-bonding interactions with a conserved aspartic acid residue (FIG. 16). Kgp-specific cleavage of K2 produces a non-covalent “native-like” folded complex of polypeptide fragments cleaved at lysines adjacent to and either side of Arg1280 (Li et al., 2010 supra). The cleaved form of K2 is not observed to be haemolytic indicating that the C-terminal half of L8 and the anchored loops (from L10 in K3 and L8 in K2) are specific determinants of haemolytic activity. Most likely however, these partially buried and anchored arginine residues are not directly involved in ligand binding, but their anchored state may be critical to the conformational stability of the entire loop region. Specific proteolysis of K2 would release the anchor and thereby alter the presentation of the other nearby loops if and when they interact with binding partners.

### Example 14

### Structural Differences Between K3 and K2

The K3 and K2 domains of Kgp from P. gingivalis W83 have 34% sequence identity (Li et al., 2010 SUPRA). Superimposition of K3 and K2 structures using the program DALI (Hasegawa and Holm, Curr Opin Struct Biol 19:341-348, 2009) shows that 158 Cα atoms can be aligned with an rmsd of 1.7 Å and Z score of 22.6. The aligned residues mainly locate to the β-strands and to the short loops on one end of the barrels (FIG. 15B). This alignment, based on observed structure similarities (FIG. 15C), reveals that most of the residues forming the β-strands are highly conserved, particularly β2, β3, β6, β8, β11 and β12 of K3 and β2, β4, β5, β7, β10 and β11 of K2, respectively. These residues are also conserved in other Cleaved_Adhesin domains K1, K3* (K3 from the 381 biovar) and A1 (FIG. 15C) [Li et al.; 2010 supra], indicating that all of the modules found in HA regions (in gingipains and Hag proteins) are likely to fold with this same β-barrel motif.

Minor differences are observed between the two structures; the K3-barrel comprises ten β-strands in two β-sheets while K2 has eleven β-strands in the β-barrel. There is no equivalent in K3 to the four residue strand β3 in K2 as there is small difference in local backbone conformation. K3 presents an extended loop L2 (residues Ala1447-Cys1473) and in total corresponding in K2 to the residues of L2 (Ala1179-Trp1187), β3 (Leu1188-Ser1191) and L3 (Ser1192-Ser1204). Two additional β-strands in K3 (β4 and β5), linked by only two H-bonds, form an independent and very small anti-parallel β-sheet adjacent to loop L10 at one end of the β-barrel. This is part of the U-shaped feature (L3-β4-L4-β5-L5) in K3 which in K2 is reduced to one shorter loop (L4) [FIG. 6A].

Major differences between the two structures exist only in the extensive loop regions at one end of the β-barrel. The long loops L2 and L10 in K3, and L1, L3 and L8 in K2, present very different conformations to possible interacting partners and this is also reflected in the variation in the residues which form these loop regions (FIGS. 15C and 17A). L3 and L8 in K2, form a flat surface with no obvious depression but L2, L3-β4-L4-β5-L5 and L10 in K3 form a hollow (Pocket-1) on the surface of the molecule (FIG. 17B). The residues which line the sides and base of this pocket in K3 are Pro1458-Phe1465 in L2, Asn1481-Phe1482 in L4, Gln1512 in L7, Ile1556-Arg1560 in L10 and Phe1590-Trp1591 in L12-β12. This pocket is partially positively charged and partially negative charged and might bind ligands such as polysaccharides or polypeptides. Additionally, another small cleft (Pocket-2) is formed by L1 and L2 in K3 and this is not observed in K2, most probably due to K2 having an additional four residues in L1 resulting in a more upright conformation of the loop (FIGS. 15 and 17B).

The Ca²⁺ ions in K3 and K2 superimpose to almost equivalent structural positions (FIG. 16A). Table 3 lists the ligating residues and the observed bond distances. Only two of the five residues at Ca²⁺ binding site-I and four of the five residues at Ca²⁺ binding site-II in K3 and K2 are conserved. A number of the common Ca²⁺ binding residues (Asp1446, ASp1448, Asp1450, Asn1490 and Asp1595) are also conserved in the sequences of other parts of HA regions such as K1, K3* and A1 (FIG. 15C) indicating that these other modules are also likely to be Ca²⁺ containing domains. The Ca²⁺ binding site-II in K3 and K2 is almost identical with the mainchain carbonyl of Asn1452 being simply substituted by that of Gly1185 in K2. The other Ca²⁺ binding sites (site-I in K3 and K2) are also similar but subtly different in one aspect. The conformations of the exposed loops which surround this binding site (L2 in K3 and loop3 in K2) are partially different adjacent to the relevant ligating residues (involving the sidechain Oy of Ser1470 in K3 and the mainchain carbonyl of Gly1202 in K2) and do not correspond in the structure, alignment. Also, the ligating mainchain carbonyls of Asp1433 and Ile1472 of K3 are simply replaced by those of Thr1162 and Asn1205 in K2. Since both K2 and K3 are observed to be structurally dependent upon Ca²⁺, the importance of Ca²⁺ in stabilizing these protein folds and their loop conformations is most likely relevant to understanding the structure and function of all of modules in this domain family.

### Example 15

### Thermal Stability Dependency of K-Domains on Ca²⁺

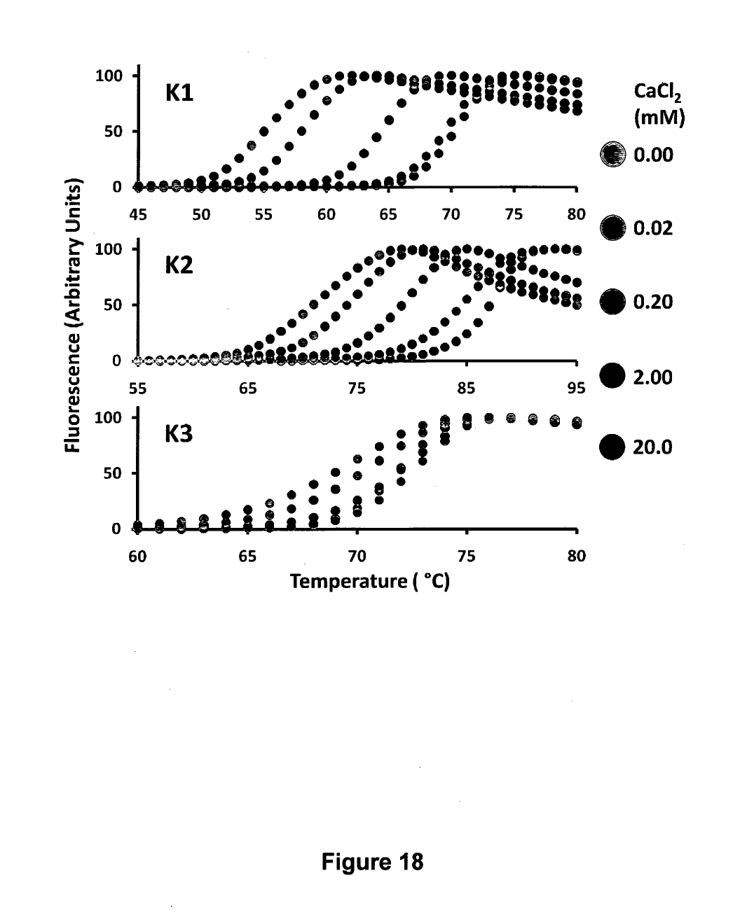

The role of Ca²⁺ in stabilising the observed folded forms of the K1, K2 and K3 adhesin modules in Kgp was investigated. The ThermoFluor technique was used to follow K1, K2 and K3 melting in response to temperature and the presence of Ca²⁺. Temperature-induced unfolding is accompanied by an increase in fluorescence in response to a fluorophore gaining access to the core hydrophobic residues. Only the K1 preparations used here contain additional non-bound Ca²⁺ at <0.5 mM post-dialysis. The melting point temperatures were observed as K3>K2>K1. K1 and K2 samples were both significantly stabilised by the addition of Ca²⁺; however, K1 shows the greatest enhancement of stability in the presence of Ca²⁺ (FIG. 18). This response confirms that the K1 module does associate with Ca²⁺ to stabilize a folded form.

### Example 16

### Haemolytic Activities

Haemolytic activity was determined as described below.

Trizma base, tris-hydrochloride (Tris-HCl) and Tween 20 were purchased from Sigma (St. Louis, Mo.). Phosphate buffered saline (PBS) was purchased from Oxoid (Basingstoke, United Kingdom). Blood was drawn from human donors into 0.1 M citrate anticoagulant. Erythrocytes were separated from platelet-rich plasma and the buffy coat by differential centrifugation at 150×g for 15 min. The erythrocytes were pelleted by centrifugation at 350×g and washed twice in PBS pH 7.4 and resuspended to 1% volume/volume in PBS. Various concentrations of K2 up to 10,000 nM were added to the erythrocytes in a total volume of 200 μL and incubated at 25° C. or 37° C. After periods of incubation, the microtiter plate was centrifuged at 1000×g for 10 min and the supernatants (100 μL) transferred into a new microtiter plate. Haemoglobin release was determined spectrophotometrically using a microtitre plate reader (absorbance at 405 nm, the peak absorbance in the Soret region).

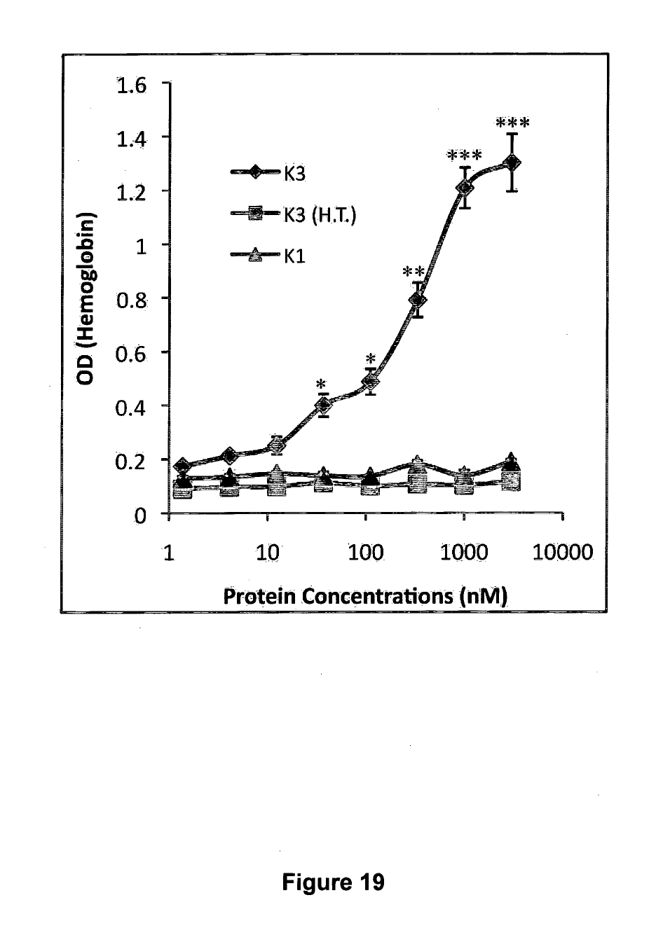

Freshly isolated human Blood Group A and O erythrocytes were studied. Haemolysis of red cells induced by K3 was concentration- and time-dependent. K3 was effective in a range of 10 to 5000 nM with 50% haemolysis observed at ˜250 nM (FIG. 19). The absence of the catalytic domain in the K3 polypeptide indicated that the haemolysis was independent of proteolytic activity. In this regard there are no data in the literature to indicate that the HA region of Kgp including K2, has proteolytic activity and K3 does not contain any recognized catalytic moiety. Haemolysis induced by K3 developed over several hours, no haemolysis was observed after heat treatment of the recombinant protein. Buffer controls did not produce detectable haemolysis. No detectable haemolysis was detected following incubation of K1 with erythrocytes for up to 48 hrs (FIG. 19).

### Example 17

### Ligand Binding Experiments

An investigation of K3 ligand binding involved dot blot arrays to probe the binding of 6×His tagged K3 to target proteins and glycans immobilized on nitrocellulose. Data indicated that the tagged K3 bound strongly to human serum albumin (HSA) and fibrinogen. Relatively strong binding was also observed for bovine maxillary mucin and hyaluronan while only weak binding was detected for the other glycans tested.

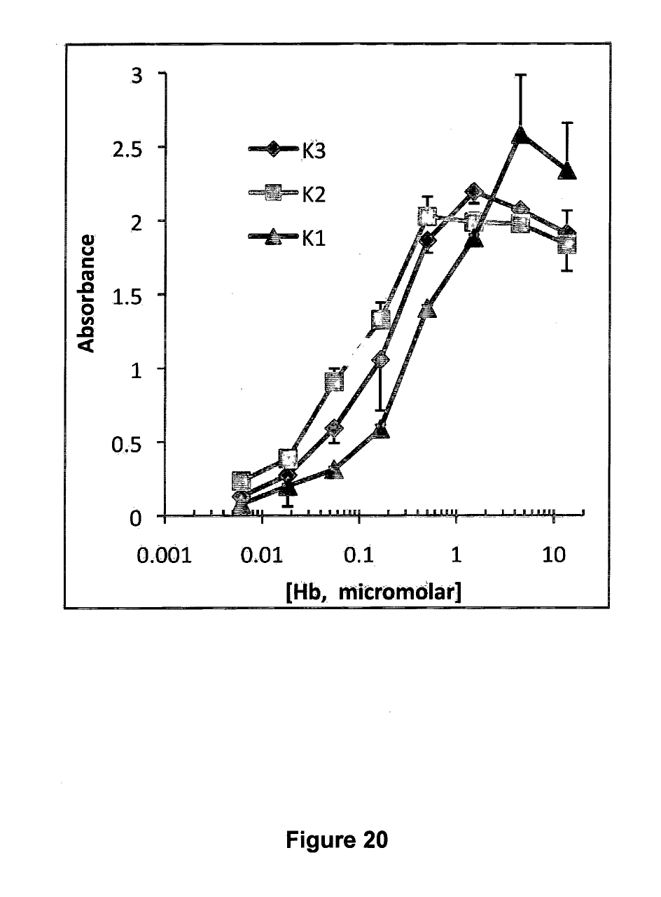

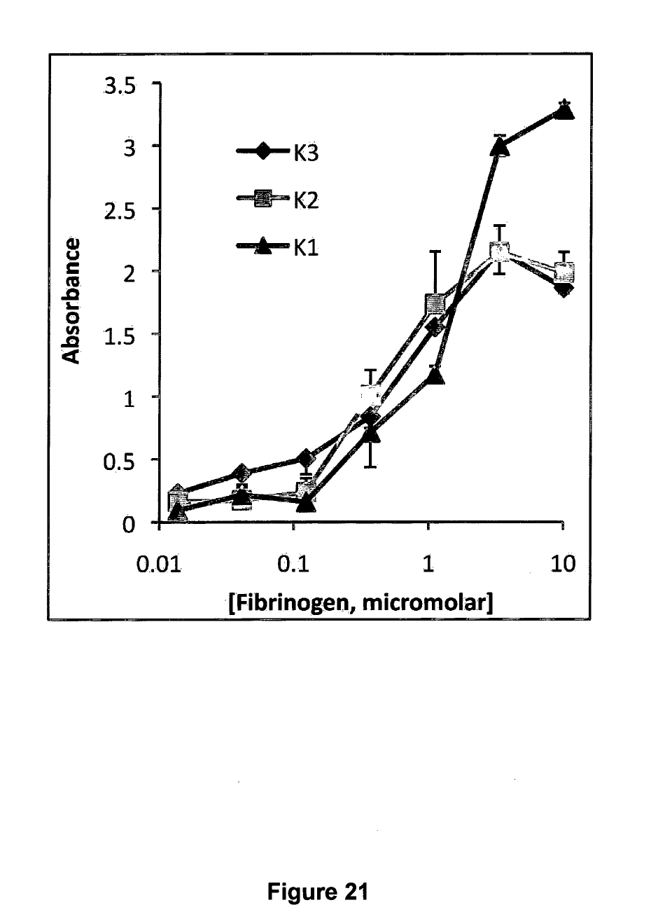

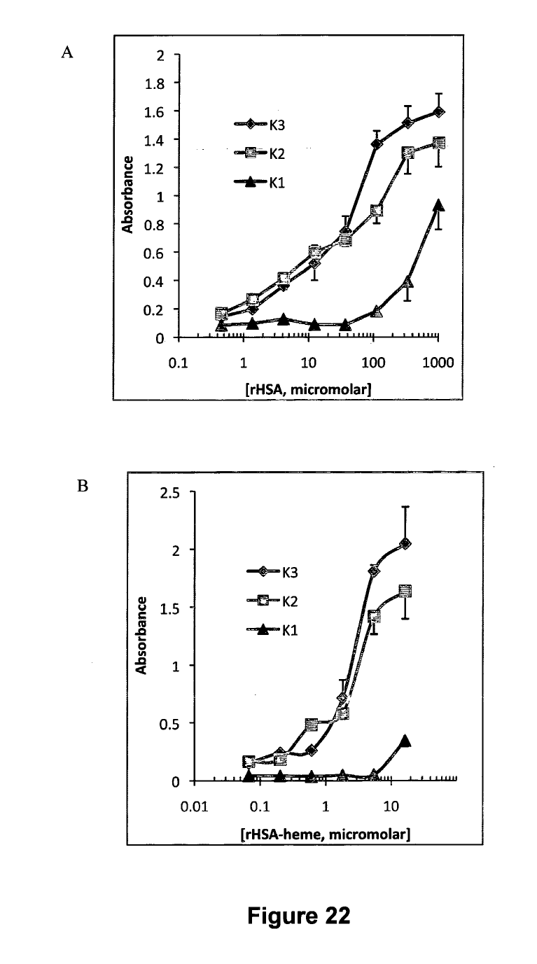

The apparent K_{d} values determined by ELISA for titrations of haemoglobin binding to immobilized adhesin domains were 154 nM for K3 polypeptide, 80 nM for K2 polypeptide and 360 nM for K1 polypeptide (FIG. 20). For comparison, the binding of fibrinogen to the three recombinant adhesin domains was also assessed. The apparent K_{d} values determined for fibrinogen were 570 nM for K3, 450 nM for K2 and 1.5 μM for K1 (FIG. 21). Hemo-proteins other than haemoglobin have been reported to support the porphyrin requirement of P. gingivalis (Bramanti and Holt, J Bacteriol 173:7330-7339, 1991; Sroka et al., J Bacteriol 183:5609-5616, 2001). The requirement for growth in culture can also be met by supplementation with heme in complex with HAS (Liu et al., Biol Chem 385:1049-1057, 2004). To assess the capacity of gingipain adhesin domains to bind other hemo-proteins, heme-loaded recombinant human serum albumin (rHSA) was compared with apo-rHSA for binding capacity. Binding affinity of rHSAheme was significantly higher (>4-fold and 9-fold increase, respectively) in comparison to rHSA affinity without heme present for both K2 and K3 but in contrast binding of albumin or heme-albumin to K1 was much weaker (FIG. 22). Further, no significant diminution to the binding affinity between K2 or K3 and rHSA was observed in the presence of anti-K2 polyclonal antibody targeting loop 1 of K2.

### Example 18

### SAXS Solution Studies

Small-angle X-ray scattering (SAXS) data were collected from solutions of K1K2 and K1K2K3. These data are sensitive to the size and shape of particles in solution and were ultimately used to probe the domain organization of both proteins. Guinier analysis (Guinier, Comptes Rendus Hebdomadaires Des Seances De L Academie Des Science 206:1374-1376, 1938) of the K1K2 and K1K2K3 data at very low-q shows excellent linear correlations (R^{2˜}0.995), consistent with samples that are free of aggregation or significant interparticle interference and, when combined with molecular weight determinations derived from the forward scattering intensity at zero angle (I(0)), indicate that K1K2 and K1K2K3 exist as systems of monodisperse monomers in solution. Under these conditions, the real-space probable distribution (P(r)) of atom pair-distances (r) within the proteins were calculated via indirect Fourier transformation of the data (Bergmann et al., 2000 supra), from which the maximum dimension (Dₘₐₓ) and radius of gyration (R_{g}) were determined. Overall, K1K2K3 has a longer maximum dimension (Dₘₐₓ, ˜150 Å) and has a larger R_{g} (˜45 Å) compared to K1K2 (Dₘₐₓ, ˜95 Å; R_{g}, ˜30 Å) suggesting that the mass representing K3 extends from K1K2.

The shape of the atom-pair distance distributions of K1K2 and K1K2K3 display characteristics of modular proteins that have discrete, well defined domains (as opposed to compact globular particles or extended rod-shapes) indicated by the “humps” in the distributions at mid-range vector lengths (˜50-80 Å) that arise due to scattering from ‘between-domain’ atom-pair distances. Ab initio shape restoration from the data (Franke and Svergun, 2009 supra) reveals that K1K2 is a ‘double-lobed’ protein, with each lobe having an approximate volume as a single K-domain (˜22-24 000 Å³). The two lobes of K1K2 are spatially positioned in tandem next to each other and this K1K2 configuration is preserved in K1K2K3 that adopts an overall “y” shaped conformation in solution. Further refinement against the SAXS data of the domain orientations within the K1K2 and K1K2K3 molecular envelopes was performed using BUNCH (Petoukhov and Svergun, 2005 supra) that employed the crystal structures of K2, K3 and a homology model of K1 as independent rigid bodies, while also incorporating dummy-atoms to represent the mass of the linker regions of unknown structure between K1-K2 and K2-K3. The rigid body refinements generated K1K2 and K1K2K3 models that fit to the data very well (K1K2, χ²=0.51-0.66; K1K2K3 χ²=0.74-0.86) and the overall “y” shape of the K1K2K3 atomic representations correspond to the shapes generated in the ab initio models.

Due to the near mass equivalency of K1 and K2, it is difficult to determine from the SAXS data the exact orientation of K1 or K2 with respect to K3 in the y-shape other than that the K1 and K2 modules comprise the two arms at the top of they and the K3 domain is positioned at the end of an extended ‘tail’ which is composed of a 93 amino acid linker between K2 and K3. Because of steric constraints imposed by the location of the N- and C-termini of each of the modules (that enter and exit on the same end of their respective -barrels—see FIG. 17A), the variable loop regions of the K1, K2 and K3 domains all face outward from the core of the “y”-shape and do not directly interact with any partner domains. These variable loop regions at each ‘tip’ of the K1K2K3 structure are solvent accessible and are thus poised for protein-protein or carbohydrate adhesin interactions.

The structural core of the adhesin modules K2 and K3 are homologous but there are significant differences in the associated -barrel end loop regions and minor differences on the flanks of the ends of the barrel. Other adhesin modules in P. gingivalis HA regions (such as K1, R1, R2 and A2-10) as identified by sequence alignment and significant identities (>70% to either K2 or K3), are by definition, homologues. The two crystal structures, when combined with multiple sequence alignments of the other modules enables structural features of the whole domain family to be predicted. These data suggest that in the extensive loop regions at the “active” end of the β-barrels there will be found a spectrum of similarities and differences in this adhesin family. L8 in K2 has previously been linked to function by a specific proteolytic cleavage of two lysines in L8 by Kgp which arrests the haemolytic and binding activities of this module. Surprisingly, despite no obvious sequence correspondence with L8 of K2, the structure of K3 reveals that the “equivalent” loop L10 does in fact partially mimic K2. In particular, the arginine anchoring site appears to be conserved and it is proposed that in both K2 and K3 the overall conformations of L8 and L10, respectively are at least partly determined by this anchoring. Sequence, alignments imply that the same anchoring site may also be found in the stabilization of the overall loop conformations present in the K1 module. This suggests that rather than a direct role in haemolysis these particular loops (L8 in K2 and L10 in K3) associate to fix conformations of other associated loops in modules K2 and K3 but that these associated loops differ in K1, which is not haemolytic.

Comparing the protein sequences of other less similar putative domains with K2 and K3 (such A 1 and K3* with <35% sequence identity), it is predicted, for example K3*, found in Kgp of strain 381, will possess a slightly different surface feature with a, longer L2 but a substantially shorter L10 when compared to K3. As the sequences of 381 Kgp and W83 Kgp only vary significantly in the K3*/K3 domain regions, this is a structural feature that might explain strain specific functional differences.

In the K2 structure, Gly1273-Lys1276 and Ser1284-Gly1289 of L8 have very weak electron densities (Li et al., 2010 supra) and equally, the electron densities in the corresponding region in the K3 structure, Ala1546-Pro1553 in L10 in chain C are not observed. Weak or missing densities in high resolution crystal structures often indicates that multiple/flexible or disordered states exist in solution, and in some cases such flexibility may be related to a functionally active binding site. Interestingly, the two flexible regions of K2 and K3 are located at the same structural position and both have charged residues in their sequences, indicating they might be the sites for a similar binding function.

Bound calcium contributes to the stability of the folded states of these adhesin modules and is a general feature of the galatose-binding domain-like (GBD) superfamily. Comparisons of the K2 and K3 structures with their closest known structural homologues in the GBD superfamily, such as the MAM domain of human receptor-type tyrosine-protein phosphatases, ephrin receptors, a number of carbohydrate binding modules (CBMs) [Li et al., 2010 supra], and modules found in the sub-repeats of reelin (Yasui et al., Structure 18:320-331, 2010) reveal that Ca²⁺ binding site-I is widely conserved. Most interestingly, the Asp acid residue in Ca²⁺ binding site-I that corresponds to Asp1595 in K3, located at the adjacent to the C-terminus of β-barrel core, is found in an equivalent position in all of these homologues. A Glu residue from site I that corresponds to Glu1435 in K3, is also conserved in most of the CBMs. Ca²⁺ binding is a common feature for many domains with a β-sandwich folding topology and while many of the GBD structural superfamily share only the site-I Ca²⁺, other secondary cation binding sites have been reported. For example, CBM36 Xylanase (PDB entry lux7) has a second bound Ca²⁺ which mediates the binding of xylotriose ligand (Jamal-Talabani et al., 2004 supra). However, structural superimposition of CBM36 and K2 reveals that this different Ca²⁺ binding site in CBM36 does not superimpose onto Ca²⁺ binding site-II (Li et al., 2010 supra).

The in vitro binding properties of recombinant K1, K2 and K3 modules with putative ligands revealed similarities and variations reflecting observed structural differences particularly those found in the extensive loop regions. While apparent binding affinities to haemoglobin and fibrinogen were comparable in each of the three modules and being consistent with equivalences in folded structure and sequence, K1 binding to human serum albumin was not significant. The enhanced binding of heme-albumin compared with albumin is sufficient to facilitate selective uptake of heme-albumin from inflammatory exudate. That is, the organism has a demonstrated capacity to bind albumin that has scavenged heme, particularly heme released within the proximity of the lesion of periodontitis. This provides a further mechanism for uptake of essential heme by the gingipains.

The putative binding pockets observed in K3 (and predicted in K1) are not observed in the K2 crystal structure. These binding data indicate that there are likely to be specific structural differences between the K1 and K3 modules. Such a difference is likely to be found in the variable loops of these modules.

Both K2 and K3 have been found to possess an ability to induce haemolysis in a dose-dependent manner but with an unknown mechanism. Previous work on K2 suggested a link to anion transport in erythrocytes and the role of K2 loop L8 in this process (Li et al., 2010 supra). In the structure of K3, the conformation of the equivalent loop, L10, is significantly different but is anchored by an observed equivalent arginine binding site formed near the surface of conserved but non-aligned residues in the extensive loop region. This conserved structural motif (which is not predicted by the sequence alignment of these two loops) is unlikely to be the only structural determinant for any haemolytic process. This is because sequence alignment predicts that this structural motif may also exist in the non-haemolytic K1. The information presented here indicates that observed binding properties and haemolytic activities of these modules is dependent on specific features of the loop regions and that anchoring of the largest loop (L8 in K2 and L10 in K3/K1) which extensively covers one end of the barrel structure determines the conformational integrity and/or the surface structure of adjacent ligand binding sites.

SAXS-derived models of the K1K2K3 protein support the proposal that the HA region of Kgp is composed principally of three globular protein modules with dimensions corresponding to those observed in the crystal structures of K2 and K3 and the associated homology model of K1. The HA region of RgpA contains two modules termed R1 and R2 with close homology to K1 and K2 of Kgp. Both the HA regions of Kgp and RgpAs also include a sequence related ˜150 residue fragment C-terminal to the protease domain. The significant sequence homologies found in these fragments suggest the presence of another type of protein module of unknown structure and function (designated by databases as: Pfam entry. DUF2436 and InterPro entry IPR018832) within the HA regions of gingipains. This indicates that the gingipains are composed of protease domains, tandem repeats of cleaved-adhesin modules, combined with a third type of domain/region (DUF2436/IPRO18832-like) of unknown structure and function.

The SAXS data clearly demonstrate, that the three adhesin modules of the HA region of Kgp do not interact to form globular dimers or trimers and that the extensive loop regions in each loosely associated module are accessible to bind ligands.

Those skilled in the art will appreciate that the disclosure herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of any two or more of said steps or features.

### BIBLIOGRAPHY

Altschul et al., Nucl. Acids Res. 25:3389, 1997
Aricescu et al., Science 317:1217-1220, 2007
Auffray et al., Blood 97:2872-2878, 2001
Ausubel et al., A Current Protocols in Molecular Biology≈John Wiley and Sons Inc, 1994-1998, Chapter 15
Bae et al., J Biol. Chem. 283:12415-12425, 2008
Bergmann et al., Journal of Applied Crystallography 33:1212-1216, 2000
Bramanti and Holt, J Bacteriol 173:7330-7339, 1991
Chu et al., Infect Immun 59:1932-1940, 1991
Curtis et al., J Periodontal Res 34:464-472, 1999
DeCarlo et al., J Bacteriol 181:3784-3791, 1999
Dominguez et al (2003) http://pubs.acs.org/servlet/reprints/DownloadReprint/ja026939x/L3tc)
Eichinger et al., Embo J 18:5453-5462, 1999
Emsley and Cowtan, Acta Crystallographica Section D-Bilogical Crystallography 60:2126-2132, 2004
Ericsson et al., Anal Biochem 357:2890-298, 2006
Fanali et al., FEBS J 274:4491-4502, 2007
Finn et al., Nucleic Acids Res 36:D281-288, 2008
Franke and Svergun, Journal of Applied Crystallography 42:342-346, 2009
Galtieria et al., Biochimica et Biphysica Acta (BBA), Biomembranes 1564:214-218, 2002
Guinier, Comptes Rendus Hebdomadaires Des Seances De L Academie Des Science 206:1374-1376, 1938
Haddock, J. Am. Chem. Soc. 125:1731-1737, 2003
Hahn, The International Tables for Crystallography, volume A, Fourth Edition, Kluwer Academic Publishers 1996
Hasegawa and Holm, Curr Opin Struct Biol 19:341-348, 2009
Himanen et al., Nature 414:933-938, 2001
Holm and Park, Bioinformatics 16:566-567, 2000
Holt et al., Science 239:55-57, 1988
Jamal-Talabani et al., Structure 12:1177-1187, 2004
Jeffries et al., Journal of Molecular Biology 377:1186-1199, 2008
Konarev et al., Journal of Applied Crystallography 36:1277-1282, 2003
Lamont and Jenkinson, Microbiol Mol Biol Rev 62:1244-1263, 1998
Larkin et al., Bioinformatics 23:2947-2948, 2007
Lewis and Macrina, Infect and Immun 66:4905-4913, 1998
Lewis et al., J Bacteriol 181:4905-4913, 1999
Li et al., Mol Microbiol 76:861-873, 2010
Liu et al., Biol Chem 385:1049-1057, 2004
Lovell et al., Proteins-Structure Function and Genetics 50:437-450, 2003
Malawski et al., Protein Sci 15:2718-2728, 2006
McCoy et al., J Appl Crystallogr 40:658-674, 2007
Murshudov et al., Acta Crystallographica Section D-Biological Crystallography 53:240-255, 1997
Nakayama et al, Mol Microbiol 27:51-61, 1998
Nguyen et al., Infect Immun 72:1374-1382, 2004
O'Brien et al., J Immunol 75:3980-3989, 2005
Orthaber et al., Journal of Applied Crystallography 33:218-225, 2000
Otwinowski and Minor, Maromolecular Crystallography, Pt A276:307-326, 1997
Pantoliano et al., J Biomol Screen 6:429-440, 2001
Paramaesvaran et al., J Bacteriol 185:2528-2537, 2003
Pavloff et al, J Biol Chem 270:1007-1010, 1995
Pavloff et al., J Biol Chem 272:15.95-1600, 1997
Perrakis, Nature Struct. Biol. 6:458-463, 1999
Petoukhov and Svergun, Biophysical Journal 89:1237-1250, 2005
Pike et al., J Biol Chem 269:406-411, 1994
Pike et al., J Bacteriol 178:2876-2882, 1996
Potempa et al., Infect Immun 63:1176-1182, 1995
Potempa et al., J Biol Chem 273:21648-21657, 1998
Potempa and Nguyen, Purification and characterization of gingipains In: Current protocols in Protein Science, Chapter 21:Unit 21.20, 2007
Roper et al., J Biol Chem 275:40316-40323 2000
Sakai et al., J Bacteriol 189:3977-3986, 2007
Sali and Blundell, Mol. Biol. 234:779-815, 1993
Sheldrick, Acta Cryst. A64:112-122, 2008
Shi et al., J Biol Chem 274:17955-17960, 1999
Shmueli, The International Tables for Crystallography, Volume B, First Edition, Kluwer Academic Publishers
Socransky et al., J Clin Periodontol 25:134-144, 1998
Sroka et al., J Bacteriol 183:5609-5616, 2001
Svergun et al., Journal of Applied Crystallography 28:768-773, 1995
Takii et al., Infect Immun 73:883-893, 2005
Veith et al., Biochem J 363:105-115, 2002
Volkov and Svergun, Journal of Applied Crystallography 36:860-864, 2003
Whitten et al., Journal of Applied Crystallography 41:222-226, 2008
Yamaguchi et al., J Biochem 118:760-764, 1995
Yasui et al., Structure 18:320-331, 2010

1.-20. (canceled)

21. A method for identifying a compound which interacts with a Cleaved_Adhesin domain homolog of the Cleaved_Adhesin domain on lysine gingipain (Kgp), arginine gingipain (Rgp) and hemagglutinin A (HagA), said method comprising: (a) providing a three dimensional representation of the atomic coordinates of the Cleaved_Adhesin domain and docking a three dimensional representation of a compound from a computer database with the three dimensional representation of the domain; (b) determining a conformation of the resulting complex having a favorable geometric fit and favorable complementary interactions; and (c) identifying compounds that best fit the domain and testing the compounds as potential modulators of the activity of a protein comprising the domain.

22. The method of claim 21 wherein the Cleaved_Adhesin domain is selected from the list consisting of K1, K1*, K2, K3 and K3* on Kgp.

23. The method of claim 21 wherein the Cleaved_Adhesin domain is selected from the list consisting of R1 and R2 on Rgp.

24. The method of claim 21 wherein the Cleaved_Adhesin domain is selected from the list consisting of A1 through A10 on HagA.

25. The method of claim 21 wherein the compound antagonizes the activity of a protein comprising the Cleaved_Adhesin domain.

26. The method of claim 25 wherein the compound is used in the treatment or prophylaxis of infection by Porphyromonas gingivalis.

27. A method for the prophylaxis or treatment of infection by a microorganism in a biological environment from where the microorganism acquires iron, heme or porphyrin, said method comprising administering to the environment an effective amount of an agent for a time and under conditions sufficient to antagonize a Cleaved_Adhesin domain homolog of the Cleaved_Adhesin domain on lysine gingipain (Kgp), arginine gingipain (Rgp) and hemagglutinin A (HagA) within the adhesin and/or carbohydrate binding region of a protease-like molecule produced by the microorganism, said domain associated with hemolysis or hemolytic activity of erythrocytes.

28. The method of claim 27 wherein the adhesin and/or carbohydrate binding region is a hemagglutinin (HA) region.

29. The method of claim 28 wherein the protease-like molecule is a gingipain or a hemagglutinin (Hag) protein.

30. The method of claim 24 wherein the protease-like molecule is selected from the list consisting of Kgp, Rgp and HagA.

31. The method of claim 27 wherein the microorganism is Porphyromonas gingivalis or a related microorganism which expresses a gene product containing a Cleaved_Adhesin domain as defined by homology to the Cleaved_Adhesin domain in Kgp, Rgp and/or HagA.

32. The method of claim 30 herein the Cleaved_Adhesin domain or Kgp is selected from the list consisting of K1, K1*, K2, K3 and K3* on Kgp.

33. The method of claim 30 wherein the Cleaved_Adhesin domain is selected from the list consisting of R1 and R2 on Rgp.

34. The method of claim 30 wherein the Cleaved_Adhesin domain is selected from the list consisting of A1 through A10 on HagA.

35. A method for the prophylaxis or treatment of infection by Porphyromonas gingivalis from where the P. gingivalis acquires iron, heme or porphyrin, said method comprising administering to the subject an effective amount of an agent for a time and under conditions sufficient to antagonise a Cleaved_Adhesin domain within the hemagglutinin-binding region of a gingipain or hemagglutinin (Hag) protein selected from lysine gingipain (Kgp), arginine gingipain (Rgp) and HagA produced by P. gingivalis wherein the Cleaved_Adhesin domain on Rgp is selected from R1 and R2, the Cleaved_Adhesin domain on Kgp is selected from K1, K1*, K2, K3 and K3* and the Cleaved_Adhesin domain on HagA is selected from A1 through A10 wherein the domain is associated with hemolysis or hemolytic activity of erythrocytes.

36. Use of a Cleaved_Adhesin domain selected from K1, K1*, K2, K3 and K3* on Kgp and R1 and R2 on Rgp and A1 through A10 on HagA in the manufacture of a medicament in the treatment or prophylaxis of infection by P. gingivalis or a related microorganism.

37. Use of a Cleaved_Adhesin domain selected from K1, K1*, K2, K3, K3* R1, R2 and one or more of A1 through A10 in the identification of a homologous Cleaved_Adhesin domain in a protein.

38. Use of a Cleaved_Adhesin domain homolog identified in claim 37 in the manufacture of an agent which interacts with the Cleaved_Adhesin domain.

39. Use of claim 38 in the manufacture of a therapeutic, prophylactic or diagnostic agent.

40. Use of the atomic coordinates provided in FIGS. 24A and B for K2 and K3, respectively, in the identification of an interacting model for use as an antagonist, agonist or diagnostic agent to identify or modulate a protein having a Cleaved_Adhesin domain.
